(19) United States
(12) Patent Application Publication
(10) Pub. No.: US 2025/0145716 A1
(43) Pub. Date: May 8, 2025

(54) ANTI- PROGRAMMED DEATH-LIGAND 1 (PD-L1) ANTIBODY MOLECULES, ENCODING POLYNUCLEOTIDES, AND METHODS OF USE

(71) Applicant: Rakuten Medical, Inc., San Diego, CA (US)

(72) Inventors: Sebastian STEINIGER, San Diego, CA (US); Nikolai SUSLOV, Needham, MA (US); Alexey TEPLYAKOV, Middletown, MD (US); Amy THORNE, San Diego, CA (US); Jason LAPETODA, San Diego, CA (US); Robert J. HOEY, Santee, CA (US)

(73) Assignee: Rakuten Medical, Inc., San Diego, CA (US)

(21) Appl. No.: 18/838,585

(22) PCT Filed: Feb. 17, 2023
(86) PCT No.: PCT/US2023/062822

§ 371 Date: Aug. 14, 2024

Related U.S. Application Data
(60) Provisional application No. 63/311,924, filed on Feb. 18, 2022.

(51) Int. Cl. C07K 16/28; A61K 41/00; A61K 47/54; A61K 47/68

(52) U.S. Cl. CPC C07K 16/2827; A61K 41/0042; A61K 47/545; A61K 47/6849; C07K 2317/732; C07K 2317/76; C07K 2317/77; C07K 2317/92

(57) ABSTRACT

Provided herein are anti-Programmed Death-Ligand (PD-L1) antibodies, antigen-binding fragments thereof, multispecific antibodies and encoding polynucleotides. Also provided are PD-L1-targeted phthalocyanine dye conjugates, compositions and articles of manufacture containing the conjugates, and methods for their administration to subjects for photoimmunotherapy. In some embodiments, the anti-PD-L1 antibodies, fragments, and conjugates specifically bind to human PD-L1. Among the anti-PD-L1 antibodies are human antibodies and humanized antibodies. Also provided are methods and uses employing the provided anti-PD-L1 antibodies, antigen-binding fragments, and conjugates such for treatment of human patients.

### RELATED APPLICATIONS

This application claims priority from U.S. Provisional Application No. U.S. 63/311,924, filed Feb. 18, 2022, entitled “ANTI-PROGRAMMED DEATH-LIGAND 1 (PD-L1) ANTIBODY MOLECULES, ENCODING POLYNUCLEOTIDES, AND METHODS OF USE,” the contents of which are incorporated by reference in their entirety.

### INCORPORATION BY REFERENCE OF SEQUENCE LISTING

The content of the electronic Sequence Listing (file name: 751702002740SeqList.xml, date created: Feb. 7, 2023, size: 418,493 bytes) is herein incorporated by reference in its entirety.

### FIELD

The present disclosure relates to Programmed Death-Ligand (PD-L1) binding molecules, in particular, to anti-PD-L1 antibodies, including antibody fragments and compositions, combinations, methods, and uses thereof. The present disclosure further relates to conjugates containing such antibodies and compositions, combinations, methods, and uses for such conjugates. The disclosure is further related to nucleic acid molecules encoding the PD-L1 antibodies and fragments described herein.

### BACKGROUND

Programmed death-ligand 1 (PD-L1), also known as cluster of differentiation 247 (CD247) or B7-H1, is a protein receptor that functions as an immune checkpoint and downregulates immune responses. While therapies involving inhibitors of the PD-1:PD-L1 pathway and have been used in the treatment of cancers, challenges remain. Improved compositions and methods are still urgently needed to address the clinical challenges. Provided are embodiments that meet such needs.

### SUMMARY

Provided herein is an antibody or antigen-binding fragment that specifically binds a Programmed Death-Ligand (PD-L1) protein. In some of any of the embodiments, the PD-L1 binding antibody or antigen-binding fragment comprises a heavy chain variable (VH) region comprising a heavy chain complementarity determining region 1 (CDR-H1), a CDR-H2, and a CDR-H3, respectively, comprising a CDR-H1, a CDR-H2, and a CDR-H3, respectively, contained within the VH region sequence set forth in SEQ ID NO:1; and a light chain variable (VL) region comprising a light chain complementarity determining region 1 (CDR-L1), a CDR-L2, and a CDR-L3, respectively, comprising a CDR-L1, a CDR-L2, and a CDR-L3, respectively, contained within the VL region sequence set forth in SEQ ID NO:17. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:18. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:3; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:19. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:4; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:19. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:5; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:20. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:1; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:21. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:22. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:6; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:238. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:7; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:24. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:8; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:25. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:9; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:26. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:10; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:27. In some of any of the embodiments, the PD-L1 binding antibody or antigen-binding fragment comprises a VH region comprising a CDR-H1 a CDR-H2, and a CDR-H3, respectively, comprising a CDR-H1, a CDR-H2, and a CDR-H3, respectively, contained within the VH region sequence set forth in SEQ ID NO:11; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO: 28. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO: 12; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:29. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:13; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:30. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:14; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:31. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:15; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO: 32. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO: 12; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:33. In some of any of the embodiments, the PD-L1 binding antibody or antigen-binding fragment comprises a VH region comprising a CDR-H1, a CDR-H2, and a CDR-H3, respectively, comprising a CDR-H1, a CDR-H2, and a CDR-H3, respectively, contained within the VH region sequence set forth in SEQ ID NO:16; and a VL region comprising a CDR-L1, a CDR-L2, and a CDR-L3, respectively, comprising a CDR-L1, a CDR-L2, and a CDR-L3, respectively, contained within the VL region sequence set forth in SEQ ID NO:34.

In some of any of the embodiments, the VH region comprises a CDR-H1 comprising SEQ ID NO:35, a CDR-H2 comprising SEQ ID NO:36, and a CDR-H3 comprising SEQ ID NO: 37; and the VL region comprises a CDR-L1 comprising SEQ ID NO:210, a CDR-L2 comprises SEQ ID NO:211, and a CDR-L3 comprising SEQ ID NO:212. In some of any of the embodiments, the VH region comprises a CDR-H1 comprising SEQ ID NO:48, a CDR-H2 comprising SEQ ID NO:49, and a CDR-H3 comprising SEQ ID NO:37; and the VL region comprises a CDR-L1 comprising SEQ ID NO:218, a CDR-L2 comprises SEQ ID NO:211, and a CDR-L3 comprising SEQ ID NO:212. In some of any of the embodiments, the VH region comprises a CDR-H1 comprising SEQ ID NO:58, a CDR-H2 comprising SEQ ID NO:59, and a CDR-H3 comprising SEQ ID NO:60; and the VL region comprises a CDR-L1 comprising SEQ ID NO: 221, a CDR-L2 comprises SEQ ID NO:222, and a CDR-L3 comprising SEQ ID NO:223. In some of any of the embodiments, the VH region comprises a CDR-H1 comprising SEQ ID NO: 71, a CDR-H2 comprising SEQ ID NO:72, and a CDR-H3 comprising SEQ ID NO:60; and the VL region comprises a CDR-L1 comprising SEQ ID NO:229, a CDR-L2 comprises SEQ ID NO: 222, and a CDR-L3 comprising SEQ ID NO:223. In some of any of the embodiments, the VH region comprises a CDR-H1 comprising SEQ ID NO:35, a CDR-H2 comprising SEQ ID NO: 36, and a CDR-H3 comprising SEQ ID NO:37; and the VL region comprises a CDR-L1 comprising SEQ ID NO:233, a CDR-L2 comprises SEQ ID NO:234, and a CDR-L3 comprising SEQ ID NO:235. In some of any of the embodiments, the VH region comprises a CDR-H1 comprising SEQ ID NO:48, a CDR-H2 comprising SEQ ID NO:49, and a CDR-H3 comprising SEQ ID NO:37; and the VL region comprises a CDR-L1 comprising SEQ ID NO:241, a CDR-L2 comprises SEQ ID NO:234, and a CDR-L3 comprising SEQ ID NO:242. In some of any of the embodiments, the VH region comprises a CDR-H1 comprising SEQ ID NO:82, a CDR-H2 comprising SEQ ID NO:83, and a CDR-H3 comprising SEQ ID NO:84; and the VL region comprises a CDR-L1 comprising SEQ ID NO:246, a CDR-L2 comprises SEQ ID NO:247, and a CDR-L3 comprising SEQ ID NO:248. In some of any of the embodiments, the VH region comprises a CDR-H1 comprising SEQ ID NO:48, a CDR-H2 comprising SEQ ID NO:95, and a CDR-H3 comprising SEQ ID NO:84; and the VL region comprises a CDR-L1 comprising SEQ ID NO: 246, a CDR-L2 comprises SEQ ID NO:254, and a CDR-L3 comprising SEQ ID NO:255. In some of any of the embodiments, the VH region comprises a CDR-H1 comprising SEQ ID NO: 48, a CDR-H2 comprising SEQ ID NO:104, and a CDR-H3 comprising SEQ ID NO:105; and the VL region comprises a CDR-L1 comprising SEQ ID NO:258, a CDR-L2 comprises SEQ ID NO:259, and a CDR-L3 comprising SEQ ID NO:260. In some of any of the embodiments, the VH region comprises a CDR-H1 comprising SEQ ID NO:116, a CDR-H2 comprising SEQ ID NO: 117, and a CDR-H3 comprising SEQ ID NO:118; and the VL region comprises a CDR-L1 comprising SEQ ID NO:265, a CDR-L2 comprises SEQ ID NO:266, and a CDR-L3 comprising SEQ ID NO:267. In some of any of the embodiments, the VH region comprises a CDR-H1 comprising SEQ ID NO:129, a CDR-H2 comprising SEQ ID NO:130, and a CDR-H3 comprising SEQ ID NO:131; and the VL region comprises a CDR-L1 comprising SEQ ID NO:246, a CDR-L2 comprises SEQ ID NO:273, and a CDR-L3 comprising SEQ ID NO: 274. In some of any of the embodiments, the VH region comprises a CDR-H1 comprising SEQ ID NO:142, a CDR-H2 comprising SEQ ID NO:143, and a CDR-H3 comprising SEQ ID NO: 144; and the VL region comprises a CDR-L1 comprising SEQ ID NO:278, a CDR-L2 comprises SEQ ID NO:279, and a CDR-L3 comprising SEQ ID NO:280. In some of any of the embodiments, the VH region comprises a CDR-H1 comprising SEQ ID NO:155, a CDR-H2 comprising SEQ ID NO: 156, and a CDR-H3 comprising SEQ ID NO:157; and the VL region comprises a CDR-L1 comprising SEQ ID NO:286, a CDR-L2 comprises SEQ ID NO:287, and a CDR-L3 comprising SEQ ID NO:288. In some of any of the embodiments, the VH region comprises a CDR-H1 comprising SEQ ID NO:168, a CDR-H2 comprising SEQ ID NO:169, and a CDR-H3 comprising SEQ ID NO:170; and the VL region comprises a CDR-L1 comprising SEQ ID NO:294, a CDR-L2 comprises SEQ ID NO:234, and a CDR-L3 comprising SEQ ID NO: 295. In some of any of the embodiments, the VH region comprises a CDR-H1 comprising SEQ ID NO:35, a CDR-H2 comprising SEQ ID NO:181, and a CDR-H3 comprising SEQ ID NO: 182; and the VL region comprises a CDR-L1 comprising SEQ ID NO:299, a CDR-L2 comprises SEQ ID NO:300, and a CDR-L3 comprising SEQ ID NO:301. In some of any of the embodiments, the VH region comprises a CDR-H1 comprising SEQ ID NO:168, a CDR-H2 comprising SEQ ID NO: 169, and a CDR-H3 comprising SEQ ID NO:193; and the VL region comprises a CDR-L1 comprising SEQ ID NO:306, a CDR-L2 comprises SEQ ID NO:234, and a CDR-L3 comprising SEQ ID NO:307. In some of any of the embodiments, the VH region comprises a CDR-H1 comprising SEQ ID NO:155, a CDR-H2 comprising SEQ ID NO:156, and a CDR-H3 comprising SEQ ID NO:157; and the VL region comprises a CDR-L1 comprising SEQ ID NO:311, a CDR-L2 comprises SEQ ID NO:312, and a CDR-L3 comprising SEQ ID NO: 313. In some of any of the embodiments, the VH region comprises a CDR-H1 comprising SEQ ID NO:197, a CDR-H2 comprising SEQ ID NO:198, and a CDR-H3 comprising SEQ ID NO: 199; and the VL region comprises a CDR-L1 comprising SEQ ID NO:319, a CDR-L2 comprises SEQ ID NO:320, and a CDR-L3 comprising SEQ ID NO:321.

In some of any of the embodiments, the VH region comprises a CDR-H1 comprising SEQ ID NO:40, a CDR-H2 comprising SEQ ID NO:41, and a CDR-H3 comprising SEQ ID NO: 37; and the VL region comprises a CDR-L1 comprising SEQ ID NO:210, a CDR-L2 comprises SEQ ID NO:211, and a CDR-L3 comprising SEQ ID NO:212. In some of any of the embodiments, the VH region comprises a CDR-H1 comprising SEQ ID NO:52, a CDR-H2 comprising SEQ ID NO:53, and a CDR-H3 comprising SEQ ID NO:37; and the VL region comprises a CDR-L1 comprising SEQ ID NO:218, a CDR-L2 comprises SEQ ID NO:211, and a CDR-L3 comprising SEQ ID NO:212. In some of any of the embodiments, the VH region comprises a CDR-H1 comprising SEQ ID NO:63, a CDR-H2 comprising SEQ ID NO:64, and a CDR-H3 comprising SEQ ID NO:60; and the VL region comprises a CDR-L1 comprising SEQ ID NO: 221, a CDR-L2 comprises SEQ ID NO:222, and a CDR-L3 comprising SEQ ID NO:223. In some of any of the embodiments, the VH region comprises a CDR-H1 comprising SEQ ID NO: 75, a CDR-H2 comprising SEQ ID NO:76, and a CDR-H3 comprising SEQ ID NO:60; and the VL region comprises a CDR-L1 comprising SEQ ID NO:229, a CDR-L2 comprises SEQ ID NO: 222, and a CDR-L3 comprising SEQ ID NO:223. In some of any of the embodiments, the VH region comprises a CDR-H1 comprising SEQ ID NO:40, a CDR-H2 comprising SEQ ID NO: 41, and a CDR-H3 comprising SEQ ID NO:37; and the VL region comprises a CDR-L1 comprising SEQ ID NO:233, a CDR-L2 comprises SEQ ID NO:234, and a CDR-L3 comprising SEQ ID NO:235. In some of any of the embodiments, the VH region comprises a CDR-H1 comprising SEQ ID NO:52, a CDR-H2 comprising SEQ ID NO:53, and a CDR-H3 comprising SEQ ID NO:37; and the VL region comprises a CDR-L1 comprising SEQ ID NO:241, a CDR-L2 comprises SEQ ID NO:234, and a CDR-L3 comprising SEQ ID NO:242. In some of any of the embodiments, the VH region comprises a CDR-H1 comprising SEQ ID NO:87, a CDR-H2 comprising SEQ ID NO:88, and a CDR-H3 comprising SEQ ID NO:84; and the VL region comprises a CDR-L1 comprising SEQ ID NO:246, a CDR-L2 comprises SEQ ID NO:247, and a CDR-L3 comprising SEQ ID NO:248. In some of any of the embodiments, the VH region comprises a CDR-H1 comprising SEQ ID NO:98, a CDR-H2 comprising SEQ ID NO:99, and a CDR-H3 comprising SEQ ID NO:84; and the VL region comprises a CDR-L1 comprising SEQ ID NO: 246, a CDR-L2 comprises SEQ ID NO:254, and a CDR-L3 comprising SEQ ID NO:255. In some of any of the embodiments, the VH region comprises a CDR-H1 comprising SEQ ID NO: 108, a CDR-H2 comprising SEQ ID NO:109, and a CDR-H3 comprising SEQ ID NO:105; and the VL region comprises a CDR-L1 comprising SEQ ID NO:258, a CDR-L2 comprises SEQ ID NO:259, and a CDR-L3 comprising SEQ ID NO:260. In some of any of the embodiments, the VH region comprises a CDR-H1 comprising SEQ ID NO:121, a CDR-H2 comprising SEQ ID NO: 122, and a CDR-H3 comprising SEQ ID NO:118; and the VL region comprises a CDR-L1 comprising SEQ ID NO:265, a CDR-L2 comprises SEQ ID NO:266, and a CDR-L3 comprising SEQ ID NO:267. In some of any of the embodiments, the VH region comprises a CDR-H1 comprising SEQ ID NO:134, a CDR-H2 comprising SEQ ID NO:135, and a CDR-H3 comprising SEQ ID NO:131; and the VL region comprises a CDR-L1 comprising SEQ ID NO:246, a CDR-L2 comprises SEQ ID NO:273, and a CDR-L3 comprising SEQ ID NO: 274. In some of any of the embodiments, the VH region comprises a CDR-H1 comprising SEQ ID NO:147, a CDR-H2 comprising SEQ ID NO:148, and a CDR-H3 comprising SEQ ID NO: 144; and the VL region comprises a CDR-L1 comprising SEQ ID NO:278, a CDR-L2 comprises SEQ ID NO:279, and a CDR-L3 comprising SEQ ID NO:280. In some of any of the embodiments, the VH region comprises a CDR-H1 comprising SEQ ID NO:160, a CDR-H2 comprising SEQ ID NO: 161, and a CDR-H3 comprising SEQ ID NO:157; and the VL region comprises a CDR-L1 comprising SEQ ID NO:286, a CDR-L2 comprises SEQ ID NO:287, and a CDR-L3 comprising SEQ ID NO:288. In some of any of the embodiments, the VH region comprises a CDR-H1 comprising SEQ ID NO:173, a CDR-H2 comprising SEQ ID NO:174, and a CDR-H3 comprising SEQ ID NO:170; and the VL region comprises a CDR-L1 comprising SEQ ID NO:294, a CDR-L2 comprises SEQ ID NO:234, and a CDR-L3 comprising SEQ ID NO: 295. In some of any of the embodiments, the VH region comprises a CDR-H1 comprising SEQ ID NO:185, a CDR-H2 comprising SEQ ID NO:186, and a CDR-H3 comprising SEQ ID NO: 182; and the VL region comprises a CDR-L1 comprising SEQ ID NO:299, a CDR-L2 comprises SEQ ID NO:300, and a CDR-L3 comprising SEQ ID NO:301. In some of any of the embodiments, the VH region comprises a CDR-H1 comprising SEQ ID NO:173, a CDR-H2 comprising SEQ ID NO: 174, and a CDR-H3 comprising SEQ ID NO:193; and the VL region comprises a CDR-L1 comprising SEQ ID NO:306, a CDR-L2 comprises SEQ ID NO:234, and a CDR-L3 comprising SEQ ID NO:307. In some of any of the embodiments, the VH region comprises a CDR-H1 comprising SEQ ID NO:160, a CDR-H2 comprising SEQ ID NO:161, and a CDR-H3 comprising SEQ ID NO:157; and the VL region comprises a CDR-L1 comprising SEQ ID NO:311, a CDR-L2 comprises SEQ ID NO:312, and a CDR-L3 comprising SEQ ID NO: 313. In some of any of the embodiments, the VH region comprises a CDR-H1 comprising SEQ ID NO:202, a CDR-H2 comprising SEQ ID NO:203, and a CDR-H3 comprising SEQ ID NO: 199; and the VL region comprises a CDR-L1 comprising SEQ ID NO:319, a CDR-L2 comprises SEQ ID NO:320, and a CDR-L3 comprising SEQ ID NO:321.

In some of any of the embodiments, the VH region comprises a sequence that has at least 95% sequence identity to SEQ ID NO:1; and the VL region comprises a sequence that has at least 95% sequence identity to SEQ ID NO:17. In some of any of the embodiments, the VH region comprises a sequence that has at least 95% sequence identity to SEQ ID NO:2; and the VL region comprises a sequence that has at least 95% sequence identity to SEQ ID NO:18. In some of any of the embodiments, the VH region comprises a sequence that has at least 95% sequence identity to SEQ ID NO:3; and the VL region comprises a sequence that has at least 95% sequence identity to SEQ ID NO:19. In some of any of the embodiments, the VH region comprises a sequence that has at least 95% sequence identity to SEQ ID NO:4; and the VL region comprises a sequence that has at least 95% sequence identity to SEQ ID NO:19. In some of any of the embodiments, the VH region comprises a sequence that has at least 95% sequence identity to SEQ ID NO:5; and the VL region comprises a sequence that has at least 95% sequence identity to SEQ ID NO:20. In some of any of the embodiments, the VH region comprises a sequence that has at least 95% sequence identity to SEQ ID NO:1; and the VL region comprises a sequence that has at least 95% sequence identity to SEQ ID NO:21. In some of any of the embodiments, the VH region comprises a sequence that has at least 95% sequence identity to SEQ ID NO:21; and the VL region comprises a sequence that has at least 95% sequence identity to SEQ ID NO:22. In some of any of the embodiments, the VH region comprises a sequence that has at least 95% sequence identity to SEQ ID NO:6; and the VL region comprises a sequence that has at least 95% sequence identity to SEQ ID NO:23. In some of any of the embodiments, the VH region comprises a sequence that has at least 95% sequence identity to SEQ ID NO:7; and the VL region comprises a sequence that has at least 95% sequence identity to SEQ ID NO:24. In some of any of the embodiments, the VH region comprises a sequence that has at least 95% sequence identity to SEQ ID NO:8; and the VL region comprises a sequence that has at least 95% sequence identity to SEQ ID NO:25. In some of any of the embodiments, the VH region comprises a sequence that has at least 95% sequence identity to SEQ ID NO:9; and the VL region comprises a sequence that has at least 95% sequence identity to SEQ ID NO:26. In some of any of the embodiments, the VH region comprises a sequence that has at least 95% sequence identity to SEQ ID NO:10; and the VL region comprises a sequence that has at least 95% sequence identity to SEQ ID NO:27. In some of any of the embodiments, the VH region comprises a sequence that has at least 95% sequence identity to SEQ ID NO:11; and the VL region comprises a sequence that has at least 95% sequence identity to SEQ ID NO:28. In some of any of the embodiments, the VH region comprises a sequence that has at least 95% sequence identity to SEQ ID NO:12; and the VL region comprises a sequence that has at least 95% sequence identity to SEQ ID NO:29. In some of any of the embodiments, the VH region comprises a sequence that has at least 95% sequence identity to SEQ ID NO:13; and the VL region comprises a sequence that has at least 95% sequence identity to SEQ ID NO:30. In some of any of the embodiments, the VH region comprises a sequence that has at least 95% sequence identity to SEQ ID NO:14; and the VL region comprises a sequence that has at least 95% sequence identity to SEQ ID NO:31. In some of any of the embodiments, the VH region comprises a sequence that has at least 95% sequence identity to SEQ ID NO:15; and the VL region comprises a sequence that has at least 95% sequence identity to SEQ ID NO:32. In some of any of the embodiments, the VH region comprises a sequence that has at least 95% sequence identity to SEQ ID NO:12; and the VL region comprises a sequence that has at least 95% sequence identity to SEQ ID NO:33. In some of any of the embodiments, the VH region comprises a sequence that has at least 95% sequence identity to SEQ ID NO:16; and the VL region comprises a sequence that has at least 95% sequence identity to SEQ ID NO:34.

In some of any of the embodiments, the VH region comprises a sequence that has at least 95% sequence identity to SEQ ID NO:330; and the VL region comprises a sequence that has at least 95% sequence identity to SEQ ID NO:335. In some of any of the embodiments, the VH region comprises a sequence that has at least 95% sequence identity to SEQ ID NO:331; and the VL region comprises a sequence that has at least 95% sequence identity to SEQ ID NO:336. In some of any of the embodiments, the VH region comprises a sequence that has at least 95% sequence identity to SEQ ID NO:332; and the VL region comprises a sequence that has at least 95% sequence identity to SEQ ID NO:337. In some of any of the embodiments, the VH region comprises a sequence that has at least 95% sequence identity to SEQ ID NO:333; and the VL region comprises a sequence that has at least 95% sequence identity to SEQ ID NO:338. In some of any of the embodiments, the VH region comprises a sequence that has at least 95% sequence identity to SEQ ID NO:330; and the VL region comprises a sequence that has at least 95% sequence identity to SEQ ID NO:339. In some of any of the embodiments, the VH region comprises a sequence that has at least 95% sequence identity to SEQ ID NO:334; and the VL region comprises a sequence that has at least 95% sequence identity to SEQ ID NO:340.

In some of any of the embodiments, the VH region comprises SEQ ID NO:1; and the VL region comprises SEQ ID NO:17. In some of any of the embodiments, the VH region comprises SEQ ID NO:2; and the VL region comprises SEQ ID NO:18. In some of any of the embodiments, the VH region comprises SEQ ID NO:3; and the VL region comprises SEQ ID NO: 19. In some of any of the embodiments, the VH region comprises SEQ ID NO:4; and the VL region comprises SEQ ID NO:19. In some of any of the embodiments, the VH region comprises SEQ ID NO:5; and the VL region comprises SEQ ID NO:20. In some of any of the embodiments, the VH region comprises SEQ ID NO:1; and the VL region comprises SEQ ID NO: 21. In some of any of the embodiments, the VH region comprises SEQ ID NO:21; and the VL region comprises SEQ ID NO:22. In some of any of the embodiments, the VH region comprises SEQ ID NO:6; and the VL region comprises SEQ ID NO:23. In some of any of the embodiments, the VH region comprises SEQ ID NO:7; and the VL region comprises SEQ ID NO: 24. In some of any of the embodiments, the VH region comprises SEQ ID NO:8; and the VL region comprises SEQ ID NO:25. In some of any of the embodiments, the VH region comprises SEQ ID NO:9; and the VL region comprises SEQ ID NO:26. In some of any of the embodiments, the VH region comprises SEQ ID NO:10; and the VL region comprises SEQ ID NO: 27. In some of any of the embodiments, the VH region comprises SEQ ID NO:11; and the VL region comprises SEQ ID NO:28. In some of any of the embodiments, the VH region comprises SEQ ID NO:12; and the VL region comprises SEQ ID NO:29. In some of any of the embodiments, the VH region comprises SEQ ID NO:13; and the VL region comprises SEQ ID NO: 30. In some of any of the embodiments, the VH region comprises SEQ ID NO:14; and the VL region comprises SEQ ID NO:31. In some of any of the embodiments, the VH region comprises SEQ ID NO:15; and the VL region comprises SEQ ID NO:32. In some of any of the embodiments, the VH region comprises SEQ ID NO: 12; and the VL region comprises SEQ ID NO: 33. In some of any of the embodiments, the VH region comprises SEQ ID NO:16; and the VL region comprises SEQ ID NO:34.

In some of any of the embodiments, the VH region comprises SEQ ID NO:330; and the VL region comprises SEQ ID NO:335. In some of any of the embodiments, the VH region comprises SEQ ID NO:331; and the VL region comprises SEQ ID NO:336. In some of any of the embodiments, the VH region comprises SEQ ID NO:332; and the VL region comprises SEQ ID NO: 337. In some of any of the embodiments, the VH region comprises SEQ ID NO:333; and the VL region comprises SEQ ID NO:338. In some of any of the embodiments, the VH region comprises SEQ ID NO:330; and the VL region comprises SEQ ID NO:339. In some of any of the embodiments, the VH region comprises SEQ ID NO:334; and the VL region comprises SEQ ID NO: 340.

In some of any of the embodiments, the PD-L1 protein is a human PD-L1 protein.

In some of any of the embodiments, the antibody or antigen-binding fragment is recombinant. In some of any of the embodiments, the antibody or antigen-binding fragment is monoclonal. In some of any of the embodiments, the antibody or antigen-binding fragment is a human, chimeric, or humanized antibody or antigen-binding fragment.

In some of any of the embodiments, the antibody or antigen binding fragment comprises an Fc region of a human immunoglobulin and/or human antibody framework regions.

In some of any of the embodiments, the antibody or antigen-binding fragment is that is a single chain antibody fragment. In some of any of the embodiments, the antibody fragment comprises a single chain Fv (scFv).

In some of any of the embodiments, the antibody is a whole or intact antibody.

In some of any of the embodiments, the antibody or antigen-binding fragment is a bispecific antibody that further specifically binds to a second antigen. In some of any of the embodiments, the second antigen is an antigen expressed on a tumor cell or an immune cell. In some of any of the embodiments, the second antigen is an antigen expressed on an immune cell that is a T cell and the antigen is CD25.

In some of any of the embodiments, the antibody or antigen-binding fragment thereof comprises an Fc region that exhibits one or more Fc-mediated effector function(s). In some of any of the embodiments, the antibody or antigen-binding fragment thereof an Fc region that lacks Fc-mediated effector function(s), exhibits substantially reduced Fc-mediated effector function(s) or does not exhibit substantial Fc-mediated effector function(s). In some of any of the embodiments, the antibody or antigen-binding fragment thereof comprises an Fc region that exhibits enhanced Fc-mediated effector function(s). In some of any of the embodiments, the Fc-mediated effector function is selected from one or more of an antibody-dependent cellular cytotoxicity (ADCC), antibody-dependent cellular phagocytosis (ADCP) or complement-dependent cytotoxicity (CDC).

In some of any of the embodiments, the antibody or antigen binding fragment comprises an IgG1 Fc region or an IgG1 isotype, an IgG2 Fc region or an IgG2 isotype, IgG3 Fc region or an IgG3 isotype, or an IgG4 Fc region or an IgG4 isotype.

Also provided is a conjugate, comprising any of the provided antibody or antigen-binding fragment and a heterologous molecule or moiety. In some of any of the embodiments, the heterologous molecule or moiety is a protein, peptide, nucleic acid, dye, or small molecule. In some of any of the embodiments, the heterologous molecule or moiety is a cytotoxic agent, a toxin, a radioisotope, a chemotherapeutic agent, a lytic peptide, a cytokine, or a photoactivatable dye.

In some of any of the embodiments, the photoactivatable dye is a phthalocyanine dye. In some of any of the embodiments, the phthalocyanine dye is a Si-phthalocyanine dye.

In some of any of the embodiments, the phthalocyanine dye is IR700.

In some of any of the embodiments, the phthalocyanine dye has the structure of Formula (I):

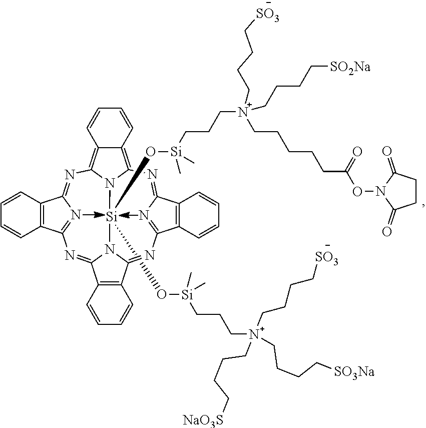

or is a salt, stereoisomer, or tautomer thereof.

In some of any of the embodiments, the conjugate is activated by illumination at a wavelength between at or at about 600 nm and at or about 850 nm to effect cell killing.

In some of any of the embodiments, the activated conjugate effects tumor growth inhibition or killing at a higher level, activity, or potency than the unconjugated antibody.

In some of any of the embodiments, the antibody or antigen-binding fragment and the moiety are linked directly or indirectly via a linker.

In some of any of the embodiments, the antibody or antigen-binding fragment is covalently attached to the heterologous molecule or moiety.

In some of any of the embodiments, when contacted with a cell expressing a PD-L1 protein, the conjugate exhibits increased internalization compared to an unconjugated antibody or antigen-binding fragment, or a conjugate comprising a reference antibody. In some of any of the embodiments, when contacted with a cell expressing a PD-L1 protein, the conjugate exhibits reduced internalization compared to an unconjugated antibody or antigen-binding fragment, or a conjugate comprising a reference antibody. In some of any of the embodiments, the reference antibody is avelumab.

In some of any of the embodiments, the conjugate does not exhibit substantially reduced binding affinity to a PD-L1 protein compared to the unconjugated antibody, or exhibits at least 50%, at least 60%, at least 70%, at least 80%, at least 90%, at least 95%, at least 96%, at least 97%, at least 98%, or at least 99% of the binding affinity of the unconjugated antibody to the PD-L1 protein. In some of any of the embodiments, the conjugate exhibits similar binding to a PD-L1 protein compared to the unconjugated antibody.

Also provided is a polynucleotide encoding any of the provided antibody or antigen-binding fragment.

Also provided is a vector, comprising any of the provided polynucleotide. In some of any of the embodiments, the vector is an expression vector.

Also provided is an engineered cell comprising any of the provided vectors.

Also provided is an engineered cell expressing any of the provided antibody or antigen-binding fragment.

Also provided is a composition comprising any of the provided antibody or antigen-binding fragments, or the any of the provided conjugates. In some of any of the embodiments, the composition also comprises a pharmaceutically acceptable excipient.

Also provided is a method of treatment of a disease or disorder. Also provided are any of the provided antibody or antigen-binding fragment, any of the provided conjugates or any of the provided compositions for use in any of such methods. Also provided are uses of any of the provided antibody or antigen-binding fragment, any of the provided conjugates or any of the provided compositions in the manufacture of a medicament for the treatment of a disease or disorder. In some of any of the embodiments, the method involves administering any of the provided antibody or antigen-binding fragment, any of the provided conjugates or any of the provided compositions, to a subject having a disease or disorder.

Also provided is a method of treatment, comprising administering any of the provided compositions to a subject having a disease or disorder. In some of any of the embodiments, the disease or disorder is a tumor or a cancer.

Also provided is a method of treating a tumor or a lesion in a subject. In some of any of the embodiments, the method involves administering to the subject the any of the provided conjugates or any of the provided compositions; and illuminating a target area within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate; whereby the growth, volume or dimensions of the tumor or the lesion is reduced or inhibited.

Also provided is a method of treating a tumor or lesion. In some of any of the embodiments, the method involves administering to the subject any of the provided conjugates or any of the provided compositions to a subject having a tumor or lesion that has had a low response to, was unresponsive to, was resistant to, was refractory to, had failed to respond to or has relapsed after, a prior immunotherapy; and illuminating a target area where the tumor or lesion is located, at a wavelength of at or about 600 nm to at or about 850 nm and at a dose of from at or about 25 J/cm² to at or about 400 J/cm² or from at or about 2 J/cm fiber length to at or about 500 J/cm fiber length; wherein the method results in the killing of a PD-L1 expressing cell in the target area.

In some of any of the embodiments, the prior immunotherapy is a treatment with an immune checkpoint inhibitor.

In some of any of the embodiments, the subject has primary resistance or acquired resistance to a prior immunotherapy that comprises a PD-1/PD-L1 blockade therapy.

Also provided is a method of treating a tumor or lesion. In some of any of the embodiments, the method involves administering to the subject any of the provided conjugates or any of the provided compositions to a subject that is treatment-naïve for an immune checkpoint inhibitor or that has not previously received a treatment with an immune checkpoint inhibitor; and illuminating a target area where a tumor or lesion is located in the subject at a wavelength of at or about 600 nm to at or about 850 nm and at a dose of from at or about 25 J/cm² to at or about 400 J/cm² or from at or about 2 J/cm fiber length to at or about 500 J/cm fiber length; wherein after the illumination, the growth, size or viability of the tumor or lesion is reduced or inhibited.

In some of any of the embodiments, the subject is administered the conjugate to treat, inhibit the growth of and/or reduce the size of a first tumor or lesion; and the method inhibits, delays or prevents the appearance, growth or establishment of one or more second tumors or lesions, located distally to the first tumor or lesion.

Also provided is a method of immunizing a subject having a first tumor or lesion. In some of any of the embodiments, the method involves administering to the subject any of the provided conjugates to a subject having a tumor or lesion; and illuminating a target area within the first tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length; wherein the first tumor or lesion is inhibited in growth and/or reduced in size; and the appearance, growth or establishment of one or more second tumors or lesions, located distally to the treated first tumor or lesion, is inhibited, delayed or prevented.

In some of any of the embodiments, the second tumor or lesion is a metastasis of the first tumor or lesion. In some of any of the embodiments, the method results in killing of a PD-L1-expressing cell in the vicinity of the first tumor or lesion and/or activates an immune cell response, thereby inhibiting, delaying or preventing the appearance, growth or establishment of the second tumor or lesion. In some of any of the embodiments, the second tumor or lesion is phenotypically and/or genotypically the same as the first tumor or lesion. In some of any of the embodiments, the second tumor or lesion is phenotypically and/or genotypically different from the first tumor or lesion.

In some of any of the embodiments, the second tumor or lesion is not derived from a metastasis of the first tumor or lesion. In some of any of the embodiments, the method results in the killing of the PD-L1-expressing cell or the PD-L1-expressing immune cell. In some of any of the embodiments, the tumor or lesion comprises a tumor cell, and the tumor cell does not express or has a reduced expression of an immune checkpoint protein. In some of any of the embodiments, the immune checkpoint protein is selected from among PD-L1, PD-1, and CTLA-4.

In some of any of the embodiments, the tumor cell does not express PD-L1 in response to an inflammatory stimulus. In some of any of the embodiments, the inflammatory stimulus is interferon.

In some of any of the embodiments, the tumor cell is not specifically recognized by an anti-PD-L1 antibody. In some of any of the embodiments, the tumor or lesion comprises PD-L1 negative tumor cells. In some of any of the embodiments, at least or at least about 40%, 50%, 60%, 70%, 80%, 90% or 95% of the tumor cells in the tumor or lesion are PD-L1 negative tumor cells.

In some of any of the embodiments, the treatment delays regrowth of the tumor or lesion, prevents a relapse of a cancer associated with the tumor or lesion or prolongs the duration of remission of a cancer associated with the tumor or lesion. In some of any of the embodiments, the inhibition of the growth of the tumor or lesion and/or killing of the PD-L1-expressing cell is dependent on the presence of CD8+ T cells.

In some of any of the embodiments, the subject is naïve to treatment with an immune checkpoint inhibitor or has not previously received treatment with an immune checkpoint inhibitor. In some of any of the embodiments, the subject has been previously treated with an immune checkpoint inhibitor. In some of any of the embodiments, the subject has had a low response to, was unresponsive to, was resistant to, was refractory to, had failed to respond to or has relapsed after the previous treatment with the immune checkpoint inhibitor. In some of any of the embodiments, the inhibition of the growth, size or viability of the tumor or lesion resulting from carrying out the method is greater compared to the inhibition resulting from the previous treatment with the immune checkpoint inhibitor.

In some of any of the embodiments, the immune checkpoint inhibitor is an inhibitor of PD-L1, PD-1 or CTLA-4. In some of any of the embodiments, immune checkpoint inhibitor is a PD-1 inhibitor. In some of any of the embodiments, the PD-1 inhibitor is an anti-PD-1 antibody. In some of any of the embodiments, the PD-1 inhibitor is an anti-CTLA-4 antibody. In some of any of the embodiments, the immune checkpoint inhibitor is PD-L1 inhibitor. In some of any of the embodiments, the PD-L1 inhibitor is an anti-PD-L1 antibody.

In some of any of the embodiments, the method increases the number or activity of immune cells in the tumor or lesion and/or in the microenvironment of the tumor or lesion.

In some of any of the embodiments, the target area comprises immune cells expressing PD-L1. In some of any of the embodiments, the PD-L1 expressing cell is an immune cell. In some of any of the embodiments, the immune cell is a monocyte, a macrophage, a dendritic cell (DC), or a myeloid-derived suppressor cell (MDSC). In some of any of the embodiments, the immune cell is selected from the group consisting of monocytes, macrophages, such as M1 macrophages, M2 macrophages and/or M2 tumor associated macrophages (M2 TAM), dendritic cells (DC), tolerogenic dendritic cells (tDC) and myeloid derived suppressor cells (MDSC). In some of any of the embodiments, the immune cell is a monocyte. In some of any of the embodiments, the immune cell is a macrophage. In some of any of the embodiments, the immune cell is an M1 macrophage. In some of any of the embodiments, the immune cell is an M2 macrophage. In some of any of the embodiments, the immune cell is an M2 tumor associated macrophages (M2 TAM). In some of any of the embodiments, the immune cell is a dendritic cell (DC). In some of any of the embodiments, the immune cell is a tolerogenic dendritic cell (tDC). In some of any of the embodiments, the immune cell is a myeloid derived suppressor cell (MDSC). In some of any of the embodiments, the immune cell is located in the tumor, the tumor microenvironment or a lymph node.

In some of any of the embodiments, prior to administering the conjugate, the subject has a tumor or lesion having a low number or level of CD8+ T cell infiltration. In some of any of the embodiments, the number, level or activity of immune cells is increased in the tumor or lesion or in the microenvironment of the tumor or lesion after the administering and the illuminating. In some of any of the embodiments, the number or level of CD8+ T cell infiltration in the tumor or lesion is increased after the administering and the illuminating. In some of any of the embodiments, the number or level of memory T cells in the vicinity of the tumor or lesion is increased after the administering and the illuminating. In some of any of the embodiments, the targeting molecule is or comprises an antibody, an antigen-binding antibody fragment or antibody-like molecule that binds PD-L1. In some of any of the embodiments, the targeting molecule is or comprises an anti-PD-L1 antibody or antigen-binding fragment thereof. In some of any of the embodiments, the target area is a lymph node or in the vicinity of a lymph node. In some of any of the embodiments, the subject exhibits a durable response, prolonged progression-free survival, a reduced chance of relapse, and/or a reduced chance of metastasis, after the administering and the illuminating.

In some of any of the embodiments, the illuminating is carried out between 30 minutes and 96 hours after administering the conjugate. In some of any of the embodiments, the illuminating is carried out 24 hours±4 hours after administering the conjugate. In some of any of the embodiments, the target area is illuminated at a wavelength of 690±40 nm. In some of any of the embodiments, the target area is illuminated at a wavelength of 670±50 nm. In some of any of the embodiments, the target area is illuminated at a dose of at or about of 50 J/cm² or at or about 100 J/cm of fiber length.

In some of any of the embodiments, the tumor, lesion or cancer is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any of the embodiments, one or more of steps of the method are repeated. In some of any of the embodiments, the administration of the antibody or antigen-binding fragment, the conjugate or the composition is repeated one or more times. In some of any of the embodiments, after each repeated administration of the conjugate or the composition, the illuminating step is repeated.

In some of any of the embodiments, the method further involves administering an additional therapeutic agent or anti-cancer treatment.

### BRIEF DESCRIPTION OF THE DRAWINGS

FIGS. 1A-1F show the binding of exemplary anti-PD-L1 antibodies to human PD-L1 (FIGS. 1A, 1C, and 1E) and cynomolgus PD-L1 (FIGS. 1B, 1D, and 1F) determined by ELISA.

FIG. 1G shows the binding specificity of an exemplary anti-PD-L1 antibody, 1P4, for PD-L1 compared to other B7 ligands, determined by ELISA. FIG. 1H shows the binding specificity of an exemplary anti-PD-L1 antibody, 1P9, for PD-L1 compared to other B7 ligands, determined by ELISA.

FIG. 2A shows the binding of exemplary anti-PD-L1 antibodies, containing a wild-type Fc or an effector knock-out Fc region, to human PD-L1 expressed on the surface of CHO cells engineered to express human PD-L1. FIG. 2B shows the binding of exemplary anti-PD-L1 antibodies, containing a wild-type Fc or an effector knock-out Fc region, to wild-type CHO cells.

FIG. 3 shows the binding of exemplary anti-PD-L1 antibodies to PD-L1-expressing A431 cancer cells.

FIGS. 4A-4B show the ADCC activity of exemplary anti-PD-L1 antibodies containing a wild-type Fc region (FIG. 4A) or an effector knock-out Fc region (FIG. 4B).

FIGS. 5A-5B show the binding of exemplary anti-PD-L1-IR700 conjugates to human PD-L1 (FIG. 5A) and cynomolgus PD-L1 (FIG. 5B).

FIG. 6A shows the binding of exemplary anti-PD-L1-IR700 conjugates, containing a wild-type Fc or an effector knock-out Fc region, to human PD-L1 expressed on the surface of CHO cells engineered to express human PD-L1. FIG. 6B shows the binding of exemplary anti-PD-L1-IR700 conjugates, containing a wild-type Fc or an effector knock-out Fc region, to wild-type CHO cells.

FIG. 7A shows the binding of exemplary anti-PD-L1-IR700 conjugates to A431 cancer cells. FIG. 7B shows the binding of an exemplary anti-PD-L1-IR700 conjugate compared to the avelumab-IR700 conjugate to interferon-gamma-stimulated A431 cells. FIG. 7C shows the binding of an exemplary anti-PD-L1-IR700 conjugate compared to the avelumab-IR700 conjugate to interferon-gamma-stimulated BxPC3 cells. FIG. 7D shows the binding of an exemplary anti-PD-L1-IR700 conjugate compared to the avelumab-IR700 conjugate to CHO cells engineered to express human PD-L1 (CHO-hPD-L1 cells). FIG. 7E shows the binding of an exemplary anti-PD-L1-IR700 conjugate and avelumab-IR700 conjugate to wild-type CHO cells.

FIG. 8A shows the photoimmunotherapy-induced killing of PD-L1-expressing A431 squamous cell carcinoma cells following illumination of 1P4-IR700, 1P9-IR700, and avelumab-IR700 conjugates.

FIGS. 8B-8C show photoimmunotherapy-induced killing of IFN-γ-stimulated A431 cancer cells following incubation with 1P9-IR700 or avelumab-IR700 conjugates for 1 hr. (FIG. 8B) or 24 hr. (FIG. 8C) followed by illumination.

FIG. 8D shows the viability of human peripheral blood mononuclear cells (PBMCs) from three donors following incubation with 1P9-IR700 or avelumab-IR700 and illumination.

FIG. 8E shows the viability of M1 and M2 primary human macrophages following incubation with 1P9-IR700 and illumination.

FIGS. 9A-9C show the photoimmunotherapy-induced killing of A431 squamous cell carcinoma cells following illumination of avelumab-IR700 (FIG. 9A), 1P9-IR700 (FIG. 9B), and 1P4-IR700 (FIG. 9C) at light fluencies ranging from 0 J to 128 J.

FIG. 10A shows the ability of exemplary anti-PD-L1 antibody, 1P4, control antibody avelumab, and exemplary conjugate, 1P4-IR700, to block the PD-1/PD-L1 interaction.

FIG. 10B shows the ability of exemplary anti-PD-L1 antibody, 1P9, control antibody avelumab, and exemplary conjugate, 1P9-IR700, to block the PD-1/PD-L1 interaction.

FIG. 11 shows the internalization of exemplary anti-PD-L1 conjugates, 1P9-IR700 and 1P4-IR700, and reference conjugate avelumab-IR700 on pancreatic BxPC3 cancer cells.

### DETAILED DESCRIPTION

Provided herein are anti-PD-L1 antibodies, anti-PD-L1 antibody fragments, conjugates, compositions, combinations, methods and uses for treating a subject having a tumor, a lesion or a cancer, for example by activating an immune response and/or by killing target tumor cells that express PD-L1. Also provided here in are anti-PD-L1 antibodies, anti-PD-L1 antibody fragments, conjugates, compositions, combinations, methods and uses for enhancing systemic immunity in a subject, such as a cancer. In some aspects the cancer is an invasive cancer, an infiltrating cancer, or a metastatic cancer. Also provided are anti-PD-L1 antibodies, anti-PD-L1 antibody fragments, conjugates, compositions, combinations and methods for generating an enhanced response, for example, an enhanced response to a second treatment or a therapy in a subject, e.g., a subject having a cancer or a tumor, such as an invasive cancer, an infiltrating cancer, or a metastatic cancer.

In some aspects, the provided embodiments involve administering to the subject an anti-PD-L1 antibody provided herein to directly treat a tumor or lesion, to indirectly treat a tumor or lesion, to enhance systemic immunity, to enhance an immune response, or to enhance response to a second treatment or therapy.

In some aspects, the provided embodiments involve administering to the subject a conjugate that contains any of the provided antibodies or antigen-binding fragments that binds programmed death-ligand 1 (PD-L1), conjugated with a phthalocyanine dye, such as a silicon phthalocyanine dye, such as IR700 or any of the dyes described in WO 2021/207691. In some aspects, the provided embodiments involve illumination of a target area, such as a target area where cells expressing PD-L1 are or may be present. In some aspects, the illumination results in death of cells expressing PD-L1 on the surface. In some aspects, the illumination results in the death of tumor cells expressing PD-L1 on the surface. In some aspects, the illumination results in the death of myeloid cells expressing PD-L1 on the surface. In some aspects, the illumination results in the killing of tumor cells and myeloid cells expressing PD-L1 on the surface.

In some of any the embodiments provided herein, the provided antibodies, antibody fragments, conjugates, compositions, combinations, methods and uses are employed for treating a subject having a tumor, a lesion (e.g., a cancerous lesion) or a cancer that has a low responsiveness or are substantially non-responsive to, has failed, has relapsed after, is refractory to and/or is resistant to prior therapeutic treatments, such as prior immunomodulatory agent treatments and/or prior anti-cancer therapeutic treatments.

In some aspects, the anti-PD-L1 antibody and, in some cases, an additional therapeutic agent, are employed in the provided compositions, combinations, methods and uses. In some aspects, the phthalocyanine dye-targeting molecule conjugate (e.g., anti-PD-L1 conjugated to IR700), and, in some cases, an additional therapeutic agent, are employed in the provided compositions, combinations, methods and uses. Uses include uses of the antibodies, antibody fragments, conjugates, compositions and combinations in such methods, such as therapeutic methods, and treatments, such as a treatment regimen, and uses of such antibodies, antibody fragments, conjugates, compositions and combinations in the preparation of a medicament, in order to carry out such therapeutic methods and treatments. Also provided are such antibodies, antibody fragments, conjugates, compositions and combinations for use in treating a tumor, a lesion or a cancer. In some aspects, such uses include performing the methods or treatments as described herein, such as any therapeutic methods or treatment regimens. In some embodiments, the methods and uses also involve illuminating a target area, such as a target area where the tumor, lesion or cancer is located in a subject, with a light, for example, as described herein. In some embodiments, the methods and uses thereby treat the tumor, lesion or cancer. In some aspects, the tumor, lesion or cancer to be treated include such as cancers that include primary tumors and secondary or metastatic tumor cells, for example, secondary or metastatic cancers, in a subject. In some aspects, the tumor, lesion or cancer can include a primary tumor or multiple primary tumors as well as metastatic tumor cells. In some cases, a treated subject may have one or more of primary tumors, metastatic tumor cells and/or invasive tumor cells.

In some aspects, also provided are methods and uses of such antibodies, antibody fragments, conjugates, compositions and combinations in enhancing, activating, inducing, provoking, augmenting, or supporting immune function, such as local and/or systemic immunity, in the subject. In some aspects, the provided embodiments can target tumor cells. In some aspects, the provided embodiments can target cells in the tumor microenvironment, including non-cancerous cells and/or immune cells, such as antigen-presenting cells or myeloid cells that have an immunosuppressive function.

One of the challenges in treating cancer patients is the lack of responsiveness of cancers to therapeutics. Compositions and methods for treating such cancers are urgently needed. In some contexts, the provided embodiments are based on the observation that, treatment with an anti-PD-L1 antibody or antibody fragment described herein, results in a marked decrease in the PD-1:PD-L1 interaction. Such blockade of the PD-1:PD-L1 interaction can lead to substantial inhibition of tumor growth and/or a complete response to the treatment with the antibody or antibody fragment. In some aspects, exemplary anti-PD-L1 antibodies or antibody fragments provided herein were observed to bind to PD-L1 expressed on the surface of a target cell at a markedly lower concentrations compared to a reference antibody, supporting an advantage of the provided antibodies or antibody fragments, such as the ability to use lower concentration of the antibody and reduced inhibition by blockade of the PD-1:PD-L1 interaction.

In some aspects, the provided anti-PD-L1 antibody or antibody fragments also provide an advantage of retaining the binding affinity after conjugation with an additional agent, such as a phthalocyanine dye. In contrast, a conjugate comprising the same agent (e.g., phthalocyanine dye) to a reference anti-PD-L1 antibody, was observed to exhibit a substantial reduction in binding affinity, for example, a reduction by almost 50-fold. Such effects of retaining the binding affinity after conjugation of the provided antibodies, are unexpected and advantageous. In some aspects, the provided antibodies would retain their activities, including binding affinity, even when additional agents are conjugated, supporting the utility and advantages of the antibodies for use in generating conjugates, such as antibody-drug conjugates and phthalocyanine dye-antibody conjugates, and uses of the conjugates, including therapeutic uses, experimental uses or imaging. The results described herein also demonstrate the advantage of the provided antibodies and antibody fragments, and conjugates comprising the provided antibodies and antibody fragments, such as antibody-phthalocyanine dye conjugates, in therapeutic applications such as in photoimmunotherapy (PIT).

In some contexts, the provided embodiments are based on the observation that, treatment with a phthalocyanine dye-targeting molecule conjugate, such as a conjugate containing an anti-PD-L1 antibody or antibody fragment described herein and a phthalocyanine dye (e.g., IR700), followed by light illumination (also referred to as “photoimmunotherapy” and “PIT”) of a target area, results in a substantial inhibition of tumor growth and/or a complete response to the treatment.

In some aspects, treatment with an anti-PD-L1 antibody provided herein can activate, induce, enhance or augment immune responses, for example, by virtue of eliminating or reducing the PD-L1:PD-1 interaction by binding PD-L1 expressed on immunosuppressive cells, such as immunosuppressive myeloid cells (e.g. myeloid-derived suppressor cells (MDSCs), tolerogenic dendritic cells (tDCs), M1 macrophages, M2 tumor associated macrophages (M2 TAMs)). In some aspects, treatment with a phthalocyanine dye-anti-PD-L1 antibody (or antibody fragment) conjugate and light illumination can activate, induce, enhance or augment immune responses, for example, by virtue of eliminating immunosuppressive cells, such as immunosuppressive myeloid cells (e.g. myeloid-derived suppressor cells (MDSCs), tolerogenic dendritic cells (tDCs), M1 macrophages, M2 tumor associated macrophages (M2 TAMs)). In some aspects, the elimination of immunosuppressive cells results in activation, induction, enhancement or augmentation of immune responses, such as anti-tumor or anti-cancer immune responses. In some aspects, any of the provided embodiments offer an advantage that they can be applied to many different tumor, lesion or cancer types, e.g., cancer types of different origin or expressing different surface antigens, or cancers can share similar immunosuppressive mechanisms. In some aspects, any of the provided embodiments can be employed to overcome such immunosuppressive mechanisms.

In addition, in some aspects, the provided embodiments can offer effective treatment of a tumor, lesion or cancer that is heterogeneous, e.g., containing various different types of tumor or cancer cells. In some aspects, the provided embodiments also offer an advantage of inducing, activating or enhancing local and/or systemic immune activity or systemic immunity in the subject, permitting treatment of tumors, lesions or cancers that are present elsewhere in the body other than the target area for illumination, such as metastasized tumor or cancer, invasive tumor or cancer, a tumor or a cancer at a different site, or a tumor, lesion or cancer of a different type. Other advantages include the treatment of metastatic cancers and/or invasive cancers without the need to locate and/or directly illuminate the metastatic tumor cells.

The provided embodiments can be also used to treat tumors, lesions or cancers that are not responsive to prior therapeutic treatments, for example, an immune checkpoint inhibitor, an anticancer agent, or a molecule against immune suppressor cells. The provided embodiments also offer other advantages in treating cancers, such as effective treatment of cancers that are not responsive to prior therapeutic treatments, including other anti-PD-L1 treatments.

The disclosure also provides unexpected features in enhancing the anti-cancer or anti-tumor immunity in a subject, for example, against a different tumor or cancer that may arise. The provided embodiments, in some contexts, are based on the observation that treatment of a cancer with a phthalocyanine dye-anti-PD-L1 antibody or antibody fragment conjugate, such as an anti-PD-L1 antibody-IR700 conjugate, followed by illumination of a tumor, results in not only treatment of that particular tumor, but also results in effective treatment of a later-arising tumor of the same or a different type. The provided embodiments also offer an effective treatment of a tumor that is introduced after the subject has a complete response following the treatment of the initial tumor, indicating an immune memory response; and/or an effective treatment for a tumor that is distal to the target area for illumination (e.g., metastasized tumor or a tumor present in a different location). The provided compositions, combinations, methods and uses can result in enhancement or improvement of the subject's immune response, e.g., systemic immune response against a cancer including immune memory response, that can be effective against tumors that may develop after the treatment.

In some aspects, also provided are methods that involve the administration of an additional therapeutic agent, such as an immunomodulatory agent, in combination with the anti-PD-L1 antibody or fragment or phthalocyanine dye conjugate thereof (e.g., anti-PD-L1-IR700 conjugate).

In some of any of the provided embodiments, treatment with or administration of an anti-PD-L1 conjugate provided herein is generally followed by illumination with a suitable wavelength of light. Such illumination is considered a part of the treatments and administrations of an anti-PD-L1 conjugate provided herein unless specifically stated that an illumination step is not performed with the method. In some cases, such illumination is referred to as photoimmunotherapy (PIT). In some contexts, exemplary anti-PD-L1 antibodies, antibody fragments, or conjugates provided herein were observed to exhibit increased or reduced internalization compared to an unconjugated antibody or antigen-binding fragment, or a conjugate comprising a reference antibody. In some contexts, exemplary anti-PD-L1 antibodies, antibody fragments, or conjugates provided herein were observed to exhibit increased internalization compared to an unconjugated antibody or antigen-binding fragment, or a conjugate comprising a reference antibody.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Pd-L1 Bindng Molecules and Encoding Polynucleotides

Provided in some aspects are PD-L1-binding molecules, such as PD-L1-binding polypeptides. Such binding molecules include antibodies (including antigen-binding fragments) that specifically bind to PD-L1 proteins, such as a human PD-L1 protein (huPD-L1). Also among the binding molecules are polypeptides containing such antibodies, including multispecific antibodies, for example bispecific antibodies, that bind PD-L1 in addition to one or more other antigens.

PD-L1 is a ligand for the immune checkpoint protein programmed cell death 1 (PD-1), expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll, 2012, Nature Reviews Cancer 12:252-264). PD-L1 expression can be induced in dendritic cells and keratinocytes upon IFN gamma stimulation. PD-L1 is expressed on activated T cells, B cells, myeloid cells, macrophages, and certain types of tumor cells. PD-L1 is expressed on certain immune cells, such as antigen-presenting cells (APCs) monocytes, dendritic cells (DC), macrophages, such as M1 macrophages, M2 macrophages, M2 tumor associated macrophages (M2 TAM), tolerogenic dendritic cells (tDCs) or myeloid derived suppressor cells (MDSCs), or certain tumor cells, to induce immunosuppression near the tumor or the tumor microenvironment (TME).

PD-L1 is a cognate ligand for PD-1. The complex of PD-1 and PD-L1 inhibits proliferation of CD8+ T cells and reduces the immune response (Topalian et al., 2012, N Engl J Med 366:2443-54; Brahmer et al., 2012, N Engl J Med 366:2455-65). The major role of PD-1 is to limit the activity of T cells in peripheral tissues during inflammation in response to infection, as well as to limit autoimmunity (Pardoll, 2012, Nature Reviews Cancer 12:252-264). PD-1 expression is induced in activated T cells and binding of PD-1 to one of its endogenous ligands, such as PD-L1, acts to inhibit T-cell activation by inhibiting stimulatory kinases (Pardoll, 2012, Nature Reviews Cancer 12:252-264). PD-1 also acts to inhibit the TCR “stop signal” (Pardoll, 2012, Nature Reviews Cancer 12:252-264). PD-1 is highly expressed on regulatory T (Treg) cells and may increase their proliferation in the presence of ligand (Pardoll, 2012, Nature Reviews Cancer 12:252-264). The binding of PD-L1 to PD-1 transmits an inhibitory signal based on interaction with phosphatases (SHP-1 or SHP-2) via an immunoreceptor tyrosine-based switch motif (ITSM).

As such, the PD-L1:PD-1 pathway serves as a checkpoint in the negative regulation of some immune responses and is a key immune-inhibitory mediator of T-cell exhaustion. Blockade of this pathway can lead to T-cell activation, expansion, and enhanced effector functions (Sakuishi et al., JEM Vol. 207, Sep. 27, 2010, pp 2187-2194).

PD-L1 is also expressed on tumor cells. Tumors known to overexpress PD-L1 include, but are not limited to, those causing breast cancer, lung cancer, bladder cancer, colon cancer, head and neck cancer, various myelomas and other cancers (Iwai et al. PNAS 99:12293-7 (2002); Ohigashi et al. Clin Cancer Res 11:2947-53 (2005)). Anti-PD-L1 antibodies have been used for treatment of cancers, such as non-small cell lung cancer, melanoma, colorectal cancer, renal-cell cancer, pancreatic cancer, gastric cancer, ovarian cancer, breast cancer, and hematologic malignancies (Brahmer et al., N Engl J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). In some aspects, use of anti-PD-L1 antibodies can reduce some of the immunosuppressive effect of PD-1/PD-L1, by blocking the binding of PD-L1 to PD-1.

Human PD-L1 is expressed as a 290 amino acid (aa) type I membrane precursor protein with a putative 18 aa signal peptide, a 221 aa extracellular domain, a 21 aa transmembrane region, and a 31 aa cytoplasmic domain. Provided are antibodies and antigen-binding fragments thereof that bind, such as specifically bind to PD-L1, such as a human PD-L1, and conjugates and molecules comprising such antibodies or antigen-binding fragments.

### A. Anti-PD-L1 Antibodies

Provided are anti-PD-L1 antibodies, including functional antigen-binding fragments. In some embodiments, the antibodies or antigen-binding fragments contain a heavy chain variable region (VH) and a light chain variable region (VL) that, together, are capable of specifically binding to PD-L1. In some embodiments, the antibodies or antigen-binding fragments include full-length IgG molecules, or fragments such as scFv, Fab, F (ab) 2, that specifically bind to PD-L1, e.g., human PD-L1. Among the provided anti-PD-L1 antibodies are human antibodies. The antibodies include isolated antibodies.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab fragments, Fv fragments, recombinant IgG (rIgG) fragments, heavy chain variable (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); A1-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given VH or VL region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given VH region amino acid sequence and a CDR-L1, a CDR-L2, and a CDR-L3 as contained within a given VL region amino acid sequence, the CDRs can be defined by any of the aforementioned schemes, such as Kabat, Chothia, AbM, IMGT, or Contact method, or other known scheme. In some embodiments, specific CDR sequences are specified. Exemplary CDR sequences of provided antibodies are described using various numbering schemes (see e.g., Table E1a and Table E1b), although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given VH region amino acid sequence and a FR-L1, a FR-L2, a FR-L3, and a FR-L4 as contained within a given VL region amino acid sequence, the FRs can be defined by any of the aforementioned schemes, such as Kabat, Chothia, AbM, IMGT, or Contact method, or other known scheme.

The term “variable region” or “variable domain” refers to the domain of an antibody heavy or light chain that is involved in binding the antibody to antigen. The variable regions of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (VH) regions, single-chain antibody molecules such as scFvs and single-domain antibodies comprising only the VH region; and multispecific antibodies formed from antibody fragments. In particular embodiments, the antibody is or comprises an antibody fragment comprising a variable heavy chain (VH) and a variable light chain (VL) region. In some embodiments, the antibodies are single-chain antibody fragments comprising a heavy chain variable (VH) region and/or a light chain variable (VL) region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided anti-PD-L1 antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues and are not limited to a minimum length. Polypeptides, including the provided antibodies and antibody conjugates may include amino acid residues including natural and/or non-natural amino acid residues. The terms also include posttranslational modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

### 1. Exemplary Antibodies

In some embodiments, the anti-PD-L1 antibody or antigen-binding fragment thereof, contains a heavy chain variable region (VH) and a light chain variable region (VL) as described herein, or a sufficient antigen-binding portion thereof. In some embodiments, the anti-PD-L1 antibody or antigen-binding fragment thereof, contains a VH region sequence or sufficient antigen-binding portion thereof that contains a CDR-H1, a CDR-H2 and/or a CDR-H3 as described herein. In some embodiments, the anti-PD-L1 antibody or antigen-binding fragment thereof, contains a VL region sequence or sufficient antigen-binding portion that contains a CDR-L1, a CDR-L2 and/or a CDR-L3 as described herein. In some embodiments, the anti-PD-L1 antibody or antigen-binding fragment thereof, contains a VH region sequence that contains a CDR-H1, a CDR-H2 and/or a CDR-H3 as described and contains a VL region sequence that contains a CDR-L1, a CDR-L2 and/or a CDR-L3 as described herein. Also among the provided antibodies are those having sequences at least at or about 90%, about 91%, about 92%, about 93%, about 94%, about 95%, about 96%, about 97%, about 98%, or about 99% identical to such sequences.

In some embodiments, the antibody or antigen-binding fragment thereof, has a heavy chain variable (VH) region having the sequence selected from any one of SEQ ID NOs: 1-16, and 330-334 or a sequence that has at least 90%, 91%, 92%, 93%, 94%, 95%, 96%, 97%, 98%, or 99% sequence identity to the VH region amino acid selected from any one of SEQ ID NOs: 1-16 and 330-334, or contains a CDR-H1, a CDR-H2, and/or a CDR-H3 present in such a VH sequence.

In some embodiments, the antibody or antigen-binding fragment thereof, has a VH having the sequence selected from any one of SEQ ID NOs: 1-11, or a sequence that has at least 90%, 91%, 92%, 93%, 94%, 95%, 96%, 97%, 98%, or 99% sequence identity to the VH region amino acid selected from any one SEQ ID NOs: 1-11, or contains a CDR-H1, a CDR-H2, and/or a CDR-H3 present in such a VH sequence.

In some embodiments, the antibody or antigen-binding fragment thereof, has a VH having the sequence selected from any one of SEQ ID NOs: 12-16, or a sequence that has at least 90%, 91%, 92%, 93%, 94%, 95%, 96%, 97%, 98%, or 99% sequence identity to the VH region amino acid selected from any one of SEQ ID NOs: 12-16, or contains a CDR-H1, a CDR-H2, and/or a CDR-H3 present in such a VH sequence.

In some embodiments, the antibody or antigen-binding fragment thereof is a humanized antibody or antigen binding fragment that has a VH region comprising a CDR-H1, a CDR-H2, and/or a CDR-H3 from the VH regions selected from any one of SEQ ID NOs: 12-16. In some embodiments, the antibody or antigen-binding fragment thereof, has a VH having the sequence selected from any one of SEQ ID NOs: 330-334 or a sequence that has at least 90%, 91%, 92%, 93%, 94%, 95%, 96%, 97%, 98%, or 99% sequence identity to the VH region amino acid selected from any one of SEQ ID NOs: 330-334, or contains a CDR-H1, a CDR-H2, and/or a CDR-H3 present in such a VH sequence.

In some embodiments, the antibody or antigen-binding fragment thereof, has a VH region having the sequence selected from any one of SEQ ID NOs: 1, 2, 6, and 7, or a sequence that has at least 90%, 91%, 92%, 93%, 94%, 95%, 96%, 97%, 98%, or 99% sequence identity to the VH region amino acid selected from any one of SEQ ID NOs: 1, 2, 6, and 7, or contains a CDR-H1, a CDR-H2, and/or a CDR-H3 present in such a VH sequence. In some embodiments, the antibody or antigen-binding fragment thereof, has a VH region set forth in SEQ ID NO:1, or a sequence that has at least 90%, 91%, 92%, 93%, 94%, 95%, 96%, 97%, 98%, or 99% sequence identity to SEQ ID NO:1, or contains a CDR-H1, a CDR-H2, and/or a CDR-H3 present in such a VH sequence. In some embodiments, the antibody or antigen-binding fragment thereof, has a VH region set forth in SEQ ID NO:2, or a sequence that has at least 90%, 91%, 92%, 93%, 94%, 95%, 96%, 97%, 98%, or 99% sequence identity to SEQ ID NO:2, or contains a CDR-H1, a CDR-H2, and/or a CDR-H3 present in such a VH sequence. In some embodiments, the antibody or antigen-binding fragment thereof, has a VH region set forth in SEQ ID NO:6, or a sequence that has at least 90%, 91%, 92%, 93%, 94%, 95%, 96%, 97%, 98%, or 99% sequence identity to SEQ ID NO:6, or contains a CDR-H1, a CDR-H2, and/or a CDR-H3 present in such a VH sequence. In some embodiments, the antibody or antigen-binding fragment thereof, has a VH region set forth in SEQ ID NO:7, or a sequence that has at least 90%, 91%, 92%, 93%, 94%, 95%, 96%, 97%, 98%, or 99% sequence identity to SEQ ID NO:7, or contains a CDR-H1, a CDR-H2, and/or a CDR-H3 present in such a VH sequence.

In some embodiments, the provided antibody or antigen-binding fragment thereof comprises a CDR-H1, a CDR-H2, and/or a CDR-H3 present in a VH comprising the sequence selected from any one of SEQ ID NOs: 1-16. In some of such embodiments, the provided antibody or antigen-binding fragment thereof comprises a CDR-H1, a CDR-H2, and/or a CDR-H3 according to Chothia numbering. In some embodiments, the provided antibody or antigen-binding fragment thereof comprises a CDR-H1, a CDR-H2, and/or a CDR-H3 according to AbM numbering. In some embodiments, the provided antibody or antigen-binding fragment thereof comprises a CDR-H1, a CDR-H2, and/or a CDR-H3 according to Kabat numbering. In some embodiments, the provided antibody or antigen-binding fragment thereof comprises a CDR-H1, a CDR-H2, and/or a CDR-H3 according to Contact numbering. In some embodiments, the provided antibody or antigen-binding fragment thereof comprises a CDR-H1, a CDR-H2, and/or a CDR-H3 according to IMGT numbering.

In some embodiments, the provided antibody or antigen-binding fragment thereof comprises a CDR-H1, a CDR-H2, and/or a CDR-H3 present in a VH comprising the sequence selected from any one of SEQ ID NOs: 1-11. In some of such embodiments, the provided antibody or antigen-binding fragment thereof comprises a CDR-H1, a CDR-H2, and/or a CDR-H3 according to Chothia numbering. In some embodiments, the provided antibody or antigen-binding fragment thereof comprises a CDR-H1, a CDR-H2, and/or a CDR-H3 according to AbM numbering. In some embodiments, the provided antibody or antigen-binding fragment thereof comprises a CDR-H1, a CDR-H2, and/or a CDR-H3 according to Kabat numbering. In some embodiments, the provided antibody or antigen-binding fragment thereof comprises a CDR-H1, a CDR-H2, and/or a CDR-H3 according to Contact numbering. In some embodiments, the provided antibody or antigen-binding fragment thereof comprises a CDR-H1, a CDR-H2, and/or a CDR-H3 according to IMGT numbering.

In some embodiments, the provided antibody or antigen-binding fragment thereof comprises a CDR-H1, a CDR-H2, and/or a CDR-H3 present in a VH comprising the sequence selected from any one of SEQ ID NOs: 12-16. In some of such embodiments, the provided antibody or antigen-binding fragment thereof comprises a CDR-H1, a CDR-H2, and/or a CDR-H3 according to Chothia numbering. In some embodiments, the provided antibody or antigen-binding fragment thereof comprises a CDR-H1, a CDR-H2, and/or a CDR-H3 according to AbM numbering. In some embodiments, the provided antibody or antigen-binding fragment thereof comprises a CDR-H1, a CDR-H2, and/or a CDR-H3 according to Kabat numbering. In some embodiments, the provided antibody or antigen-binding fragment thereof comprises a CDR-H1, a CDR-H2, and/or a CDR-H3 according to Contact numbering. In some embodiments, the provided antibody or antigen-binding fragment thereof comprises a CDR-H1, a CDR-H2, and/or a CDR-H3 according to IMGT numbering.

In some embodiments, the provided antibody or antigen-binding fragment thereof comprises a CDR-H1, a CDR-H2, and/or a CDR-H3 present in a VH comprising the sequence selected from any one of SEQ ID NOs: 330-334. In some of such embodiments, the provided antibody or antigen-binding fragment thereof comprises a CDR-H1, a CDR-H2, and/or a CDR-H3 according to Chothia numbering. In some embodiments, the provided antibody or antigen-binding fragment thereof comprises a CDR-H1, a CDR-H2, and/or a CDR-H3 according to AbM numbering. In some embodiments, the provided antibody or antigen-binding fragment thereof comprises a CDR-H1, a CDR-H2, and/or a CDR-H3 according to Kabat numbering. In some embodiments, the provided antibody or antigen-binding fragment thereof comprises a CDR-H1, a CDR-H2, and/or a CDR-H3 according to Contact numbering. In some embodiments, the provided antibody or antigen-binding fragment thereof comprises a CDR-H1, a CDR-H2, and/or a CDR-H3 according to IMGT numbering.

In some embodiments, the VH region of a provided antibody or antigen-binding fragment thereof comprises a CDR-H1, a CDR-H2, and/or a CDR-H3 according to Chothia numbering as shown in Table E1a. In some embodiments, the VH region of a provided antibody or antigen-binding fragment thereof comprises a CDR-H1, a CDR-H2, and/or a CDR-H3 according to AbM numbering as shown in Table E1a. In some embodiments, the VH region of a provided antibody or antigen-binding fragment thereof comprises a CDR-H1, a CDR-H2, and/or a CDR-H3 according to Kabat numbering as shown in Table E1a. In some embodiments, the VH region of a provided antibody or antigen-binding fragment thereof comprises a CDR-H1, a CDR-H2, and/or a CDR-H3 according to Contact numbering as shown in Table E1a. In some embodiments, the VH region of a provided antibody or antigen-binding fragment thereof comprises a CDR-H1, a CDR-H2, and/or a CDR-H3 according to IMGT numbering as shown in Table E1a.

In some embodiments, the provided antibody or antigen-binding fragment thereof comprises a VH region comprising a CDR-H1, a CDR-H2, and a CDR-H3 selected from the group consisting of: a CDR-H1, a CDR-H2, and a CDR-H3 comprising the sequence of SEQ ID NOs: 35, 36, and 37, respectively; a CDR-H1, a CDR-H2, and a CDR-H3 comprising the sequence of SEQ ID NOs: 48, 49, and 37, respectively; a CDR-H1, a CDR-H2, and a CDR-H3 comprising the sequence of SEQ ID NOs: 58, 59, and 60, respectively; a CDR-H1, a CDR-H2, and a CDR-H3 comprising the sequence of SEQ ID NOs: 71, 72, and 60, respectively; a CDR-H1, a CDR-H2, and a CDR-H3 comprising the sequence of SEQ ID NOs: 82, 83, and 84, respectively; a CDR-H1, a CDR-H2, and a CDR-H3 comprising the sequence of SEQ ID NOs: 48, 95, and 84, respectively; a CDR-H1, a CDR-H2, and a CDR-H3 comprising the sequence of SEQ ID NOs: 48, 104, and 105, respectively; a CDR-H1, a CDR-H2, and a CDR-H3 comprising the sequence of SEQ ID NOs: 116, 117, 118, respectively; a CDR-H1, a CDR-H2, and a CDR-H3 comprising the sequence of SEQ ID NOs: 129, 130, and 131, respectively; a CDR-H1, a CDR-H2, and a CDR-H3 comprising the sequence of SEQ ID NOs: 142, 143, and 144, respectively; a CDR-H1, a CDR-H2, and a CDR-H3 comprising the sequence of SEQ ID NOs: 155, 156, and 157, respectively; a CDR-H1, a CDR-H2, and a CDR-H3 comprising the sequence of SEQ ID NOs: 168, 169, and 170, respectively; a CDR-H1, a CDR-H2, and a CDR-H3 comprising the sequence of SEQ ID NOs: 35, 181, and 182, respectively; a CDR-H1, a CDR-H2, and a CDR-H3 comprising the sequence of SEQ ID NOs: 168, 169, and 193, respectively; a CDR-H1, a CDR-H2, and a CDR-H3 comprising the sequence of SEQ ID NOs: 197, 198, and 199, respectively; according to Chothia numbering.

For example, the antibody or antigen-binding fragment thereof provided herein comprises a VH region comprising a CDR-H1, a CDR-H2, and a CDR-H3 comprising the sequences selected from among: SEQ ID NOs: 35, 36, and 37; SEQ ID NOs: 48, 49, and 37; SEQ ID NOs: 58, 59, and 60; SEQ ID NOs: 71, 72, and 60; SEQ ID NOs: 82, 83, and 84; SEQ ID NOs: 48, 95, and 84; SEQ ID NOs: 48, 104, and 105; SEQ ID NOs: 116, 117, 118; SEQ ID NOs: 129, 130, and 131; SEQ ID NOs: 142, 143, and 144, respectively, according to Chothia numbering.

In some embodiments, the antibody or antigen-binding fragment thereof provided herein comprises a VH region comprising a CDR-H1, a CDR-H2, and a CDR-H3 comprising the sequence selected from among: SEQ ID NOs: 35, 36, and 37; SEQ ID NOs: 48, 49, and 37; SEQ ID NOs: 82, 83, and 84; SEQ ID NOs: 48, 95, and 84, respectively, according to Chothia numbering. In some embodiments, the antibody or antigen-binding fragment thereof provided herein comprises a VH region comprising a CDR-H1, a CDR-H2, and a CDR-H3 comprising SEQ ID NOs: 35, 36, and 37, respectively, according to Chothia numbering. In some embodiments, the antibody or antigen-binding fragment thereof provided herein comprises a VH region comprising a CDR-H1, a CDR-H2, and a CDR-H3 comprising SEQ ID NOs: 82, 83, and 84, respectively, according to Chothia numbering.

In some embodiments, the antibody or antigen-binding fragment thereof provided herein comprises a VH region comprising a CDR-H1, a CDR-H2, and a CDR-H3 comprising SEQ ID NOs: 40, 41, and 37, respectively, according to Kabat numbering. In some embodiments, the antibody or antigen-binding fragment thereof provided herein comprises a VH region comprising a CDR-H1, a CDR-H2, and a CDR-H3 comprising SEQ ID NOs: 87, 88, and 84, respectively, according to Kabat numbering.

In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence selected from any one of SEQ ID NOs: 1-16. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence selected from any one of SEQ ID NOs: 1-11. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence selected from any one of SEQ ID NOs: 12-16. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence selected from any one of SEQ ID NOs: 330-334. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence of SEQ ID NO: 1. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence of SEQ ID NO: 2. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence of SEQ ID NO: 6. In some embodiments, the provided antibody or antigen-binding fragment thereof comprises a CDR-H1, a CDR-H2, and a CDR-H3, respectively, comprising the sequence of a CDR-H1, a CDR-H2, and a CDR-H3 contained within the VH region sequence of SEQ ID NO: 7.

Also provided are antibodies and antigen-binding fragments thereof having sequences at least at or about at least 90%, 91%, 92%, 93%, 94%, 95%, 96%, 97%, 98%, or 99% identical to such sequences. For example, provided herein is an antibody or antigen-binding fragment comprising a VH region comprising a sequence having at least 90%, 91%, 92%, 93%, 94%, 95%, 96%, 97%, 98%, or 99% sequence identity to a VH region sequence selected from any one of SEQ ID NOs: 1-16 and 330-334.

In some embodiments, the antibody is a single domain antibody (sdAb) comprising only a VH region sequence or a sufficient antigen-binding portion thereof, such as any of the above described VH sequences (e.g., a CDR-H1, a CDR-H2, and/or a CDR-H3).

In some embodiments, an antibody provided herein (e.g., an anti-PD-L1 antibody) or antigen-binding fragment thereof comprising a VH region further comprises a light chain or a sufficient antigen binding portion thereof. For example, in some embodiments, the antibody or antigen-binding fragment thereof contains a VH region and a VL region, or a sufficient antigen-binding portion of a VH and VL region. In such embodiments, a VH region sequence can be any of the above described VH sequence. In some such embodiments, the antibody is an antigen-binding fragment, such as a Fab or an scFv. In some such embodiments, the antibody is a full-length antibody that also contains a constant region.

In some embodiments, the antibody or antigen-binding fragment thereof, has a light chain variable (VL) region having the sequence selected from any one of SEQ ID NOs: 17-34, and 335-340, or a sequence that has at least 90%, 91%, 92%, 93%, 94%, 95%, 96%, 97%, 98%, or 99% sequence identity to a VL region sequence selected from any one of SEQ ID NOs: 17-34, and 335-340, or contains a CDR-L1, a CDR-L2, and/or a CDR-L3 present in such a VL sequence.

In some embodiments, the antibody or antigen-binding fragment thereof, has a VL having the sequence selected from any one of SEQ ID NOs: 17-28, or a sequence that has at least 90%, 91%, 92%, 93%, 94%, 95%, 96%, 97%, 98%, or 99% sequence identity to the VL region selected from any one of SEQ ID NOs: 17-28, or contains a CDR-L1, a CDR-L2, and/or a CDR-L3 present in such a VL sequence.

In some embodiments, the antibody or antigen-binding fragment thereof, has a light chain variable (VL) region having the sequence selected from any one of SEQ ID NOs: 29-34, or a sequence that has at least 90%, 91%, 92%, 93%, 94%, 95%, 96%, 97%, 98%, or 99% sequence identity to the VL region selected from any one of SEQ ID NOs: 29-34, or contains a CDR-L1, a CDR-L2, and/or a CDR-L3 present in such a VL sequence.

In some embodiments, the antibody or antigen-binding fragment thereof is a humanized antibody or antigen binding fragment that has a VL region comprising a CDR-L1, a CDR-L2, and/or a CDR-L3 from the VL regions selected from any one of SEQ ID NOs: 29-34. In some embodiments, the antibody or antigen-binding fragment thereof, has a light chain variable (VL) region having the sequence selected from any one of SEQ ID NOs: 335-340, or a sequence that has at least 90%, 91%, 92%, 93%, 94%, 95%, 96%, 97%, 98%, or 99% sequence identity to the VL region selected from any one of SEQ ID NOs: 335-340, or contains a CDR-L1, a CDR-L2, and/or a CDR-L3 present in such a VL sequence.

In some embodiments, the antibody or antigen-binding fragment thereof, has a VL having the sequence selected from any one of SEQ ID NOs: 21-24, or a sequence that has at least 90%, 91%, 92%, 93%, 94%, 95%, 96%, 97%, 98%, or 99% sequence identity to the VL region selected from any one of SEQ ID NOs: 21-24, or contains a CDR-L1, a CDR-L2, and/or a CDR-L3 present in such a VL sequence. In some embodiments, the antibody or antigen-binding fragment thereof, has a VL region set forth in SEQ ID NO:21, or a sequence that has at least 90%, 91%, 92%, 93%, 94%, 95%, 96%, 97%, 98%, or 99% sequence identity to SEQ ID NO: 21, or contains a CDR-L1, a CDR-L2, and/or a CDR-L3 present in such a VL sequence. In some embodiments, the antibody or antigen-binding fragment thereof, has a VL region set forth in SEQ ID NO:22, or a sequence that has at least 90%, 91%, 92%, 93%, 94%, 95%, 96%, 97%, 98%, or 99% sequence identity to SEQ ID NO:22, or contains a CDR-L1, a CDR-L2, and/or a CDR-L3 present in such a VL sequence. In some embodiments, the antibody or antigen-binding fragment thereof, has a VL region set forth in SEQ ID NO:23, or a sequence that has at least 90%, 91%, 92%, 93%, 94%, 95%, 96%, 97%, 98%, or 99% sequence identity to SEQ ID NO: 23, or contains a CDR-L1, a CDR-L2, and/or a CDR-L3 present in such a VL sequence. In some embodiments, the antibody or antigen-binding fragment thereof, has a VL region set forth in SEQ ID NO:24, or a sequence that has at least 90%, 91%, 92%, 93%, 94%, 95%, 96%, 97%, 98%, or 99% sequence identity to SEQ ID NO:24, or contains a CDR-L1, a CDR-L2, and/or a CDR-L3 present in such a VL sequence.

In some embodiments, the provided antibody or antigen-binding fragment thereof comprises a CDR-L1, a CDR-L2, and/or a CDR-L3, according to Chothia numbering in a VL comprising the sequence selected from any one of SEQ ID NOs: 17-34. In some embodiments, the provided antibody or antigen-binding fragment thereof comprises a CDR-L1, a CDR-L2, and/or a CDR-L3, according to AbM numbering in a VL comprising the sequence selected from any one of SEQ ID NOs: 17-34. In some embodiments, the provided antibody or antigen-binding fragment thereof comprises a CDR-L1, a CDR-L2, and/or a CDR-L3, according to Kabat numbering in a VL comprising the sequence selected from any one of SEQ ID NOs: 17-34. In some embodiments, the provided antibody or antigen-binding fragment thereof comprises a CDR-L1, a CDR-L2, and/or a CDR-L3, according to Contact numbering in a VL comprising the sequence selected from any one of SEQ ID NOs: 17-34. In some embodiments, the provided antibody or antigen-binding fragment thereof comprises a CDR-L1, a CDR-L2, and/or a CDR-L3, according to IMGT numbering in a VL comprising the sequence selected from any one of SEQ ID NOs: 17-34.

In some embodiments, the provided antibody or antigen-binding fragment thereof comprises a CDR-L1, a CDR-L2, and/or a CDR-L3, according to Chothia numbering in a VL comprising the sequence selected from any one of SEQ ID NOs: 17-28. In some embodiments, the provided antibody or antigen-binding fragment thereof comprises a CDR-L1, a CDR-L2, and/or a CDR-L3, according to AbM numbering in a VL comprising the sequence selected from any one of SEQ ID NOs: 17-28. In some embodiments, the provided antibody or antigen-binding fragment thereof comprises a CDR-L1, a CDR-L2, and/or a CDR-L3, according to Kabat numbering in a VL comprising the sequence selected from any one of SEQ ID NOs: 17-28. In some embodiments, the provided antibody or antigen-binding fragment thereof comprises a CDR-L1, a CDR-L2, and/or a CDR-L3, according to Contact numbering in a VL comprising the sequence selected from any one of SEQ ID NOs: 17-28. In some embodiments, the provided antibody or antigen-binding fragment thereof comprises a CDR-L1, a CDR-L2, and/or a CDR-L3, according to IMGT numbering in a VL comprising the sequence selected from any one of SEQ ID NOs: 17-28.

In some embodiments, the provided antibody or antigen-binding fragment thereof comprises a CDR-L1, a CDR-L2, and/or a CDR-L3, according to Chothia numbering in a VL comprising the sequence selected from any one of SEQ ID NOs: 29-34. In some embodiments, the provided antibody or antigen-binding fragment thereof comprises a CDR-L1, a CDR-L2, and/or a CDR-L3, according to AbM numbering in a VL comprising the sequence selected from any one of SEQ ID NOs: 29-34. In some embodiments, the provided antibody or antigen-binding fragment thereof comprises a CDR-L1, a CDR-L2, and/or a CDR-L3, according to Kabat numbering in a VL comprising the sequence selected from any one of SEQ ID NOs: 29-34. In some embodiments, the provided antibody or antigen-binding fragment thereof comprises a CDR-L1, a CDR-L2, and/or a CDR-L3, according to Contact numbering in a VL comprising the sequence selected from any one of SEQ ID NOs: 29-34. In some embodiments, the provided antibody or antigen-binding fragment thereof comprises a CDR-L1, a CDR-L2, and/or a CDR-L3, according to IMGT numbering in a VL comprising the sequence selected from any one of SEQ ID NOs: 29-34.

In some embodiments, the provided antibody or antigen-binding fragment thereof comprises a CDR-L1, a CDR-L2, and/or a CDR-L3, according to Chothia numbering in a VL comprising the sequence selected from any one of SEQ ID NOs: 335-340. In some embodiments, the provided antibody or antigen-binding fragment thereof comprises a CDR-L1, a CDR-L2, and/or a CDR-L3, according to AbM numbering in a VL comprising the sequence selected from any one of SEQ ID NOs: 335-340. In some embodiments, the provided antibody or antigen-binding fragment thereof comprises a CDR-L1, a CDR-L2, and/or a CDR-L3, according to Kabat numbering in a VL comprising the sequence selected from any one of SEQ ID NOs: 335-340. In some embodiments, the provided antibody or antigen-binding fragment thereof comprises a CDR-L1, a CDR-L2, and/or a CDR-L3, according to Contact numbering in a VL comprising the sequence selected from any one of SEQ ID NOs: 335-340. In some embodiments, the provided antibody or antigen-binding fragment thereof comprises a CDR-L1, a CDR-L2, and/or a CDR-L3, according to IMGT numbering in a VL comprising the sequence selected from any one of SEQ ID NOs: 335-340.

In some embodiments, the VL region of a provided antibody or antigen-binding fragment thereof comprises a CDR-L1, a CDR-L2, and/or a CDR-L3 according to Chothia numbering as shown in Table E1b. In some embodiments, the VL region of a provided antibody or antigen-binding fragment thereof comprises a CDR-L1, a CDR-L2, and/or a CDR-L3 according to AbM numbering as shown in Table E1b. In some embodiments, the VL region of a provided antibody or antigen-binding fragment thereof comprises a CDR-L1, a CDR-L2, and/or a CDR-L3 according to Kabat numbering as shown in Table E1b. In some embodiments, the VL region of a provided antibody or antigen-binding fragment thereof comprises a CDR-L1, a CDR-L2, and/or a CDR-L3 according to Contact numbering as shown in Table E1b. In some embodiments, the VL region of a provided antibody or antigen-binding fragment thereof comprises a CDR-L1, a CDR-L2, and/or a CDR-L3 according to IMGT numbering as shown in Table E1b.

In some embodiments of the antibody or antigen-binding fragment thereof provided herein, the VL region comprises a CDR-L1, a CDR-L2, and a CDR-L3 selected from among: a CDR-L1, a CDR-L2, and a CDR-L3 comprising the sequence of SEQ ID NOs: 210, 211, and 212, respectively; a CDR-L1, a CDR-L2, and a CDR-L3 comprising the sequence of SEQ ID NOs: 218, 211, and 212, respectively; a CDR-L1, a CDR-L2, and a CDR-L3 comprising the sequence of SEQ ID NOs: 221, 222, and 223, respectively; a CDR-L1, a CDR-L2, and a CDR-L3 comprising the sequence of SEQ ID NOs: 229, 222, and 223, respectively; a CDR-L1, a CDR-L2, and a CDR-L3 comprising the sequence of SEQ ID NOs: 233, 234, and 235, respectively; a CDR-L1, a CDR-L2, and a CDR-L3 comprising the sequence of SEQ ID NOs: 241, 234, and 242, respectively; a CDR-L1, a CDR-L2, and a CDR-L3 comprising the sequence of SEQ ID NOs: 246, 247, and 248, respectively; a CDR-L1, a CDR-L2, and a CDR-L3 comprising the sequence of SEQ ID NOs: 246, 254, and 255, respectively; a CDR-L1, a CDR-L2, and a CDR-L3 comprising the sequence of SEQ ID NOs: 258, 259, and 260, respectively; a CDR-L1, a CDR-L2, and a CDR-L3 comprising the sequence of SEQ ID NOs: 265, 266, and 267, respectively; a CDR-L1, a CDR-L2, and a CDR-L3 comprising the sequence of SEQ ID NOs: 246, 273, and 274, respectively; a CDR-L1, a CDR-L2, and a CDR-L3 comprising the sequence of SEQ ID NOs: 278, 279, and 280, respectively; a CDR-L1, a CDR-L2, and a CDR-L3 comprising the sequence of SEQ ID NOs: 286, 287, and 288, respectively; a CDR-L1, a CDR-L2, and a CDR-L3 comprising the sequence of SEQ ID NOs: 294, 234, and 295, respectively; a CDR-L1, a CDR-L2, and a CDR-L3 comprising the sequence of SEQ ID NOs: 299, 300, and 301, respectively; a CDR-L1, a CDR-L2, and a CDR-L3 comprising the sequence of SEQ ID NOs: 306, 234, and 307, respectively; a CDR-L1, a CDR-L2, and a CDR-L3 comprising the sequence of SEQ ID NOs: 311, 312, and 313, respectively; a CDR-L1, a CDR-L2, and a CDR-L3 comprising the sequence of SEQ ID NOs: 319, 320, and 321, respectively.

In some embodiments, the antibody or antigen-binding fragment thereof provided herein comprises a VL region comprising a CDR-L1, a CDR-L2, and a CDR-L3 comprising the sequence selected from among: SEQ ID NOs: 210, 211, and 212; SEQ ID NOs: 218, 211, and 212; SEQ ID NOs: 221, 222, and 223; SEQ ID NOs: 229, 222, and 223; SEQ ID NOs: 233, 234, and 235; SEQ ID NOs: 241, 234, and 242; SEQ ID NOs: 246, 247, and 248; SEQ ID NOs: 246, 254, and 255; SEQ ID NOs: 258, 259, and 260; SEQ ID NOs: 265, 266, and 267; SEQ ID NOs: 246, 273, and 274; SEQ ID NOs: 278, 279, and 280, respectively, according to Chothia numbering. In some embodiments, the antibody or antigen-binding fragment thereof provided herein comprises a VL region comprising a CDR-L1, a CDR-L2, and a CDR-L3 comprising SEQ ID NOs: 233, 234, and 235, respectively, according to Chothia numbering. In some embodiments, the antibody or antigen-binding fragment thereof provided herein comprises a VL region comprising a CDR-L1, a CDR-L2, and a CDR-L3 comprising SEQ ID NOs: 246, 247, and 248, respectively, according to Chothia numbering.

In some embodiments, the antibody or antigen-binding fragment thereof provided herein comprises a VL region comprising a CDR-L1, a CDR-L2, and a CDR-L3 comprising SEQ ID NOs: 233, 234, and 235, respectively, according to Kabat numbering. In some embodiments, the antibody or antigen-binding fragment thereof provided herein comprises a VL region comprising a CDR-L1, a CDR-L2, and a CDR-L3 comprising SEQ ID NOs: 246, 247, and 248, respectively, according to Kabat numbering.

In some embodiments, the antibody or antigen-binding fragment thereof provided herein comprises a VL region comprising a CDR-L1, a CDR-L2, and a CDR-L3 comprising the sequence selected from among: SEQ ID NOs: 233, 234, and 235; SEQ ID NOs: 241, 234, and 242; SEQ ID NOs: 246, 247, and 248; SEQ ID NOs: 246, 254, and 255, according to Kabat numbering, Chothia numbering or AbM numbering.

In some embodiments, the provided antibody or antigen-binding fragment thereof contains a CDR-L1, a CDR-L2, and a CDR-L3, respectively, contained within the VL region sequence selected from any one of SEQ ID NOs: 17-34. In some embodiments, the provided antibody or antigen-binding fragment thereof contains a CDR-L1, a CDR-L2, and a CDR-L3, respectively, contained within the VL region sequence selected from any one of SEQ ID NOs: 17-28. In some embodiments, the provided antibody or antigen-binding fragment thereof contains a CDR-L1, a CDR-L2, and a CDR-L3, respectively, contained within the VL region sequence selected from any one of SEQ ID NOs: 29-34. In some embodiments, the provided antibody or antigen-binding fragment thereof contains a CDR-L1, a CDR-L2, and a CDR-L3, respectively, contained within the VL region sequence selected from any one of SEQ ID NOs: 335-340. In some embodiments, the provided antibody contains a CDR-L1, a CDR-L2, and a CDR-L3, respectively, contained within the VL region sequence of SEQ ID NO:21. In some embodiments, the provided antibody contains a CDR-L1, a CDR-L2, and a CDR-L3, respectively, contained within the VL region sequence of SEQ ID NO:22. In some embodiments, the provided antibody contains a CDR-L1, a CDR-L2, and a CDR-L3, respectively, contained within the VL region sequence of SEQ ID NO:23. In some embodiments, the provided antibody contains a CDR-L1, a CDR-L2, and a CDR-L3, respectively, contained within the VL region sequence of SEQ ID NO:24.

In some embodiments, the provided antibody or antigen-binding fragment thereof comprises a VL region comprising an sequence selected from any one of SEQ ID NOs: 17-34. In some embodiments, the provided antibody or antigen-binding fragment thereof comprises a VL region comprising an sequence selected from any one of SEQ ID NOs: 17-28. In some embodiments, the provided antibody or antigen-binding fragment thereof comprises a VL region comprising an sequence selected from any one of SEQ ID NOs: 29-34. In some embodiments, the provided antibody or antigen-binding fragment thereof contains a VL region comprises the sequence of SEQ ID NO:21. In some embodiments, the provided antibody or antigen-binding fragment thereof contains a VL region comprises the sequence of SEQ ID NO:22. In some embodiments, the provided antibody or antigen-binding fragment thereof contains a VL region comprises the sequence of SEQ ID NO:23. In some embodiments, the provided antibody or antigen-binding fragment thereof contains a VL region comprises the sequence of SEQ ID NO: 24. Also provided are antibodies having sequences at least at or about at least 90%, 91%, 92%, 93%, 94%, 95%, 96%, 97%, 98%, or 99% identical to any of such sequences.

In some embodiments, the VH region of the antibody or fragment comprises the sequence selected from any one of SEQ ID NOs: 1-16 and the VL region of the antibody or fragment comprises the sequence selected from any one of SEQ ID NOs: 17-34. In some embodiments, the VH region of the antibody or fragment comprises the sequence selected from any one of SEQ ID NOs: 1-11 and the VL region of the antibody or fragment comprises the sequence selected from any one of SEQ ID NOs: 17-28. In some embodiments, the VH region of the antibody or fragment comprises the sequence selected from any one of SEQ ID NOs: 330-334 and the VL region of the antibody or fragment comprises the sequence selected from any one of SEQ ID NOs: 335-340. In some embodiments, the VH region of the antibody or fragment comprises the sequence selected from any one of SEQ ID NOs: 1, 2, 6, and 7 and the VL region of the antibody or fragment comprises the sequence selected from any one of SEQ ID NOs: 21-24. In some embodiments, the VH region of the antibody or fragment comprises the sequence set forth in SEQ ID NO:1 and the VL region of the antibody or fragment comprises the sequence set forth in SEQ ID NO:21. In some embodiments, the VH region of the antibody or fragment comprises the sequence set forth in SEQ ID NO:2 and the VL region of the antibody or fragment comprises the sequence set forth in SEQ ID NO:22. In some embodiments, the VH region of the antibody or fragment comprises the sequence set forth in SEQ ID NO:6 and the VL region of the antibody or fragment comprises the sequence set forth in SEQ ID NO:23. In some embodiments, the VH region of the antibody or fragment comprises the sequence set forth in SEQ ID NO:7 and the VL region of the antibody or fragment comprises the sequence set forth in SEQ ID NO:24.

Also provided are antibodies and antigen-binding fragments thereof having sequences at least at or about at least 90%, 91%, 92%, 93%, 94%, 95%, 96%, 97%, 98%, or 99% identical to such sequences. For example, provided herein is an antibody or antigen-binding fragment containing a VH region comprising an sequence having at least 90%, 91%, 92%, 93%, 94%, 95%, 96%, 97%, 98%, or 99% sequence identity to a VH region sequence selected from any one of SEQ ID NOs: 1-16 and/or comprising an sequence having at least 90%, 91%, 92%, 93%, 94%, 95%, 96%, 97%, 98%, or 99% sequence identity to a VH region sequence selected from any one of SEQ ID NOs: 17-34. In some embodiments, the antibody or antigen-binding fragment contains a VH region comprising an sequence having at least 90%, 91%, 92%, 93%, 94%, 95%, 96%, 97%, 98%, or 99% sequence identity to a VH region sequence selected from any one of SEQ ID NOs: 1-11 and a VL region having at least 90%, 91%, 92%, 93%, 94%, 95%, 96%, 97%, 98%, or 99% sequence identity to a VL region sequence selected from any one of SEQ ID NOs: 17-28. In some embodiments, the antibody or antigen-binding fragment contains a VH region comprising an sequence having at least 90%, 91%, 92%, 93%, 94%, 95%, 96%, 97%, 98%, or 99% sequence identity to a VH region sequence selected from any one of SEQ ID NOs: 330-334 and a VL region having at least 90%, 91%, 92%, 93%, 94%, 95%, 96%, 97%, 98%, or 99% sequence identity to a VL region sequence selected from any one of SEQ ID NOs: 335-340. In some embodiments, the antibody or antigen-binding fragment contains a VH region comprising an sequence having at least 90%, 91%, 92%, 93%, 94%, 95%, 96%, 97%, 98%, or 99% sequence identity to a VH region sequence selected from any one of SEQ ID NOs: 1, 2, 6, and 7 and a VL region having at least 90%, 91%, 92%, 93%, 94%, 95%, 96%, 97%, 98%, or 99% sequence identity to a VL region sequence selected from any one of SEQ ID NOs: 21-24. In some embodiments, the antibody or antigen-binding fragment contains a VH region comprising an sequence having at least 90%, 91%, 92%, 93%, 94%, 95%, 96%, 97%, 98%, or 99% sequence identity to the sequence set forth in SEQ ID NO:1 and a VL region having at least 90%, 91%, 92%, 93%, 94%, 95%, 96%, 97%, 98%, or 99% sequence identity to the sequence set forth in SEQ ID NO:21. In some embodiments, the antibody or antigen-binding fragment contains a VH region comprising an sequence having at least 90%, 91%, 92%, 93%, 94%, 95%, 96%, 97%, 98%, or 99% sequence identity to the sequence set forth in SEQ ID NO:2 and a VL region having at least 90%, 91%, 92%, 93%, 94%, 95%, 96%, 97%, 98%, or 99% sequence identity to the sequence set forth in SEQ ID NO:22. In some embodiments, the antibody or antigen-binding fragment contains a VH region comprising an sequence having at least 90%, 91%, 92%, 93%, 94%, 95%, 96%, 97%, 98%, or 99% sequence identity to the sequence set forth in SEQ ID NO:6 and a VL region having at least 90%, 91%, 92%, 93%, 94%, 95%, 96%, 97%, 98%, or 99% sequence identity to the sequence set forth in SEQ ID NO:23. In some embodiments, the antibody or antigen-binding fragment contains a VH region comprising an sequence having at least 90%, 91%, 92%, 93%, 94%, 95%, 96%, 97%, 98%, or 99% sequence identity to the sequence set forth in SEQ ID NO:7 and a VL region having at least 90%, 91%, 92%, 93%, 94%, 95%, 96%, 97%, 98%, or 99% sequence identity to the sequence set forth in SEQ ID NO:24.

In some embodiments, the antibody or antigen-binding fragment comprises a VH having at least 90% sequence identity to the VH sequence selected from any of SEQ ID NOs: 1-16; and a VL having at least 90% sequence identity to the VL sequence selected from any of SEQ ID NOs: 17-34.

In some embodiments, the antibody or antigen-binding fragment comprises a VH having at least 90% sequence identity to the VH sequence selected from any of SEQ ID NOs: 1, 2, 6, and 7; and a VL having at least 90% sequence identity to the VL sequence selected from any of SEQ ID NOs: 21-24. In some embodiments, said antibody or antigen-binding fragment comprises a VH having at least 90% sequence identity to the VH sequence of SEQ ID NO:1; and a VL having at least 90% sequence identity to the VL sequence of SEQ ID NO:21. In some embodiments, said antibody or antigen-binding fragment comprises a VH having at least 90% sequence identity to the VH sequence of SEQ ID NO:2; and a VL having at least 90% sequence identity to the VL sequence of SEQ ID NO:22. In some embodiments, said antibody or antigen-binding fragment comprises a VH having at least 90% sequence identity to the VH sequence of SEQ ID NO:6; and a VL having at least 90% sequence identity to the VL sequence of SEQ ID NO: 23. In some embodiments, said antibody or antigen-binding fragment comprises a VH having at least 90% sequence identity to the VH sequence of SEQ ID NO:7; and a VL having at least 90% sequence identity to the VL sequence of SEQ ID NO:24.

In some embodiments, the VH region of the antibody or antigen-binding fragment thereof comprises a CDR-H1, a CDR-H2, a CDR-H3, respectively, comprising the sequences of a CDR-H1, a CDR-H2, and a CDR-H3 contained within the VH region sequence selected from any one of SEQ ID NOs: 1-16; and the VL region of the antibody or antigen-binding fragment thereof comprises a CDR-L1, a CDR-L2, a CDR-L3, respectively, comprising the sequences of a CDR-L1, a CDR-L2, and a CDR-L3, respectively contained within the VL region sequence selected from any one of SEQ ID NOs: 17-34.

In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence selected from any one of SEQ ID NOs: 1-11 and the VL region of the antibody or fragment comprises a CDR-L1, a CDR-L2, a CDR-L3, respectively, comprising the sequences of a CDR-L1, a CDR-L2, and a CDR-L3, respectively contained within the VL region sequence selected from any one of SEQ ID NOs: 17-28.

In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence selected from any one of SEQ ID NOs: 12-16 and the VL region of the antibody or fragment comprises a CDR-L1, a CDR-L2, a CDR-L3, respectively, comprising the sequences of a CDR-L1, a CDR-L2, and a CDR-L3, respectively contained within the VL region sequence selected from any one of SEQ ID NOs: 29-34.

In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence selected from any one of SEQ ID NOs: 330-334 and the VL region of the antibody or fragment comprises a CDR-L1, a CDR-L2, a CDR-L3, respectively, comprising the sequences of a CDR-L1, a CDR-L2, and a CDR-L3, respectively contained within the VL region sequence selected from any one of SEQ ID NOs: 335-340.

In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence selected from any one of SEQ ID NOs: 1, 2, 6, and 7 and the VL region of the antibody or fragment comprises a CDR-L1, a CDR-L2, a CDR-L3, respectively, comprising the sequences of a CDR-L1, a CDR-L2, and a CDR-L3, respectively contained within the VL region sequence selected from any one of SEQ ID NOs: 21-24.

In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:1 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:17. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:2 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:18. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:3 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:19. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:4 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:19. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO: 5 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:20. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:1 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:21. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:2 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:22. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:6 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:23. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:7 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:24. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:8 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:25. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:9 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:26. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:10 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:27. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:11 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:28. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:12 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:29. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:13 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:30. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:14 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:31. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:15 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:32. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:12 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:33. In some embodiments, the VH region of the antibody or fragment comprises a CDR-H1, a CDR-H2, a CDR-H3, respectively, comprising the sequences of a CDR-H1, a CDR-H2, and a CDR-H3 contained within the VH region sequence set forth in SEQ ID NO:16 and the VL region of the antibody or fragment comprises a CDR-L1, a CDR-L2, a CDR-L3, respectively, comprising the sequences of a CDR-L1, a CDR-L2, and a CDR-L3, respectively, contained within the VL region sequence set forth in SEQ ID NO:34.

In some embodiments, the antibody or antigen-binding fragment thereof comprises a VH comprising a CDR-H1, a CDR-H2, and a CDR-H3, wherein: the CDR-H1 comprises the sequence selected from any of SEQ ID NOs: 35, 38, 40, 42, 45, 48, 50, 52, 54, 56, 58, 61, 63, 65, 68, 71, 73, 75, 77, 79, 82, 85, 87, 89, 92, 96, 98, 100, 102, 106, 108, 110, 113, 116, 119, 121, 123, 126, 129, 132, 134, 136, 139, 142, 145, 147, 149, 152, 155, 158, 160, 162, 165, 168, 171, 173, 175, 178, 183, 185, 187, 190, 197, 200, 202, and 204, and 207; the CDR-H2 comprises the sequence selected from any of SEQ ID NOs: 36, 39, 41, 43, 46, 49, 51, 53, 55, 57, 59, 62, 64, 66, 69, 72, 74, 76, 78, 80, 83, 86, 88, 90, 93, 95, 97, 99, 101, 103, 104, 107, 109, 111, 114, 117, 120, 122, 124, 127, 130, 133, 135, 137, 140, 143, 146, 148, 150, 153, 156, 159, 161, 163, 166, 169, 172, 174, 176, 179, 181, 184, 186, 188, 191, 194, 198, 201, 203, and 205208; and the CDR-H3 comprises the sequence selected from any of SEQ ID NOs: 37, 44, 47, 60, 67, 70, 81, 84, 91, 94, 105, 112, 115, 118, 125, 128, 131, 138, 141, 144, 151, 154, 157, 164, 167, 170, 177, 180, 182, 189, 192, 193, 195, 196, 199, 206, and 209; and a VL comprising a CDR-L1, a CDR-L2, and a CDR-L3, wherein: the CDR-L1 comprises the sequence selected from any of SEQ ID NOs: 210, 213, 216, 218, 219, 220, 221, 224, 227, 229, 230, 232, 233, 236, 239, 241, 243, 245, 246, 249, 252, 258, 261, 264, 265, 268, 271, 278, 281, 284, 286, 289, 292, 294, 296, 298, 299, 302, 306, 308, 310, 311, 314, 317, 319, 322, and 325; the CDR-L2 comprises the sequence selected from any of SEQ ID NOs: 211, 214, 217, 222, 225, 228, 231, 234, 237, 240, 247, 250, 253, 254, 256, 259, 262, 266, 269, 272, 273, 275, 277, 279, 282, 285, 287, 290, 293, 300, 303, 305, 312, 315, 318, 320, 323, and 326; and the CDR-L3 comprises the sequence selected from any of SEQ ID NOs: 212, 215, 223, 226, 235, 238, 242, 244, 248, 251, 255, 257, 260, 263, 267, 270, 274, 276, 280, 283, 288, 291, 295, 297, 301, 304, 307, 309, 313, 316, 321, and 324.

In some embodiments, the CDR-H1 comprises the sequence selected from any of SEQ ID NOs: 35, 38, 40, 42, 45, 48, 50, 52, 54, 56, 82, 85, 87, 89, 92, 96, 98, 100, and 102; the CDR-H2 comprises the sequence selected from any of SEQ ID NOs: 36, 39, 41, 43, 46, 49, 51, 53, 55, 57, 83, 86, 88, 90, 93, 95, 97, 99, 101, and 103; and the CDR-H3 comprises the sequence selected from any of SEQ ID NOs: 37, 44, 47, 84, 91, and 94; and the CDR-L1 comprises the sequence selected from any of SEQ ID NOs: 233, 236, 239, 241, 243, 245, 246, 249, and 252; the CDR-L2 comprises the sequence selected from any of SEQ ID NOs: 217, 234, 237, 240, 247, 250, 253, 254, and 256; and the CDR-L3 comprises the sequence selected from any of SEQ ID NOs: 235, 238, 242, 244, 248, 251, 255, and 257. In some embodiments, the CDR-H1 comprises the sequence of SEQ ID NO:35; the CDR-H2 comprises the sequence of SEQ ID NO:36; and the CDR-H3 comprises the sequence of SEQ ID NO:37; and the CDR-L1 comprises the sequence of SEQ ID NO:233; the CDR-L2 comprises the sequence of SEQ ID NO:234; and the CDR-L3 comprises the sequence of SEQ ID NO:235. In some embodiments, the CDR-H1 comprises the sequence of SEQ ID NO:82; the CDR-H2 comprises the sequence of SEQ ID NO: 83; and the CDR-H3 comprises the sequence of SEQ ID NO:84; and the CDR-L1 comprises the sequence of SEQ ID NO:246; the CDR-L2 comprises the sequence of SEQ ID NO:247; and the CDR-L3 comprises the sequence of SEQ ID NO:248.

In some embodiments, the antibody or antigen-binding fragment thereof comprises a VH and a VL, wherein: the VH comprises a CDR-H1, a CDR-H2, and a CDR-H3 comprising SEQ ID NOS: 35, 36, and 37, respectively, and the VL comprises a CDR-L1, a CDR-L2, and a CDR-L3 comprising SEQ ID NOS: 210, 211, and 212, respectively; the VH comprises a CDR-H1, a CDR-H2, and a CDR-H3 comprising SEQ ID NOS: 48, 49, and 37, respectively, and the VL comprises a CDR-L1, a CDR-L2, and a CDR-L3 comprising SEQ ID NOS: 218, 211, and 212, respectively; the VH comprises a CDR-H1, a CDR-H2, and a CDR-H3 comprising SEQ ID NOS: 58, 59, and 60, respectively, and the VL comprises a CDR-L1, a CDR-L2, and a CDR-L3 comprising SEQ ID NOS: 221, 222, and 223, respectively; the VH comprises a CDR-H1, a CDR-H2, and a CDR-H3 comprising SEQ ID NOS: 58, 59, and 60, respectively, and the VL comprises a CDR-L1, a CDR-L2, and a CDR-L3 comprising SEQ ID NOS: 221, 222, and 223, respectively; the VH comprises a CDR-H1, a CDR-H2, and a CDR-H3 comprising SEQ ID NOS: 71, 72, and 60, respectively, and the VL comprises a CDR-L1, a CDR-L2, and a CDR-L3 comprising SEQ ID NOS: 229, 222, and 223, respectively; the VH comprises a CDR-H1, a CDR-H2, and a CDR-H3 comprising SEQ ID NOS: 35, 36, and 37, respectively, and the VL comprises a CDR-L1, a CDR-L2, and a CDR-L3 comprising SEQ ID NOS: 233, 234, and 235, respectively; the VH comprises a CDR-H1, a CDR-H2, and a CDR-H3 comprising SEQ ID NOS: 48, 49, and 37, respectively, and the VL comprises a CDR-L1, a CDR-L2, and a CDR-L3 comprising SEQ ID NOS: 241, 234, and 242, respectively; the VH comprises a CDR-H1, a CDR-H2, and a CDR-H3 comprising SEQ ID NOS: 82, 83, and 84, respectively, and the VL comprises a CDR-L1, a CDR-L2, and a CDR-L3 comprising SEQ ID NOS: 246, 247, and 248, respectively; the VH comprises a CDR-H1, a CDR-H2, and a CDR-H3 comprising SEQ ID NOS: 48, 95, and 84, respectively, and the VL comprises a CDR-L1, a CDR-L2, and a CDR-L3 comprising SEQ ID NOS: 246, 254, and 255, respectively; the VH comprises a CDR-H1, a CDR-H2, and a CDR-H3 comprising SEQ ID NOS: 48, 104, and 105, respectively, and the VL comprises a CDR-L1, a CDR-L2, and a CDR-L3 comprising SEQ ID NOS: 258, 259, and 260, respectively; the VH comprises a CDR-H1, a CDR-H2, and a CDR-H3 comprising SEQ ID NOS: 116, 117, and 118, respectively, and the VL comprises a CDR-L1, a CDR-L2, and a CDR-L3 comprising SEQ ID NOS: 265, 266, and 267, respectively; the VH comprises a CDR-H1, a CDR-H2, and a CDR-H3 comprising SEQ ID NOS: 129, 130, and 131, respectively, and the VL comprises a CDR-L1, a CDR-L2, and a CDR-L3 comprising SEQ ID NOS: 246, 273, and 274, respectively; the VH comprises a CDR-H1, a CDR-H2, and a CDR-H3 comprising SEQ ID NOS: 142, 143, and 144, respectively, and the VL comprises a CDR-L1, a CDR-L2, and a CDR-L3 comprising SEQ ID NOS: 278, 279, and 280, respectively; the VH comprises a CDR-H1, a CDR-H2, and a CDR-H3 comprising SEQ ID NOS: 155, 156, and 157, respectively, and the VL comprises a CDR-L1, a CDR-L2, and a CDR-L3 comprising SEQ ID NOS: 286, 287, and 288, respectively; the VH comprises a CDR-H1, a CDR-H2, and a CDR-H3 comprising SEQ ID NOS: 168, 169, and 170, respectively, and the VL comprises a CDR-L1, a CDR-L2, and a CDR-L3 comprising SEQ ID NOS: 294, 234, and 295, respectively; the VH comprises a CDR-H1, a CDR-H2, and a CDR-H3 comprising SEQ ID NOS: 35, 181, and 182, respectively, and the VL comprises a CDR-L1, a CDR-L2, and a CDR-L3 comprising SEQ ID NOS: 299, 300, and 301, respectively; the VH comprises a CDR-H1, a CDR-H2, and a CDR-H3 comprising SEQ ID NOS: 168, 169, and 193, respectively, and the VL comprises a CDR-L1, a CDR-L2, and a CDR-L3 comprising SEQ ID NOS: 306, 234, and 307, respectively; the VH comprises a CDR-H1, a CDR-H2, and a CDR-H3 comprising SEQ ID NOS: 155, 156, and 157, respectively, and the VL comprises a CDR-L1, a CDR-L2, and a CDR-L3 comprising SEQ ID NOS: 311, 312, and 313, respectively; the VH comprises a CDR-H1, a CDR-H2, and a CDR-H3 comprising SEQ ID NOS: 197, 198, and 199, respectively, and the VL comprises a CDR-L1, a CDR-L2, and a CDR-L3 comprising SEQ ID NOS: 319, 320, and 321, respectively.

In some embodiments, the antibody or antigen-binding fragment thereof comprises a VH and a VL, wherein: the VH comprises a CDR-H1, a CDR-H2, and a CDR-H3 comprising SEQ ID NOS: 35, 36, and 37, respectively, and the VL comprises a CDR-L1, a CDR-L2, and a CDR-L3 comprising SEQ ID NOS: 233, 234, and 235, respectively; the VH comprises a CDR-H1, a CDR-H2, and a CDR-H3 comprising SEQ ID NOS: 48, 49, and 37, respectively, and the VL comprises a CDR-L1, a CDR-L2, and a CDR-L3 comprising SEQ ID NOS: 241, 234, and 242, respectively; the VH comprises a CDR-H1, a CDR-H2, and a CDR-H3 comprising SEQ ID NOS: 82, 83, and 84, respectively, and the VL comprises a CDR-L1, a CDR-L2, and a CDR-L3 comprising SEQ ID NOS: 246, 247, and 248, respectively; the VH comprises a CDR-H1, a CDR-H2, and a CDR-H3 comprising SEQ ID NOS: 48, 95, and 84, respectively, and the VL comprises a CDR-L1, a CDR-L2, and a CDR-L3 comprising SEQ ID NOS: 246, 254, and 255, respectively. In some embodiments, the antibody or antigen-binding fragment thereof provided herein comprises a VH region comprising a CDR-H1, a CDR-H2, and a CDR-H3 comprising SEQ ID NOs: 35, 36, and 37, respectively, and a VL region comprising a CDR-L1, a CDR-L2, and a CDR-L3 comprising SEQ ID NOs: 233, 234, and 235, respectively, according to Chothia numbering. In some embodiments, the antibody or antigen-binding fragment thereof provided herein comprises a VH region comprising a CDR-H1, a CDR-H2, and a CDR-H3 comprising SEQ ID NOs: 82, 83, and 84, respectively, and a VL region comprising a CDR-L1, a CDR-L2, and a CDR-L3 comprising SEQ ID NOs: 246, 247, and 248, respectively, according to Chothia numbering.

In some embodiments, the antibody or antigen-binding fragment thereof provided herein comprises a VH region comprising a CDR-H1, a CDR-H2, and a CDR-H3 comprising SEQ ID NOs: 40, 41, and 37, respectively, and a VL region comprising a CDR-L1, a CDR-L2, and a CDR-L3 comprising SEQ ID NOs: 233, 234, and 235, respectively, according to Kabat numbering. In some embodiments, the antibody or antigen-binding fragment thereof provided herein comprises a VH region comprising a CDR-H1, a CDR-H2, and a CDR-H3 comprising SEQ ID NOs: 87, 88, and 84, respectively, and a VL region comprising a CDR-L1, a CDR-L2, and a CDR-L3 comprising SEQ ID NOs: 246, 247, and 248, respectively, according to Kabat numbering.

In some embodiments, the antibody or antigen-binding fragment thereof comprises a VH and a VL, wherein: the VH comprises a CDR-H1, a CDR-H2, and a CDR-H3 comprising SEQ ID NOS: 40, 41, and 37, respectively, and the VL comprises a CDR-L1, a CDR-L2, and a CDR-L3 comprising SEQ ID NOS: 210, 211, and 212, respectively; the VH comprises a CDR-H1, a CDR-H2, and a CDR-H3 comprising SEQ ID NOS: 52, 53, and 37, respectively, and the VL comprises a CDR-L1, a CDR-L2, and a CDR-L3 comprising SEQ ID NOS: 218, 211, and 212, respectively; the VH comprises a CDR-H1, a CDR-H2, and a CDR-H3 comprising SEQ ID NOS: 63, 64, and 60, respectively, and the VL comprises a CDR-L1, a CDR-L2, and a CDR-L3 comprising SEQ ID NOS: 221, 222, and 223, respectively; the VH comprises a CDR-H1, a CDR-H2, and a CDR-H3 comprising SEQ ID NOS: 63, 64, and 60, respectively, and the VL comprises a CDR-L1, a CDR-L2, and a CDR-L3 comprising SEQ ID NOS: 221, 222, and 223, respectively; the VH comprises a CDR-H1, a CDR-H2, and a CDR-H3 comprising SEQ ID NOS: 75, 76, and 60, respectively, and the VL comprises a CDR-L1, a CDR-L2, and a CDR-L3 comprising SEQ ID NOS: 229, 222, and 223, respectively; the VH comprises a CDR-H1, a CDR-H2, and a CDR-H3 comprising SEQ ID NOS: 40, 41, and 37, respectively, and the VL comprises a CDR-L1, a CDR-L2, and a CDR-L3 comprising SEQ ID NOS: 233, 234, and 235, respectively; the VH comprises a CDR-H1, a CDR-H2, and a CDR-H3 comprising SEQ ID NOS: 52, 53, and 37, respectively, and the VL comprises a CDR-L1, a CDR-L2, and a CDR-L3 comprising SEQ ID NOS: 241, 234, and 242, respectively; the VH comprises a CDR-H1, a CDR-H2, and a CDR-H3 comprising SEQ ID NOS: 87, 88, and 84, respectively, and the VL comprises a CDR-L1, a CDR-L2, and a CDR-L3 comprising SEQ ID NOS: 246, 247, and 248, respectively; the VH comprises a CDR-H1, a CDR-H2, and a CDR-H3 comprising SEQ ID NOS: 98, 99, and 84, respectively, and the VL comprises a CDR-L1, a CDR-L2, and a CDR-L3 comprising SEQ ID NOS: 246, 254, and 255, respectively; the VH comprises a CDR-H1, a CDR-H2, and a CDR-H3 comprising SEQ ID NOS: 108, 109, and 105, respectively, and the VL comprises a CDR-L1, a CDR-L2, and a CDR-L3 comprising SEQ ID NOS: 258, 259, and 260, respectively; the VH comprises a CDR-H1, a CDR-H2, and a CDR-H3 comprising SEQ ID NOS: 121, 122, and 118, respectively, and the VL comprises a CDR-L1, a CDR-L2, and a CDR-L3 comprising SEQ ID NOS: 265, 266, and 267, respectively; the VH comprises a CDR-H1, a CDR-H2, and a CDR-H3 comprising SEQ ID NOS: 134, 135, and 131, respectively, and the VL comprises a CDR-L1, a CDR-L2, and a CDR-L3 comprising SEQ ID NOS: 246, 273, and 274, respectively; the VH comprises a CDR-H1, a CDR-H2, and a CDR-H3 comprising SEQ ID NOS: 147, 148, and 144, respectively, and the VL comprises a CDR-L1, a CDR-L2, and a CDR-L3 comprising SEQ ID NOS: 278, 279, and 280, respectively; the VH comprises a CDR-H1, a CDR-H2, and a CDR-H3 comprising SEQ ID NOS: 160, 161, and 157, respectively, and the VL comprises a CDR-L1, a CDR-L2, and a CDR-L3 comprising SEQ ID NOS: 286, 287, and 288, respectively; the VH comprises a CDR-H1, a CDR-H2, and a CDR-H3 comprising SEQ ID NOS: 173, 174, and 170, respectively, and the VL comprises a CDR-L1, a CDR-L2, and a CDR-L3 comprising SEQ ID NOS: 294, 234, and 295, respectively; the VH comprises a CDR-H1, a CDR-H2, and a CDR-H3 comprising SEQ ID NOS: 185, 186, and 182, respectively, and the VL comprises a CDR-L1, a CDR-L2, and a CDR-L3 comprising SEQ ID NOS: 299, 300, and 301, respectively; the VH comprises a CDR-H1, a CDR-H2, and a CDR-H3 comprising SEQ ID NOS: 173, 174, and 193, respectively, and the VL comprises a CDR-L1, a CDR-L2, and a CDR-L3 comprising SEQ ID NOS: 306, 234, and 307, respectively; the VH comprises a CDR-H1, a CDR-H2, and a CDR-H3 comprising SEQ ID NOS: 160, 161, and 157, respectively, and the VL comprises a CDR-L1, a CDR-L2, and a CDR-L3 comprising SEQ ID NOS: 311, 312, and 313, respectively; the VH comprises a CDR-H1, a CDR-H2, and a CDR-H3 comprising SEQ ID NOS: 202, 203, and 199, respectively, and the VL comprises a CDR-L1, a CDR-L2, and a CDR-L3 comprising SEQ ID NOS: 319, 320, and 321, respectively.

In some embodiments, the antibody or antigen-binding fragment thereof comprises a VH and a VL, wherein: the VH comprises a CDR-H1, a CDR-H2, and a CDR-H3 comprising SEQ ID NOS: 40, 41, and 37, respectively, and the VL comprises a CDR-L1, a CDR-L2, and a CDR-L3 comprising SEQ ID NOS: 233, 234, and 235, respectively; the VH comprises a CDR-H1, a CDR-H2, and a CDR-H3 comprising SEQ ID NOS: 52, 53, and 37, respectively, and the VL comprises a CDR-L1, a CDR-L2, and a CDR-L3 comprising SEQ ID NOS: 241, 234, and 242, respectively; the VH comprises a CDR-H1, a CDR-H2, and a CDR-H3 comprising SEQ ID NOS: 87, 88, and 84, respectively, and the VL comprises a CDR-L1, a CDR-L2, and a CDR-L3 comprising SEQ ID NOS: 246, 247, and 248, respectively; the VH comprises a CDR-H1, a CDR-H2, and a CDR-H3 comprising SEQ ID NOS: 98, 99, and 84, respectively, and the VL comprises a CDR-L1, a CDR-L2, and a CDR-L3 comprising SEQ ID NOS: 246, 254, and 255, respectively

In some embodiments, the VH and VL regions of the antibody or antigen-binding fragment thereof comprise the sequences of SEQ ID NOs: 1 and 17, respectively; the VH and VL regions of the antibody or antigen-binding fragment thereof comprise the sequences of SEQ ID NOs: 2 and 18, respectively; the VH and VL regions of the antibody or antigen-binding fragment thereof comprise the sequences of SEQ ID NOs: 3 and 19, respectively; the VH and VL regions of the antibody or antigen-binding fragment thereof comprise the sequences of SEQ ID NOs: 4 and 19, respectively; the VH and VL regions of the antibody or antigen-binding fragment thereof comprise the sequences of SEQ ID NOs: 5 and 20, respectively; the VH and VL regions of the antibody or antigen-binding fragment thereof comprise the sequences of SEQ ID NOs: 1 and 21, respectively; the VH and VL regions of the antibody or antigen-binding fragment thereof comprise the sequences of SEQ ID NOs: 2 and 22, respectively; the VH and VL regions of the antibody or antigen-binding fragment thereof comprise the sequences of SEQ ID NOs: 6 and 23, respectively; the VH and VL regions of the antibody or antigen-binding fragment thereof comprise the sequences of SEQ ID NOs: 7 and 24, respectively; the VH and VL regions of the antibody or antigen-binding fragment thereof comprise the sequences of SEQ ID NOs: 8 and 25, respectively; the VH and VL regions of the antibody or antigen-binding fragment thereof comprise the sequences of SEQ ID NOs: 9 and 26, respectively; the VH and VL regions of the antibody or antigen-binding fragment thereof comprise the sequences of SEQ ID NOs: 10 and 27, respectively; the VH and VL regions of the antibody or antigen-binding fragment thereof comprise the sequences of SEQ ID NOs: 11 and 28, respectively.

In some embodiments, the VH and VL regions of the antibody or antigen-binding fragment thereof comprise the sequences of SEQ ID NOs: 13 and 30, respectively, or humanized sequences thereof; the VH and VL regions of the antibody or antigen-binding fragment thereof comprise the sequences of SEQ ID NOs: 14 and 31, respectively, or humanized sequences thereof; the VH and VL regions of the antibody or antigen-binding fragment thereof comprise the sequences of SEQ ID NOs: 15 and 32, respectively, or humanized sequences thereof; the VH and VL regions of the antibody or antigen-binding fragment thereof comprise the sequences of SEQ ID NOs: 12 and 33, respectively, or humanized sequences thereof; the VH and VL regions of the antibody or antigen-binding fragment thereof comprise the sequences of SEQ ID NOs: 16 and 34, respectively, or humanized sequences thereof.

In some embodiments, the antibody or antigen binding fragment comprises one or more VH and one or more VL, in any order or orientation. In some embodiments, the antibody or antigen-binding fragment comprises a VH region and a VL region, and the VH region is amino-terminal to the VL region. In some embodiments, the antibody or antigen-binding fragment comprises a VH region and a VL region, and the VH region is carboxy-terminal to the VL region. In some embodiments, the VH region(s) and the VL region(s) are linked directly or indirectly, optionally via a linker.

In some embodiments, the antibody or antigen-binding fragment, e.g., scFv, may include a VH region or portion thereof, followed by the linker, followed by a VL region or portion thereof. In some embodiments, the antibody or antigen-binding fragment, e.g., the scFv, may include the VL region or portion thereof, followed by the linker, followed by the VH region or portion thereof.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region. In some embodiments, the single-chain antibody fragment (e.g. scFv) includes one or more linkers joining two antibody domains or regions, such as a VH region and a VL region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided anti-PD-L1 antibodies include single-chain antibody fragments, such as scFvs and diabodies, particularly human single-chain antibody fragments, typically comprising linker(s) joining two antibody domains or regions, such VH and VL regions. The linker typically is a peptide linker, e.g., a flexible and/or soluble peptide linker, such as one rich in glycine and serine.

The antibody or antigen-binding fragment thereof, may contain at least a portion of an immunoglobulin constant region, such as one or more constant region domain. In some embodiments, the constant regions include a light chain constant region and/or a heavy chain constant region 1 (CH1). In some embodiments, the antibody includes a CH2 and/or CH3 domain, such as an Fc region. In some embodiments, the Fc region is an Fc region of a human IgG, such as an IgG1 or IgG4.

Also provided are nucleic acids, e.g., polynucleotides, encoding the antibodies and/or portions, e.g., chains, thereof. Among the provided nucleic acids are those encoding the anti-PD-L1 antibodies and fragments described herein. Also provided are nucleic acids, e.g., polynucleotides, encoding one or more antibodies and/or portions thereof, e.g., those encoding one or more of the anti-PD-L1 antibodies or antigen-binding fragments described herein and/or other antibodies and/or portions thereof, e.g., antibodies and/or portions thereof that binds other target antigens. The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Also provided are vectors containing the nucleic acids, e.g., polynucleotides, and host cells containing the vectors, e.g., for producing the antibodies or antigen-binding fragments thereof. Also provided are methods for producing the antibodies or antigen-binding fragments thereof. The nucleic acid may encode an amino acid sequence comprising the VL region and/or an amino acid sequence comprising the VH region of the antibody (e.g., the light and/or heavy chains of the antibody). The nucleic acid may encode one or more amino acid sequence comprising the VL region and/or an amino acid sequence comprising the VH region of the antibody (e.g., the light and/or heavy chains of the antibody). In some embodiments, the nucleic acid, e.g., polynucleotide encodes one or more VH region and/or one or more VL region of the antibody, in any order or orientation. In some embodiments, the nucleic acid, e.g., polynucleotide encodes a VH region and a VL region, and the coding sequence for the VH region is upstream of the coding sequence for the VL region. In some embodiments, the nucleic acid, e.g., polynucleotide encodes a VH region and a VL region, and the coding sequence for the VL region is upstream of the coding sequence for the VH region.

In some embodiments, one or more vectors (e.g., expression vectors) comprising such nucleic acids are provided. In a further embodiment, a host cell comprising such nucleic acids is provided. In some embodiments, a host cell comprises (e.g., has been transformed with) (1) a vector comprising a nucleic acid that encodes an amino acid sequence comprising the VL region of the antibody and an amino acid sequence comprising the VH region of the antibody, or (2) a first vector comprising a nucleic acid that encodes an amino acid sequence comprising the VL region of the antibody and a second vector comprising a nucleic acid that encodes an amino acid sequence comprising the VH region of the antibody. In some embodiments, a host cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such host cells are provided. In some embodiments, a composition containing one or more such host cells are provided. In some embodiments, the one or more host cells can express different antibodies, or the same antibody. In some embodiments, each of the host cells can express more than one antibody.

Also provided are methods of making the anti-PD-L1 antibodies (including antigen-binding fragments). For recombinant production of the anti-PD-L1 antibody, a nucleic acid sequence or a polynucleotide encoding an antibody, e.g., as described above, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-PD-L1 antibody is provided, wherein the method comprises culturing a host cell comprising a nucleic acid sequence encoding the antibody, as provided above, under conditions suitable for expression of the antibody, and optionally recovering the antibody from the host cell (or host cell culture medium).

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., VH region and/or VL region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., VH region and/or VL region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

The provided embodiments further include vectors and host cells and other expression systems for expressing and producing the antibodies and other antigen-binding proteins, including eukaryotic and prokaryotic host cells, including bacteria, filamentous fungi, and yeast, as well as mammalian cells such as human cells, as well as cell-free expression systems.

### 2. Exemplary Features of Anti-PD-L1 Antibodies

In some aspects, the provided antibodies or antigen-binding fragments thereof have one or more specified functional features, such as binding properties, including binding to particular epitopes.

In some embodiments, the antibodies or antigen-binding fragments thereof specifically bind to PD-L1 protein. In some embodiments provided herein, PD-L1 protein refers to human PD-L1, a non-human primate (e.g., cynomolgus monkey) PD-L1 protein, or a mouse PD-L1 protein. In some embodiments provided herein, PD-L1 protein refers to human PD-L1 or a non-human primate (e.g., cynomolgus monkey) PD-L1 protein. In some embodiments of any of the embodiments herein, PD-L1 protein refers to human PD-L1 protein. The observation that an antibody or other binding molecule binds to PD-L1 protein or specifically binds to PD-L1 protein does not necessarily mean that it binds to a PD-L1 protein of every species. For example, in some embodiments, features of binding to PD-L1 protein, such as the ability to specifically bind thereto and/or to bind with a particular affinity to a particular degree, in some embodiments, refers to the ability with respect to a human PD-L1 protein and the antibody may not have this feature with respect to a PD-L1 protein of another species, such as mouse.

In some embodiments, the antibody or antigen-binding fragment binds to a mammalian PD-L1 protein, including to naturally occurring variants of PD-L1, such as certain splice variants or allelic variants.

In some embodiments, the antibodies specifically bind to the extracellular domain of human PD-L1 protein, such as to an epitope or region of human PD-L1 protein, such as the human PD-L1 extracellular domain comprising the amino acid sequence of SEQ ID NO:327 (amino acid residues 19-239 of UniProt Accession No. Q9NZQ7), or an allelic variant or splice variant thereof. In some embodiments, the antibodies bind to cynomolgus monkey PD-L1 protein, such as the cynomolgus monkey PD-L1 extracellular domain set forth in SEQ ID NO: 328 (amino acid residues 19-239 of UniProt Accession No.: G7PSE7). In some embodiments, the antibodies bind to human PD-L1 but bind at a lower level or to a lesser degree or affinity to cynomolgus monkey PD-L1 protein, such as the cynomolgus monkey PD-L1 extracellular domain set forth in SEQ ID NO:328 (amino acid residues 19-239 of UniProt Accession No.: G7PSE7). In some embodiments, the antibodies do not bind to or bind at a lower level or to a lesser degree or affinity to mouse PD-L1 protein, such as the mouse PD-L1 extracellular domain set forth in SEQ ID NO:329 (amino acid residues 19-238 of UniProt Accession No. Q9EP73). In some embodiments, the antibodies bind to mouse PD-L1 protein, with lower affinity than its binding to a human PD-L1 protein and/or a cynomolgus monkey PD-L1 protein. In some embodiments, the antibodies bind to mouse PD-L1 protein and/or a cynomolgus monkey PD-L1 protein with lower affinity than its binding to a human PD-L1 protein. In some embodiments, the antibodies bind to cynomolgus monkey PD-L1 protein with similar binding affinity compared to its binding to a human PD-L1 protein.

In one embodiment, the extent of binding of an anti-PD-L1 antibody to an unrelated, non-PD-L1 protein, such as a non-human PD-L1 protein or other non-PD-L1 protein, is less than at or about 10% of the binding of the antibody to human PD-L1 protein as measured, e.g., by a radioimmunoassay (RIA), ELISA, or surface plasmon resonance (SPR). In some embodiments, among provided antibodies are antibodies in which binding to mouse PD-L1 protein is less than or at or about 10% of the binding of the antibody to human PD-L1 protein. In some embodiments, among provided antibodies are antibodies in which binding to cynomolgus monkey PD-L1 protein is less than or at or about 50%, 40%, 30%, 20%, or 10% of the binding of the antibody to human PD-L1 protein.

In some embodiments, the provided antibodies are capable of binding PD-L1 protein, such as human PD-L1 protein, with at least a certain affinity, as measured by any of a number of known methods. In some embodiments, the affinity is represented by an equilibrium dissociation constant (K_{D}); in some embodiments, the affinity is represented by EC₅₀.

A variety of assays can be used to assess binding affinity and/or determine whether a binding molecule (e.g., an antibody or fragment thereof) specifically binds to a particular ligand (e.g., an antigen, such as a PD-L1 protein). It is within the level of a skilled artisan to determine the binding affinity of a binding molecule, e.g., an antibody, for an antigen, e.g., PD-L1, such as human PD-L1 or cynomolgus PD-L1 or mouse PD-L1, such as by using any of a number of binding assays that are well known in the art (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

For example, in some embodiments, a BIAcore® instrument can be used to determine the binding kinetics and constants of a complex between two proteins (e.g., an antibody, such as an anti-PD-L1 antibody, or fragment thereof, and an antigen, such as a PD-L1 protein), using surface plasmon resonance (SPR) analysis. SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip.

Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, and other methods for detection of protein binding.

In some embodiments, the binding affinity of the provided antibody, fragment, or conjugate to PD-L1 protein, such as human PD-L1 protein, is similar to or higher than a reference antibody (e.g., avelumab). In some embodiments, the binding affinity is measured as the half maximal effective concentration (EC₅₀) or the dissociation constant (K_{D}) of the antibody, fragment, or conjugate. The EC₅₀ or K_{D} can be determined for binding free or immobilized PD-L1, such as the extracellular domain of PD-L1 or PD-L1 expressed on the surface of a cell. In some embodiments, the EC₅₀ or K_{D} value of the provided antibody, fragment, or conjugate to PD-L1 protein, such as human PD-L1 protein, is similar to or lower (e.g., corresponding to higher binding affinity) than a reference antibody (e.g., avelumab).

In some embodiments, the binding affinity is measured as the half maximal effective concentration (EC₅₀) of the antibody, fragment, or conjugate. In some embodiments, the EC₅₀ of the antibody, fragment, or conjugate for binding PD-L1 protein, such as human PD-L1 protein, is similar to or less than the EC₅₀ of a reference antibody, fragment or conjugate (e.g., avelumab or corresponding fragment or conjugate thereof). In some embodiments, the EC₅₀ of the antibody, fragment, or conjugate is reduced by about 0.5 fold, about 1 fold, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, about 30 fold, about 35 fold, about 40 fold, about 45 fold, about 50 fold, or more.

In some embodiments, the binding affinity is measured as the K_{D} of the antibody, fragment, or conjugate. In some embodiments, the K_{D} of the antibody, fragment, or conjugate for binding PD-L1 protein, such as human PD-L1 protein, is similar to or less than the K_{D} of a reference antibody, fragment or conjugate (e.g., avelumab or corresponding fragment or conjugate thereof). In some embodiments, the K_{D} of the antibody, fragment, or conjugate is reduced by about 0.5 fold, about 1 fold, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, about 30 fold, about 35 fold, about 40 fold, about 45 fold, about 50 fold, or more.

In some embodiments, the binding affinity of a binding molecule, such as an anti-PD-L1 antibody, for different antigens, e.g., PD-L1 proteins from different species can be compared to determine the species cross-reactivity. For example, species cross-reactivity can be classified as high cross reactivity or low cross reactivity. In some embodiments, the equilibrium dissociation constant, K_{D}, or EC₅₀ for different antigens, e.g., PD-L1 proteins from different species such as human, cynomolgus monkey or mouse, can be compared to determine species cross-reactivity. In some embodiments, the species cross-reactivity of an anti-PD-L1 antibody can be high, e.g., the anti-PD-L1 antibody binds to human PD-L1 and a species variant PD-L1 to a similar degree, e.g., the ratio of the EC₅₀ or K_{D} for human PD-L1 and EC₅₀ or K_{D} for the species variant PD-L1 is or is about 1. In some embodiments, the species cross-reactivity of an anti-PD-L1 antibody can be low, e.g., the anti-PD-L1 antibody has a high affinity for human PD-L1 but a low affinity for a species variant PD-L1, or vice versa. The degree of species cross-reactivity can also be compared with the species cross-reactivity of a known antibody.

In some embodiments, the provided antibodies or antigen binding fragments thereof bind to a similar degree to a human PD-L1 protein and a non-human PD-L1 protein or other non-PD-L1 proteins. For example, in some embodiments, the provided antibodies or antigen binding fragments thereof bind to a human PD-L1 protein, such as the human PD-L1 protein comprising the amino acid sequence of SEQ ID NO:327 (amino acid residues 19-239 of UniProt Accession No. Q9NZQ7) or an allelic variant or splice variant thereof, with an EC₅₀ or K_{D}, and a non-human PD-L1, such as a cynomolgus monkey PD-L1, such as the cynomolgus monkey PD-L1 protein set forth in SEQ ID NO: 328 (amino acid residues 19-239 of UniProt Accession No.: G7PSE7), have an EC₅₀ or K_{D} that is similar, or about the same, or less than 2-fold different, or less than 5-fold different. In other embodiments, the provided antibodies or antigen binding fragments thereof bind to a human PD-L1 protein, such as the human PD-L1 protein comprising the amino acid sequence of SEQ ID NO:327 (amino acid residues 19-239 of UniProt Accession No. Q9NZQ7) or an allelic variant or splice variant thereof, has an EC₅₀ or K_{D}, that is lower than its EC₅₀ or K_{D} for a non-human PD-L1, such as a mouse PD-L1, such as the mouse PD-L1 protein set forth in SEQ ID NO:329 (amino acid residues 19-238 of UniProt Accession No. Q9EP73).

In some embodiments, the antibodies (e.g., antigen-binding fragment) display a binding preference for PD-L1-expressing cells as compared to PD-L1-negative cells, such as particular cells known and/or described herein to express PD-L1 and known not to express PD-L1. In some embodiments, the binding preference is observed where a significantly greater degree of binding is measured to the PD-L1-expressing, as compared to the non-expressing cells. In some embodiments, the fold change in degree of binding detected, for example, as measured by mean fluorescence intensity in a flow cytometry-based assay and/or dissociation constant or EC₅₀, to the PD-L1-expressing cells as compared to the non-PD-L1-expressing cells, is at least at or about 1.5, 2, 3, 4, 5, 6, or more, and/or is about as great, about the same, at least as great or at least about as great, or greater, than the fold change observed for the corresponding form of the reference antibody.

Anti-PD-L1 antibodies (e.g., antigen-binding fragments) provided herein may be identified, screened for, or characterized for their physical/chemical properties and/or biological activities by various known assays. In one aspect, the antibody is tested for its antigen binding activity, e.g., by known methods such as ELISA, Western blotting, and/or flow cytometric assays, including cell-based binding assays, for example, assessing binding of the antibody (e.g., conjugated to a fluorescent marker or tagged) to a cell expressing the target antigen, e.g., PD-L1, in some cases compared to results using cells that do not express the target antigen, e.g., PD-L1. Binding affinity may be measured as K_{D} or EC₅₀. In some examples, binding affinity, binding kinetics, and/or binding constants can be measured using assays to determine molecular interaction, such as surface plasmon resonance analysis.

In some aspects, the anti-PD-L1 antibodies (e.g., antigen-binding fragments) provided herein can be conjugated with one or more agents, such as one or more drugs, small molecules, dyes (e.g., phthalocyanine dyes such as IR700) and/or therapeutic agents, for example, as described in Section II.

In some aspects, after conjugation with the one or more agents, the conjugate comprising the provided antibodies or antigen-binding fragments retain or substantially retain their physical/chemical properties and/or biological activities, such that the physical/chemical properties and/or biological activities of the conjugate are equal to or similar to the physical/chemical properties and/or biological activities of the unconjugated (naked) antibody or antigen-binding fragment, or they are reduced by no more than 50%, no more than 40%, no more than 30%, no more than 20%, no more than 10%, no more than 5%, no more than 4%, no more than 3%, no more than 2%, or no more than 1%, or is at least 50%, at least 60%, at least 70%, at least 80%, at least 90%, at least 95%, at least 96%, at least 97%, at least 98%, or at least 99% of the physical/chemical properties and/or biological activities of the unconjugated (naked) antibody.

In some aspects, after conjugation with the one or more agents, such as a phthalocyanine dye such as IR700, the conjugate comprising the provided antibodies or antigen-binding fragments retain or substantially retain the binding affinity to the target antigen PD-L1, such that the binding affinity of the conjugate is equal to or similar to the binding affinity of the unconjugated (naked) antibody or antigen-binding fragment, or the binding affinity is reduced by no more than 50%, no more than 40%, no more than 30%, no more than 20%, no more than 10%, no more than 5%, no more than 4%, no more than 3%, no more than 2%, or no more than 1%, or exhibits at least 50%, at least 60%, at least 70%, at least 80%, at least 90%, at least 95%, at least 96%, at least 97%, at least 98%, or at least 99% of the binding affinity of the unconjugated (naked) antibody. In some embodiments, the conjugate exhibits similar binding to a PD-L1 protein compared to the unconjugated antibody.

Competition assays may be used to identify an antibody that competes with any of the antibodies (e.g., antigen-binding fragments) described herein or with other known anti-PD-L1 antibodies. Assays for mapping epitopes bound by the antibodies and other known anti-PD-L1 antibodies also may be used and are known.

### B. Multispecific Antibodies

In certain embodiments, the PD-L1-binding molecules, e.g., antibodies or polypeptides such as chimeric receptors containing the same, are multispecific. Among the multispecific binding molecules are multispecific antibodies, including, e.g. bispecific or trispecific antibodies. Multispecific binding partners, e.g., antibodies, have binding specificities for at least two different sites, which may be in the same or different antigens. In certain embodiments, one of the binding specificities is for PD-L1 and the other is for another antigen. In certain embodiments, the bispecific antibody comprises an additional binding domain that is specific for a second or additional antigen. In some embodiments, additional binding molecules bind to and/or recognize a third, or more antigens. In certain embodiments, bispecific antibodies may bind to two different epitopes of PD-L1. Bispecific antibodies may also be used to localize fluorescent, phototoxic, and/or cytotoxic agents to cells which express PD-L1. Bispecific antibodies can be prepared as full length antibodies or antibody fragments. Among the multispecific antibodies are multispecific single-chain antibodies, e.g., diabodies, triabodies, and tetrabodies, tandem di-scFvs, and tandem tri-scFvs.

In some embodiments, among the second or additional antigens for multi-targeting strategies includes those in which at least one of the antigens is a universal tumor antigen, or a family member thereof. In some embodiments, the second or additional antigen is an antigen expressed on a tumor. In some embodiments, the PD-L1-binding molecules provided herein target an antigen on the same tumor type as the second or additional antigen. In some embodiments, the second or additional antigen may also be a universal tumor antigen or may be a tumor antigen specific to a tumor type.

Exemplary second or additional antigens include CD4, CD5, CD8, CD14, CD15, CD19, CD20, CD21, CD22, CD23, CD25, CD33, CD37, CD38, CD40, CD40L, CD46, CD47, CD52, CD54, CD74, CD80, CD126, CD138, CTLA-4, B7, MUC-1, Ia, HM1.24, HLA-DR, tenascin, an angiogenesis factor, VEGF, PIGF, SIRPalpha, ED-B fibronectin, an oncogene, an oncogene product, CD66a-d, necrosis antigens, Ii, IL-2, T101, TAC, IL-6, ROR1, TRAIL-R1 (DR4), TRAIL-R2 (DR5), tEGFR, Her2, L1-CAM, mesothelin, CEA, hepatitis B surface antigen, anti-folate receptor, CD24, CD30, CD44, EGFR, EGP-2, EGP-4, EPHa2, ErbB2, ErbB3, ErbB4, erbB dimers, EGFR vIII, FBP, FCRL5, FCRH5, fetal acetylcholine receptor, GD2, GD3, G protein-coupled receptor class C group 5 member D (GPRC5D), HMW-MAA, IL-22R-alpha, IL-13R-alpha2, kdr, kappa light chain, Lewis Y, L1-cell adhesion molecule (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Fetal AchR, NKG2D ligands, dual antigen, an antigen associated with a universal tag, a cancer-testes antigen, MUC1, MUC16, NY-ESO-1, MART-1, gp100, oncofetal antigen, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, hTERT, MDM2, CYP1B, WT1, livin, AFP, p53, cyclin (D1), CS-1, BAFF-R, TACI, CD56, TIM-3, CD123, L1-cell adhesion molecule, MAGE-A1, MAGE A3, a cyclin, such as cyclin A1 (CCNA1) and/or a pathogen-specific antigen, biotinylated molecules, molecules expressed by HIV, HCV, HBV and/or other pathogens, and/or in some aspects, neoepitopes or neoantigens thereof. In some embodiments, the antigen is associated with or is a universal tag.

In some aspects, the antigen, e.g., the second or additional antigen, such as the disease-specific antigen and/or related antigen, is expressed on multiple myeloma, such as G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, B2-Microglobulin, HM1.24, IGF-1R, IL-6R, TRAIL-R1, and the activin receptor type IIA (ActRIIA). See Benson and Byrd, J. Clin. Oncol. (2012) 30 (16): 2013-15; Tao and Anderson, Bone Marrow Research (2011): 924058; Chu et al., Leukemia (2013) 28 (4): 917-27; Garfall et al., Discov Med. (2014) 17 (91): 37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g., scFv) are contained in multispecific antibodies, multispecific chimeric receptors, and/or multispecific cells.

### C. Variants

In certain embodiments, the antibodies (e.g., antigen-binding fragment) include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies (e.g., antigen-binding fragment) include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC).

In some embodiments, one or more residues within a CDR of a parent antibody (e.g. a humanized or human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH region and VL region sequences provided above, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### D. Modifications

In certain embodiments, the antibody is altered to increase or decrease the extent to which the antibody is glycosylated, for example, by removing or inserting one or more glycosylation sites by altering the amino acid sequence and/or by modifying the oligosaccharide(s) attached to the glycosylation sites, e.g., using certain cell lines. In some embodiments, an N-linked glycosylation, which is a glycosylation site that occurs at asparagine residues in the consensus sequence-Asn-Xaa-Ser/Thr is removed or inserted.

Exemplary modifications, variants, and cell lines are described, e.g., in Patent Publication Nos. US 2003/0157108, US 2004/0093621, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Ripka et al. Arch. Biochem. Biophys. 249:533-545 (1986); US 2003/0157108 A1, Presta, L; and WO 2004/056312 A1, Yamane-Ohnuki et al. Biotech. Bioeng. 87:614 (2004); Kanda, Y. et al., Biotechnol. Bioeng., 94 (4): 680-688 (2006); and WO2003/085107); WO 2003/011878 (Jean-Mairet et al.); U.S. Pat. No. 6,602,684 (Umana et al.); and US 2005/0123546 (Umana et al.); WO 1997/30087 (Patel et al.); WO 1998/58964 (Raju, S.); and WO 1999/22764 (Raju, S.).

Among the modified antibodies are those having one or more amino acid modifications in the Fc region, such as those having a human Fc region sequence or other portion of a constant region (e.g., a human IgG1, IgG2, IgG3 or IgG4 Fc region) comprising an amino acid modification (e.g., a substitution) at one or more amino acid positions. Such modifications can be made, e.g., to improve half-life, alter binding to one or more types of Fc receptors, and/or alter effector functions.

In some of any provided embodiments, the anti-PD-L1 antibody comprises a functional Fc region. In some of any provided embodiments, the anti-PD-L1 antibody comprises a full-length Fc region. In some embodiments, the anti-PD-L1 antibody comprises an IgG1 Fc region. In some embodiments, the anti-PD-L1 antibody comprises an IgG2 Fc region. In some embodiments, the IgG2 Fc region is an IgG2a Fc region. In some embodiments, the IgG2 Fc region is an IgG2a/b Fc region. In some embodiments, the IgG2 Fc region is an IgG2a Fc region. In some of any provided embodiments, the anti-PD-L1 antibody comprises an IgG3 Fc region. In some of any provided embodiments, the anti-PD-L1 antibody comprises an IgG4 Fc region. In some embodiments, the Fc region is modified to modulate effector functions of the antibody portion of the conjugate. Any of such modifications, such as those described in Wang et al., (2018) Protein Cell. 9 (1): 63-73, are contemplated for the antibodies, antibody fragments, and conjugates described herein.

In some of any provided embodiments, the anti-PD-L1 antibody does not comprise a functional Fc region. In some of such examples, the anti-PD-L1 antibody does not contain an Fc region or comprises an Fc region that has been modified such that the Fc region does not bind an Fc receptor and/or does not elicit substantial Fc effector function (e.g., ADCC, ADCP, and/or CDC). In some of such embodiments, the anti-PD-L1 antibody comprises an Fc receptor that contains an amino acid substitution to eliminate the glycosylation site at a position corresponding to position 297 of the heavy chain based on EU numbering described by Edelman et al., (1969) Proc Natl Acad Sci USA. 63 (1): 78-85. For example, the anti-PD-L1 antibody can comprise an Fc receptor that contains an asparagine to glutamine substitution at or corresponding to position 297 (N297Q) with respect to EU numbering of the antibody sequence, an asparagine to alanine substitution at or corresponding to position 297 (N297A) with respect to EU numbering of the antibody sequence, or an asparagine to glycine substitution at or corresponding to position 297 (N297G) with respect to EU numbering of the antibody sequence.

In some of any provided embodiments, the anti-PD-L1 antibody comprises an Fc region that exhibits enhanced Fc-mediated effector function, such as ADCC, ADCP, and/or CDC activity, and/or exhibits preferential binding to the Fc gamma receptor. In some embodiments, the anti-PD-L1 antibody exhibits enhanced function due to increased Fc receptor engagement. In some embodiments, the Fc region contains one or more of the following mutations: a glycine to alanine substitution at position 236 (G236A) a serine to aspartic acid substitution at position 239 (S239D), an alanine to leucine substitution at position 330 (A330L), an isoleucine to glutamic acid substitution at position 332 (1332E), a glutamic acid to alanine substitution at position 333 (E333A), a lysine to alanine substitution at position 334 (K334A), an arginine to alanine substitution at position 255 (S255A), a threonine to alanine substitution at position 256 (T256A), a serine to alanine substitution at position 267 (S267A), a serine to alanine substitution at position 298 (S298A), an asparagine to serine substitution at position 325 (N325S), a leucine to phenylalanine at position 328 (L328F), an alanine to leucine substitution at position 330 (A330L), an isoleucine to glutamic acid substitution at position 333 (E333A), a glutamic acid to alanine substitution at position 333 (E333A), a lysine to alanine substitution at position 334 (K334), and/or an alanine to glutamine substitution at position 378 (A378Q) with respect to EU numbering of the antibody heavy chain. In some embodiments, the Fc region contains a glycine to alanine substitution at position 236, a serine to aspartic acid substitution at position 239, and isoleucine to glutamic acid at position 332 (G236A/S239D/1332E) with respect to EU numbering of the antibody heavy chain. In some embodiments, the Fc region contains a glycine to alanine substitution at position 236, an alanine to leucine substitution at position 330, and isoleucine to glutamic acid at position 332 (G236A/A330L/1332E) with respect to EU numbering of the antibody heavy chain. In some embodiments, the Fc region contains a glycine to alanine substitution at position 236, a serine to aspartic acid substitution at position 239, an alanine to leucine substitution at position 330, and isoleucine to glutamic acid at position 332 (G236A/S239D/A330L/1332E) with respect to EU numbering of the antibody heavy chain. In some embodiments, the Fc region contains a serine to aspartic acid substitution at position 239 and isoleucine to glutamic acid at position 332 (S239D/1332E) with respect to EU numbering of the antibody heavy chain. In some embodiments, the Fc region contains a serine to alanine substitution at position 298 and a lysine to alanine substitution at position 334 (S298A/K334) with respect to EU numbering of the antibody heavy chain. In some embodiments, the Fc region contains a glutamic acid to alanine substitution at position 333 and a lysine to alanine substitution at position 334 (E333A/K334) with respect to EU numbering of the antibody heavy chain. In some embodiments, the Fc region contains a serine to alanine substitution at position 298, a glutamic acid to alanine substitution at position 333, and a lysine to alanine substitution at position 334 (S298A/E333A/K334) with respect to EU numbering of the antibody heavy chain. In some embodiments, the Fc region contains an arginine to alanine substitution at position 255 and a serine to alanine substitution at position 267 (R255A/S267A) with respect to EU numbering of the antibody heavy chain. In some embodiments, the FC region contains a threonine to alanine substitution at position 256 (T256A) with respect to EU numbering of the antibody heavy chain. In some embodiments, the Fc region contains a lysine to alanine substitution at position 334 and an alanine to glutamine substitution at position 378 (K334/A378) with respect to EU numbering of the antibody heavy chain. In some embodiments, the Fc region contains a serine to aspartic acid substitution at position 239, an alanine to leucine substitution at position 330, and an isoleucine to glutamic acid substitution at position 332 (S239D/A330L/1332E) with respect to EU numbering of the antibody heavy chain. In some embodiments, the Fc region contains a glycine to alanine substitution at position 236, a serine to aspartic substitution at position 239, and an isoleucine to glutamic acid substitution at position 332 (G236A/S239D/1332E) with respect to EU numbering of the antibody heavy chain. In some embodiments, the Fc region contains an arginine to serine substitution at position 325 and a leucine to phenylalanine substitution at position 328 (N325S/L328F) with respect to EU numbering of the antibody heavy chain. In some embodiments, the Fc region contains a phenylalanine to leucine substitution at position 243, an arginine to proline substitution at position 292, a tyrosine to leucine substitution at position 300, a valine to isoleucine substitution at position 305, and a proline to leucine substitution at position 396 (F243L/R292P/Y300L/V305I/P396L) with respect to EU numbering of the antibody heavy chain. In some embodiments, the Fc region contains a leucine to valine substitution at position 235, phenylalanine to leucine substitution at position 243, an arginine to proline substitution at position 292, a tryptophan to leucine substitution at position 300, and a proline to leucine substitution at position 396 (L235V/F243L/R292P/Y300L/P396L) with respect to EU numbering of the antibody heavy chain. In some embodiments, the Fc regions contains a leucine to tyrosine substitution at position 234, a leucine to glutamine substitution at position 235, a glycine to tryptophan substitution at position 236, a serine to methionine substitution at position 239, a histidine to aspartic acid substitution at position 268, an aspartic acid to glutamic acid substitution at position 270, and a serine to alanine substitution at position 298 (L234Y/L235Q/G236W/S239M/H268D/D270E/S298A) according to EU numbering of one of the antibody heavy chains and an aspartic acid to glutamic acid substitution at position 270, a lysine to aspartic acid substitution at position 326, an alanine to methionine substitution at position 330, and a lysine to glutamic acid substitution at position 334 (D270E/K326D/A330M/K334E), according to EU numbering of the opposing heavy chain.

Also among the variants are cysteine engineered antibodies such as “thioMAbs” and other cysteine engineered variants, in which one or more residues of an antibody are substituted with cysteine residues, in order to generate reactive thiol groups at accessible sites, e.g., for use in conjugation of agents and linker-agents, to produce immunoconjugates. Cysteine engineered antibodies are described, e.g., in U.S. Pat. Nos. 7,855,275 and 7,521,541.

In some embodiments, the antibodies (e.g., antigen-binding fragment) are modified to contain additional non-proteinaceous moieties, including water soluble polymers. Exemplary polymers include, but are not limited to, polyethylene glycol (PEG), copolymers of ethylene glycol/propylene glycol, carboxymethylcellulose, dextran, polyvinyl alcohol, polyvinylpyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, the polynucleotides are optimized, or contain certain features designed for optimization, such as for codon usage and/or to modify, e.g., increase or render more consistent among cell product lots, expression, such as surface expression, of the encoded antibody or fragment. In some embodiments, polynucleotides, encoding an anti-PD-L1 antibody, are modified to remove cryptic or hidden splice sites

### II. Conjugates

In some embodiments, the anti-PD-L1 antibody or antibody fragment (e.g., antigen-binding fragment) is or is part of an immunoconjugate, in which the antibody is conjugated to one or more heterologous molecules or moieties, such as, but not limited to, a phototoxic agent, a cytotoxic agent or an imaging agent. In particular embodiments, the antibody is conjugated to a phototoxic agent, such as a phthalocyanine dye. In some embodiments, the antibody is conjugated to a phototoxic agent, such a phthalocyanine dye, and a second phototoxic, cytotoxic, or imaging agent.

### A. Cytotoxic Agents

Cytotoxic agents include, but are not limited to, radioactive isotopes (e.g., At211, I131, 1125, Y90, Re186, Re188, Sm153, Bi212, P32, Pb212 and radioactive isotopes of Lu); chemotherapeutic agents (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as a chemotherapeutic agent or drug, a growth inhibitory agent, a toxins (e.g., a protein toxin, an enzymatically active toxin of bacterial, fungal, plant, or animal origin), or radioactive isotopes.

Among the immunoconjugates are antibody-drug conjugates (ADCs), in which an antibody is conjugated to one or more drugs, including but not limited to a maytansinoid; an auristatin such as monomethyl auristatin drug moieties DE and DF (MMAE and MMAF; a dolastatin; a calicheamicin or derivative thereof; an anthracycline such as daunomycin or doxorubicin; methotrexate; vindesine; a taxane such as docetaxel, paclitaxel, larotaxel, tesetaxel, and ortataxel; a trichothecene; and CC1065.

Also among the immunoconjugates are those in which the antibody (e.g., antigen-binding fragment) is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes.

Non-limiting, exemplary cytotoxic agents include aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-II), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribomiclease, onconase, rapLR1, DNase I, Staphylococcal enterotoxin-A, pokeweed antiviral protein, gelonin, diphtheria toxin, Pseudomonas exotoxin, and Pseudomonas endotoxin.

Also among the immunoconjugates are those in which the antibody (e.g., antigen-binding fragment) is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu.

### B. Phototoxic Agents

In particular aspects, the anti-PD-L1 antibody or antibody fragment (e.g., antigen-binding fragment) is conjugated to a phototoxic agent. In some aspects, the phototoxic agent is a phthalocyanine dye. Phthalocyanines are a group of photosensitizer compounds having the phthalocyanine ring system. Phthalocyanines are azaporphyrins that contain four benzoindole groups connected by nitrogen bridges in a 16-membered ring of alternating carbon and nitrogen atoms (i.e., C32H18N8) which form stable chelates with a metal or metalloid cation. In these compounds, the ring center is occupied by a metal ion (either a diamagnetic or a paramagnetic ion) that may, depending on the ion, carry zero, one or two ligands. In addition, the ring periphery may be either unsubstituted or substituted.

In some embodiments, phthalocyanines strongly absorb red or near IR radiation with absorption peaks falling between about 600 nm and 810 nm, which, in some cases, allow deep penetration of tissue by the light. Phthalocyanines are generally photostable. This photostability is typically advantageous in pigments and dyes and in many of the other applications of phthalocyanines.

In some embodiments, the phthalocyanine dye is water soluble and contains a luminescent fluorophore moiety having at least one aqueous-solubilizing moiety. In some embodiments, the aqueous solubilizing moiety contains silicon. In some embodiments, the phthalocyanine dye has a core atom such as Si, Ge, Sn, Al, or Zn. In some embodiments, the phthalocyanine dye contains a linker that has a reactive group, which is able to form a bond between the linker and another molecule, i.e., to form a conjugate. In some embodiments, the phthalocyanine dye can be tailored to fluoresce at a particular wavelength.

In some embodiments, the phthalocyanine dye contains a linker, i.e., is a linker-phthalocyanine dye moiety (L-D).

In some embodiments, the phthalocyanine dye is a phthalocyanine dye with a silicon coordinating metal (Si-phthalocyanine dye). In some embodiments, the phthalocyanine dye comprises the formula:

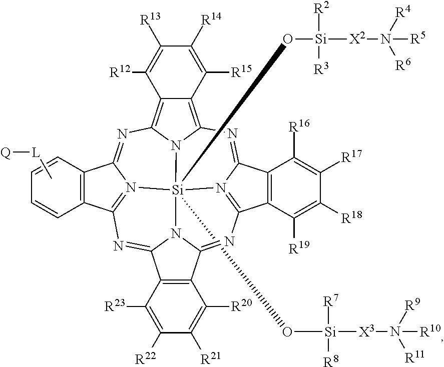

or a salt, stereoisomer, or tautomer thereof, wherein:
L is a linker;
Q is a reactive group for attachment of the dye to the targeting molecule;
R², R³, R⁷, and R⁸ are each independently selected from among optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;
R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and
X² and X³ are each independently C₁-C₁₀ alkylene, optionally interrupted by a heteroatom.

In some embodiments, the phthalocyanine dye comprises the formula:

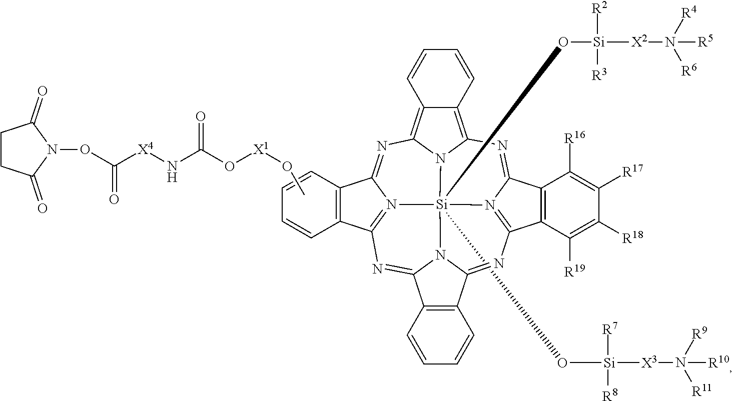

a salt, stereoisomer, or tautomer thereof, wherein:
X¹ and X⁴ are each independently a C₁-C₁₀ alkylene optionally interrupted by a heteroatom;
R², R³, R⁷, and R⁸ are each independently selected from optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and
R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments of the methods and uses provided herein, a Si-phthalocyanine dye is IRDye 700DX (IR700). In some embodiments, the phthalocyanine dye containing the reactive group is IR700 NHS ester, such as IRDye 700DX NHS ester (LiCor 929-70010, 929-70011). In some embodiments, the dye is a compound having the following formula:

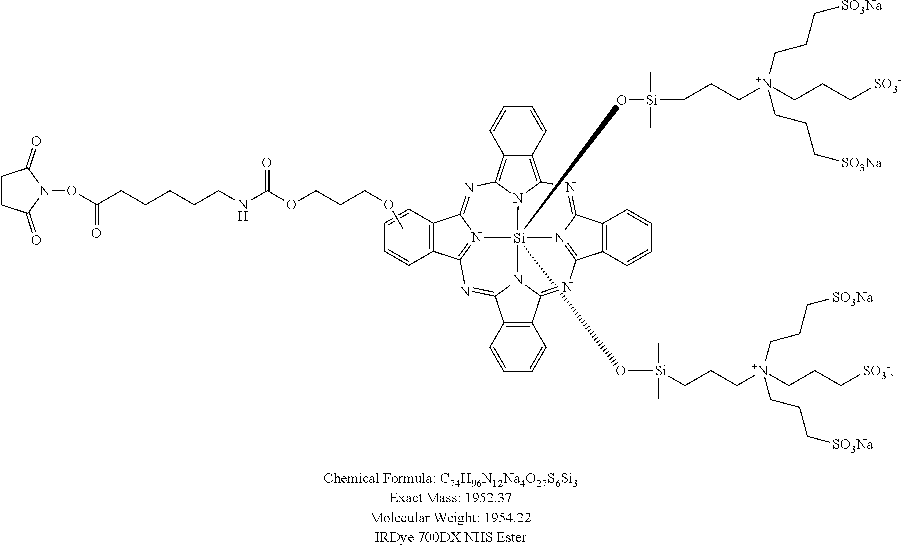

or is a salt, stereoisomer, or tautomer thereof.

For purposes herein, the term “IR700,” “IRDye 700” or “IRDye 700DX” includes the above formula when the dye is conjugated such as to an antibody, e.g. via a reactive group.

In some embodiments, the phthalocyanine dye contains a linker, i.e., is a linker-phthalocyanine dye moiety (L-D). In some embodiments, the linker contains a reactive group. In one aspect, the phthalocyanine dye compound has the Formula (X):

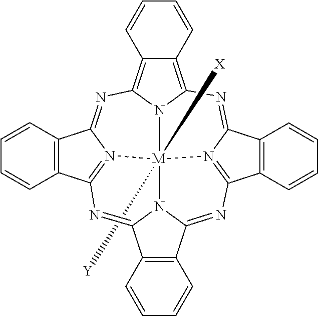

(X) or a salt, stereoisomer, or tautomer thereof, wherein:
M is a metal or metalloid;
X is

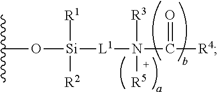

Y is

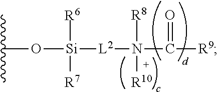

R¹ and R² are each independently optionally substituted alkyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl;
R³, R⁴ or R⁵ are selected from substituent group (a) or substituent group (b) wherein,
(a) R³ is hydrogen, -L³-H, -L³-A, or -L³-Z;
R⁴ is -L⁴-H, —(NH)ₘ-L⁴-A, —(NH)ₘ-L⁴-Z, —(O)ₘ-L⁴-A or —(O)ₘ-L⁴-Z;
R⁵ is -L⁵-H or -L⁵-A; and
(b) R³ is -L³-H, or -L³-A;
R⁴ is -L⁴-H, —(NH)ₘ-L⁴-A, or —(O)ₘ-L⁴-A; wherein R³ and R⁴ are connected with a bond to form a heterocyclyl substituted with -L⁴-A; and
R⁵ is -L⁵-H or -L⁵-A;
provided at least one of R³, R⁴ and R⁵ is a group containing A;
A is a reactive group capable of forming a covalent bond with a thiol, hydroxyl, carboxyl or amino group of a second moiety, or a protected form thereof or a reacted form thereof;
R⁶ and R⁷ are each independently optionally substituted alkyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl or optionally substituted heteroaralkyl;
R⁸, R⁹ or R¹⁰ are selected from substituent group (a) or substituent group (b) wherein,
(a) R⁸ is hydrogen, -L⁸-H or -L⁸-Z;
R¹⁰ is -L¹⁰-H, —(NH)ₙ-L⁹-Z or —(O)ₙ-L⁹-Z;
R¹⁰ is -L¹⁰-Z; and
(b) R⁸ and R⁹ are connected with a bond to form a heterocyclyl substituted with -L⁹-Z and R¹⁰ is -L¹⁰-H or -L¹⁰-Z;
provided at least one of R⁸, R⁹ and R¹⁰ is a group containing Z;
Z is a water soluble group optionally substituted with A or L′-A;
L¹ and L² are each independently optionally substituted alkylene, optionally substituted heteroalkylene, optionally substituted alkenylene, optionally substituted heteroalkenylene, optionally substituted cycloalkyl, or optionally substituted heterocyclyl;
L³, L⁴, L⁵, L⁸, L⁹ and L¹⁰ are each independently optionally substituted alkylene, optionally substituted heteroalkylene, optionally substituted alkenylene, optionally substituted heteroalkenylene, optionally substituted cycloalkylene, optionally substituted heterocyclene, optionally substituted arylene, optionally substituted aralkylene, optionally substituted heteroaralkylene, or optionally substituted heteroarylene, where the carbon atom of the alkylene, heteroalkylene, alkenylene, heteroalkenylene, cycloalkylene, heterocyclene, arylene, aralkylene, heteroaralkylene, or optionally substituted heteroarylene is further optionally substituted with Z and each nitrogen atom of the heteroalkylene or heteroalkenylene is optionally substituted with one or two L′-Z;
L′ is each independently optionally substituted alkylene, optionally substituted heteroalkylene, optionally substituted alkenylene, optionally substituted heteroalkenylene, optionally substituted cycloalkylene, optionally substituted heterocyclene, optionally substituted arylene, optionally substituted aralkylene, optionally substituted heteroaralkylene, or optionally substituted heteroarylene;
a is 0 or 1;
b is 0 or 1;
c is 0 or 1;
d is 0 or 1;
m is 0 or 1;
n is 0 or 1;
provided that if b is 1, then a is 0;
if d is 1, then c is 0;
if m is 1, b is 1; and
if n is 1, c is 1.

In certain embodiments, M is Si, Ge, Sn or Al. In yet certain embodiments, M is Si or Ge.

In certain embodiments, provided herein are conjugates that contain compounds of Formula (X) selected from the group consisting of:

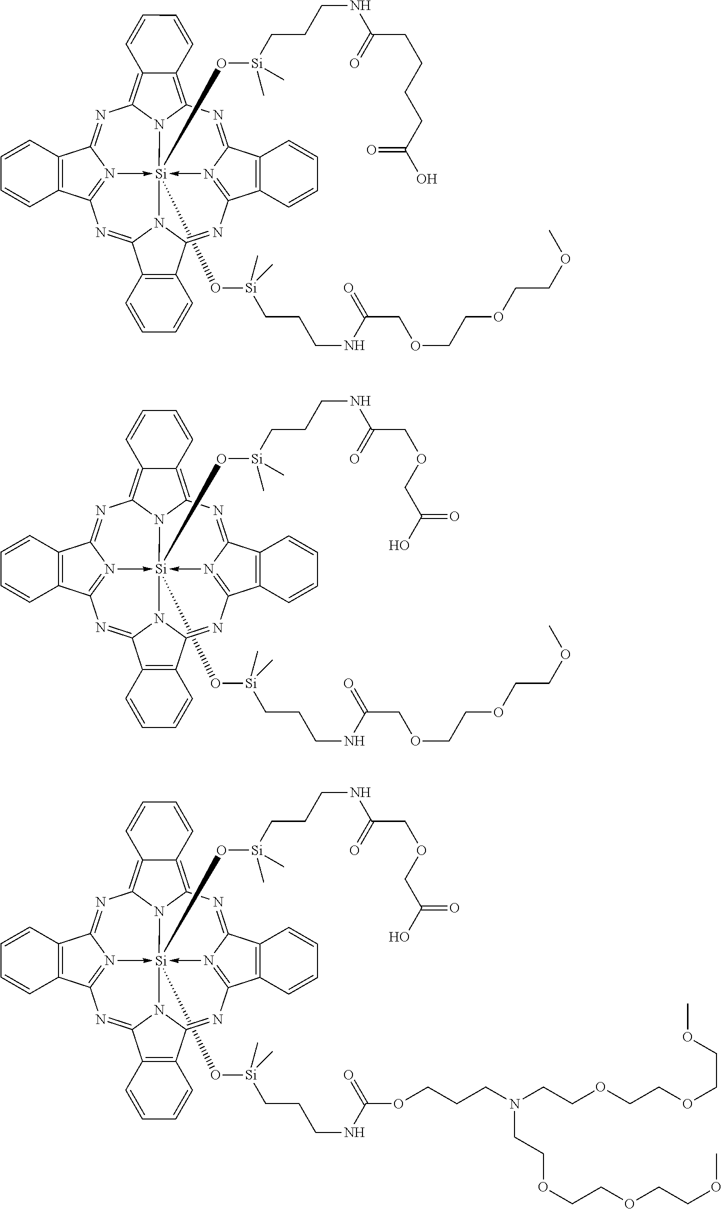

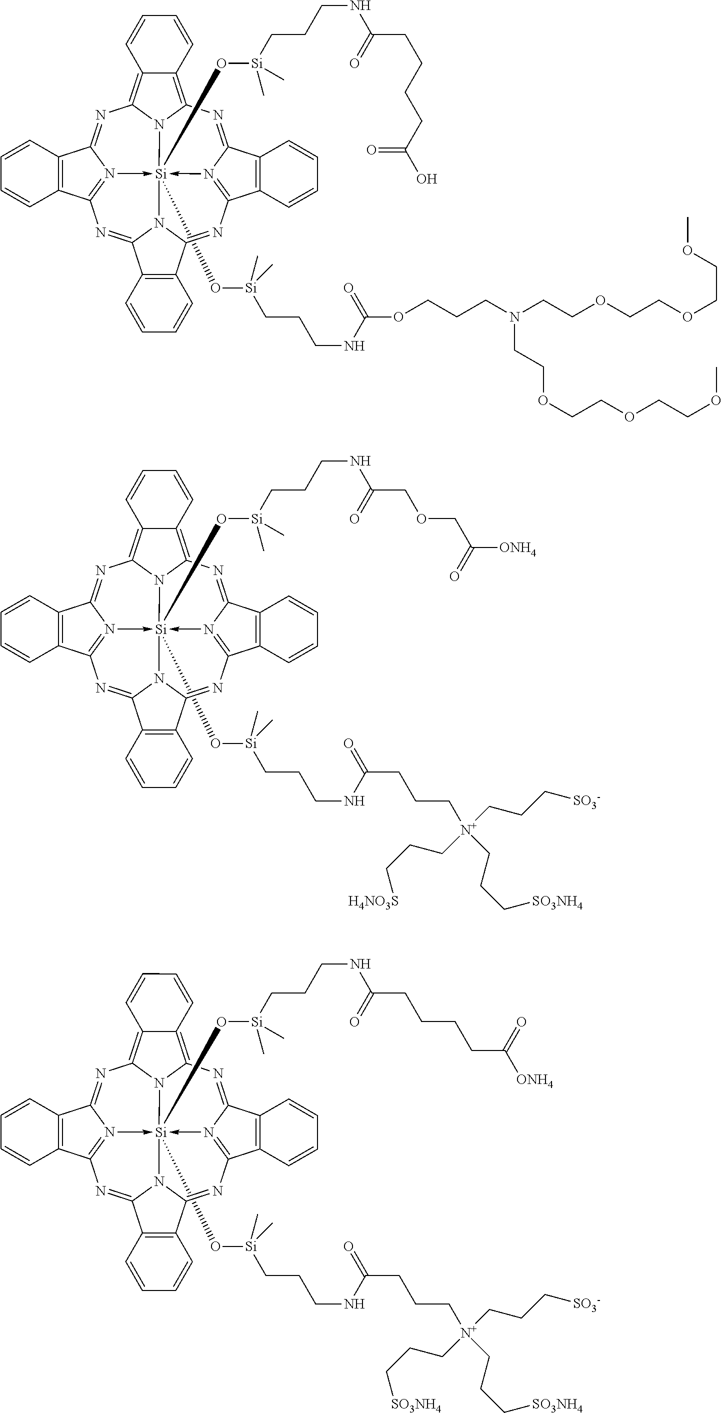

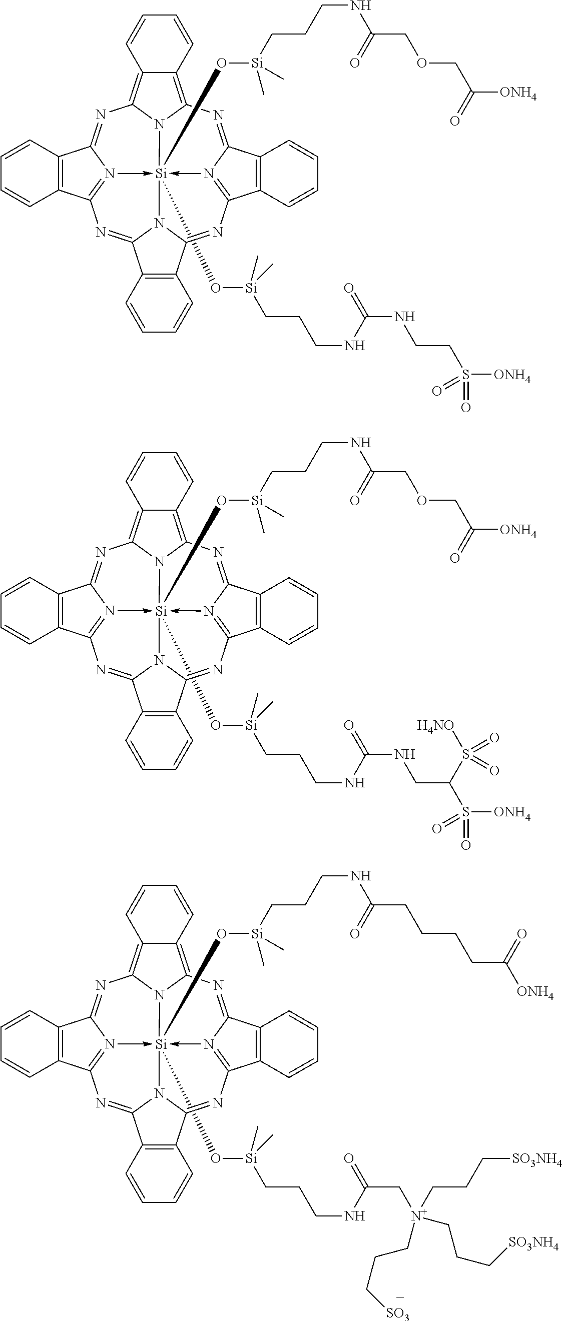

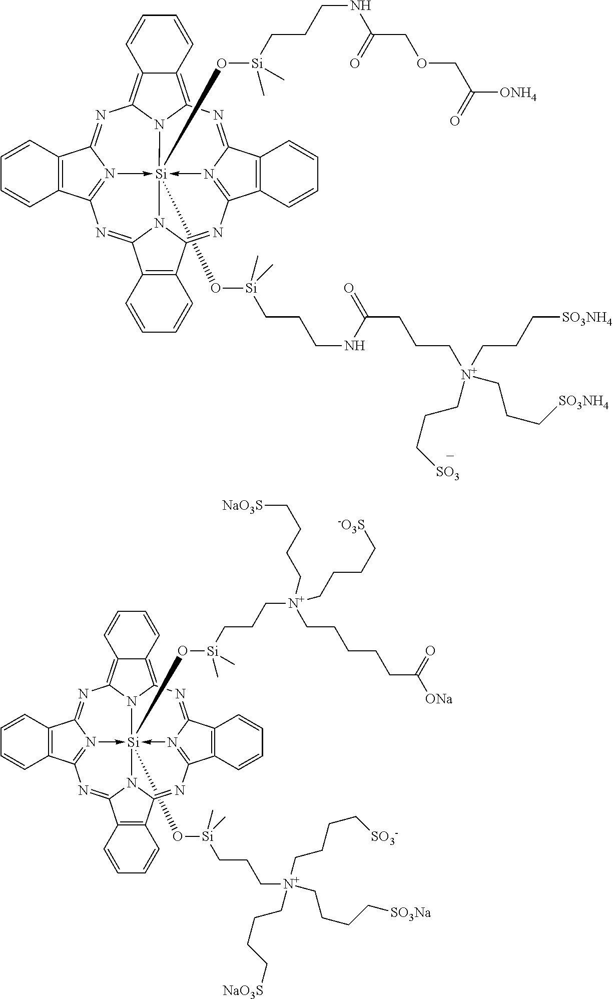

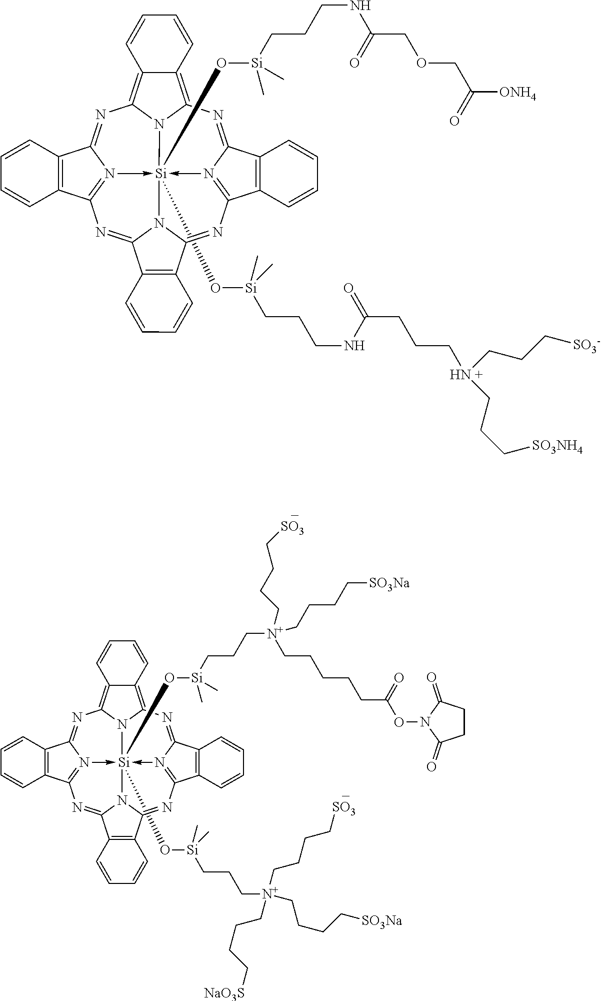

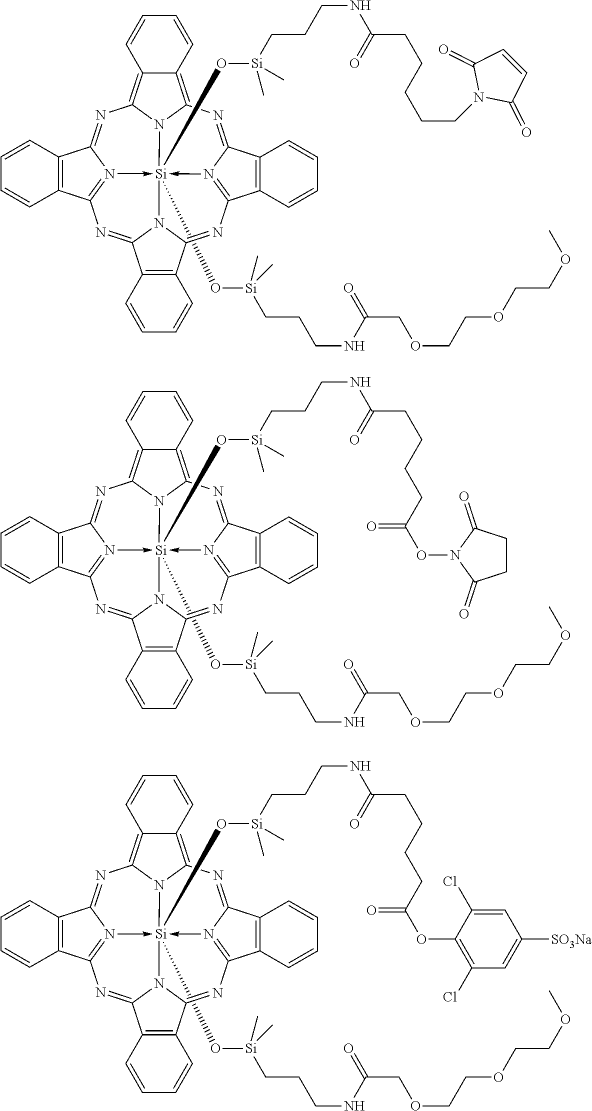

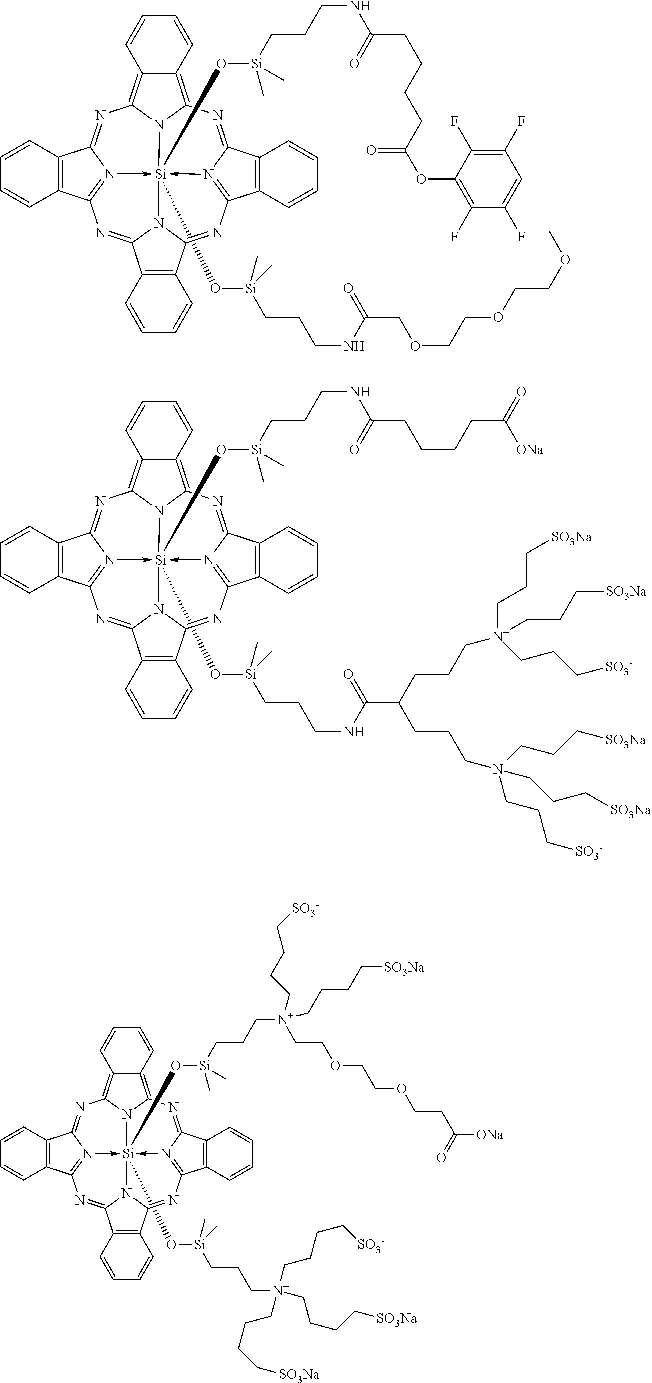

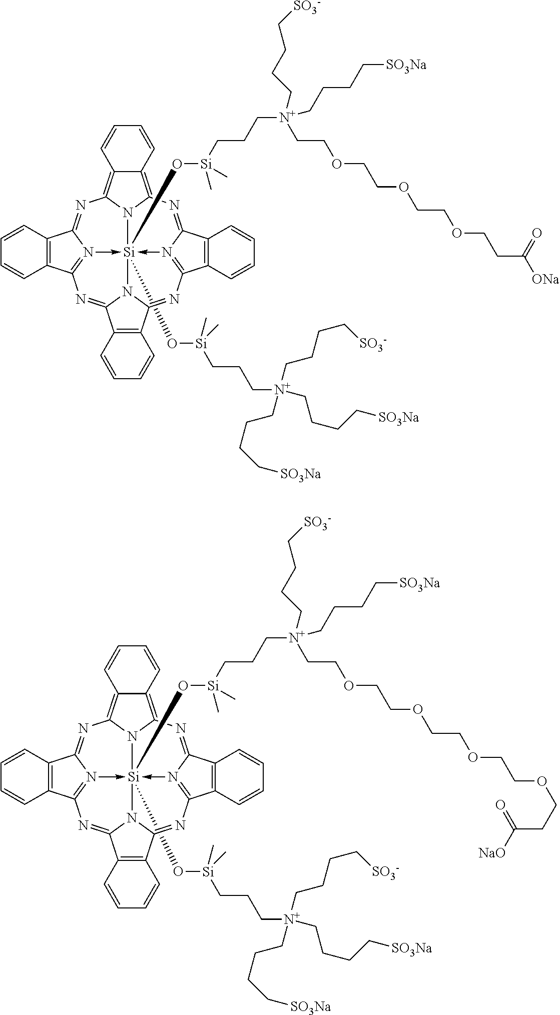

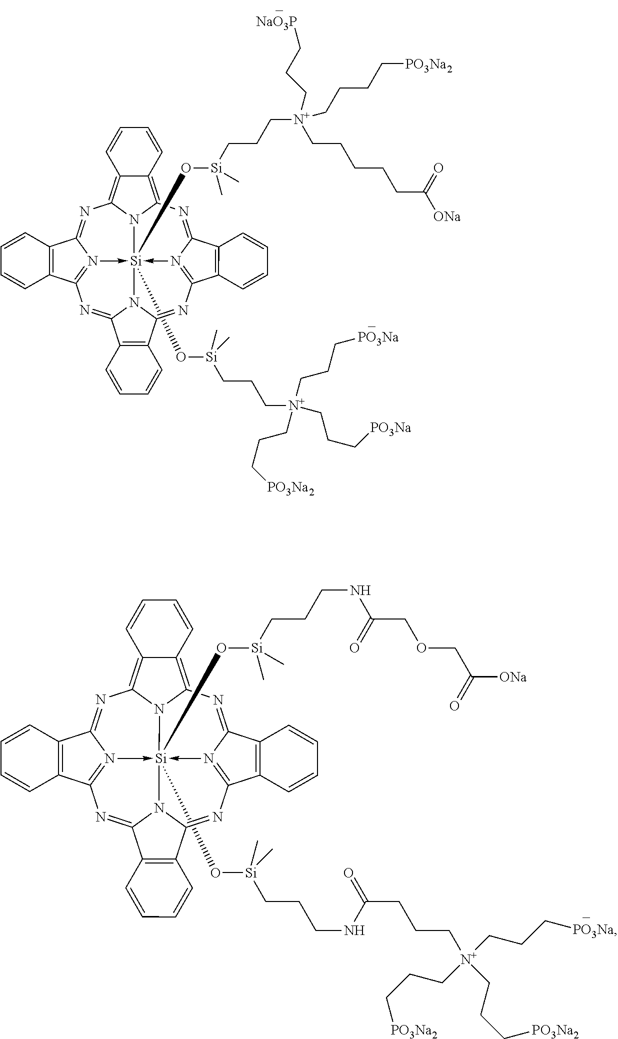

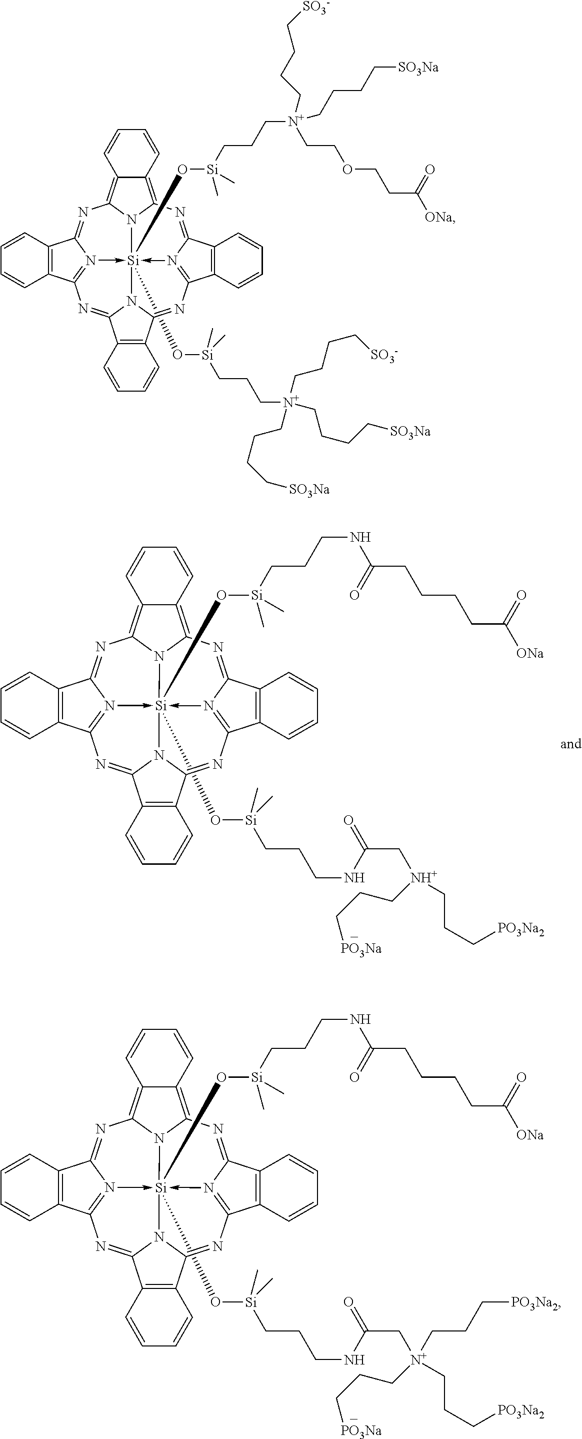

or a salt, stereoisomer, or tautomer thereof.

In particular embodiments, the compound of Formula (X) is selected from the group consisting of:

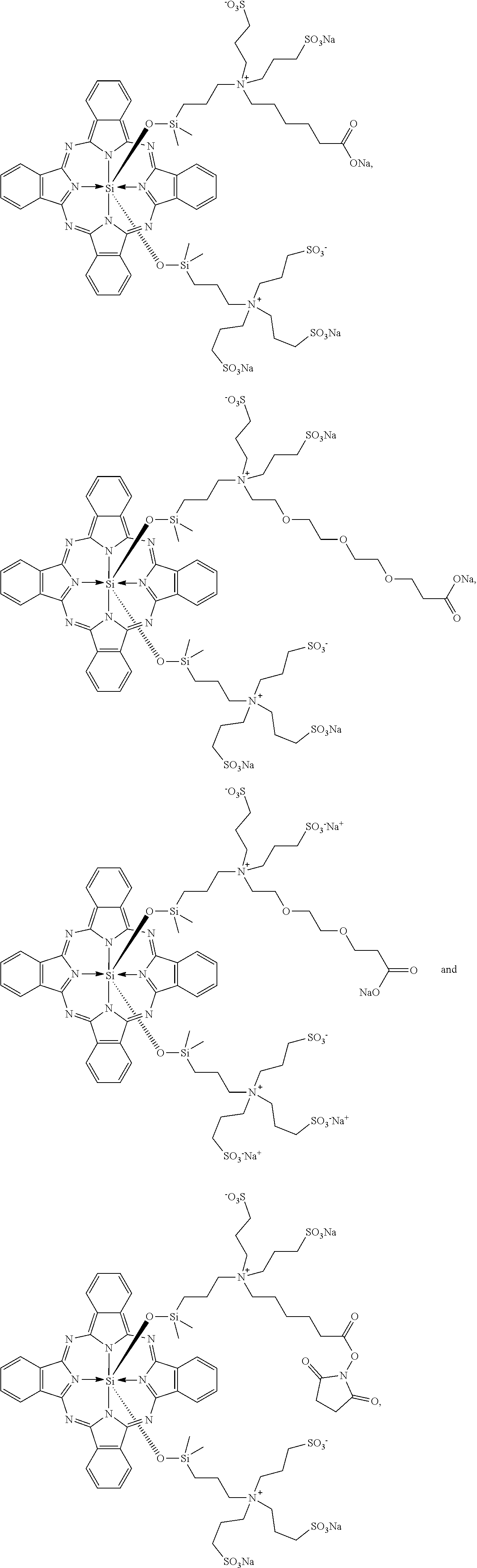

or a salt, stereoisomer, or tautomer thereof.

In some embodiments, the phthalocyanine dye is selected from Table A.

TABLE A

| Dye | |
|---|---|
| Compound | Structure |
| No. | Name |
| 1 | 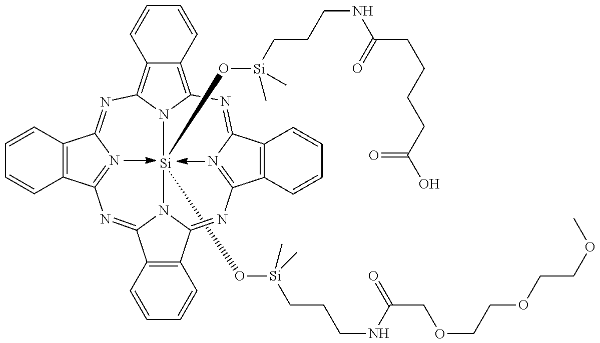 [SMILES: COCCOCCOCC(=O)NCCC[Si](C)(C)O[Si@@]1(O[Si](C)(C)CCCNC(=O)CCCCC(=O)O)n2c3c4ccccc4c2/N=C2N=C(/N=c4/c5ccccc5/c(n41)=N/C1=NC(=N\3)/c3ccccc31)c1ccccc1\2] |
| | 6-((3-(dimethyl((19-((15-methyl-10-oxo-2,5,8-trioxa-11-aza-15-silahexadecan- |
| | 15-yl)oxy)-19H-6,11-(azeno)-17,21- |
| | (azeno[3]episoindoloazeno)benzo[7,8][1,3,5,10]tetraaza[2]silacycloundecino[4,3- |
| | a:11,1-a′]diisoindol-19-yl)oxy)silyl)propyl)amino)-6-oxohexanoic acid |
| 5 | 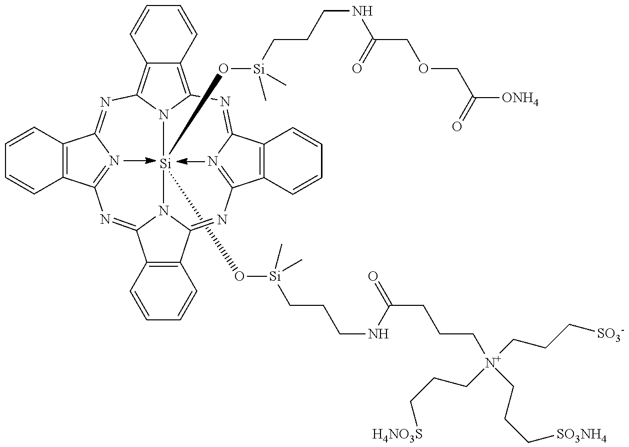 |
| | Ammonium 3-((4-((3-(((19-(((3-(2- |
| | (carboxymethoxy)acetamido)propyl)dimethylsilyl)oxy)-19H-6,11-(azeno)- |
| | 17,21- |
| | (azeno[3]episoindoloazeno)benzo[7,8][1,3,5,10]tetraaza[2]silacycloundecino[4,3- |
| | a:11,1-a′]diisoindol-19-yl)oxy)dimethylsilyl)propyl)amino)-4-oxobutyl)bis(3- |
| | sulfopropyl)ammonio)propane-1-sulfonate |
| 12 | 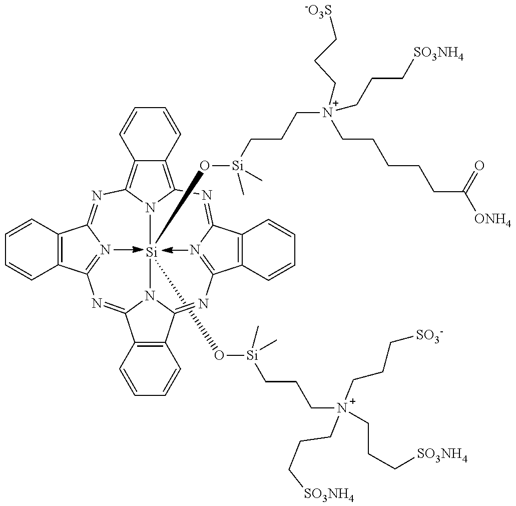 |
| | 3-((6-((λ5-azaneyl)oxy)-6-oxohexyl)(3-(((λ5-azaneyl)oxy)sulfonyl)propyl)(3- |
| | (((19-(((3-(bis(3-((λ5-azaneyl)oxy)sulfonyl)propyl)(3- |
| | sulfonatopropyl)ammonio)propyl)dimethylsilyl)oxy)-19H-6,11-(azeno)-17,21- |
| | (azeno[3]episoindoloazeno)benzo[7,8][1,3,5,10]tetraaza[2]silacycloundecino[4,3- |
| | a:11.1-a′]diisoindol-19-yl)oxy)dimethylsilyl)propyl)ammonio)propane-1- |
| | sulfonate |
| 13 | 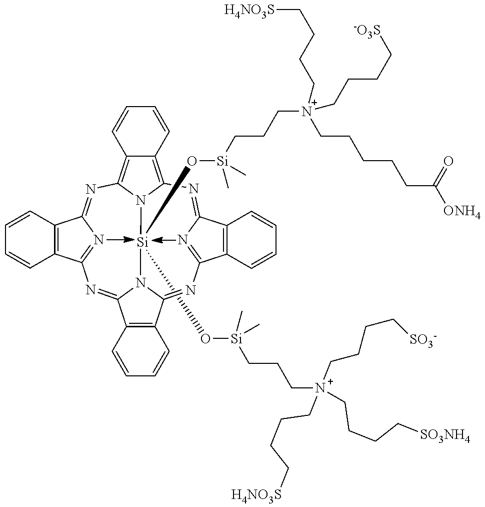 |
| | 4-((6-((λ5-azaneyl)oxy)-6-oxohexyl)(4-(((λ5-azaneyl)oxy)sulfonyl)butyl)(3- |
| | (((19-(((3-(bis(4-(((λ5-azaneyl)oxy)sulfonyl)butyl)(4- |
| | sulfonatobutyl)ammonio)propyl)dimethylsilyl)oxy)-19H-6,11-(azeno)-17,21- |
| | (azeno[3]episoindoloazeno)benzo[7,8][1,3,5,10]tetraaza[2]silacycloundecino[4,3- |
| | a:11,1-a′]diisoindol-19-yl)oxy)dimethylsilyl)propyl)ammonio)butane-1- |
| | sulfonate |
| 14 | 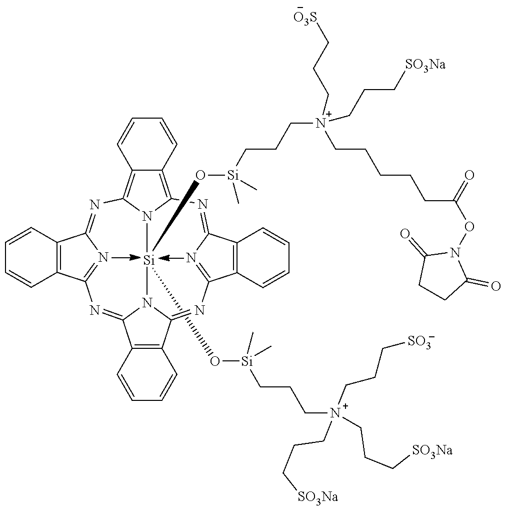 |
| | Sodium 3,3′,3″-((3-(((19-(((3-((6-((2,5-dioxopyrrolidin-1-yl)oxy)-6- |
| | oxohexyl)bis(3-sulfonatopropyl)ammonio)propyl)dimethylsilyloxy)-19H-6,11- |
| | (azeno)-17,21- |
| | (azeno[1]episoindoloazeno)benzo[7,8][1,3,5,10]tetraaza[2]silacycloundecino[4,3- |
| | a:11.1-a′]diisoindol-19-yl)oxy)dimethylsilyl)propyl)ammonio)butane-1- |
| | sulfonate) |
| 16 | 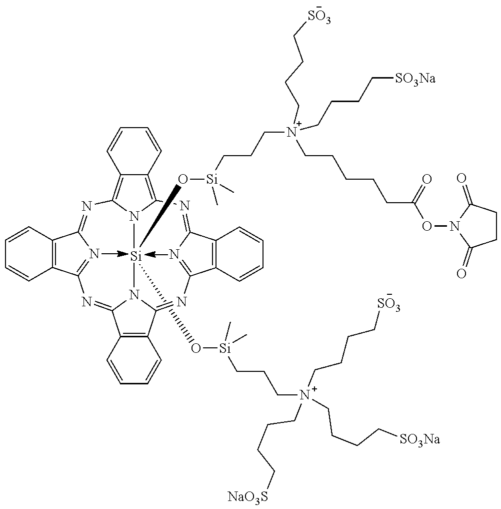 |
| | Sodium 4,4′,4″-((3-(((19-(((3-((6-((2,5-dioxopyrrolidin-1-yl)oxy)-6- |
| | oxohexyl)bis(4-sulfonatobutyl)ammonio)propyl)dimethylsilyl)oxy)-19H-6,11- |
| | (azeno)-17,21- |
| | (azeno[1]episoindoloazeno)benzo[7,8][1,3,5,10]tetraaza[2]silacyclundecino[4,3- |
| | a:11,1-a′]diisoindol-19-yl)oxy)dimethylsilyl)propyl)ammonio)tris(butane-1- |
| | sulfonate) |
| 17 | 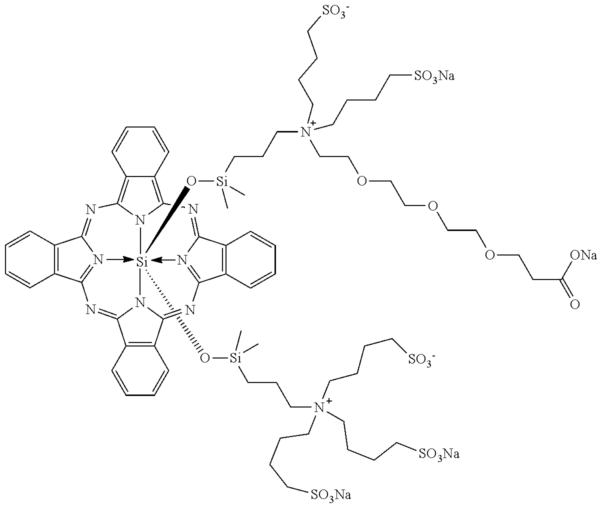 |
| | Sodium 2-((19-((dimethyl(3-(tris(4-sulfonatobutyl)ammonio)propyl)silyl)oxy)- |
| | 19H-6,11-(azeno)-17,21- |
| | (azeno[3]episoindoloazeno)benzo[7,8][1,3,5,10]tetraaza[2]silacycloundecino[4,3- |
| | a:11,1-a′]diisoindol-19-yl)oxy)-2-methyl-6,6-bis(4-sulfonatobutyl)-9,12,15- |
| | trioxa-6-aza-2-silaoctadecan-6-ium-18-oate |
| 18 | 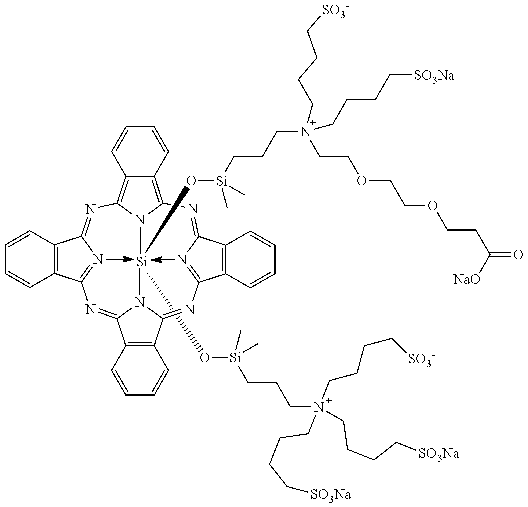 |
| | Sodium 2-((19-((dimethyl(3-(tris(4-sulfonatobutyl)ammonio)propyl)silyl)oxy)- |
| | 19H-6,11-(azeno)-17,21- |
| | (azeno[3]episoindoloazeno)benzo[7,8][1,3,5,10]tetraaza[2]silacycloundecino[4,3- |
| | a:11,1-a′]diisoindol-19-yl)oxy)-2-methyl-6,6-bis(4-sulfonatobutyl)-9,12-dioxa- |
| | 6-aza-2-silapentadecan-6-ium-15-oate |
| 19 | 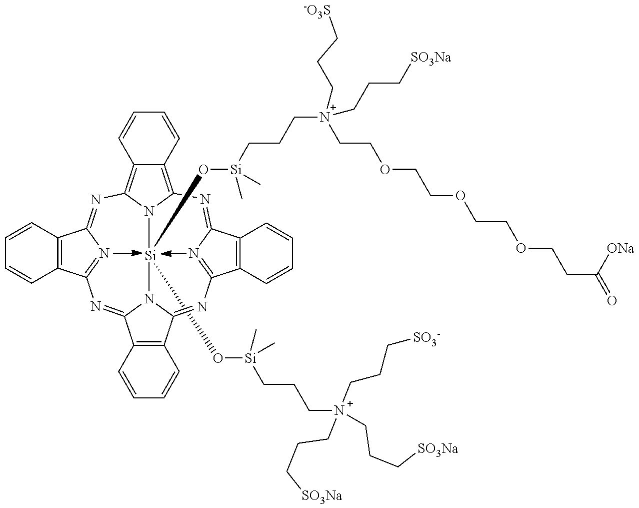 |
| | Sodium 2-((19-((dimethyl(3-(tris(3-sulfonatopropyl)ammonio)propylsilyl)oxy)- |
| | 19H-6,11-(azeno)-17,21- |
| | (azeno[3]episoindoloazeno)benzo[7,8][1,3,5,10]tetraaza[2]silacycloundecino[4,3- |
| | a:11,1-a′]diisoindol-19-yl)oxy)-2-methyl-6,6-bis(3-sulfonatopropyl)-9,12,15- |
| | trioxa-6-aza-2-silaoctadecan-6-ium-18-oate |

In particular embodiments, the phthalocyanine dye containing the reactive group has the structure of Formula (I):

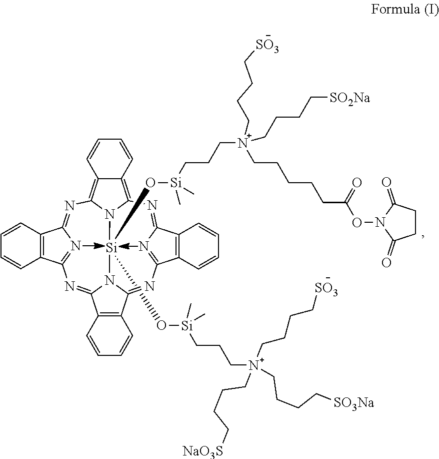

or a salt, stereoisomer, or tautomer thereof.

In particular embodiments, the phthalocyanine dye containing the reactive group has the structure of Formula (II):

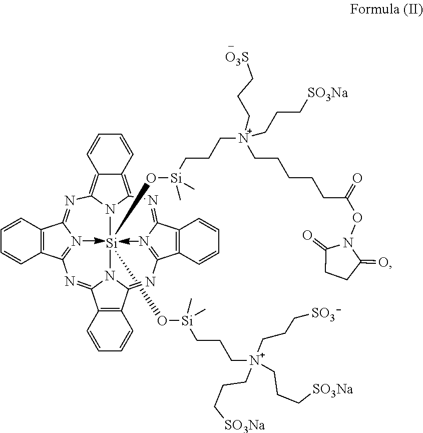

or a salt, stereoisomer, or tautomer thereof.

### C. Other Features

In some embodiments, provided herein are conjugates and conjugates for use with the methods herein comprising a Si-phthalocyanine dye linked to an anti-PD-L1 antibody or fragment provided herein. In some embodiments, the conjugate is an anti-PD-L1 antibody-Si-phthalocyanine dye conjugate. In some embodiments, the conjugate is an anti-PD-L1 antibody-IR 700 conjugate.

In some embodiments, the provided anti-PD-L1 antibodies or anti-PD-L1 antibody conjugates contain a functional Fc region. In some embodiments, the anti-PD-L1 antibody or conjugate does not comprise a functional Fc region. A non-functional Fc region includes an absent or truncated Fc region, or an Fc region the contains amino acid substitutions or deletions that reduces or eliminates Fc-mediated activities, such as effector functions (e.g., ADCC or ADCP activities). In some embodiments, the Fc region is engineered to exhibit ADCC and/or ADCP activity or engineered to exhibit enhanced effector function, such as enhanced ADCC and/or ADCP activity. Modifications to the Fc region that result in enhanced ADCC and/or ADCP activity can be any known modifications or can be empirically determined. Exemplary modifications include, but are not limited to the modifications to the Fc region described herein. In some embodiments, the antibodies are produced such that they are afucosylated.

In some aspects, the provided conjugates of an anti-PD-L1 antibody or antibody fragment and one or more phototoxic, cytotoxic, or fluorescent agent(s) may be made using any of a number of known protein coupling agents, e.g., linkers, (see Vitetta et al., Science 238:1098 (1987), WO94/11026). In some embodiments, the linker is a peptide or a polypeptide or is a chemical linker. In some embodiments, the linker is a releasable linker or a cleavable linker. The linker may be a “cleavable linker” or a “releasable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52:127-131 (1992); U.S. Pat. No. 5,208,020). In some embodiments, the releasable linker or the cleavable linker is released or cleaved in the presence of one or more conditions or factors present in the tumor microenvironment (TME), including includes matrix metalloproteinase (MMP), hypoxic conditions or acidic conditions.

### III. Methods of Treatment with and Uses of Anti-Pd-L1 Antibodies and Conjugates

In some embodiments, the provided anti-PD-L1 antibodies, fragment or conjugates are used in methods of treatment and uses of treating a lesion or a tumor. In some embodiments, the provided methods and uses involve administering an anti-PD-L1 antibody or fragment, or conjugate. In particular embodiments, the conjugate contains photoactivatable phthalocyanine dye, and the methods and uses involve administering the conjugate and illumination of a target area, with a wavelength of light suitable for use with the phthalocyanine dye, such that the light excites the dye and results in killing of a cell that expresses PD-L1 on its surface, for example, as described herein. Any of such methods and uses result in enhancing, activating, inducing, provoking, augmenting, or supporting immune function, such as local and/or systemic immunity, reducing or eliminating a lesion (e.g., tumor), reducing or inhibiting tumor growth, reducing, inhibiting, or eliminating tumor cell metastasis, or any combination thereof.

In some aspects, however, the provided compositions, methods and uses can further enhance, activate, induce, provoke, augment, or support immune function, such as local and/or systemic immunity, by inhibiting the PD-1:PD-L1 interaction and/or killing and eliminating cells that express PD-L1 on the surface of the cell. Thus, elimination or killing of cells expressing PD-L1 on the surface, particularly immune cells with an immunosuppressive function such as M2 tumor associated macrophages (M2 TAM), M1 or M2 macrophages, tolerogenic dendritic cells (tDCs) or myeloid derived suppressor cells (MDSCs), can function to enhance, activate, induce, provoke, augment, or support immune function, such as local and/or systemic immunity, such as anti-tumor or anti-cancer immunity. The elimination or killing of cells expressing PD-L1 on the surface can be effected by Fc-mediated effector function(s), delivery of a toxic payload to the PD-L1-expressing cell, and/or following illumination of a target area with a wavelength of light suitable for use with a photoactivatable dye, such that the light excites the dye and results in killing of the cell.

In some aspects the provided methods and uses can enhance, activate, induce, recruit, or support infiltration of lymphocytes into the tumor or lesion. In some embodiments, the provided methods and uses activate the intratumoral innate response, resulting increased activation of intratumoral dendritic cells (e.g., activated dendritic cells). In some aspects, the provided methods and uses activate the adaptive immune response, resulting in increased infiltration of CD8+ T cells. In some embodiments, the provided methods and uses lead to a reduction in the number of exhausted CD8+ T cells within the tumor. In some embodiments, the provided methods and uses lead to increased intratumoral infiltration of newly primed CD8+ T cells. In some embodiments, the provided methods and uses can result in improved therapeutic effect, such as by selecting subjects for treatment that has a higher level or number of non-exhausted effector cells, e.g., CD8+ T cells, and/or by improving the activity or response of non-exhausted effector cells.

In some aspects, the provided compositions, methods and uses can be applied to tumors resistant or refractory to checkpoint inhibitor immunotherapy. In some aspects, the provided compositions, methods and uses can be applied to tumors resistant or refractory to anti-PD-L1 immunotherapy. Tumors, such as solid tumors, can develop resistance to checkpoint inhibitor immunotherapy by several mechanisms including, but not limited to, irreversible T cell exhaustion, insufficient T cell priming, tumor cell immunoediting leading to the upregulation of compensatory inhibitory signaling of T cells, creating an immune suppressive tumor microenvironment, such as by increasing infiltration of Tregs, MDSCs, tumor associated macrophages (e.g., M2 macrophages), elevated levels of tumor -derived cytokines and chemokines (e.g., TGF-β, CXCL8), silencing Th1 type chemokines, indoleamine 2,3-dioxygenase (IDO) production, and excessive extracellular adenosine. The provided compositions, methods and uses find use in overcoming one or more checkpoint inhibitor resistance mechanisms employed by some tumors.

In addition, administration of the antibody, cytotoxic conjugates, or photoactivatable conjugates followed by illumination can also result in direct killing of cancer cells expressing PD-L1, thereby resulting in inhibition or reduction of tumor growth. The PD-L1 antibody, cytotoxic conjugates, or photoactivatable conjugates, such as anti-PD-L1-phthalocyanine conjugates, can affect and kill, directly or indirectly, a tumor cell or cells present in a tumor or the microenvironment of a tumor (also referred to as tumor microenvironment; TME), including tumor cells at a different location than the primary tumor, metastasized tumors, newly arising tumor cells and/or tumors of different types or cell surface antigen expression. Thus, the provided compositions, methods and uses can provide an effective treatment even for tumor cells that do not express cell surface PD-L1, tumors, lesions or cancers that has a low responsiveness or are substantially non-responsive to, has failed, has relapsed after, is refractory to and/or is resistant to prior therapies, such as prior immunomodulatory agent therapies. In particular embodiments, the provided compositions, methods, and uses can treat tumors or lesions that are non-responsive, resistant, or refractory to anti-PD-L1, anti-PD-1, and/or anti-CTLA-4 therapy.

In some embodiments, the compositions, methods and uses provided herein are also effective for treating tumors that are larger in size, and which exhibit greater immune suppression than smaller tumors. Such tumors may be less responsive or non-responsive to other treatments, such as to treatment with an immunomodulatory agent, such as an immune checkpoint inhibitor (e.g., anti-PD-L1, anti-PD-1, and/or anti-CTLA-4 therapy). In such cases, anti-PD-L1 photoimmunotherapy, rendered by the administration of the anti-PD-L1 conjugate described herein followed by illumination, can be effective in inhibiting or substantially reducing growth of larger tumors that, in some cases are not effectively inhibited by other immunomodulatory agent therapies and/or anti-cancer therapies. In some embodiments, the compositions, methods and uses provided herein are effective for treating tumors that are larger in size and resistant to anti-PD-L1, anti-PD-1, and/or anti-CTLA-4 therapy.

In some aspects, provided are methods and uses of treating a tumor or a lesion in a subject by activating an immune cell response. The immune cell activation can be direct or indirect activation. In some aspects, provided are methods and uses of treating a subject having a low response to, was unresponsive to, was resistant to, was refractory to, had failed to respond to or has relapsed after, a prior immunotherapy (e.g., an anti-PD-L1, anti-PD-1, and/or anti-CTLA-4 therapy) for a tumor or a lesion.

In some aspects, the methods involve administering to a subject having a tumor or a lesion a conjugate comprising a phthalocyanine dye, such as IR700, linked to an anti-PD-L1 antibody described herein. In some aspects, the methods also involve illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing cell, e.g., PD-L1 expressing immune cell, is located, such that the method can lead to the killing of the PD-L1 expressing cell and thereby inhibits the growth of the tumor or the lesion. In some aspects, the methods can lead to the killing of a PD-L1 expressing cell and thereby increases the number or activity of immune cells in the tumor or lesion and/or in the microenvironment of the tumor or lesion.

In some embodiments, provided are methods and uses of treating a tumor or a lesion in a subject by activating an immune cell response that includes illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, in a subject having a tumor or a lesion that had been administered a conjugate comprising a phthalocyanine dye linked to a targeting molecule that binds to PD-L1; wherein the method leads to the killing of the PD-L1 expressing cell and thereby inhibits the growth of the tumor or the lesion. In some embodiments, the PD-L1 expressing cell is an immune cell. In some embodiments, the PD-L1 expressing cell is a tumor cell.

In some embodiments, provided are methods and uses of treating a subject having a low response to, was unresponsive to, was resistant to, was refractory to, had failed to respond to or has relapsed after, a prior immunotherapy for a tumor or a lesion comprising illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, in a subject having a tumor or a lesion that has had a low response or that was unresponsive to a prior immunotherapy that had been administered a conjugate comprising a phthalocyanine dye linked to a targeting molecule that binds to PD-L1; wherein the method leads to the killing of a PD-L1 expressing cell and thereby increases the number or activity of immune cells in the tumor and/or in the tumor microenvironment. In some of any of the embodiments, the PD-L1 expressing cell is an immune cell.

In some embodiments, provided are methods and uses of enhancing a response to an anti-cancer agent in a subject having a tumor or a lesion. In some aspects, the methods involve administering an anti-cancer agent to a subject having a tumor or a lesion. In some aspects, administering a conjugate comprising a phthalocyanine dye linked to a targeting molecule that binds to PD-L1 to the subject. In some aspects, illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, leading to a greater inhibition of growth of the tumor or the lesion compared to the inhibition by treatment with the anti-cancer agent alone.

In some embodiments, provided are methods and uses of enhancing a response to an anti-cancer agent in a subject having a tumor or a lesion that involves: administering to a subject a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, wherein the subject had been administered an anti-cancer agent; leading to a greater inhibition of growth of the tumor or the lesion compared to the inhibition by treatment with the anti-cancer agent alone.

In some embodiments, provided are methods and uses of enhancing a response to an anti-cancer agent in a subject having a tumor or a lesion involves: administering an anti-cancer agent to a subject; wherein the subject had received a treatment comprising administering a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, and wherein the administration of the anti-cancer agent and the treatment leads to a greater inhibition of growth of the tumor or the lesion compared to the inhibition with the anti-cancer agent alone.

In some embodiments, provided are methods and uses of vaccinating or immunizing a subject to generate an anti-cancer immune response. In some aspects, vaccinating or immunizing a subject to generate an anti-cancer immune response can inhibit the growth and/or reduce the size of a first tumor or lesion; and also delay or prevent the appearance, growth or establishment of one or more second tumors or lesions, for example located distally to the treated first tumor or lesion. In some aspects, the methods involve administering a conjugate comprising a phthalocyanine dye linked to a targeting molecule that binds to PD-L1 to a subject. In some aspects, the methods involve illuminating a target area, leading to an anti-cancer response selected from a delay or inhibition in the appearance of or growth of a tumor in the subject or an appearance or increase in T memory cells in the vicinity of a tumor.

In some embodiments, provided are methods and uses of vaccinating or immunizing a subject to generate an anti-cancer immune response that involves: illuminating a target area in a subject that had been administered a conjugate comprising a phthalocyanine dye linked to a targeting molecule that binds to PD-L1; leading to an anti-cancer response selected from a delay or inhibition in the appearance of or growth of a tumor in the subject or an appearance or increase in T memory cells in the vicinity of a tumor.

In some embodiments, one or more of steps of the method are repeated. In some embodiments, the administration of the conjugate is repeated one or more times, optionally wherein after each repeated administration of the conjugate, the illuminating step is repeated. In some embodiments, further comprising administering an additional therapeutic agent or anti-cancer treatment.

### A. Methods for Stimulating or Enhancing Anti-Cancer Immune Responses

In some aspects, the provided methods and uses employing compositions including an anti-PD-L1 conjugate can result in an enhancement of an immune response, such as systemic and/or local immune response in the subject, which in turn can result in an enhanced response to the therapy or treatment for a tumor, a lesion or a cancer. In some aspects, the methods and uses herein include administering to the subject anti-PD-L1 conjugate, and after administration of the conjugate, illuminating the target area, such as a target area where PD-L1-expressing cells are present, e.g., a tumor, the vicinity of a tumor, a lymph node, the vicinity of the lymph node, or the tumor microenvironment.

In some aspects, the provided embodiments can stimulate, enhance, activate, induce, provoke, boost, augment, or support an immune response, such as a systemic immune response, in a subject having a tumor, a lesion or a cancer. In some embodiments, the provided method and uses results in enhancing a systemic immune response in a subject having a tumor, a lesion or a cancer. “Systemic immune response” refers to the ability of a subject's immune system to respond to an immunologic challenge or immunologic challenges, including those associated with a tumor, a lesion or a cancer, in a systemic manner. Systemic immune response can include systemic response of the subject's adaptive immune system and/or innate immune system. Systemic immune response can include anti-tumor or anti-cancer response from the subject's adaptive immune system and/or innate immune system. In some aspects, systemic immune response includes an immune response across different tissues, including the blood stream, lymph node, bone marrow, spleen and/or the tumor microenvironment, and in some cases, includes a coordinated response among the tissues and organs and various cells and factors of the tissues and organs. In some embodiments, the provided embodiments can stimulate, enhance, activate, induce, provoke, boost, augment, or support the anti-cancer or anti-tumor immune response of the subject's own immune system, including the adaptive immune system and/or innate immune system. In some aspects, the provided methods and uses can result in enhancement of an innate immune response in the subject.

In some aspects, the provided embodiments can also exhibit an abscopal effect. In some aspects, “abscopal effect” refers to a treatment effect in which a tumor that is not directly treated or is away from the site of localized treatment, e.g., a distal or a metastatic tumor, is also treated, for example, reduced in tumor volume.

In some aspects, the provided embodiments can effect tumor immunity. In such aspects, the provided embodiments prevent or impede growth of a new tumor or a metastasis. In some embodiments, inhibition of tumor growth effected by the provided embodiments leads to a durable anti-tumor response. In some embodiments, inhibition of tumor growth effected by the provided embodiments leads to prolonged progression-free survival. In some embodiments, inhibition of tumor growth effected by the provided embodiments leads to a reduced chance of relapse and/or a reduced chance of metastasis. In some aspects, the provided embodiments can effect immunity for the same tumor type or a different tumor type in the treated subject. In some aspects, the provided embodiments can inhibit growth of tumors from a different tumor lineage, i.e., a different type of tumor that arises or could arise in a treated subject.

In some aspects, the target area is an area that comprises PD-L1 expressing cells. In some embodiments, the PD-L1 expressing cell is an immune cell. In some of any of the embodiments, the methods lead to the killing of the PD-L1 expressing cells, such as PD-L1 expressing immune cells. In some embodiments, the PD-L1 expressing immune cell is selected from the group consisting of monocytes, macrophages, dendritic cells (DC), M2 tumor associated macrophages (M2 TAM), M1 or M2 macrophages, tolerogenic dendritic cells (tDC) and myeloid derived suppressor cells (MDSC). In some embodiments, the PD-L1 expressing immune cell is located in the tumor, the tumor microenvironment or a lymph node.

In some aspects, the target area to be illuminated in accordance with the provided embodiments is a tumor, such as a primary tumor, the vicinity of a tumor, such as a primary tumor, or the tumor microenvironment (TME). In some embodiments, the target area is near the tumor or proximal to the tumor, or the vicinity of the tumor or tumor cells. In some embodiments, the target area is a tumor. In some embodiments, the target area is a primary tumor. In some embodiments, the target area is a secondary tumor or a metastasized tumor. In some embodiments, the target area is the tumor microenvironment.

In some embodiments, the target area is a lymph node or the vicinity of the lymph node. In some embodiments, the target area is a lymph node, for example, containing PD-L1 expressing cells, or the vicinity of the lymph node. In some embodiments, the target area is a lymph node. In some embodiments, the target area is a vicinity of a lymph node.

In some aspects, the provided embodiments can stimulate or enhance a systemic response, such as a systemic immune response, against one or more primary tumors or lesions and/or one or more second tumors or lesions, such as metastatic tumors or lesions, or tumors or lesions of a different type.

In some aspects, the provided embodiments stimulate or enhance the subject's immune response, such as the subject's anti-cancer immune response, in some cases by removing PD-L1 expressing immune cells, such as those that may have an immunosuppressive function, such as monocytes, macrophages, such as M1 macrophages, M2 macrophages and/or M2 tumor associated macrophages (M2 TAM), dendritic cells (DC), tolerogenic dendritic cells (tDCs), or myeloid derived suppressor cells (MDSCs). In some aspects, the provided embodiments stimulate or enhance the subject's immune response, such as the systemic and/or local immune response that target the tumor, lesion or cancer, by killing and eliminating PD-L1 expressing immunosuppressive cells, such as M2 TAM, tDCs or MDSCs. As exemplified herein, inhibition of tumor growth following administration of an PD-L1-phthalocyanine dye conjugate and light illumination requires the presence and/or activity of the subject's CD8+ T cells, as depletion of the subject's CD8+ T cells resulted in tumor growth similar to the growth in saline administered control. In some aspects, the immunosuppressive cells, e.g., M2 TAM, tDCs or MDSCs, such as those that express PD-L1, inhibit the function and/or activity of the subject's immune cells, such as CD8+ T cells or natural killer (NK) cells. By virtue of killing and eliminating the immunosuppressive cells, such as PD-L1 expressing immunosuppressive cells, including M2 TAM, tDCs or MDSCs, the provided embodiments can stimulate and enhance the subject's immune response. As exemplified herein, such treatment according to the provided embodiments results in inhibition of growth, such as a complete response to the treatment, of one or more primary tumors, and inhibition of growth of one or more second tumor, such as a second tumor of the same or different types and/or origin and/or a second tumor present at a different site, such as a distal site, from the primary tumor or lesion.

In some aspects, inhibition of the growth of the tumor or the lesion and/or killing of the PD-L1 expressing cell is dependent on the presence of CD8+ T cells. In some embodiments, prior to the administering, the subject has a tumor or a lesion having a low number or level of CD8+ T cell infiltration. In some embodiments, the number, level or activity of immune cells is increased in the tumor or in the tumor microenvironment after the administering and the illuminating. In some embodiments, the number or level of CD8+ T cell infiltration in the tumor or the lesion is increased after the administering and the illuminating. In some embodiments, the number of memory T cells in the vicinity of the tumor is increased after the administering and the illuminating.

In some aspects, the stimulated or enhanced systemic immune response includes an increase in the number and/or activity of systemic CD8⁺ T effector cells, an increase in systemic T cell cytotoxicity against tumor cells as measured using a CTL assay using cells from the spleen, the peripheral blood, the bone marrow, or the lymph nodes, an increase in the number, activity and/or priming of intratumoral CD8⁺ T effector cells in the primary or secondary (e.g., metastatic or new) tumors or lesions, an increase in systemic CD8⁺ T cell activation, an increase in systemic dendritic cell activation, an increase in dendritic cell activation in the primary or secondary (e.g., metastatic or new) tumors or lesions, an increase in intratumoral dendritic cell infiltration in the primary or secondary (e.g., metastatic or new) tumors or lesions, an increase in new T cell priming in the primary or secondary (e.g., metastatic or new) tumors or lesions, an increase in T cell diversity in the primary or secondary (e.g., metastatic or new) tumors or lesions, a decrease in systemic regulatory T cells, a decrease in regulatory T cells in the primary or secondary (e.g., metastatic or new) tumors or lesions, a decrease in systemic myeloid derived suppressor cells, a decrease in intratumoral myeloid derived suppressor cells in the primary or secondary (e.g., metastatic or new) tumors or lesions, a decrease in tumor associated fibroblasts or cancer associated fibroblasts (CAFs), in the primary or secondary (e.g., metastatic or new) tumors or lesions, or any combination thereof in the subject. In some instances, a systemic response can be assessed by sampling blood, tissue, cells or other fluid from a subject and assessing an increase in pro-inflammatory cytokines, an increase or appearance of immune cell activation markers and/or T cell diversity. In some aspects, a systemic response may be assessed by assaying cells affected directly or indirectly by the methods. For example, cell can be collected from the subject between day 4 and day 28 after treatment or any time after the step of illumination of the primary tumor in the subject.

In some aspects, the provided embodiments can stimulate, enhance, boost, augment, or support an immune response, such as a local response, such as a local immune response, in a subject having a tumor, a lesion or a cancer. In some embodiments, the provided method and uses results in enhancing a local response in a subject having a tumor, a lesion or a cancer. “Local immune response” refers to the immune response in a tissue or an organ to an immunologic challenge or immunologic challenges including those associated with a tumor, a lesion or a cancer. A local immune response can include the adaptive immune system and/or innate immune system. In some aspects, local immunity includes immune response concurrently occurring at different tissues, such as the blood stream, lymph node, bone marrow, spleen and/or the tumor microenvironment.

In some aspects, the stimulated or enhanced local immune response includes an increase in the number and/or activity of intratumoral CD8⁺ T effector cells (e.g., CD3⁺ CD8⁺ cells), an increase in CD8⁺ T effector cell activation, an increase in intratumoral dendritic (CD11c⁺) cell infiltration, an increase in intratumoral dendritic cell activation (e.g., CD11c⁺ CD80⁺ and/or CD11c⁺ CD40⁺), an increase in intratumoral antigen-presenting dendritic cells (CD11b⁺ CD103⁺ CD11c⁺), an increase in intratumoral new T cell priming (e.g., CD3⁺ CD8⁺ PD1⁻ cells), an increase in intratumoral T cell diversity, an increase in intratumoral neutrophils (CD11b⁺ Cy6C^{-/low} Ly6G⁺ cells), a decrease in intratumoral macrophages (e.g., CD11b⁺ F4/80⁺ cells), a decrease in intratumoral regulatory T cells (Tregs), a decrease in intratumoral myeloid derived suppressor cells (MDSCs; e.g., CD11b⁺ Ly6C⁺ Ly6G⁻ cells), a decrease in intratumoral tumor associated fibroblasts or cancer associated fibroblasts (CAFs), a decrease in the number and/or activity of intratumoral exhausted T cells, such as exhausted CD8+ T cells (e.g., PD-1⁺CTLA-4⁺CD3⁺CD8⁺ cells), or any combination thereof in the subject. In some aspects, the stimulated or enhanced local immune response is effected by any of the provided embodiments. In some aspects, the cell surface phenotype of cells, such as immune cells indicative of local immune response or innate immune response, is assessed by staining with reagents, such as labelled antibodies, that can be used to detect the expression of the marker(s) on the surface. In some aspects, the cell surface phenotype of cells, such as immune cells indicative of local immune response or innate immune response, is detected using flow cytometry.

In some cases, a local response, such as a local immune response, can be assessed by taking a blood, tissue or other sample from a subject and assessing for an increase in an anti-immune cell type in the tumor or TME and/or assessing for an increase or appearance of local immune activation markers. In some aspects, a local response, such as a local immune response, may be assessed by assaying cells affected directly or indirectly by the methods. For example, cell can be collected from the subject between day 4 and day 28 after treatment or any time after the step of illumination of the primary tumor in the subject.

In some aspects, the methods and uses also involve administering an additional therapeutic agent, such as an immunomodulatory agent, e.g., an immune checkpoint inhibitor. The immunomodulatory agent can be administered prior to, concurrent with or subsequent to the administration of the conjugate. In some aspects, administration of the additional therapeutic agent, such as an immunomodulatory agent, can also contribute to stimulating, enhancing, activating, inducing, augmenting or supporting an immune response, such as the subject's systemic and/or local immune response, including anti-cancer or anti-tumor responses. Exemplary additional therapeutic agents, compositions, combinations, methods and uses include those described herein, e.g., in Section V.

### B. Tumors and Lesions for Anti-PD-L1 Conjugate Therapy

The methods described herein include administration of an anti-PD-L1 conjugate and illuminating a target area, such as a tumor or a lesion, the vicinity of a tumor, a lymph node, the vicinity of a lymph node, or the tumor microenvironment (TME) of a tumor or a lesion, in a subject with a wavelength of light to activate the phthalocyanine dye moiety of the conjugate to achieve cell killing, for example, of cells expressing PD-L1 on the surface. In some embodiments, the methods and uses provided herein include treating a subject that has one or more tumors or lesions, such as one or more primary tumors or lesions (or first tumors or lesions), one or more secondary tumors or lesions (or second tumors or lesions), one or more newly arising tumors or lesions and/or one or more metastasized tumors or lesions. The subject may have one, two, three, or more than three tumors. Such tumors can be in one or more tissues or organs, such as in one tissue or organ, in two different tissues or organs, in three different tissues or organs, or in more than three different tissues or organs. In some aspects one or more of the tumors to be treated expresses PD-L1 on the surface of the cells of which the tumor is comprised. In some aspects, one or more of the tumors to be treated contain, are primarily composed of, have a substantial number of, or are entirely composed of cells that do not express PD-L1, have low PD-L1 expression, or are PD-L1-negative. In some aspects, one or more of the tumors to be treated contain, are primarily composed of, have a substantial number of, or are entirely composed of cells that have a reduced response, are resistant to, or become resistant to (i.e., acquire resistance to) PD1/PD-L1 checkpoint blockade.

In some aspects, the tumor or lesion treated according to the provided embodiments is treatment-naïve for, or has not previously received a treatment with, an immune checkpoint inhibitor, such as naïve for anti-PD-1, anti-PD-L1 and/or anti-CTLA-4 therapy/therapies. In some embodiments, the tumor or lesion has not received (is naïve to) anti-PD-1 treatment. In some embodiments, the tumor or lesion has not received (is naïve to) anti-PD-L1 treatment. In some embodiments, the tumor or lesion has not received (is naïve to) anti-CTLA-4 treatment. In some embodiments, the subject having a tumor or lesion to be treated according to the provided embodiments is treatment-naïve for an immune checkpoint inhibitor. In some embodiments, the subject to be treated is naïve to anti-PD-1 treatment. In some embodiments, the subject to be treated is naïve to anti-PD-L1 treatment. In some embodiments, the subject to be treated is naïve to anti-CTLA-4 treatment. Treating a subject with an immune checkpoint inhibitor, such as an anti-PD-1 antibody, can lead to CD8+ effector T cell exhaustion in the tumor, its periphery, and/or systemically. This can lead to the inability of the CD8+ T cells to recognize and localize to the tumor, or it can render the CD8+ T cells ineffective despite localization to the tumor or to the vicinity of the tumor, leading to checkpoint inhibitor (e.g., PD-1/PD-L1) resistance. Hence, in some instances, ineffective or insufficient CD8+ effector T cell activity can be mitigated by avoiding immune checkpoint inhibitor therapy (e.g., anti-PD-1, anti-PD-L1 and/or anti-CTLA-4 therapy) prior to employing the provided compositions, methods, or uses. In some embodiments, the response of a tumor or lesion to treatments herein is effected by first treating the tumor or lesion with the administration of an anti-PD-L1 conjugate followed by illumination, prior to any treatment of the tumor or lesion with an immune checkpoint inhibitor, such as a PD-1, PD-L1, and/or CTLA-4 directed therapy (such as an anti-PD-1 antibody, and anti-PD-L1 antibody, and/or anti-CTLA-4 antibody). In some embodiments, methods of treatment include selecting subjects that have not received treatment with an immune checkpoint inhibitor (e.g., anti-PD-1, anti-PD-L1, and/or anti-CTLA-4) therapy and treating such subjects (i.e. a tumor or lesion of such subject) with an anti-PD-L1 conjugate followed by illumination.

In some aspects, tumor or the lesion for treatment in accordance with the provided embodiments is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some aspects, the tumor or lesion treated according to the provided embodiments include one or more primary (e.g., first) tumor or lesion. In some aspects, a primary tumor or lesion can include the first or original tumor or lesion in a subject. In some aspects, the subject can have one or more primary tumors or lesions. In some embodiments, a primary tumor or primary tumors may be a solid tumor or solid tumors, may be lymphomas, or may be leukemias. The tumor can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown primary origin.

In some aspects, the tumor or lesion treated according to the provided embodiments include one or more second tumor or lesion, such as a metastatic tumor or lesion, or a newly arising tumor or lesion. In some aspects, the one or more second tumor or lesion is derived from a metastasis of the first tumor or lesion. In some embodiments, the one or more second tumor or lesion is a tumor that is not derived from a metastasis of the first tumor or lesion. In some aspects, the one or more second tumor or lesion is phenotypically and/or genotypically different from the first tumor or lesion. In some aspects, the one or more second tumor or lesion is phenotypically different from the first tumor or lesion. In some aspects, the one or more second tumor or lesion is genotypically different from the first tumor or lesion. In some aspects, the one or more second tumor or lesion is a newly arising tumor or lesion. In some aspects, the one or more second tumor or lesion is from a different origin compared to the first tumor or lesion. In some aspects, the one or more second tumor or lesion arises from a different organ or a different cell compared to the first tumor or lesion. In some embodiments, the one or more second tumor or lesion may be a solid tumor or solid tumors, may be lymphomas, or may be leukemias. The one or more second tumor or lesion can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown origin.

In some embodiments, immunity to a second tumor or lesion is effected when the first tumor is illuminated, following administration of the provided anti-PD-L1 conjugate, and the volume of the first tumor is reduced. In such embodiments, the volume of the first tumor is reduced by at least or at least about 10%, 15%, 20%, 25%, 30%, 35%, 40%, 45%, 50%, 55%, 60%, 65%, 70%, 75%, 80%, 85%, 90%, 95%, or 100%. In some embodiments, the volume of the first tumor is reduced by at least or at least about 50%. In some embodiments, the volume of the first tumor is reduced by at least or at least about 75%. In some embodiments, immunity to a second tumor or lesion is effected when the first tumor achieves partial or complete response (PR or CR) following treatment of the first tumor. In some embodiments, immunity to a second tumor or lesion is effected when the first tumor achieves CR following treatment of the first tumor.

In some cases, the target area for illumination can be the primary tumor or lesion, or the vicinity of the primary tumor or lesion. In other cases, the target area for illumination is not the primary tumor or lesion, but a different area where PD-L1 expressing cells are present, such as the lymph node, or a secondary tumor or lesion or the vicinity of a secondary tumor or lesion.

In some aspects, following treatment according to the provided embodiments, with anti-PD-L1 conjugate treatments and light illumination, the growth of the one or more primary tumors or lesions is inhibited, the volume of the one or more primary tumors or lesions is reduced or both tumor growth and volume are reduced. In some aspects, following treatment according to the provided embodiments, with anti-PD-L1 conjugate treatments and light illumination, the growth of the one or more secondary or metastatic tumors or lesions is inhibited, the volume of the one or more secondary or metastatic tumors or lesions is reduced or both tumor growth and volume are reduced. In some aspects, treatment according to the provided embodiments delays regrowth of the tumor or the lesion, prevents a relapse of a cancer or prolongs the duration of remission of a cancer, such as a cancer associated with the tumor or lesion, prevents or inhibits the generation and/or growth of one or more second tumor or lesion, including a second tumor or lesion of a different type compared to the primary tumor or lesion, and/or prevents or inhibits the generation and/or growth of a metastasis.

In some embodiments, the subject is administered the anti-PD-L1 conjugate to treat and/or inhibit the growth of a first tumor or a first lesion; and the method inhibits or delays the appearance of one or more second tumors or lesions or a metastasis of the first tumor or the first lesion.

In some aspects, the primary tumor or lesion contains cells that express PD-L1 on the surface. In some aspects, the cells that express PD-L1 is an immune cell, such as an immunosuppressive cell, e.g., M2 TAM, tDCs or MDSCs. In some aspects, the PD-L1 expressing cell is a tumor associated fibroblast or a cancer associated fibroblasts (CAF). In some cases, the cells that express PD-L1 is a tumor cell or a cancer cell. In some of any of the embodiments, the subject to be treated has one or more of the PD-L1 expressing cells, such as one or more PD-L1 expressing cells that are associated with the tumor, lesion or cancer.

In some embodiments, the tumor, lesion or cancer to be treated is contains tumor or cancer cells that do not express PD-L1. In some embodiments, the tumor or the lesion comprises PD-L1 negative tumor cells. In some embodiments, more than at or about 40%, 50%, 60%, 70%, 80%, 90% or 95% of the tumor cells in the tumor or the lesion are PD-L1 negative tumor cells. In some aspects, PD-L1 negative tumor cells can refer to tumor cells that do not express detectable levels of PD-L1 on its surface or tumor cells that express PD-L1 at a level less than a threshold level, such as a detectable threshold level. In some embodiments, a PD-L1 negative tumor cell includes a tumor cell is not specifically recognized by an anti-PD-L1 antibody. In some cases, the level of PD-L1 expression is determined by flow cytometry. In some aspects, the provided embodiments result in indirect killing of tumor cells or cancer cells, such as by eliminating immunosuppressive cells, e.g., M2 TAM, tDCs or MDSCs, and enhancing the function and/or activity of effector cells in the immune system, such as CD8+ T cells, which can exert an anti-tumor or anti-cancer response to eliminate the tumor cells or cancer cells.

In some embodiments, the tumor, lesion or cancer to be treated is contains tumor or cancer cells that express PD-L1. In some aspects, administration of the anti-PD-L1 conjugate followed by light illumination can directly kill cells that express PD-L1. In some aspects, the provided embodiments result in a direct killing of PD-L1-expressing tumor cells.

In some embodiments, the methods and uses provided herein include treating a subject that has invasive tumor cells, such as when cells originating from a primary tumor have invaded into surrounding tissues. The methods include administering to a subject having invasive tumor cells, an anti-PD-L1 conjugate and after administration of the conjugate, illuminating the target area with a wavelength suitable for the selected phthalocyanine dye. In some embodiments, the methods include the administration of an immunomodulatory agents, such as an immune checkpoint inhibitor prior to, concurrent with, or subsequent to the administration of the conjugate. In some aspects, invasive tumor cells refer to cells originated from a primary tumor and have invaded into surrounding tissues of the same organ or neighboring organs or body cavities of the primary tumor within the body of a subject having the primary tumor.

In some instances, the methods and uses provided herein include illumination of a target area. In some aspects, the target area the one or more primary tumors, and some or all of the invasive tumor cells are not illuminated, and in such methods, the growth of invasive tumor cells is inhibited, reduced or eliminated, the volume of one or more invasive tumors is reduced or any combination thereof. In some embodiments, the growth of the primary tumor also is inhibited, reduced or eliminated, the volume of one or more primary tumors also is reduced along with the effect(s) on the one or more invasive tumor cells.

In some embodiments, invasive tumor cells are contained in a solid tumor. In some embodiments, invasive tumor cells are contained in body fluids, including but not limited to peritoneal fluid, pleural fluid, and cerebrospinal fluid. In some embodiments, invasive tumor cells are contained in the effusion of a body cavity or body cavities, including but not limited to peritoneal effusion (ascites), pleural effusion, and pericardial effusion.

In some embodiments, the methods and uses provided herein include treating a subject that has one or more primary tumors and also metastatic tumor cells. The methods include administering to a subject having primary tumor(s) and metastatic tumor cells, an anti-PD-L1 conjugate and after administration of the conjugate, illuminating the target area with a wavelength suitable for the selected phthalocyanine dye. In such methods, the growth of metastatic tumor cells is inhibited, reduced or eliminated, the volume of one or more metastatic tumors is reduced or any combination thereof.

In some embodiments of the methods and uses provided herein, metastatic tumor cells are distal to the primary tumor and some or all of the metastatic tumor cells are not illuminated, e.g., not directly illuminated.

In some embodiments of the methods and uses, only a target area, such as a target area containing and/or is in the vicinity of a lymph node or a primary tumor or lesion, is illuminated. In some aspects, the second tumor or lesion, such as a metastatic tumor or lesion, is not illuminated.

In some aspects, metastatic tumor cells include cells originated from a primary tumor and spread to distal tissue or organ, or distal tissues or organs within the body of a subject having the primary tumor. The metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells or are not associated with a tumor mass.

In some embodiments, the methods and uses include the administration of an immunomodulatory agent, such as a checkpoint inhibitor prior to, concurrent with or subsequent to the administration of the conjugate. In some embodiments, the methods and uses include the administration of a second conjugate, such as a second immunoconjugate, followed by illumination, concurrent with, prior to, or subsequent to the administration of the instantly provided conjugate. In some embodiments, the methods and uses include the administration of one or more additional anti-cancer treatments, such one or more of a chemotherapy, antiangiogenic therapy, a kinase inhibitor, a radiotherapy, small molecule therapy or other treatment, such as any treatment described in the Section entitled “Combination Therapy” herein.

C. Methods and Compositions for Treating a Tumor or Tumor Cells that are Less Responsive to, Refractory to, or Not Responsive to Prior Therapeutic Treatments

In some embodiments, provided are compositions containing an anti-PD-L1 conjugate, i.e., a phthalocyanine dye-targeting molecule conjugate, in which the targeting molecule binds to PD-L1 (e.g., an anti-PD-L1 antibody-IR700 conjugate), and methods and uses involving the anti-PD-L1 conjugate for therapy or treatment of a tumor or a cancer that has failed, that was less responsive to, that has not achieved a desired level of response, that achieved a less than desired level of response to (e.g., poorly responsive or not therapeutically effective) or was not responsive to one or more prior treatments, such as with an immunomodulatory agent, such as an immune checkpoint inhibitor and/or an anticancer agent, such as an anti-cancer agent that directly targets tumor or cancer cells. In some embodiments, the tumor or cancer achieved a less than desired level of response or is predicted to be resistant to anti-PD-L1, anti-PD-1, and/or anti-CTLA-4 therapy. In some embodiments, the tumor or cancer achieved a less than desired level of response or is predicted to be resistant to anti-PD-L1 therapy. In some embodiments, the tumor or cancer achieved a less than desired level of response or is predicted to be resistant to anti-PD-1 therapy. In some embodiments, the tumor or cancer achieved a less than desired level of response or is predicted to be resistant to anti-CTLA-4 therapy.

The cancers include a primary tumor or multiple primary tumors as well as metastatic tumor cells, for example metastatic cancers; newly arising tumors or cancers; a cancer that includes a primary tumor or multiple primary tumors; and/or invasive tumor cells, for example, invasive cancers. In some aspects, the provided compositions, methods, uses and combinations can also sensitize cold tumors, including primary cold tumors and secondary cold tumors (e.g., metastatic tumors), to immunomodulatory agents or other anti-cancer therapy.

Such methods and uses include, for example, administration of an anti-PD-L1 conjugate to a subject having a tumor or tumor cells followed by illumination of a target area (e.g., sites where PD-L1 expressing cells are present), using a suitable light wavelength and dose for the phthalocyanine dye. In some aspects, the illumination results in an illumination-dependent lysis and death of cells expressing the target molecule (e.g., PD-L1) on the surface, resulting in a therapeutic effect or treatment of the cancer. In some cases, cells expressing PD-L1, such as monocytes, macrophages, dendritic cells (DC), M2 tumor associated macrophages (M2 TAM), tolerogenic dendritic cells (tDCs) or myeloid derived suppressor cells (MDSCs), or certain tumor cells, are killed and thus rapidly deplete. As a result, necrosis of the tumor cells can occur.

In some aspects, the tumor, lesion or cancer to be treated in accordance with the provided embodiments include those that have failed, had a low responsiveness or were substantially non-responsive to, that was less responsive to, that had not achieved a desired level of response, that achieved a less than desired level of response to (e.g., poorly responsive or not therapeutically effective), has relapsed after, were refractory to and/or were resistant to one or more prior treatments, such as with an immunomodulatory agent, e.g., an immune checkpoint inhibitor and/or an anticancer agent, such as an anti-PD-L1, anti-PD-1, or anti-CTLA-4 therapy.

In some embodiments, the subject to be treated in accordance with the provided embodiments, has been previously treated with an anti-cancer treatment and/or an immune checkpoint inhibitor. In some embodiments, the subject to be treated in accordance with the provided embodiments, has been previously treated with an immune checkpoint inhibitor. In some embodiments, the subject to be treated in accordance with the provided embodiments, has failed or has relapsed after the previous treatment with the anti-cancer treatment and/or immune checkpoint inhibitor. In some embodiments, the subject to be treated in accordance with the provided embodiments, has failed or has relapsed after the previous treatment with the immune checkpoint inhibitor.

In some embodiments, the inhibition of tumor growth resulting from carrying out the method is greater compared to the inhibition of tumor growth as a result of the previous treatment with the anti-cancer treatment and/or immune checkpoint inhibitor (e.g., anti-PD-L1, anti-PD-1, and/or anti-CTLA-4 therapy). In some embodiments, the inhibition of tumor growth resulting from carrying out the method is greater compared to the inhibition of tumor growth as a result of the previous treatment with the immune checkpoint inhibitor (e.g., anti-PD-L1, anti-PD-1, and/or anti-CTLA-4 therapy).

In some aspects, the prior therapeutic treatment or treatments to which the cancers are not responsive include using an anticancer agent. The prior anticancer agent can be one or more of: a chemotherapeutic agent, an antibody treatment, and/or a radiotherapeutic agent. In some embodiments, the prior therapy is therapy with an anti-cancer agent selected from a checkpoint inhibitor, an immune adjuvant, a chemotherapeutic agent, radiation, and a biologic comprising an anti-cancer targeting molecule that binds to a tumor cell. In some embodiments, the prior therapy is therapy with an anti-cancer agent that is an antibody conjugate. In some embodiments, the prior therapy is therapy with an antibody conjugate comprising a phthalocyanine dye, a toxin, or a TLR agonist.

In some aspects, the prior therapeutic treatment or treatments to which a cancer, tumor, or tumor cells are not responsive can be treatment with an immune checkpoint inhibitor (also known immune checkpoint blockade therapy). The prior immune checkpoint inhibitor can be a PD-1 inhibitor, a PD-L1 inhibitor, a CTLA-4 inhibitor or combination thereof. The prior immune checkpoint inhibitor can be a small molecule inhibitor, an antibody inhibitor, or other molecule that binds to and inhibits an immune checkpoint protein, such as PD-1 or PD-L1. Exemplary antibody inhibitors for PD-1 include, but are not limited to, any of pembrolizumab (MK-3475, Keytruda), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab. Exemplary antibody inhibitors for PD-L1 include, but are not limited to, any of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some aspects, the tumor, lesion or cancer to be treated include tumors or cancers that were resistant to, refractory to, or not responsive to, anti-PD-1 antibody or anti-PD-Llantibody treatment. In some aspects, the tumor, lesion or cancer to be treated include tumors or cancers that were resistant to, refractory to, or not responsive to anti-PD-L1 antibody treatment or are predicted to be unresponsive, resistant, or refractory to anti-PD-L1 antibody treatment. In some aspects, the tumor, lesion or cancer to be treated include tumors or cancers that were resistant to, refractory to, or not responsive to anti-PD-1 antibody treatment or are predicted to be unresponsive, resistant, or refractory to anti-PD-1 antibody treatment.

In some aspects, the prior treatment is treatment with anti-CTLA-4 antibody, such as ipilimumab (YERVOY), tremelimumab, AGEN1181, AGEN1884, ADU-1064, BCD-145, and BCD-217. In some aspects, the tumor, lesion or cancer to be treated include tumors or cancers that were resistant to, refractory to, or not responsive to anti-CTLA-4 antibody treatment or are predicted to be unresponsive, resistant, or refractory to anti-CTLA-4 antibody treatment.

In some aspects, the prior therapeutic treatment or treatments to which a cancer, tumor or tumor cells are not responsive can be treatment with an immunomodulatory agent such as a cytokine, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway, or such as an adjuvant, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some aspects, the prior therapeutic treatment or treatments to which the cancers are not responsive include using a therapeutic agent targeted against immunosuppressive cells. The agent can be an antibody, for example, anti-CD25 antibodies, such as basiliximab (Simulect®), daclizumab or PC61, that target regulatory T cells; a small molecule inhibitor or combination thereof. Immunosuppressive cells include regulatory T cells, M2 macrophages, tumor associated fibroblasts or cancer associated fibroblasts (CAFs), or combination thereof.

In some instances, a tumor, lesion or cancer to be treated in accordance with the provided embodiments include a “cold tumor” or a “cold cancer,” such as a tumor that has an immunosuppressive phenotype. Such cold tumors can have features including, but not limited to, a substantial reduction in numbers and/or activities or absence of intratumoral CD8+T effector cells and/or substantial increase in numbers and/or activities of intratumoral immune suppressor cells. In some cases, a cold tumor or cancer has a high tumor mutational burden (TMB), an immune score indicative of low immunoresponsiveness, a programmed cell death protein 1 (PD-1) or programmed death-ligand 1 (PD-L1) marker status (e.g., cell surface expression), which could be indicative of low immunoresponsiveness. In some instances, a cold tumor or cancer does not respond to PD-1 or PD-L1 inhibitor monotherapy.

In some embodiments, cold tumors or cancers can be treated with anti-PD-L1 conjugate, followed by illumination, as described herein. In some embodiments, a combination treatment with an anti-PD-L1 conjugate, followed by illumination, and an immunomodulatory agent, such as an immune checkpoint inhibitor, results in enhanced inhibitory effects on the growth of both illuminated primary tumor and a distal tumor.

Furthermore, for tumors that are resistant to a treatment with an immune modulatory therapy, such as treatment with an immune checkpoint inhibitor, treatment with an anti-PD-L1 conjugate followed by light illumination and/or in combination with an immune checkpoint inhibitor can result in enhanced inhibitory effects on the growth of both illuminated primary tumor and a distal tumor, a primary tumor and a newly arising tumor and/or a primary tumor and a secondary tumor of a different type, indicating a sensitization effect of the anti-PD-L1 photoimmunotherapy on immune checkpoint inhibitors in treating cancers and tumor cells.

### IV. Methods of Administration and Formulations

Also provided are compositions including the PD-L1 antibodies, antigen-binding fragments, and immunoconjugates including pharmaceutical compositions and formulations.

In some embodiments, the anti-PD-L1 conjugate may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the anti-PD-L1 conjugate is administered intravenously. In some embodiments, the anti-PD-L1 conjugate is administered parenterally. In some embodiments, the anti-PD-L1 conjugate is administered enterally. In some embodiments, the conjugate is administered by local injection. In some embodiments, the conjugate is administered as a topical application.

The compositions comprising the anti-PD-L1 conjugate can be administered locally or systemically using any method known in the art, for example to subjects having a tumor, such as a cancer, or who has had a tumor previously removed, for example via surgery. Although specific examples are provided, one skilled in the art will appreciate that alternative methods of administration of the disclosed agents can be used. Such methods may include for example, the use of catheters or implantable pumps to provide continuous infusion over a period of several hours to several days into the subject in need of treatment.

In some embodiments, the anti-PD-L1 conjugate is administered by parenteral means, including direct injection or infusion into a tumor, such as intratumorally. In some embodiments, the anti-PD-L1 conjugate is administered to the tumor by applying the agent to the tumor, for example by bathing the tumor in a solution containing the anti-PD-L1 conjugate, or by pouring the agent onto the tumor.

In addition, or alternatively, the anti-PD-L1 conjugate can be administered systemically, for example intravenously, intramuscularly, subcutaneously, intradermally, intraperitoneally, subcutaneously, or orally, to a subject having a tumor, such as cancer.

Also provided herein are compositions, such as pharmaceutical compositions, containing the anti-PD-L1 conjugate, and uses of such compositions, such as therapeutic uses and/or uses as a medicament. In some aspects, the compositions comprise the anti-PD-L1 conjugate and a pharmaceutically acceptable carrier. In some embodiments, the composition containing the anti-PD-L1 conjugate is for use in treatment or therapy, in accordance with any of the provided embodiments, such as for administration to a subject having a disease or condition, for the treatment of the disease or condition. The dosages of the anti-PD-L1 conjugate to be administered to a subject are not subject to absolute limits but will depend on the nature of the composition and its active ingredients and its unwanted side effects, such as immune response against the agent, the subject being treated, and the type of condition being treated and the manner of administration. Generally, the dose will be a therapeutically effective amount, such as an amount sufficient to achieve a desired biological effect, for example an amount that is effective to decrease the size, such as volume and/or weight, of the tumor, or attenuate further growth of the tumor, or decrease undesired symptoms of the tumor.

In some embodiments, the compositions used for administration of the anti-PD-L1 conjugate contain an effective amount of the agent along with conventional pharmaceutical carriers and excipients appropriate for the type of administration contemplated. For example, in some embodiments, parenteral formulations may contain a sterile aqueous solution or suspension of the conjugate. In some embodiments, compositions for enteral administration may contain an effective amount of the anti-PD-L1 conjugate in aqueous solution or suspension that may optionally include buffers, surfactants, thixotropic agents, and flavoring agents.

In some embodiments, the anti-PD-L1 conjugate or conjugate in combination with an additional therapeutic agent, can be formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. Pharmaceutically acceptable compositions generally are prepared in view of approvals for a regulatory agency or other agency prepared in accordance with generally recognized pharmacopeia for use in animals and in humans.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In some embodiments, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In some cases, pharmaceutical preparations can be presented in lyophilized form for reconstitution with water or other suitable vehicle before use.

In some embodiments, the nature of the pharmaceutically acceptable buffer, or carrier, depends on the particular mode of administration being employed. For instance, in some embodiments, parenteral formulations may comprise injectable fluids that include pharmaceutically and physiologically acceptable fluids such as water, physiological saline, balanced salt solutions, aqueous dextrose, or glycerol as a vehicle. In some embodiments, for solid compositions, for example powder, pill, tablet, or capsule forms, non-toxic solid carriers can include, for example, pharmaceutical grades of mannitol, lactose, starch, or magnesium stearate. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In addition to biologically neutral carriers, pharmaceutical compositions to be administered can in some embodiments contain minor amounts of non-toxic auxiliary substances, such as wetting or emulsifying agents, preservatives, and pH buffering agents, for example sodium acetate or sorbitan monolaurate.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the antibody, antigen-binding fragment, or conjugate, preferably those with activities complementary to the antibody, antigen-binding fragment, or conjugate, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the antibody, antigen-binding fragment, or conjugate in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant.

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

Injectables are designed for local and systemic administration. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or non-aqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Non-aqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The composition can be formulated for single dosage administration or for multiple dosage administration. The agents can be formulated for direct administration. The composition can be provided as a liquid or lyophilized formulation. Where the composition is provided in lyophilized form it can be reconstituted just prior to use by an appropriate buffer, for example, a sterile saline solution.

Compositions also can be administered with other biologically active agents, either sequentially, intermittently or in the same composition. Administration also can include controlled release systems including controlled release formulations and device-controlled release, such as by means of a pump.

The most suitable route in any given case depends on a variety of factors, such as the nature of the disease, the progress of the disease, the severity of the disease and the particular composition which is used. For example, compositions are administered systemically, for example, via intravenous administration. Subcutaneous methods also can be employed, although increased absorption times can be necessary to ensure equivalent bioavailability compared to intravenous methods.

Pharmaceutical compositions can be formulated in dosage forms appropriate for each route of administration. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. Pharmaceutical compositions can be formulated in dosage forms appropriate for each route of administration.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. In some embodiments, the compositions can be provided as a lyophilized powder, which can be reconstituted for administration as solutions, emulsions and other mixtures. They may also be reconstituted and formulated as solids or gels. The lyophilized powders can be prepared from any of the solutions described above.

The sterile, lyophilized powder can be prepared by dissolving a phthalocyanine dye-targeting molecule conjugate in a buffer solution. The buffer solution may contain an excipient which improves the stability of other pharmacological components of the powder or reconstituted solution, prepared from the powder.

In some embodiments, subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to ensure sterility and apportioned into vials for lyophilization. Each vial can contain a single dosage (1 mg-1 g, generally 1-100 mg, such as 1-5 mg) or multiple dosages of the compound. The lyophilized powder can be stored under appropriate conditions, such as at about 4° C. to room temperature. Reconstitution of this lyophilized powder with a buffer solution provides a formulation for use in parenteral administration. The precise amount depends upon the indication treated and selected compound. Such amount can be empirically determined.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

The compositions can be formulated for single dosage administration or for multiple dosage administration. The agents can be formulated for direct administration.

In some embodiments, the compositions provided herein are formulated in an amount for direct administration of the anti-PD-L1 conjugate, in a range from at or about 0.01 mg to at or about 3000 mg, from at or about 0.01 mg to at or about 1000 mg, from at or about 0.01 mg to at or about 500 mg, from at or about 0.01 mg to at or about 100 mg, from at or about 0.01 mg to at or about 50 mg, from at or about 0.01 mg to at or about 10 mg, from at or about 0.01 mg to at or about 1 mg, from at or about 0.01 mg to at or about 0.1 mg, from at or about 0.1 mg to at or about 2000 mg, from at or about 0.1 mg to at or about 1000 mg, from at or about 0.1 mg to at or about 500 mg, from at or about 0.1 mg to at or about 100 mg, from at or about 0.1 mg to at or about 50 mg, from at or about 0.1 mg to at or about 10 mg, from at or about 0.1 mg to at or about 1 mg, from at or about 1 mg to at or about 2000 mg, from at or about 1 mg to at or about 1000 mg, from at or about 1 mg to at or about 500 mg, from at or about 1 mg to at or about 100 mg, from at or about 1 mg to at or about 10 mg, from at or about 10 mg to at or about 2000 mg, from at or about 10 mg to at or about 1000 mg, from at or about 10 mg to at or about 500 mg, from at or about 10 mg to at or about 100 mg, from at or about 100 mg to at or about 2000 mg, from at or about 100 mg to at or about 1000 mg, from at or about 100 mg to at or about 500 mg, from at or about 500 mg to at or about 2000 mg, from at or about 500 mg to at or about 1000 mg, and from about 1000 mg to at or about 3000 mg. In some embodiments, the volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. For example, the composition is formulated for single dosage administration of an amount between at or about 100 mg and at or about 500 mg, or between at or about 200 mg and at or about 400 mg. In some embodiments, the composition is formulated for single dosage administration of an amount between at or about 500 mg and at or about 1500 mg, at or about 800 mg and at or about 1200 mg or at or about 1000 mg and at or about 1500 mg. In some embodiments, the volume of the composition is between at or about 10 mL and at or about 1000 mL or at or about 50 mL and at or about 500 mL; or the volume of the composition is at least at or about 10 mL, 20 mL, 30 mL, 40 mL, 50 mL, 75 mL, 100 mL, 150 mL, 200 mL, 250 mL, 300 mL, 400 mL, 500 mL or 1000 mL.

In some embodiments, the entire vial contents of the formulations can be withdrawn for administration or can be divided up into a plurality of dosages for multiple administrations. Upon withdrawal of an amount of drug for administration, the formulation can be further diluted if desired, such as diluted in water, saline (e.g., 0.9%) or other physiological solution.

In some embodiments, also provided are compositions containing an additional therapeutic agent, such as an immunomodulatory agent or anti-cancer agent, for use in combination with the anti-PD-L1 conjugate, in accordance with the provided embodiments. In some aspects, the additional therapeutic agent can be prepared in accord with known or standard formulation guidelines, such as described above. In some embodiments, the immunomodulatory agent, anti-cancer agent and/or anti-PD-L1 conjugate are formulated as separate compositions. In some embodiments, the immunomodulatory agent is provided as a separate composition from the anti-PD-L1 conjugate, and the two compositions are administered separately. In some embodiments, the anti-cancer agent is provided as a separate composition from the anti-PD-L1 conjugate, and the two compositions are administered separately. The compositions can be formulated for parenteral delivery (i.e. for systemic delivery). For example, the compositions or combination of compositions are formulated for subcutaneous delivery or for intravenous delivery. The agents, such as an anti-PD-L1 conjugate, and an immunomodulatory agent and/or an anti-cancer agent can be administered by different routes of administration.

In some aspects, exemplary additional therapeutic agents, such as immunomodulatory agents, can be administered as directed for a monotherapy or on other administration schedules and dose for the particular therapeutic agent. In some embodiments of the methods and uses that involve administration of with an anti-PD-L1 conjugate and an additional therapeutic agent, the additional therapeutic agent is administered at the recommended dose and/or schedule of administration. In some embodiments, an additional therapeutic agent can be administered in the methods herein at a dose lower than the recommended amount or on an alternate schedule, such as when anti-PD-L1 conjugate sensitizes a tumor or cancer or the TME to the additional therapeutic agent and/or when the combination of an anti-PD-L1 conjugate and an additional therapeutic agent results in a synergistic response.

V. Devices and Illumination Methods for Use with the Anti-Pd-L1 Conjugates

In some aspects, devices that can be used with the provided embodiments include light diffusing devices that provide illumination (in some cases, also referred to as irradiation) at a wavelength (or wavelengths) of light wavelength suitable for use with the dye conjugate composition, such as a phthalocyanine dye conjugate (e.g., an anti-PD-L1 conjugate such as those described herein). Illumination devices can include a light source (for example, a laser), and a means of conveying the light to the area of interest (for example, one or more fibers to illuminate an isolated area of a subject or an isolated lesion or tumor). Exemplary illumination devices are described in Patent Nos. U.S. Pat. Nos. 10,295,719; 10,527,771; and 10,416,366, incorporated herein by reference. Such devices deliver light to a target region of a subject using a light diffusing device, containing, a non-circular core optic fiber that is operably connected to a laser. In some embodiments, the core optic fiber is circular and is coiled or bent prior to interfacing with a light diffusing device. In particular aspects, the device delivers a “top hat” core irradiance distribution to deliver uniform light to the illuminated area. The light diffusing device can be as cylindrical diffuser for use, for example, for intratumor or intratissue irradiation. In some embodiments, the light diffusing device is a frontal diffuser, with a lens, where the illumination is projected through the lens of the frontal diffuser at the end of the optic fiber. The projected light can be a collimated or dispersing beam of light.

In some embodiments, the target area, such as a tumor, the vicinity of a tumor, a lymph node, the vicinity of the lymph node, is illuminated with light at a wavelength within a range from at or about 400 nm to at or about 900 nm, such as from or from at or about 500 nm to at or about 900 nm, such as from or from at or about 600 nm to at or about 850 nm, such as from or from at or about 600 nm to at or about 810 nm, such as from or from at or about 600 nm to at or about 740 nm, such as from or from at or about 620 nm to at or about 720 nm, such as from or from at or about 640 nm to at or about 700 nm, such as from or from at or about 660 nm to at or about 680 nm, such as from at or about 660 nm to at or about 740 nm, from at or about 660 nm to at or about 710 nm, from at or about 660 nm to at or about 700 nm, from at or about 660 to at or about 685, from at or about 665 to at or about 680, from at or about 670 to at or about 685, from at or about 670 nm to at or about 690 nm, from at or about 670 to at or about 680, from at or about 680 nm to at or about 740 nm, or from at or about 690 nm to at or about 710 nm. In some embodiments, the target area, such as a tumor, the vicinity of a tumor, a lymph node, the vicinity of the lymph node, or the tumor microenvironment, is illuminated with light at a wavelength of at or about 600 nm to at or about 850 nm, such as at or about 660 nm to at or about 740 nm. In some embodiments, the target area, such as a tumor, the vicinity of a tumor, a lymph node, the vicinity of the lymph node, or the tumor microenvironment, is illuminated with light at wavelength of at least at or about 600 nm, 610 nm, 620 nm, 630, nm, 640 nm, 650 nm, 660 nm, 670 nm, 680 nm, 690 nm 700 nm, 720 nm or 740 nm, such as at or about 690±50 nm, or at or about 690±40 nm, for example at or about 690 nm or at or about 680 nm. In some embodiments, the phthalocyanine dye in the conjugate (e.g., anti-PD-L1-phthalocyanine dye conjugate) is IR700, and the target area, such as a tumor, the vicinity of a tumor, a lymph node, the vicinity of the lymph node, or the tumor microenvironment, is illuminated with light at wavelength of at or about 690±50 nm, or at or about 690±40 nm, for example at or about 690 nm or at or about 680 nm.

In some embodiments, the target area, such as a tumor, the vicinity of a tumor, a lymph node, the vicinity of the lymph node, or the tumor microenvironment, is illuminated with light at wavelength that is at or about 675±50 nm, at or about 675±40 nm, at or about 675±20 nm, or at or about 675±10 nm, for example at or about 675 nm. In some embodiments, the target area, such as a tumor, the vicinity of a tumor, a lymph node, the vicinity of the lymph node, or the tumor microenvironment, is illuminated with light at wavelength that is at or about 670±50 nm, or at or about 670±40 nm, for example at or about 670 nm. In some embodiments, the target area, such as a tumor, the vicinity of a tumor, a lymph node, the vicinity of the lymph node, or the tumor microenvironment, is illuminated with light at wavelength that is at or about 660±50 nm, or at or about 660±40 nm, for example at or about 660 nm. In some embodiments, the target area, such as a tumor, the vicinity of a tumor, a lymph node, the vicinity of the lymph node, or the tumor microenvironment, is illuminated with light at a wavelength of less than or less than about 685 nm or 680 nm. In some embodiments, the phthalocyanine dye in the conjugate (e.g., anti-PD-L1-phthalocyanine dye conjugate) has the structure of Formula (I):

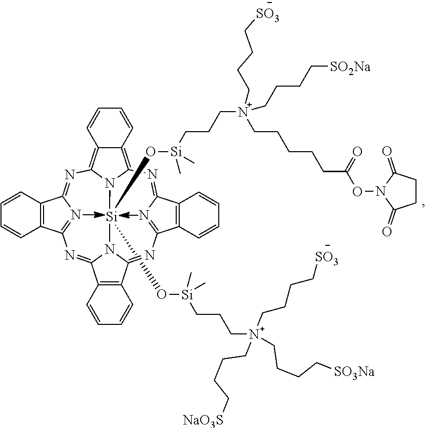

or is a salt, stereoisomer, or tautomer thereof, and the target area, such as a tumor, the vicinity of a tumor, a lymph node, the vicinity of the lymph node, or the tumor microenvironment, is illuminated with light at wavelength of at or about 675±50 nm, or at or about 675±40 nm, for example at or about 675 nm.

In some embodiments of the methods and uses provided herein, illumination is carried out using cylindrical diffusing fibers that includes a diffuser length of at or about 0.5 cm to at or about 10 cm and spaced at or about 1.8±0.2 cm apart. In some embodiments, the light illumination dose is from at or about 20 J/cm fiber length to at or about 500 J/cm fiber length. In some embodiments, the tumor is greater than at or about 10 mm deep or is a subcutaneous tumor.

In some embodiments, the provided methods include illuminating a target area that is an interstitial tumor in a subject with cylindrical diffusing fibers that includes a diffuser length of at or about 0.5 cm to at or about 10 cm and spaced at or about 1.8±0.2 cm apart with a light dose of at or about 100 J/cm fiber length or with a fluence rate of at or about 400 mW/cm. In some embodiments, the target area is a tumor that is greater than at or about 10 mm deep or is a subcutaneous tumor. In some embodiments, the cylindrical diffusing fibers are placed in a catheter positioned in the tumor at or about 1.8±0.2 cm apart. In some embodiments, the catheter is optically transparent.

In some embodiments, the target area, such as a tumor, the vicinity of a tumor, a lymph node, the vicinity of the lymph node, or the tumor microenvironment, is illuminated with light dose of at least at or about 1 J/cm², such as at least at or about 10 J/cm², at least at or about 30 J/cm², at least at or about 50 J/cm², at least at or about 75 J/cm², at least at or about 100 J/cm², at least at or about 150 J/cm², or at least at or about 500 J/cm². In some embodiments, the dose of illumination is from at or about 1 to at or about J/cm², from at or about 1 to at or about 500 J/cm², from at or about 5 to at or about 200 J/cm², from at or about 10 to at or about 100 J/cm², or from at or about 10 to at or about 50 J/cm², from at or about 30 to at or about 200 J/cm², from at or about 30 to at or about 150 J/cm², or from at or about 30 J/cm² to at or about 100 J/cm². In some embodiments, the target area is illuminated at a dose of at least at or about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm².

In some embodiments, the target area is a tumor that is a superficial tumor. In some embodiments, the tumor is less than 10 mm thick. In some embodiments, illumination is carried out using a microlens-tipped fiber for surface illumination. In some embodiments, the light illumination dose is from at or about 5 J/cm² to at or about 200 J/cm².

In some embodiments, the target area, such as a tumor, the vicinity of a tumor, a lymph node, the vicinity of the lymph node, or the tumor microenvironment, are illuminated at a dose of at least at or about 1 J/cm fiber length, such as at least at or about 10 J/cm fiber length, at least at or about 50 J/cm fiber length, at least at or about 100 J/cm fiber length, at least at or about 250 J/cm fiber length, or at least at or about 500 J/cm fiber length. In some embodiments, the dose of illumination is from at or about 1 to at or about 1000 J/cm fiber length, from at or about 1 to at or about 500 J/cm fiber length, from at or about 2 to at or about 500 J/cm fiber length, from at or about 50 to at or about 300 J/cm fiber length, from at or about 10 to at or about 100 J/cm fiber length, or from at or about 10 to at or about 50 J/cm fiber length. In some embodiments, the target area, such as a tumor, the vicinity of a tumor, a lymph node, the vicinity of the lymph node, or the tumor microenvironment, are illuminated at a dose of at least at or about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the provided methods include illuminating a target area that is a superficial tumor in a subject with a microlens-tipped fiber for surface illumination with a light dose of from at or about 5 J/cm² to at or about 200 J/cm². In some embodiments, the light illumination dose is at or about 50 J/cm².

In some cases, it is found that a dose of illumination in a human subject to achieve PIT can be less than is necessary for PIT in a mouse. For example, in some cases, at or about 50 J/cm² (50 J/cm²) light dosimetry in an in vivo tumor mouse model is not effective for PIT, which is in contrast to what has been observed in the clinic with human patients.

In some embodiments, the dose of illumination following administration of the composition comprising the phthalocyanine dye-targeting molecule conjugate is at least at or about 1 J/cm² or at least at or about 1 J/cm of fiber length at a wavelength of at or about 660-740 nm, for example, at least at or about 10 J/cm² or at least at or about 10 J/cm of fiber length at a wavelength of at or about 660-740 nm, at least at or about 50 J/cm² or at least at or about 50 J/cm of fiber length at a wavelength of at or about 660-740 nm, or at least at or about 100 J/cm² or at least at or about 100 J/cm of fiber length at a wavelength of at or about 660-740 nm. In some embodiments, the wavelength is 660-710 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 690 nm, for example, at least at or about 10 J/cm² or at least at or about 10 J/cm of fiber length, at a wavelength of at or about 690 nm, at least at or about 50 J/cm² or at least at or about 50 J/cm of fiber length, at a wavelength of at or about 690 nm, or at least at or about 100 J/cm² or at least at or about 100 J/cm of fiber length, at a wavelength of at or about 690 nm, for example 1.0 to 500 J/cm² or 1.0 to 500 J/cm of fiber length, at a wavelength of at or about 690 nm. Exemplary illumination after administration of the conjugates or compositions provided herein, for example, a conjugate comprising an anti-PD-L1 antibody and IR700, include illuminating the target area at a wavelength of at or about 660 nm to at or about 740 nm at a dose of at least at or about 1 J/cm² or at least at or about 1 J/cm of fiber length.

In some embodiments, the dose of illumination following administration of the composition comprising the phthalocyanine dye-targeting molecule conjugate is at least at or about 1 J/cm² or at least at or about 1 J/cm of fiber length at a wavelength of at or about 600-800 nm, for example, at least at or about 1 J/cm² or at least at or about 1 J/cm of fiber length at a wavelength of at or about 620-720 nm, at least at or about 10 J/cm² or at least at or about 10 J/cm of fiber length at a wavelength of at or about 620-720 nm, at least at or about 50 J/cm² or at least at or about 50 J/cm of fiber length at a wavelength of at or about 620-720 nm, or at least at or about 100 J/cm² or at least at or about 100 J/cm of fiber length at a wavelength of at or about 620-720 nm. In some embodiments, the wavelength is 640-700 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 670 nm, for example, at least at or about 10 J/cm² or at least at or about 10 J/cm of fiber length, at a wavelength of at or about 670 nm, at least at or about 50 J/cm² or at least at or about 50 J/cm of fiber length, at a wavelength of at or about 670 nm, or at least at or about 100 J/cm² or at least at or about 100 J/cm of fiber length, at a wavelength of at or about 670 nm, for example 1.0 to 500 J/cm² or 1.0 to 500 J/cm of fiber length, at a wavelength of at or about 670 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 675 nm, for example, at least at or about 10 J/cm² or at least at or about 10 J/cm of fiber length, at a wavelength of at or about 675 nm, at least at or about 50 J/cm² or at least at or about 50 J/cm of fiber length, at a wavelength of at or about 675 nm, or at least at or about 100 J/cm² or at least at or about 100 J/cm of fiber length, at a wavelength of at or about 675 nm, for example 1.0 to 500 J/cm² or 1.0 to 500 J/cm of fiber length, at a wavelength of at or about 675 nm. Exemplary illumination after administration of the conjugates or compositions provided herein, for example, a conjugate comprising an anti-PD-L1 antibody and a phthalocyanine dye having the structure of Formula (I):

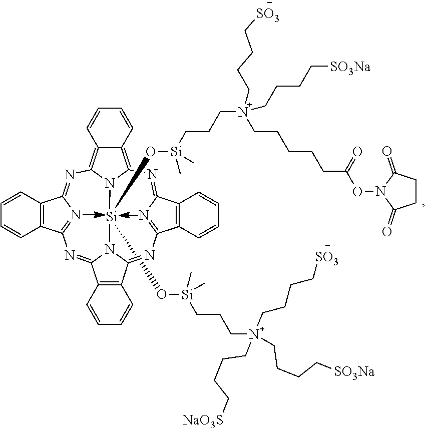

or is a salt, stereoisomer, or tautomer thereof, include illuminating the target area at a wavelength of at or about 620 nm to at or about 720 nm at a dose of at least at or about 1 J/cm² or at least at or about 1 J/cm of fiber length.

In some embodiments, illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some embodiments, the target area is illuminated at a wavelength of 690±40 nm. In some embodiments, target area is illuminated at a dose of at or about of 50 J/cm² or at or about 100 J/cm of fiber length.

In some embodiments, illuminating is carried out at a wavelength of at or about 580 nm to at or about 830 nm and at a dose of from at or about 25 J/cm² to at or about 400 J/cm² or from at or about 2 J/cm fiber length to at or about 500 J/cm fiber length. In some embodiments, the target area is illuminated at a wavelength of 670±40 nm. In some embodiments, target area is illuminated at a dose of at or about of 50 J/cm² or at or about 100 J/cm of fiber length. In some embodiments, illuminating is carried out at a wavelength of at or about 580 nm to at or about 830 nm and at a dose of from at or about 25 J/cm² to at or about 400 J/cm² or from at or about 2 J/cm fiber length to at or about 500 J/cm fiber length. In some embodiments, the target area is illuminated at a wavelength of 675±50 nm. In some embodiments, target area is illuminated at a dose of at or about of 50 J/cm² or at or about 100 J/cm of fiber length.

In some embodiments, a light or laser may be applied to the conjugate molecules, such as cells containing the conjugate, for a duration of from at or about 5 seconds to at or about 5 minutes. For example, in some embodiments, the light or laser is applied for at or about 5, 10, 15, 20, 25, 30, 35, 40, 45 50 or 55 seconds, or for within a range between any of two such values, to activate the dye molecule(s) of the conjugate. In some embodiments, the light or laser is applied for at or about 1, 1.5, 2, 2.5, 3, 3.5, 4, 4.5 or 5 minutes, or more, or within a range between any two of such values. In some embodiments, the length of time a light or laser is applied can vary depending, for example, on the energy, such as wattage, of the light or laser. For example, lights or lasers with a lower wattage may be applied for a longer period of time in order to activate the dye molecule.

In some embodiments, a light or laser may be applied for at or about 30 minutes to at or about 96 hours after administering the conjugate. For example, in some embodiments, the light or laser is applied at or at about 30, 35, 40, 45, 50 or 55 minutes after administering the conjugate, or within a range between any two of such values. In some embodiments, the light or laser is applied at or at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administering the conjugate, or is administered within a range between about any two of such values, such as, for example between at or about 20 hours to at or about 28 hours, or about 24 hours±4 hours. In some embodiments, the light or laser is applied between or between about 1 and 24 hours, such as between at or about 1 and at or about 12 hours, at or about 12 and at or about 24 hours, at or about 6 and at or about 12 hours, or may be administered more than at or about 24 hours following administration of the conjugate. In some embodiments, the light or laser is applied at or about 36, 48, 72 or 96 hours after administering the conjugate. In some embodiments, the light or laser is applied at or at about 24 hours±4 hours after administering the conjugate.

In some embodiments, the target area, such as a tumor, the vicinity of a tumor, a lymph node, the vicinity of the lymph node, or the tumor microenvironment, or subjects, can be illuminated one or more times. Thus, illumination can be completed in a single day, or can be done repeatedly on multiple days with the same or a different dosage, such as illumination at least at or about 2 different times, 3 different times, 4 different times 5 different times or 10 different times. In some embodiments, repeated illuminations may be done on the same day, on successive days, or every 1-3 days, every 3-7 days, every 1-2 weeks, every 2-4 weeks, every 1-2 months, or at even longer intervals. In some embodiments, multiple illuminations are performed, such as at least 2, at least 3, or at least 4 illuminations, such as 2, 3, 4, 5, 6, 7, 8, 9 or 10 separate administrations.

In some embodiments, the dose or method of illumination differs depending on the type or morphology of the target area, such as a tumor, the vicinity of a tumor, a lymph node, the vicinity of the lymph node.

For example, in some embodiments, the illumination employs a device with “top hat” irradiance distribution profile, such as those described in WO2018/080952 and US20180239074.

### VI. Combination Therapy

In some embodiments, also provided are methods and uses that include combination therapies, and combinations, such as combinations for use in accordance with the combination therapy. In some aspects, the combinations include an anti-PD-L1 antibody or antigen binding fragment thereof provided herein and an additional therapeutic agent, such as an immunomodulatory agent or an anti-cancer agent. In some aspects, the combinations include an anti-PD-L1 antibody conjugate provided herein and an additional therapeutic agent, such as an immunomodulatory agent or an anti-cancer agent. In some embodiments, the conjugate is an anti-PD-L1 antibody, or an antibody fragment that binds to PD-L1, provided herein linked to a Si-phthalocyanine dye, such as an IR700 dye. In some aspects, the combination therapy includes administration of the anti-PD-L1 conjugate and the additional therapeutic, e.g., an immunomodulatory agent or an anti-cancer agent. In such methods, the primary tumors, newly arising tumors, invasive tumor cells, and metastatic tumor cells can be sensitized to the treatment with the additional therapeutic agent, such as an immunomodulatory agent or an anti-cancer agent. In such methods, the growth of primary tumors, newly arising tumors, invasive tumor cells, and metastatic tumor cells can be inhibited, reduced or eliminated, and/or the volume of one or more tumors is reduced.

The increase in sensitivity as a result of such combination treatments can include, but not limited to, a reduction of inhibition of tumor growth of a primary tumor or a tumor distal to the site of administration, a reduction in tumor cell invasion and/or metastasis, an increase in tumor cell killing, an increase in systemic immune response, an increase in new T cell priming, an increase in the diversity of intratumoral CD8+ T cells, an increase in the number and/or activity of intratumoral CD8+ T effector cells, a decrease in the number and/or activity of intratumoral regulatory T cells, a decrease in the number and/or activity of intratumoral myeloid derived suppressor cells, a decrease in the number and/or activity of intratumoral tumor associated fibroblasts or cancer associated fibroblasts (CAFs), or any combination thereof.

In some embodiments the additional therapeutic agent is an anticancer agent. In some embodiments, the anticancer agent can be one or more chemotherapeutic agent(s), an antibody treatment, and a radiotherapeutic agent. In some embodiments, the additional therapeutic agent is an anti-cancer agent selected from a checkpoint inhibitor, an immune adjuvant, a chemotherapeutic agent, radiation, and a biologic comprising an anti-cancer targeting molecule that binds to a tumor cell.

In some aspects, the additional therapeutic agent is an immunomodulatory agent (also called immune modulating agent), such as an immune checkpoint inhibitor. In some aspects, such combination is employed for treatment of the tumor, lesion or cancer. In some embodiments, the methods include the administration of the immunomodulatory agent, such as an immune checkpoint inhibitor, prior to, concurrent with or subsequent to the administration of an anti-PD-L1 conjugate.

In some embodiments, the additional therapeutic agent, such as an immunomodulatory agent, used in such combination therapies herein can include an adjuvant, immune checkpoint inhibitor, cytokine or any combination thereof. A cytokine for use in the combinations can be, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway. An adjuvant for use in the combinations can be, for example, IL-15, IL-2, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some embodiments, the additional therapeutic agent is an immune checkpoint inhibitor that is a PD-1 inhibitor, such as a small molecule, antibody or antigen binding fragment. Exemplary anti-PD-1 antibodies include, but are not limited to, pembrolizumab (MK-3475, Keytruda), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, RO7121661, CX-188, and spartalizumab.

In some embodiments, the additional therapeutic agent is an immune checkpoint inhibitor that is a CTLA-4 inhibitor, such as a small molecule, antibody or antigen binding fragment. In some of any of the embodiments, the anti-CTLA-4 antibody is selected from the group consisting of ipilimumab (YERVOY), tremelimumab, AGEN1181, AGEN1884, ADU-1064, BCD-145, and BCD-217.

In some embodiments, the additional therapeutic agent is a CD25 inhibitor, such as a small molecule, antibody or antigen binding fragment. In some of any of the embodiments, the anti-CD25 antibody is selected from the group consisting of basiliximab (Simulect®), daclizumab, PC61.

The administration of an additional therapeutic agent, such as a checkpoint inhibitor, adjuvant or cytokine, can be administered prior to, concurrent with, or subsequent to the administration of the anti-PD-L1 conjugate. For example, the methods can include administering one or more doses of an immune checkpoint inhibitor, administering an anti-PD-L1 conjugate, and after administration of the conjugate, illuminating with a suitable wavelength of light a target area. The methods can include first administering the conjugate, and after administration of the conjugate, illuminating a target area, and then administering an additional therapeutic agent, such as an immune checkpoint inhibitor, subsequently either to administration of the conjugate or subsequently to the illumination step. The methods can also include the administration of an additional therapeutic agent, such as an immune checkpoint inhibitor, concurrently with administration of the conjugate, followed by illuminating a target area. In some embodiments, an additional therapeutic agent, such as an immune checkpoint inhibitor, adjuvant or cytokine, is administered one or more times, prior to when an anti-PD-L1 conjugate is administered, followed by illuminating a target area, and then one or more additional administrations of an additional therapeutic agent (the same or a different an additional therapeutic agent).

### VII. Articles of Manufacture or Kits

Also provided are articles of manufacture or kit containing the provided anti-PD-L1 antibodies, conjugates, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., multiple myeloma). Alternatively, or additionally, the article of manufacture or kit may further include another or the same container comprising a pharmaceutically-acceptable buffer. It may further include other materials such as other buffers, diluents, filters, needles, and/or syringes.

In particular embodiments, wherein the container contains a conjugate comprising a phthalocyanine dye, the container is a light-protected container, such that the contents are only exposed to a wavelengths within the range of about 400 nm to about 650 nm, or light with an intensity of less than 500 lux. In some embodiments, the container comprising the conjugate protects from transmission of light having a wavelength from or from about 250 nm to about 800 nm, from about 250 nm to about 450 nm, from about 400 nm to about 800 nm, from about 450 nm to about 650 nm, or from about 600 nm to about 720 nm. In some embodiments, container protects from transmission of light such that the percentage of light transmission by the container is less than 50%, less than 40%, less than 30%, less than 20%, less than 10% or less than 5%. In some embodiments, the container is green, blue, amber, translucent, opaque, or is wrapped in an opaque foil. In some embodiments, the container is green, blue, amber, translucent, opaque, or is covered by material with light transmission of less than 50%, less than 40%, less than 30%, less than 20%, less than 10% or less than 5%. In some embodiments of the methods provided herein, the container is selected from among a vial, a tube, a syringe, a bag, a pouch, and a box.

In some embodiments, the light-protected container is a first light-protected container and the method further includes packing the first light-protected container into a second light-protected container. In some embodiments, the second container protects from transmission of light having a wavelength from or from about 250 nm to about 800 nm, from about 250 nm to about 450 nm, from about 400 nm to about 800 nm, from about 450 nm to about 650 nm, or from about 600 nm to about 720 nm. In some embodiments, the second container protects from transmission of light such that the percentage of light transmission is less than 50%, less than 40%, less than 30%, less than 20%, less than 10% or less than 5%. In some embodiments, the second container is green, blue, amber, translucent, opaque, or is covered by a material with light transmission of less than 50%, less than 40%, less than 30%, less than 20%, less than 10% or less than 5%. In some embodiments, the second container is selected from among a vial, a tube, a syringe, a bag, a pouch, and a box.

In some embodiments, the method provided herein further includes packaging the second container into a third light-protected container. In some embodiments, the third container protects from transmission of light having a wavelength from or from about 250 nm to about 800 nm, from about 250 nm to about 450 nm, from about 400 nm to about 800 nm, from about 450 nm to about 650 nm, or from about 600 nm to about 720 nm. In some embodiments, the third container protects from transmission of light such that the percentage of light transmission is less than 50%, less than 40%, less than 30%, less than 20%, less than 10% or less than 5%. In some embodiments, the third container is green, blue, amber, translucent, opaque, or is covered by a material with light transmission of less than 50%, less than 40%, less than 30%, less than 20%, less than 10% or less than 5%. In some embodiments, the third container is selected from among a vial, a tube, a syringe, a bag, a pouch, and a box.

The label or package insert may indicate that the composition is used for treating a PD-L1-expressing or PD-L1-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a PD-L1-expressing or PD-L1-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the anti-PD-L1 antibody or antigen-binding fragment thereof or conjugate; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VIII. Definitions

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-PD-L1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers and PD-L1-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### IX. Exemplary Embodiments

### Among the Provided Embodiments are:

1. An antibody or antigen-binding fragment that specifically binds a Programmed Death-Ligand (PD-L1) protein, wherein the antibody or antigen-binding fragment comprises:
(a) a heavy chain variable (VH) region comprising a heavy chain complementarity determining region 1 (CDR-H1), a CDR-H2, and a CDR-H3, respectively, comprising a CDR-H1, a CDR-H2, and a CDR-H3, respectively, contained within the VH region amino acid sequence set forth in SEQ ID NO:1; and a light chain variable (VL) region comprising a light chain complementarity determining region 1 (CDR-L1), a CDR-L2, and a CDR-L3, respectively, comprising a CDR-L1, a CDR-L2, and a CDR-L3, respectively, contained within the VL region amino acid sequence set forth in SEQ ID NO:17;
(b) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:18;
(c) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:3; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:19;
(d) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:4; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:19;
(e) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:5; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:20;
(f) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:1; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:21;
(g) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:22;
(h) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:6; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:23;
(i) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:7; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:24;
(j) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:8; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:25;
(k) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:9; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:26;
(l) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:10; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:27;
(m) a VH region comprising a CDR-H1 a CDR-H2, and a CDR-H3, respectively, comprising a CDR-H1, a CDR-H2, and a CDR-H3, respectively, contained within the VH region amino acid sequence set forth in SEQ ID NO:11; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:28;
(n) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:12; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:29;
(o) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:13; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:30;
(p) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:14; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:31;
(q) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:15; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:32;
(r) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:12; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:33; or
(s) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:16; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:34.

2. The antibody or antigen-binding fragment of embodiment 1, wherein:
(a) the VH region comprises a CDR-H1 comprising the amino acid sequence set forth in SEQ ID NO:35, a CDR-H2 comprising the amino acid sequence set forth in SEQ ID NO:36, and a CDR-H3 comprising the amino acid sequence set forth in SEQ ID NO:37; and the VL region comprises a CDR-L1 comprising the amino acid sequence set forth in SEQ ID NO:210, a CDR-L2 comprises the amino acid sequence set forth in SEQ ID NO:211, and a CDR-L3 comprising the amino acid sequence set forth in SEQ ID NO:212;
(b) the VH region comprises a CDR-H1 comprising the amino acid sequence set forth in SEQ ID NO:48, a CDR-H2 comprising the amino acid sequence set forth in SEQ ID NO:49, and a CDR-H3 comprising the amino acid sequence set forth in SEQ ID NO:37; and the VL region comprises a CDR-L1 comprising the amino acid sequence set forth in SEQ ID NO:218, a CDR-L2 comprises the amino acid sequence set forth in SEQ ID NO:211, and a CDR-L3 comprising the amino acid sequence set forth in SEQ ID NO:212;
(c) the VH region comprises a CDR-H1 comprising the amino acid sequence set forth in SEQ ID NO:58, a CDR-H2 comprising the amino acid sequence set forth in SEQ ID NO:59, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(d) the VH region comprises a CDR-H1 comprising the amino acid sequence set forth in SEQ ID NO:58, a CDR-H2 comprising the amino acid sequence set forth in SEQ ID NO:59, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(e) the VH region comprises a CDR-H1 comprising the amino acid sequence set forth in SEQ ID NO:71, a CDR-H2 comprising the amino acid sequence set forth in SEQ ID NO:72, and a CDR-H3 comprising the amino acid sequence set forth in SEQ ID NO:60; and the VL region comprises a CDR-L1 comprising the amino acid sequence set forth in SEQ ID NO:229, a CDR-L2 comprises the amino acid sequence set forth in SEQ ID NO:222, and a CDR-L3 comprising the amino acid sequence set forth in SEQ ID NO:223;
(f) the VH region comprises a CDR-H1 comprising the amino acid sequence set forth in SEQ ID NO:35, a CDR-H2 comprising the amino acid sequence set forth in SEQ ID NO:36, and a CDR-H3 comprising the amino acid sequence set forth in SEQ ID NO:37; and the VL region comprises a CDR-L1 comprising the amino acid sequence set forth in SEQ ID NO:233, a CDR-L2 comprises the amino acid sequence set forth in SEQ ID NO:234, and a CDR-L3 comprising the amino acid sequence set forth in SEQ ID NO:235;
(g) the VH region comprises a CDR-H1 comprising the amino acid sequence set forth in SEQ ID NO:48, a CDR-H2 comprising the amino acid sequence set forth in SEQ ID NO:49, and a CDR-H3 comprising the amino acid sequence set forth in SEQ ID NO:37; and the VL region comprises a CDR-L1 comprising the amino acid sequence set forth in SEQ ID NO:241, a CDR-L2 comprises the amino acid sequence set forth in SEQ ID NO:234, and a CDR-L3 comprising the amino acid sequence set forth in SEQ ID NO:242;
(h) the VH region comprises a CDR-H1 comprising the amino acid sequence set forth in SEQ ID NO:82, a CDR-H2 comprising the amino acid sequence set forth in SEQ ID NO:83, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:247, and a CDR-L3 comprising the amino acid sequence set forth in SEQ ID NO:248;
(i) the VH region comprises a CDR-H1 comprising the amino acid sequence set forth in SEQ ID NO:48, a CDR-H2 comprising the amino acid sequence set forth in SEQ ID NO:95, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:254, and a CDR-L3 comprising the amino acid sequence set forth in SEQ ID NO:255;
(j) the VH region comprises a CDR-H1 comprising the amino acid sequence set forth in SEQ ID NO:48, a CDR-H2 comprising the amino acid sequence set forth in SEQ ID NO:104, and a CDR-H3 comprising the amino acid sequence set forth in SEQ ID NO:105; and the VL region comprises a CDR-L1 comprising the amino acid sequence set forth in SEQ ID NO:258, a CDR-L2 comprises the amino acid sequence set forth in SEQ ID NO:259, and a CDR-L3 comprising the amino acid sequence set forth in SEQ ID NO:260;
(k) the VH region comprises a CDR-H1 comprising the amino acid sequence set forth in SEQ ID NO:116, a CDR-H2 comprising the amino acid sequence set forth in SEQ ID NO:117, and a CDR-H3 comprising the amino acid sequence set forth in SEQ ID NO:118; and the VL region comprises a CDR-L1 comprising the amino acid sequence set forth in SEQ ID NO:265, a CDR-L2 comprises the amino acid sequence set forth in SEQ ID NO:266, and a CDR-L3 comprising the amino acid sequence set forth in SEQ ID NO:267;
(l) the VH region comprises a CDR-H1 comprising the amino acid sequence set forth in SEQ ID NO:129, a CDR-H2 comprising the amino acid sequence set forth in SEQ ID NO:130, and a CDR-H3 comprising the amino acid sequence set forth in SEQ ID NO:131; and the VL region comprises a CDR-L1 comprising the amino acid sequence set forth in SEQ ID NO:246, a CDR-L2 comprises the amino acid sequence set forth in SEQ ID NO:273, and a CDR-L3 comprising the amino acid sequence set forth in SEQ ID NO:274;
(m) the VH region comprises a CDR-H1 comprising the amino acid sequence set forth in SEQ ID NO:142, a CDR-H2 comprising the amino acid sequence set forth in SEQ ID NO:143, and a CDR-H3 comprising the amino acid sequence set forth in SEQ ID NO:144; and the VL region comprises a CDR-L1 comprising the amino acid sequence set forth in SEQ ID NO:278, a CDR-L2 comprises the amino acid sequence set forth in SEQ ID NO:279, and a CDR-L3 comprising the amino acid sequence set forth in SEQ ID NO:280;
(n) the VH region comprises a CDR-H1 comprising the amino acid sequence set forth in SEQ ID NO:155, a CDR-H2 comprising the amino acid sequence set forth in SEQ ID NO:156, and a CDR-H3 comprising the amino acid sequence set forth in SEQ ID NO:157; and the VL region comprises a CDR-L1 comprising the amino acid sequence set forth in SEQ ID NO:286, a CDR-L2 comprises the amino acid sequence set forth in SEQ ID NO:287, and a CDR-L3 comprising the amino acid sequence set forth in SEQ ID NO:288;
(o) the VH region comprises a CDR-H1 comprising the amino acid sequence set forth in SEQ ID NO:168, a CDR-H2 comprising the amino acid sequence set forth in SEQ ID NO:169, and a CDR-H3 comprising the amino acid sequence set forth in SEQ ID NO:170; and the VL region comprises a CDR-L1 comprising the amino acid sequence set forth in SEQ ID NO:294, a CDR-L2 comprises the amino acid sequence set forth in SEQ ID NO:234, and a CDR-L3 comprising the amino acid sequence set forth in SEQ ID NO:295;
(p) the VH region comprises a CDR-H1 comprising the amino acid sequence set forth in SEQ ID NO:35, a CDR-H2 comprising the amino acid sequence set forth in SEQ ID NO:181, and a CDR-H3 comprising the amino acid sequence set forth in SEQ ID NO:182; and the VL region comprises a CDR-L1 comprising the amino acid sequence set forth in SEQ ID NO:299, a CDR-L2 comprises the amino acid sequence set forth in SEQ ID NO:300, and a CDR-L3 comprising the amino acid sequence set forth in SEQ ID NO:301;
(q) the VH region comprises a CDR-H1 comprising the amino acid sequence set forth in SEQ ID NO:168, a CDR-H2 comprising the amino acid sequence set forth in SEQ ID NO:169, and a CDR-H3 comprising the amino acid sequence set forth in SEQ ID NO:193; and the VL region comprises a CDR-L1 comprising the amino acid sequence set forth in SEQ ID NO:306, a CDR-L2 comprises the amino acid sequence set forth in SEQ ID NO:234, and a CDR-L3 comprising the amino acid sequence set forth in SEQ ID NO:307;
(r) the VH region comprises a CDR-H1 comprising the amino acid sequence set forth in SEQ ID NO:155, a CDR-H2 comprising the amino acid sequence set forth in SEQ ID NO:156, and a CDR-H3 comprising the amino acid sequence set forth in SEQ ID NO:157; and the VL region comprises a CDR-L1 comprising the amino acid sequence set forth in SEQ ID NO:311, a CDR-L2 comprises the amino acid sequence set forth in SEQ ID NO:312, and a CDR-L3 comprising the amino acid sequence set forth in SEQ ID NO:313; or(s) the VH region comprises a CDR-H1 comprising the amino acid sequence set forth in SEQ ID NO:197, a CDR-H2 comprising the amino acid sequence set forth in SEQ ID NO:198, and a CDR-H3 comprising the amino acid sequence set forth in SEQ ID NO:199; and the VL region comprises a CDR-L1 comprising the amino acid sequence set forth in SEQ ID NO:319, a CDR-L2 comprises the amino acid sequence set forth in SEQ ID NO:320, and a CDR-L3 comprising the amino acid sequence set forth in SEQ ID NO:321.

3. The antibody or antigen-binding fragment of embodiment 1, wherein:
(a) the VH region comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:37; and the VL region comprises a CDR-L1 comprising the amino acid sequence set forth in SEQ ID NO:210, a CDR-L2 comprises the amino acid sequence set forth in SEQ ID NO:211, and a CDR-L3 comprising the amino acid sequence set forth in SEQ ID NO:212;
(b) the VH region comprises a CDR-H1 comprising the amino acid sequence set forth in SEQ ID NO:52, a CDR-H2 comprising the amino acid sequence set forth in SEQ ID NO:53, and a CDR-H3 comprising the amino acid sequence set forth in SEQ ID NO:37; and the VL region comprises a CDR-L1 comprising the amino acid sequence set forth in SEQ ID NO:218, a CDR-L2 comprises the amino acid sequence set forth in SEQ ID NO:211, and a CDR-L3 comprising the amino acid sequence set forth in SEQ ID NO:212;
(c) the VH region comprises a CDR-H1 comprising the amino acid sequence set forth in SEQ ID NO:63, a CDR-H2 comprising the amino acid sequence set forth in SEQ ID NO:64, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(d) the VH region comprises a CDR-H1 comprising the amino acid sequence set forth in SEQ ID NO:63, a CDR-H2 comprising the amino acid sequence set forth in SEQ ID NO:64, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(e) the VH region comprises a CDR-H1 comprising the amino acid sequence set forth in SEQ ID NO:75, a CDR-H2 comprising the amino acid sequence set forth in SEQ ID NO:76, and a CDR-H3 comprising the amino acid sequence set forth in SEQ ID NO:60; and the VL region comprises a CDR-L1 comprising the amino acid sequence set forth in SEQ ID NO:229, a CDR-L2 comprises the amino acid sequence set forth in SEQ ID NO:222, and a CDR-L3 comprising the amino acid sequence set forth in SEQ ID NO:223;
(f) the VH region comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:37; and the VL region comprises a CDR-L1 comprising the amino acid sequence set forth in SEQ ID NO:233, a CDR-L2 comprises the amino acid sequence set forth in SEQ ID NO:234, and a CDR-L3 comprising the amino acid sequence set forth in SEQ ID NO:235;
(g) the VH region comprises a CDR-H1 comprising the amino acid sequence set forth in SEQ ID NO:52, a CDR-H2 comprising the amino acid sequence set forth in SEQ ID NO:53, and a CDR-H3 comprising the amino acid sequence set forth in SEQ ID NO:37; and the VL region comprises a CDR-L1 comprising the amino acid sequence set forth in SEQ ID NO:241, a CDR-L2 comprises the amino acid sequence set forth in SEQ ID NO:234, and a CDR-L3 comprising the amino acid sequence set forth in SEQ ID NO:242;
(h) the VH region comprises a CDR-H1 comprising the amino acid sequence set forth in SEQ ID NO:87, a CDR-H2 comprising the amino acid sequence set forth in SEQ ID NO:88, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:247, and a CDR-L3 comprising the amino acid sequence set forth in SEQ ID NO:248;
(i) the VH region comprises a CDR-H1 comprising the amino acid sequence set forth in SEQ ID NO:98, a CDR-H2 comprising the amino acid sequence set forth in SEQ ID NO:99, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:254, and a CDR-L3 comprising the amino acid sequence set forth in SEQ ID NO:255;
(j) the VH region comprises a CDR-H1 comprising the amino acid sequence set forth in SEQ ID NO:108, a CDR-H2 comprising the amino acid sequence set forth in SEQ ID NO:109, and a CDR-H3 comprising the amino acid sequence set forth in SEQ ID NO:105; and the VL region comprises a CDR-L1 comprising the amino acid sequence set forth in SEQ ID NO:258, a CDR-L2 comprises the amino acid sequence set forth in SEQ ID NO:259, and a CDR-L3 comprising the amino acid sequence set forth in SEQ ID NO:260;
(k) the VH region comprises a CDR-H1 comprising the amino acid sequence set forth in SEQ ID NO:121, a CDR-H2 comprising the amino acid sequence set forth in SEQ ID NO:122, and a CDR-H3 comprising the amino acid sequence set forth in SEQ ID NO:118; and the VL region comprises a CDR-L1 comprising the amino acid sequence set forth in SEQ ID NO:265, a CDR-L2 comprises the amino acid sequence set forth in SEQ ID NO:266, and a CDR-L3 comprising the amino acid sequence set forth in SEQ ID NO:267;
(l) the VH region comprises a CDR-H1 comprising the amino acid sequence set forth in SEQ ID NO:134, a CDR-H2 comprising the amino acid sequence set forth in SEQ ID NO:135, and a CDR-H3 comprising the amino acid sequence set forth in SEQ ID NO:131; and the VL region comprises a CDR-L1 comprising the amino acid sequence set forth in SEQ ID NO:246, a CDR-L2 comprises the amino acid sequence set forth in SEQ ID NO:273, and a CDR-L3 comprising the amino acid sequence set forth in SEQ ID NO:274;
(m) the VH region comprises a CDR-H1 comprising the amino acid sequence set forth in SEQ ID NO:147, a CDR-H2 comprising the amino acid sequence set forth in SEQ ID NO:148, and a CDR-H3 comprising the amino acid sequence set forth in SEQ ID NO:144; and the VL region comprises a CDR-L1 comprising the amino acid sequence set forth in SEQ ID NO:278, a CDR-L2 comprises the amino acid sequence set forth in SEQ ID NO:279, and a CDR-L3 comprising the amino acid sequence set forth in SEQ ID NO:280;
(n) the VH region comprises a CDR-H1 comprising the amino acid sequence set forth in SEQ ID NO:160, a CDR-H2 comprising the amino acid sequence set forth in SEQ ID NO:161, and a CDR-H3 comprising the amino acid sequence set forth in SEQ ID NO:157; and the VL region comprises a CDR-L1 comprising the amino acid sequence set forth in SEQ ID NO:286, a CDR-L2 comprises the amino acid sequence set forth in SEQ ID NO:287, and a CDR-L3 comprising the amino acid sequence set forth in SEQ ID NO:288;
(o) the VH region comprises a CDR-H1 comprising the amino acid sequence set forth in SEQ ID NO:173, a CDR-H2 comprising the amino acid sequence set forth in SEQ ID NO:174, and a CDR-H3 comprising the amino acid sequence set forth in SEQ ID NO:170; and the VL region comprises a CDR-L1 comprising the amino acid sequence set forth in SEQ ID NO:294, a CDR-L2 comprises the amino acid sequence set forth in SEQ ID NO:234, and a CDR-L3 comprising the amino acid sequence set forth in SEQ ID NO:295;
(p) the VH region comprises a CDR-H1 comprising the amino acid sequence set forth in SEQ ID NO:185, a CDR-H2 comprising the amino acid sequence set forth in SEQ ID NO:186, and a CDR-H3 comprising the amino acid sequence set forth in SEQ ID NO:182; and the VL region comprises a CDR-L1 comprising the amino acid sequence set forth in SEQ ID NO:299, a CDR-L2 comprises the amino acid sequence set forth in SEQ ID NO:300, and a CDR-L3 comprising the amino acid sequence set forth in SEQ ID NO:301;
(q) the VH region comprises a CDR-H1 comprising the amino acid sequence set forth in SEQ ID NO:173, a CDR-H2 comprising the amino acid sequence set forth in SEQ ID NO:174, and a CDR-H3 comprising the amino acid sequence set forth in SEQ ID NO:193; and the VL region comprises a CDR-L1 comprising the amino acid sequence set forth in SEQ ID NO:306, a CDR-L2 comprises the amino acid sequence set forth in SEQ ID NO:234, and a CDR-L3 comprising the amino acid sequence set forth in SEQ ID NO:307;
(r) the VH region comprises a CDR-H1 comprising the amino acid sequence set forth in SEQ ID NO:160, a CDR-H2 comprising the amino acid sequence set forth in SEQ ID NO:161, and a CDR-H3 comprising the amino acid sequence set forth in SEQ ID NO:157; and the VL region comprises a CDR-L1 comprising the amino acid sequence set forth in SEQ ID NO:311, a CDR-L2 comprises the amino acid sequence set forth in SEQ ID NO:312, and a CDR-L3 comprising the amino acid sequence set forth in SEQ ID NO:313; or
(s) the VH region comprises a CDR-H1 comprising the amino acid sequence set forth in SEQ ID NO:202, a CDR-H2 comprising the amino acid sequence set forth in SEQ ID NO:203, and a CDR-H3 comprising the amino acid sequence set forth in SEQ ID NO:199; and the VL region comprises a CDR-L1 comprising the amino acid sequence set forth in SEQ ID NO:319, a CDR-L2 comprises the amino acid sequence set forth in SEQ ID NO:320, and a CDR-L3 comprising the amino acid sequence set forth in SEQ ID NO:321.

4. The antibody or antigen-binding fragment of any of embodiments 1-3, wherein:
(a) the VH region comprises an amino acid sequence that has at least 95% sequence identity to the amino acid sequence set forth in SEQ ID NO:1; and the VL region comprises an amino acid sequence that has at least 95% sequence identity to the amino acid sequence set forth in SEQ ID NO:17;
(b) the VH region comprises an amino acid sequence that has at least 95% sequence identity to the amino acid sequence set forth in SEQ ID NO:2; and the VL region comprises an amino acid sequence that has at least 95% sequence identity to the amino acid sequence set forth in SEQ ID NO:18;
(c) the VH region comprises an amino acid sequence that has at least 95% sequence identity to the amino acid sequence set forth in SEQ ID NO:3; and the VL region comprises an amino acid sequence that has at least 95% sequence identity to the amino acid sequence set forth in SEQ ID NO:19;
(d) the VH region comprises an amino acid sequence that has at least 95% sequence identity to the amino acid sequence set forth in SEQ ID NO:4; and the VL region comprises an amino acid sequence that has at least 95% sequence identity to the amino acid sequence set forth in SEQ ID NO:19;
(e) the VH region comprises an amino acid sequence that has at least 95% sequence identity to the amino acid sequence set forth in SEQ ID NO:5; and the VL region comprises an amino acid sequence that has at least 95% sequence identity to the amino acid sequence set forth in SEQ ID NO:20;
(f) the VH region comprises an amino acid sequence that has at least 95% sequence identity to the amino acid sequence set forth in SEQ ID NO:1; and the VL region comprises an amino acid sequence that has at least 95% sequence identity to the amino acid sequence set forth in SEQ ID NO:21;
(g) the VH region comprises an amino acid sequence that has at least 95% sequence identity to the amino acid sequence set forth in SEQ ID NO:2; and the VL region comprises an amino acid sequence that has at least 95% sequence identity to the amino acid sequence set forth in SEQ ID NO:22;
(h) the VH region comprises an amino acid sequence that has at least 95% sequence identity to the amino acid sequence set forth in SEQ ID NO:6; and the VL region comprises an amino acid sequence that has at least 95% sequence identity to the amino acid sequence set forth in SEQ ID NO:23;
(i) the VH region comprises an amino acid sequence that has at least 95% sequence identity to the amino acid sequence set forth in SEQ ID NO:7; and the VL region comprises an amino acid sequence that has at least 95% sequence identity to the amino acid sequence set forth in SEQ ID NO:24;
(j) the VH region comprises an amino acid sequence that has at least 95% sequence identity to the amino acid sequence set forth in SEQ ID NO:8; and the VL region comprises an amino acid sequence that has at least 95% sequence identity to the amino acid sequence set forth in SEQ ID NO:25;
(k) the VH region comprises an amino acid sequence that has at least 95% sequence identity to the amino acid sequence set forth in SEQ ID NO:9; and the VL region comprises an amino acid sequence that has at least 95% sequence identity to the amino acid sequence set forth in SEQ ID NO:26;
(l) the VH region comprises an amino acid sequence that has at least 95% sequence identity to the amino acid sequence set forth in SEQ ID NO:10; and the VL region comprises an amino acid sequence that has at least 95% sequence identity to the amino acid sequence set forth in SEQ ID NO:27;
(m) the VH region comprises an amino acid sequence that has at least 95% sequence identity to the amino acid sequence set forth in SEQ ID NO:11; and the VL region comprises an amino acid sequence that has at least 95% sequence identity to the amino acid sequence set forth in SEQ ID NO:28;
(n) the VH region comprises an amino acid sequence that has at least 95% sequence identity to the amino acid sequence set forth in SEQ ID NO:12; and the VL region comprises an amino acid sequence that has at least 95% sequence identity to the amino acid sequence set forth in SEQ ID NO:29;
(o) the VH region comprises an amino acid sequence that has at least 95% sequence identity to the amino acid sequence set forth in SEQ ID NO:13; and the VL region comprises an amino acid sequence that has at least 95% sequence identity to the amino acid sequence set forth in SEQ ID NO:30;
(p) the VH region comprises an amino acid sequence that has at least 95% sequence identity to the amino acid sequence set forth in SEQ ID NO:14; and the VL region comprises an amino acid sequence that has at least 95% sequence identity to the amino acid sequence set forth in SEQ ID NO:31;
(q) the VH region comprises an amino acid sequence that has at least 95% sequence identity to the amino acid sequence set forth in SEQ ID NO:15; and the VL region comprises an amino acid sequence that has at least 95% sequence identity to the amino acid sequence set forth in SEQ ID NO:32;
(r) the VH region comprises an amino acid sequence that has at least 95% sequence identity to the amino acid sequence set forth in SEQ ID NO:12; and the VL region comprises an amino acid sequence that has at least 95% sequence identity to the amino acid sequence set forth in SEQ ID NO:33; or(s) the VH region comprises an amino acid sequence that has at least 95% sequence identity to the amino acid sequence set forth in SEQ ID NO:16; and the VL region comprises an amino acid sequence that has at least 95% sequence identity to the amino acid sequence set forth in SEQ ID NO:34.

5. The antibody or antigen-binding fragment of any of embodiments 1-4, wherein:
1) the VH region comprises an amino acid sequence that has at least 95% sequence identity to the amino acid sequence set forth in SEQ ID NO:330; and the VL region comprises an amino acid sequence that has at least 95% sequence identity to the amino acid sequence set forth in SEQ ID NO:335;
2) the VH region comprises an amino acid sequence that has at least 95% sequence identity to the amino acid sequence set forth in SEQ ID NO:331; and the VL region comprises an amino acid sequence that has at least 95% sequence identity to the amino acid sequence set forth in SEQ ID NO:336;
3) the VH region comprises an amino acid sequence that has at least 95% sequence identity to the amino acid sequence set forth in SEQ ID NO:332; and the VL region comprises an amino acid sequence that has at least 95% sequence identity to the amino acid sequence set forth in SEQ ID NO:337;
4) the VH region comprises an amino acid sequence that has at least 95% sequence identity to the amino acid sequence set forth in SEQ ID NO:333; and the VL region comprises an amino acid sequence that has at least 95% sequence identity to the amino acid sequence set forth in SEQ ID NO:338;
5) the VH region comprises an amino acid sequence that has at least 95% sequence identity to the amino acid sequence set forth in SEQ ID NO:330; and the VL region comprises an amino acid sequence that has at least 95% sequence identity to the amino acid sequence set forth in SEQ ID NO:339; or
6) the VH region comprises an amino acid sequence that has at least 95% sequence identity to the amino acid sequence set forth in SEQ ID NO:334; and the VL region comprises an amino acid sequence that has at least 95% sequence identity to the amino acid sequence set forth in SEQ ID NO:340.

6. The antibody or antigen-binding fragment of any of embodiments 1-4, wherein:
(a) the VH region comprises the amino acid sequence set forth in SEQ ID NO:1; and the VL region comprises the amino acid sequence set forth in SEQ ID NO:17;
(b) the VH region comprises the amino acid sequence set forth in SEQ ID NO:2; and the VL region comprises the amino acid sequence set forth in SEQ ID NO:18;
(c) the VH region comprises the amino acid sequence set forth in SEQ ID NO:3; and the VL region comprises the amino acid sequence set forth in SEQ ID NO:19;
(d) the VH region comprises the amino acid sequence set forth in SEQ ID NO:4; and the VL region comprises the amino acid sequence set forth in SEQ ID NO:19;
(e) the VH region comprises the amino acid sequence set forth in SEQ ID NO:5; and the VL region comprises the amino acid sequence set forth in SEQ ID NO:20;
(f) the VH region comprises the amino acid sequence set forth in SEQ ID NO: 1; and the VL region comprises the amino acid sequence set forth in SEQ ID NO:21;
(g) the VH region comprises the amino acid sequence set forth in SEQ ID NO:2; and the VL region comprises the amino acid sequence set forth in SEQ ID NO:22;
(h) the VH region comprises the amino acid sequence set forth in SEQ ID NO:6; and the VL region comprises the amino acid sequence set forth in SEQ ID NO:23;
(i) the VH region comprises the amino acid sequence set forth in SEQ ID NO:7; and the VL region comprises the amino acid sequence set forth in SEQ ID NO:24;
(j) the VH region comprises the amino acid sequence set forth in SEQ ID NO:8; and the VL region comprises the amino acid sequence set forth in SEQ ID NO:25;
(k) the VH region comprises the amino acid sequence set forth in SEQ ID NO:9; and the VL region comprises the amino acid sequence set forth in SEQ ID NO:26;
(l) the VH region comprises the amino acid sequence set forth in SEQ ID NO:10; and the VL region comprises the amino acid sequence set forth in SEQ ID NO:27;
(m) the VH region comprises the amino acid sequence set forth in SEQ ID NO:11; and the VL region comprises the amino acid sequence set forth in SEQ ID NO:28;
(n) the VH region comprises the amino acid sequence set forth in SEQ ID NO: 12; and the VL region comprises the amino acid sequence set forth in SEQ ID NO:29;
(o) the VH region comprises the amino acid sequence set forth in SEQ ID NO:13; and the VL region comprises the amino acid sequence set forth in SEQ ID NO:30;
(p) the VH region comprises the amino acid sequence set forth in SEQ ID NO: 14; and the VL region comprises the amino acid sequence set forth in SEQ ID NO:31;
(q) the VH region comprises the amino acid sequence set forth in SEQ ID NO: 15; and the VL region comprises the amino acid sequence set forth in SEQ ID NO:32;
(r) the VH region comprises the amino acid sequence set forth in SEQ ID NO: 12; and the VL region comprises the amino acid sequence set forth in SEQ ID NO:33; or(s) the VH region comprises the amino acid sequence set forth in SEQ ID NO:16; and the VL region comprises the amino acid sequence set forth in SEQ ID NO:34.

7. The antibody or antigen-binding fragment of any of embodiments 1-6, wherein:
1) the VH region comprises the amino acid sequence set forth in SEQ ID NO:330; and the VL region comprises the amino acid sequence set forth in SEQ ID NO:335
2) the VH region comprises the amino acid sequence set forth in SEQ ID NO:331; and the VL region comprises the amino acid sequence set forth in SEQ ID NO:336
3) the VH region comprises the amino acid sequence set forth in SEQ ID NO:332; and the VL region comprises the amino acid sequence set forth in SEQ ID NO:337
4) the VH region comprises the amino acid sequence set forth in SEQ ID NO:333; and the VL region comprises the amino acid sequence set forth in SEQ ID NO:338
5) the VH region comprises the amino acid sequence set forth in SEQ ID NO:330; and the VL region comprises the amino acid sequence set forth in SEQ ID NO:339; or
6) the VH region comprises the amino acid sequence set forth in SEQ ID NO:334; and the VL region comprises the amino acid sequence set forth in SEQ ID NO:340.

8. The antibody or antigen-binding fragment of embodiment 1, wherein the VH region comprises a CDR-H1, a CDR-H2, and a CDR-H3, respectively, comprising a CDR-H1, a CDR-H2, and a CDR-H3, respectively, contained within the VH region amino acid sequence set forth in SEQ ID NO:6; and the VL region comprises a CDR-L1, a CDR-L2, and a CDR-L3, respectively, comprising a CDR-L1, a CDR-L2, and a CDR-L3, respectively, contained within the VL region amino acid sequence set forth in SEQ ID NO:23.

9. The antibody or antigen-binding fragment of embodiment 1 or 8, wherein the VH region comprises a CDR-H1 comprising the amino acid sequence set forth in SEQ ID NO:82, a CDR-H2 comprising the amino acid sequence set forth in SEQ ID NO:83, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:247, and a CDR-L3 comprising the amino acid sequence set forth in SEQ ID NO:248.

10. The antibody or antigen-binding fragment of embodiment 1 or 8, wherein the VH region comprises a CDR-H1 comprising the amino acid sequence set forth in SEQ ID NO:87, a CDR-H2 comprising the amino acid sequence set forth in SEQ ID NO:88, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:247, and a CDR-L3 comprising the amino acid sequence set forth in SEQ ID NO:248.

11. The antibody or antigen-binding fragment of any of embodiments 1 and 8-10, wherein the VH region comprises an amino acid sequence that has at least 95% sequence identity to the amino acid sequence set forth in SEQ ID NO:6; and the VL region comprises an amino acid sequence that has at least 95% sequence identity to the amino acid sequence set forth in SEQ ID NO: 23.

12. The antibody or antigen-binding fragment of any of embodiments 1 and 8-11, wherein the VH region comprises the amino acid sequence set forth in SEQ ID NO:6; and the VL region comprises the amino acid sequence set forth in SEQ ID NO:23.

13. The antibody or antigen-binding fragment of embodiment 1, wherein the VH region comprises a CDR-H1, a CDR-H2, and a CDR-H3, respectively, comprising a CDR-H1, a CDR-H2, and a CDR-H3, respectively, contained within the VH region amino acid sequence set forth in SEQ ID NO:1; and the VL region comprises a CDR-L1, a CDR-L2, and a CDR-L3, respectively, comprising a CDR-L1, a CDR-L2, and a CDR-L3, respectively, contained within the VL region amino acid sequence set forth in SEQ ID NO:21.

14. The antibody or antigen-binding fragment of embodiment 1 or 13, wherein the VH region comprises a CDR-H1 comprising the amino acid sequence set forth in SEQ ID NO:35, a CDR-H2 comprising the amino acid sequence set forth in SEQ ID NO:36, and a CDR-H3 comprising the amino acid sequence set forth in SEQ ID NO:37; and the VL region comprises a CDR-L1 comprising the amino acid sequence set forth in SEQ ID NO:233, a CDR-L2 comprises the amino acid sequence set forth in SEQ ID NO:234, and a CDR-L3 comprising the amino acid sequence set forth in SEQ ID NO:235.

15. The antibody or antigen-binding fragment of embodiment 1 or 13, wherein the VH region comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:37; and the VL region comprises a CDR-L1 comprising the amino acid sequence set forth in SEQ ID NO:233, a CDR-L2 comprises the amino acid sequence set forth in SEQ ID NO:234, and a CDR-L3 comprising the amino acid sequence set forth in SEQ ID NO:235.

16. The antibody or antigen-binding fragment of any of embodiments 1 and 13-15, wherein the VH region comprises an amino acid sequence that has at least 95% sequence identity to the amino acid sequence set forth in SEQ ID NO:1; and the VL region comprises an amino acid sequence that has at least 95% sequence identity to the amino acid sequence set forth in SEQ ID NO: 21.

17. The antibody or antigen-binding fragment of any of embodiments 1 and 13-16, wherein the VH region comprises the amino acid sequence set forth in SEQ ID NO: 1; and the VL region comprises the amino acid sequence set forth in SEQ ID NO:21.

18. The antibody or antigen-binding fragment of any of embodiments 1-17, wherein the PD-L1 protein is a human PD-L1 protein.

19. The antibody or antigen-binding fragment of any of embodiments 1-18, wherein the antibody or antigen-binding fragment is recombinant.

20. The antibody or antigen-binding fragment of any of embodiments 1-19, wherein the antibody or antigen-binding fragment is monoclonal.

21. The antibody or antigen-binding fragment of any of embodiments 1-20, wherein the antibody or antigen-binding fragment is a human, chimeric, or humanized antibody or antigen-binding fragment.

22. The antibody or antigen-binding fragment of any of embodiments 1-21, wherein the antibody or antigen binding fragment comprises an Fc region of a human immunoglobulin and/or human antibody framework regions.

23. The antibody or antigen-binding fragment of any of embodiments 1-22, that is a single chain antibody fragment.

24. The antibody or antigen-binding fragment of embodiment 23, wherein the antibody fragment comprises a single chain Fv (scFv).

25. The antibody or antigen-binding fragment of any of embodiments 1-22, that is a whole or intact antibody.

26. The antibody or antigen-binding fragment of any of embodiments 1-25, that is a bispecific antibody that further specifically binds to a second antigen.

27. The antibody or antigen-binding fragment of embodiment 26, wherein the second antigen is an antigen expressed on a tumor cell or an immune cell.

28. The antibody or antigen-binding fragment of embodiment 26 or 27, wherein the second antigen is an antigen expressed on an immune cell that is a T cell and the antigen is CD25.

29. The antibody or antigen-binding fragment of any of embodiments 1-28, wherein the antibody or antigen-binding fragment thereof comprises an Fc region that exhibits one or more Fc-mediated effector function(s).

30. The antibody or antigen-binding fragment of any of embodiments 1-28, wherein the antibody or antigen-binding fragment thereof comprises an Fc region that lacks Fc-mediated effector function(s), exhibits substantially reduced Fc-mediated effector function(s) or does not exhibit substantial Fc-mediated effector function(s).

31. The antibody or antigen-binding fragment of any of embodiments 1-28, wherein the antibody or antigen-binding fragment thereof comprises an Fc region that exhibits enhanced Fc-mediated effector function(s).

32. The antibody or antigen-binding fragment of any of embodiments 29-31, wherein the Fc-mediated effector function is selected from one or more of an antibody-dependent cellular cytotoxicity (ADCC), antibody-dependent cellular phagocytosis (ADCP) or complement-dependent cytotoxicity (CDC).

33. The antibody or antigen-binding fragment of any of embodiments 1-32, wherein the antibody or antigen binding fragment comprises an IgG1 Fc region or an IgG1 isotype, an IgG2 Fc region or an IgG2 isotype, IgG3 Fc region or an IgG3 isotype, or an IgG4 Fc region or an IgG4 isotype.

34. A conjugate, comprising the antibody or antigen-binding fragment of any of embodiments 1-33 and a heterologous molecule or moiety.

35. The conjugate of embodiment 34, wherein the heterologous molecule or moiety is a protein, peptide, nucleic acid, dye, or small molecule.

36. The conjugate of embodiment 34 or 35, wherein the heterologous molecule or moiety is a cytotoxic agent, a toxin, a radioisotope, a chemotherapeutic agent, a lytic peptide, a cytokine, or a photoactivatable dye.

37. The conjugate of embodiment 36, wherein the photoactivatable dye is a phthalocyanine dye.

38. The conjugate of embodiment 37, wherein the phthalocyanine dye is a Si-phthalocyanine dye.

39. The conjugate of embodiment 37 or 38, wherein the phthalocyanine dye is IR700.

40. The conjugate of embodiment 37 or 38, wherein the phthalocyanine dye has the structure of Formula (I):

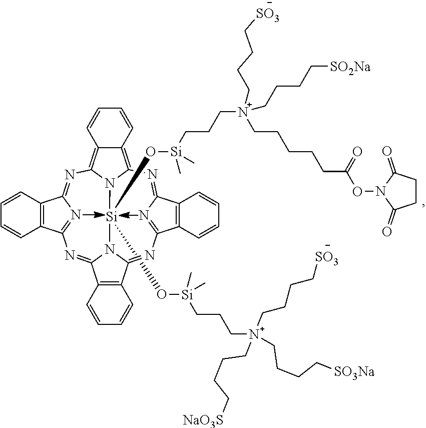

or is a salt, stereoisomer, or tautomer thereof.

41. The conjugate of any of embodiments 34-40, wherein the conjugate is activated by illumination at a wavelength between at or at about 600 nm and at or about 850 nm to effect cell killing.

42. The conjugate of embodiment 41, wherein the activated conjugate affects tumor growth inhibition or killing at a higher level, activity, or potency than the unconjugated antibody.

43. The conjugate of any of embodiments 34-41, wherein the antibody or antigen-binding fragment and the moiety are linked directly or indirectly via a linker.

44. The conjugate of any of embodiments 34-41, wherein the antibody or antigen-binding fragment is covalently attached to the heterologous molecule or moiety.

45. The conjugate of any of embodiments 34-44, wherein when contacted with a cell expressing a PD-L1 protein, the conjugate exhibits increased internalization compared to an unconjugated antibody or antigen-binding fragment, or a conjugate comprising a reference antibody.

46. The conjugate of any of embodiments 34-44, wherein when contacted with a cell expressing a PD-L1 protein, the conjugate exhibits reduced internalization compared to an unconjugated antibody or antigen-binding fragment, or a conjugate comprising a reference antibody.

47. The conjugate of embodiment 45 or 46, wherein the reference antibody is avelumab.

48. The conjugate of any of embodiments 34-47, wherein the conjugate does not exhibit substantially reduced binding affinity to a PD-L1 protein compared to the unconjugated antibody, or exhibits at least 50%, at least 60%, at least 70%, at least 80%, at least 90%, at least 95%, at least 96%, at least 97%, at least 98%, or at least 99% of the binding affinity of the unconjugated antibody to the PD-L1 protein.

49. The conjugate of any of embodiments 34-48, wherein the conjugate exhibits similar binding to a PD-L1 protein compared to the unconjugated antibody.

50. A polynucleotide encoding the antibody or antigen-binding fragment of any of embodiments 1-33.

51. A vector, comprising the polynucleotide of embodiment 50.

52. The vector of embodiment 51, wherein the vector is an expression vector.

53. An engineered cell comprising the vector of embodiment 51 or 52.

54. An engineered cell expressing the antibody or antigen binding fragment of any of embodiments 1-33.

55. A composition comprising the antibody or antigen-binding fragment thereof of any of embodiments 1-33, or the conjugate of any of embodiments 34-49.

56. The composition of embodiment 55, further comprising a pharmaceutically acceptable excipient.

57. A method of treatment, comprising administering the antibody or antigen-binding fragment thereof of any of embodiments 1-33 or the conjugate of any of embodiments 34-49, to a subject having a disease or disorder.

58. A method of treatment, comprising administering a composition of embodiment 55 or 56 to a subject having a disease or disorder.

59. The method of embodiment 58, wherein the disease or disorder is a tumor or a cancer.

60. A method of treating a tumor or a lesion in a subject, comprising:
a) administering to the subject the conjugate of any of embodiments 34-49 or the composition of embodiment 55 or 56; and
b) illuminating a target area within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.

61. A method of treating a tumor or lesion, comprising:
(a) administering to the subject the conjugate of any of embodiments 34-49 or the composition of embodiment 55 or 56 to a subject having a tumor or lesion that has had a low response to, was unresponsive to, was resistant to, was refractory to, had failed to respond to or has relapsed after, a prior immunotherapy; and
(b) illuminating a target area awhere the tumor or lesion is located, at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein the method results in the killing of a PD-L1 expressing cell in the target area.

62. The method of embodiment 61, wherein the prior immunotherapy is a treatment with an immune checkpoint inhibitor.

63. The method of any of embodiments 57-62, wherein the subject has primary resistance or acquired resistance to a prior immunotherapy that comprises a PD-1/PD-L1 blockade therapy.

64. A method of treating a tumor or lesion, the method comprising:
(a) administering to the subject the conjugate of any of embodiments 34-49 or the composition of embodiment 55 or 56 to a subject that is treatment-naïve for an immune checkpoint inhibitor or that has not previously received a treatment with an immune checkpoint inhibitor; and
(b) illuminating a target area where a tumor or lesion is located in the subject at a wavelength of at or about 600 nm to at or about 850 nm and at a dose of from at or about 25 J/cm² to at or about 400 J/cm² or from at or about 2 J/cm fiber length to at or about 500 J/cm fiber length; wherein after the illumination, the growth, size or viability of the tumor or lesion is reduced or inhibited.

65. The method of any of embodiments 57-64, wherein the subject is administered the conjugate to treat, inhibit the growth of and/or reduce the size of a first tumor or lesion; and the method inhibits, delays or prevents the appearance, growth or establishment of one or more second tumors or lesions, located distally to the first tumor or lesion.

66. A method of immunizing a subject having a first tumor or lesion, the method comprising:
(a) administering to the subject the conjugate of any of embodiments 34-49 or the composition of embodiment 55 or 56 to a subject having a tumor or lesion; and
(b) illuminating a target area within the first tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein the first tumor or lesion is inhibited in growth and/or reduced in size; and the appearance, growth or establishment of one or more second tumors or lesions, located distally to the treated first tumor or lesion, is inhibited, delayed or prevented.

67. The method of embodiment 65 or 66, wherein the second tumor or lesion is a metastasis of the first tumor or lesion.

68. The method of any of embodiments 65-67, wherein the method results in killing of a PD-L1-expressing cell in the vicinity of the first tumor or lesion and/or activates an immune cell response, thereby inhibiting, delaying or preventing the appearance, growth or establishment of the second tumor or lesion.

69. The method of any of embodiments 65-68, wherein the second tumor or lesion is phenotypically and/or genotypically the same as the first tumor or lesion.

70. The method of any of embodiments 65-69, wherein the second tumor or lesion is phenotypically and/or genotypically different from the first tumor or lesion.

71. The method of embodiment 65 or 66, wherein the second tumor or lesion is not derived from a metastasis of the first tumor or lesion.

72. The method of any of embodiments 57-71, wherein the method results in the killing of the PD-L1-expressing cell or the PD-L1-expressing immune cell.

73. The method of any of embodiments 57-72, wherein the tumor or lesion comprises a tumor cell, and the tumor cell does not express or has a reduced expression of an immune checkpoint protein.

74. The method of embodiment 73, wherein the immune checkpoint protein is selected from among PD-L1, PD-1, and CTLA-4.

75. The method of embodiment 73 or 74, wherein the tumor cell does not express PD-L1 in response to an inflammatory stimulus.

76. The method of embodiment 75, wherein the inflammatory stimulus is interferon.

77. The method of any of embodiments 73-76, wherein the tumor cell is not specifically recognized by an anti-PD-L1 antibody.

78. The method of any of embodiments 59-77, wherein the tumor or lesion comprises PD-L1 negative tumor cells.

79. The method of embodiment 78, wherein at least or at least about 40%, 50%, 60%, 70%, 80%, 90% or 95% of the tumor cells in the tumor or lesion are PD-L1 negative tumor cells.

80. The method of any of embodiments 57-79, wherein the treatment delays regrowth of the tumor or lesion, prevents a relapse of a cancer associated with the tumor or lesion or prolongs the duration of remission of a cancer associated with the tumor or lesion.

81. The method of any of embodiments 57-80, wherein the inhibition of the growth of the tumor or lesion and/or killing of the PD-L1-expressing cell is dependent on the presence of CD8+ T cells.

82. The method of any of embodiments 57-81, wherein the subject is naïve to treatment with an immune checkpoint inhibitor or has not previously received treatment with an immune checkpoint inhibitor.

83. The method of any of embodiments 57-82, wherein the subject has been previously treated with an immune checkpoint inhibitor.

84. The method of embodiment 83, wherein the subject has had a low response to, was unresponsive to, was resistant to, was refractory to, had failed to respond to or has relapsed after the previous treatment with the immune checkpoint inhibitor.

85. The method of embodiment 83 or 84, wherein the inhibition of the growth, size or viability of the tumor or lesion resulting from carrying out the method is greater compared to the inhibition resulting from the previous treatment with the immune checkpoint inhibitor.

86. The method of any of embodiments 62-85, wherein the immune checkpoint inhibitor is an inhibitor of PD-L1, PD-1 or CTLA-4.

87. The method of any of embodiments 62-86, wherein immune checkpoint inhibitor is a PD-1 inhibitor.

88. The method of embodiment 87, wherein the PD-1 inhibitor is an anti-PD-1 antibody.

89. The method of any of embodiments 62-86, wherein the immune checkpoint inhibitor is PD-L1 inhibitor.

90. The method of embodiment 89, wherein the PD-L1 inhibitor is an anti-PD-L1 antibody.

91. The method of any of embodiments 57-90, wherein the method increases the number or activity of immune cells in the tumor or lesion and/or in the microenvironment of the tumor or lesion.

92. The method of any of embodiments 57-91, wherein the target area comprises immune cells expressing PD-L1.

93. The method of any of embodiments 61-92, wherein the PD-L1 expressing cell is an immune cell.

94. The method of embodiment 92 or 93, wherein the immune cell is selected from the group consisting of monocytes, macrophages, dendritic cells (DC), M2 tumor associated macrophages (M2 TAM), tolerogenic dendritic cells (tDC) and myeloid derived suppressor cells (MDSC).

95. The method of any of embodiments 92-94, wherein the immune cell is located in the tumor, the tumor microenvironment or a lymph node.

96. The method of any of embodiments 57-95, wherein prior to administering the conjugate, the subject has a tumor or lesion having a low number or level of CD8+ T cell infiltration.

97. The method of any of embodiments 57-96, wherein the number, level or activity of immune cells is increased in the tumor or lesion or in the microenvironment of the tumor or lesion after the administering and the illuminating.

98. The method of embodiment 96 or 97, wherein the number or level of CD8+ T cell infiltration in the tumor or lesion is increased after the administering and the illuminating.

99. The method of any of embodiments 96-98, wherein the number or level of memory T cells in the vicinity of the tumor or lesion is increased after the administering and the illuminating.

100. The method of any of embodiments 57-99, wherein the targeting molecule is or comprises an antibody, an antigen-binding antibody fragment or antibody-like molecule that binds PD-L1.

101. The method of embodiment 100, wherein the targeting molecule is or comprises an anti-PD-L1 antibody or antigen-binding fragment thereof.

102. The method of any of embodiments 60-101, wherein the target area is a lymph node or in the vicinity of a lymph node.

103. The method of any of embodiments 60-102, wherein the subject exhibits a durable response, prolonged progression-free survival, a reduced chance of relapse, and/or a reduced chance of metastasis, after the administering and the illuminating.

104. The method of any of embodiments 60-103, wherein the illuminating is carried out between 30 minutes and 96 hours after administering the conjugate.

105. The method of any of embodiments 60-104, wherein the illuminating is carried out 24 hours±4 hours after administering the conjugate.

106. The method of any of embodiments 60-105, wherein the target area is illuminated at a wavelength of 690±40 nm.

107. The method of any of embodiments 60-106, wherein the target area is illuminated at a wavelength of 670±50 nm

108. The method of any of embodiments 60-107, wherein the target area is illuminated at a dose of at or about of 50 J/cm² or at or about 100 J/cm of fiber length.

109. The method of any of embodiments 59-108, wherein the tumor, lesion or cancer is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

110. The method of any of embodiments 59-109, wherein one or more of steps of the method are repeated.

111. The method of any of embodiments 60-110, wherein the administration of the antibody or antigen-binding fragment, the conjugate or the composition is repeated one or more times, optionally wherein after each repeated administration of the conjugate or the composition, the illuminating step is repeated.

112. The method of any of embodiments 59-111, further comprising administering an additional therapeutic agent or anti-cancer treatment.

### X. Examples

The following examples are included for illustrative purposes only and are not intended to limit the scope of the invention.

### Example 1: Generation and Assessment of Human Anti-PD-L1 Antibodies

Exemplary human anti-PD-L1 antibodies containing a heavy chain variable (V_{H}) region and a light chain variable (V_{L}) region that specifically bound to PD-L1 were generated and assessed.

### A. Library Selection and Antibody Generation

ATX-GK+ mice on a Balb/c background (Alloy Therapeutics) and ATX-GK mice on a C₅₇BL/6NTac background (Alloy Therapeutics), which produce fully human antibodies, Balb/c wild-type mice (Charles River) and wild-type C57BL/6 mice (Charles River), which produce mouse antibodies, were immunized against 10 μg of the extracellular domain of human PD-L1 (huPD-L1) fused to a His tag (RD Systems Cat. No. 9049-B7) or human IgG1 Fc domain (RD Systems Cat. No. 156-B7) by injection twice per week (10 μg peptide per injection) for 4 weeks with a final boost injection before the popliteal and inguinal lymph nodes and spleen were harvested, and hybridoma libraries were generated from isolated lymphocytes. The hybridoma supernatants were then tested for binding to human PD-L1 by ELISA.

Hybridomas producing supernatant yielding anti-PD-L1 antibodies were then isolated by single cell sorting, and selected individual clones were expanded and underwent further screening for PD-L1 binding by ELISA. Of the individual clones tested, a subset of clones were selected for further characterization, which included ELISA evaluation of binding to huPD-L1, cynomolgus PD-L1 (cyPD-L1), and mouse PD-L1 (muPD-L1), and flow cytometric assessment of binding CHO cells engineered to express huPD-L1 and assessed by flow cytometry as described below.

### 1. ELISA Assay to Determine PD-L1 Binding

Hybridoma supernatants were tested for binding to huPD-L1, cyPD-L1, and muPD-L1 by ELISA. Briefly, 30 μL 6xHis-tagged huPD-L1, cyPD-L1, or muPD-L1 (1 μg/mL in PBS) were immobilized in wells of a 96-well assay plate overnight at 4° C. After washing and blocking steps, 30 μL of antibody supernatant were added to the plate and incubated at room temperature for 1 hr. Bound human antibody was detected using horseradish peroxidase (HRP)-conjugated goat anti-human Fc (Invitrogen Cat. No. 109-035-097; diluted 1:5000) or goat anti-mouse Fc HRP (Invitrogen Cat. No. 115-035-071) as a control. Bound antibody was detected with 1-Step Ultra™ TMB-ELISA Substrate (ThermoFisher Scientific Cat. No. 34029). When the reaction was complete the reaction was stopped, and the plate was read at 450 nm. All 280 clones bound to huPD-L1, and most of the 280 clones also bound cyPD-L1, though some exhibited weaker binding to cyPD-L1. The clones had little to no cross-reactivity with muPD-L1.

### 2. Cell Surface PD-L1 Binding

Wild-type (wt) Chinese hamster ovary (CHO) cells (ATCC) and CHO cells engineered to express huPD-L1 (BPS Bioscience) were seeded at 200,000 cells in 50 μL FACS buffer (PBS containing 1% FBS and 5 mM EDTA) per well of a 96-well plate. Cell viability was assessed by adding 50 μL 2×Zombie Violet (BioLegend). Dead cell controls were also prepared and 2×Zombie violet was added. After incubating 20-30 minutes at room temperature, protected from light, 150 μL FACS buffer were added to each well. The plates were centrifuged, and the supernatant was aspirated. The cells were resuspended in 100 μL of FACS buffer alone (control), 1 μg/mL anti-PD-L1 control antibody (Avelumab) in FACS buffer, 1 μg/mL isotype control antibody in FACS buffer, or undiluted hybridoma clone supernatants. The plates were incubated 30 minutes on ice, protected from light. 150 μL FACS buffer were then added to each well and the plate was centrifuged. The supernatant was removed and the cells were washed three times in 200 μL FACS buffer. After the third wash, cells were resuspended FACS buffer (control), FACS buffer containing 2.5 μg/mL goat-anti-human IgG secondary antibody conjugated to AlexaFluor 488, or FACS buffer containing 0.3 μg/mL goat-anti-mouse IgG Fcγ secondary antibody conjugated to AlexaFluor 647 (control). The cells were incubated 30 minutes on ice, protected from light. 150 μL FACS buffer were then added to each well and the plate was centrifuged. Supernatant was removed and the cells were washed twice with 200 μL FACS buffer. The cells were then resuspended in FACS buffer and analyzed by CytoFLEX using the appropriate channels. After gating for whole, single, live cells, the MFI in the APC channel (or FITC channel for human control samples) was normalized to the signal from hybridoma media alone (or isotype control for human control). MFI fold over media alone was determined for each clone supernatant tested. Clones that showed similar binding signal to the huPD-L1 expressing CHO cells as the anti-PD-L1 antibody control, with negligible binding to the wt CHO cells, were selected for sequencing.

### 3. Inhibition of PD-1:PD-L1 Interaction

Supernatants of the hybridoma clones were tested for the ability to block the interaction of PD-1 with PD-L1 using a PD-1:PD-L1 Blockade Bioassay (Promega). Briefly, PD-L1 aAPC/CHO-K1 cells, CHO-K1 cells expressing human PD-L1 and an engineered cell surface protein designed to activate cognate TCRs in an antigen-independent manner, were thawed and plated in flat-bottom, white 96-well plates in 200 μL 90% Haim's F-12 medium, 10% FBS. After incubating overnight at 37° C., 5% CO₂, the culture medium was removed, and 40 μL undiluted hybridoma clone supernatant or control PD-1 antibody (at 25 μg/mL) were added. The plates were covered with a lid and kept at room temperature while the PD-1 Effector cells, Jurkat T cells expressing human PD-1 and a luciferase reporter driven by an NFAT response element (NFAT-RE), were thawed and resuspended in Assay Buffer. 40 μL of PD-1 Effector cells were added to the assay wells, and the plates were incubated at 37° C., 5% CO₂ for 6 hours. The plates were removed from the incubator and equilibrated to room temperature for 5-10 minutes before adding 80 μL Bio-Glo™ Reagent. The plates incubated at room temperature for 15 minutes and then the luminescence was measured using a Tecan Spark plate reader.

Of the 276 hybridomas derived from the ATX-GK+ mice, 262 blocked the PD-1:PD-L1 interaction at similar or higher than the anti-PD-1 control antibody. 13 did not block the PD-1:PD-L1 interaction, and 1 clone exhibited partial blocking. Of the 83 hybridomas derived from ATX-GK mice or BALB/c wild-type mice, 13 blocked, 65 did not block, and 5 weakly blocked the PD-1:PD-L1 interaction.

### 4. Sequencing

Clones that were confirmed to bind PD-L1 and confirmed to inhibit the PDL1:PD1 interaction were sequenced. Sequences were manually inspected and optimized for example, to remove identified glycosylation sites. The resulting amino acid sequences of the variable heavy chain (V_{H}) and variable light chain (VL) of the anti-PD-L1 antibodies, and their respective complementarity determining regions (CDRs) according to Chothia, AbM, Kabat, Contact, and IMGT numbering, are provided below in Tables E1a and E1b, respectively.

TABLE E1a
Variable heavy chain (VH) and CDR sequences

| | VH | | CDR-H1 | | CDR-H2 | | CDR-H3 | |
|---|---|---|---|---|---|---|---|---|
| | SEQ | | | SEQ | | SEQ | | SEQ |
| | ID | Numbering | | ID | | ID | | ID |
| Clone | NO | Scheme | AA sequence | NO | AA sequence | NO | AA sequence | NO |
| 1P1 | 1 | Chothia | GFTFSNY--- | 35 | -----LYDGSN--------- | 36 | --DRVWLDV | 37 |
| | | AbM | GFTFSNYGMH | 38 | ---LILYDGSNK-------- | 39 | --DRVWLDV | 37 |
| | | Kabat | -----NYGMH | 40 | ---LILYDGSNKYYADSVKG | 41 | --DRVWLDV | 37 |
| | | Contact | ----SNYGMH | 42 | WVSLILYDGSNKY------- | 43 | ARDRVWLD- | 44 |
| | | IMGT | GFTFSNYG-- | 45 | ----ILYDGSNK-------- | 46 | ARDRVWLDV | 47 |
| 1P1.2 | 2 | Chothia | GFTFSSY--- | 48 | -----WYDGSN--------- | 49 | --DRVWLDV | 37 |
| | | AbM | GFTFSSYGMH | 50 | ---VIWYDGSNKY------- | 51 | --DRVWLDV | 37 |
| | | Kabat | -----SYGMH | 52 | ---VIWYDGSNKYYADSVKG | 53 | --DRVWLDV | 37 |
| | | Contact | ----SSYGMH | 54 | WVAVIWYDGSNKY------- | 55 | ARDRVWLD- | 44 |
| | | IMGT | GFTFSSYG-- | 56 | ----IWYDGSNK-------- | 57 | ARDRVWLDV | 47 |
| 1P2 | 3 | Chothia | GFTFSRY--- | 58 | ------NPNNGN-------- | 59 | --GWTFFDY | 60 |
| | | AbM | GFTFSRYDEN | 61 | ----WMNPNNGNTG------ | 62 | --GWTFFDY | 60 |
| | | Kabat | -----RYDEN | 63 | ---WMNPNNGNTGYSQKFQG | 64 | --GWTFFDY | 60 |
| | | Contact | ----SRYDEN | 65 | WMGWMNPNNGNTG------- | 66 | ARGWTFFD- | 67 |
| | | IMGT | GFTFSRYD-- | 68 | ----MNPNNGNT-------- | 69 | ARGWTFFDY | 70 |
| 1P2_N73D | 4 | Chothia | GFTFSRY--- | 58 | -----NPNNGN--------- | 59 | --GWTFFDY | 60 |
| | | AbM | GFTFSRYDEN | 61 | ---WMNPNNGNTG------- | 62 | --GWTFFDY | 60 |
| | | Kabat | -----RYDEN | 63 | ---WMNPNNGNTGYSQKFQG | 64 | --GWTFFDY | 60 |
| | | Contact | ---SRYDEN | 65 | WMGWMNPNNGNTG------- | 66 | ARGWTFFD- | 67 |
| | | IMGT | GFTFSRYD-- | 68 | ----MNPNNGNT-------- | 69 | ARGWTFFDY | 70 |
| 1P2.2 | 5 | Chothia | GYTFTSY--- | 71 | -----NPNSGN--------- | 72 | --GWTFFDY | 60 |
| | | AbM | GYTFTSYDIN | 73 | ---WMNPNSGNTG------- | 74 | --GWTFFDY | 60 |
| | | Kabat | -----SYDIN | 75 | ---WMNPNSGNTGYAQKFQG | 76 | --GWTFFDY | 60 |
| | | Contact | ----TSYDIN | 77 | WMGWMNPNSGNTG------- | 78 | ARGWTFFD- | 67 |
| | | IMGT | GYTFTSYD-- | 79 | ----MNPNSGNT-------- | 80 | ARGWTFFDY | 81 |
| 1P4 | 1 | Chothia | GFTFSNY--- | 35 | -----LYDGSN--------- | 36 | --DRVWLDV | 37 |
| | | AbM | GFTFSNYGMH | 38 | ---LILYDGSNKY------- | 39 | --DRVWLDV | 37 |
| | | Kabat | -----NYGMH | 40 | ---LILYDGSNKYYADSVKG | 41 | --DRVWLDV | 37 |
| | | Contact | ----SNYGMH | 42 | WVSLILYDGSNKY------- | 43 | ARDRVWLD- | 44 |
| | | IMGT | GFTFSNYG-- | 45 | ----ILYDGSNK-------- | 46 | ARDRVWLDV | 47 |
| 1P4.2 | 2 | Chothia | GFTESSY--- | 48 | -----WYDGSN--------- | 49 | --DRVWLDV | 37 |
| | | AbM | GFTFSSYGMH | 50 | ---VIWYDGSNKY------- | 51 | --DRVWLDV | 37 |
| | | Kabat | -----SYGMH | 52 | ---VIWYDGSNKYYADSVKG | 53 | --DRVWLDV | 37 |
| | | Contact | ----SSYGMH | 54 | WVAVIWYDGSNKY------- | 55 | ARDRVWLD- | 44 |
| | | IMGT | GFTFSSYG-- | 56 | ----IWYDGSNK-------- | 57 | ARDRVWLDV | 47 |
| 1P9 | 6 | Chothia | GFTESTY--- | 82 | -----KQDGGE--------- | 83 | --LRLYSDYEDY | 84 |
| | | AbM | GFTFSTYWMS | 85 | ---NIKQDGGERY------- | 86 | --LRLYSDYEDY | 84 |
| | | Kabat | -----TYWMS | 87 | ---NIKQDGGERYYVDSVKG | 88 | --LRLYSDYEDY | 84 |
| | | Contact | ----STYWMS | 89 | WVANIKQDGGERY------- | 90 | ARLRLYSDYED- | 91 |
| | | IMGT | GFTFSTYW-- | 92 | ----IKQDGGER-------- | 93 | ARLRLYSDYEDY | 94 |
| 1P9.2 | 7 | Chothia | GFTFSSY--- | 48 | -----KQDGSE--------- | 95 | --LRLYSDYEDY | 84 |
| | | AbM | GFTFSSYWMS | 96 | ---NIKQDGSEKY------- | 97 | --LRLYSDYEDY | 84 |
| | | Kabat | -----SYWMS | 98 | ---NIKQDGSEKYYVDSVKG | 99 | --LRLYSDYEDY | 84 |
| | | Contact | ----SSYWMS | 100 | WVANIKODGSEKY------- | 101 | ARLRLYSDYED- | 91 |
| | | IMGT | GFTFSSYW-- | 102 | ----IKQDGSEK-------- | 103 | ARLRLYSDYEDY | 94 |
| 2M1 | 8 | Chothia | GFTFSSY--- | 48 | -----TSSTSY--------- | 104 | --DWSIAARGAWFDP | 105 |
| | | AbM | GFTFSSYTMN | 106 | ---SITSSTSYIY------- | 107 | --DWSIAARGAWFDP | 105 |
| | | Kabat | -----SYTMN | 108 | ---SITSSTSYIYYADSVKG | 109 | --DWSIAARGAWFDP | 105 |
| | | Contact | ---SSYTMN | 110 | WVSSITSSTSYIY------- | 111 | VRDWSIAARGAWFD- | 112 |
| | | IMGT | GFTFSSYT-- | 113 | ----ITSSTSYI-------- | 114 | VRDWSIAARGAWFDP | 115 |
| 2M2 | 9 | Chothia | GYTFTNY--- | 116 | -----SAYNGH--------- | 117 | --GTSGDY | 118 |
| | | AbM | GYTFTNYGIS | 119 | ---WISAYNGHTN------- | 120 | --GTSGDY | 118 |
| | | Kabat | -----NYGIS | 121 | ---WISAYNGHTNYAQKVQG | 122 | --GTSGDY | 118 |
| | | Contact | ----TNYGIS | 123 | WMGWISAYNGHTN------- | 124 | ARGTSGD- | 125 |
| | | IMGT | GYTFTNYG-- | 126 | ----ISAYNGHT-------- | 127 | ARGTSGDY | 128 |
| 2M3 | 10 | Chothia | GENFRSY--- | 129 | ------GPGGD--------- | 130 | --EQFEGHYFDY | 131 |
| | | AbM | GENFRSYDMH | 132 | ----GIGPGGDTY------- | 133 | --EQFEGHYFDY | 131 |
| | | Kabat | -----SYDMH | 134 | ----GIGPGGDTYYPGSVKG | 135 | --EQFEGHYFDY | 131 |
| | | Contact | ----RSYDMH | 136 | WVSGIGPGGDTY-------- | 137 | AREQFEGHYFD- | 138 |
| | | IMGT | GFNFRSYD-- | 139 | ----IGPGGDT--------- | 140 | AREQFEGHYFDY | 141 |
| 2M5 | 11 | Chothia | GFTLSSY--- | 142 | -----SSSSSY--------- | 143 | --EGGAARPSWEDP | 144 |
| | | AbM | GFTLSSYSMN | 145 | ---SISSSSSYIY------- | 146 | --EGGAARPSWFDP | 144 |
| | | Kabat | -----SYSMN | 147 | ---SISSSSSYIYYADSVKG | 148 | --EGGAARPSWFDP | 144 |
| | | Contact | ----SSYSMN | 149 | WVSSISSSSSYIY------- | 150 | AREGGAARPSWFD- | 151 |
| | | IMGT | GFTLSSYS-- | 152 | ----ISSSSSYI-------- | 153 | AREGGAARPSWFDP | 154 |
| 3B1 | 12 | Chothia | GYDFTNY--- | 155 | -----NTYTGE--------- | 156 | --RAYYGHYLAY | 157 |
| | | AbM | GYDFTNYGMN | 158 | ---YINTYTGEPA------- | 159 | --RAYYGHYLAY | 157 |
| | | Kabat | -----NYGMN | 160 | ---YINTYTGEPAYADDFKG | 161 | --RAYYGHYLAY | 157 |
| | | Contact | ----TNYGMN | 162 | WMGYINTYTGEPA------- | 163 | ATRAYYGHYLA- | 164 |
| | | IMGT | GYDFTNYG-- | 165 | ----INTYTGEP-------- | 166 | ATRAYYGHYLAY | 167 |
| 3B2 | 13 | Chothia | GDSITSG--- | 168 | -----SYTGS--------- | 169 | --KGGWLLPFAY | 170 |
| | | AbM | GDSITSGYWN | 171 | ---YISYTGSTY------- | 172 | --KGGWLLPFAY | 170 |
| | | Kabat | -----SGYWN | 173 | ---YISYTGSTYYNPSLKS | 174 | --KGGWLLPFAY | 170 |
| | | Contact | ----TSGYWN | 175 | YMGYISYTGSTY------- | 176 | ARKGGWLLPFA- | 177 |
| | | IMGT | GDSITSGY-- | 178 | ----ISYTGST-------- | 179 | ARKGGWLLPFAY | 180 |
| 3B3 | 14 | Chothia | GFTFSNY--- | 35 | -----TVKSDNYG--------- | 181 | --GRYFDY | 182 |
| | | AbM | GFTFSNYRMH | 183 | ---VITVKSDNYGAN------- | 184 | --GRYFDY | 182 |
| | | Kabat | -----NYRMH | 185 | ---VITVKSDNYGANYAESVKG | 186 | --GRYFDY | 182 |
| | | Contact | ----SNYRMH | 187 | WIAVITVKSDNYGAN------- | 188 | SLGRYFD- | 189 |
| | | IMGT | GFTFSNYR-- | 190 | ----ITVKSDNYGA-------- | 191 | SLGRYFDY | 192 |
| 3B4 | 15 | Chothia | GDSITSG--- | 168 | -----SYTGS--------- | 169 | --GLLWLAWFAY | 193 |
| | | AbM | GDSITSGYWN | 171 | ---YISYTGSTY------- | 172 | --GLLWLAWFAY | 193 |
| | | Kabat | -----SGYWN | 173 | ---YISYTGSTYYNPSLKS | 174 | --GLLWLAWFAY | 193 |
| | | Contact | ----TSGYWN | 175 | SMGYISYTGSTY------- | 194 | ASGLLWLAWFA- | 195 |
| | | IMGT | GDSITSGY-- | 178 | ----ISYTGST-------- | 179 | ASGLLWLAWFAY | 196 |
| 3B5 | 12 | Chothia | GYDFTNY--- | 155 | -----NTYTGE--------- | 156 | --RAYYGHYLAY | 157 |
| | | AbM | GYDFTNYGMN | 158 | ---YINTYTGEPA------- | 159 | --RAYYGHYLAY | 157 |
| | | Kabat | -----NYGMN | 160 | ---YINTYTGEPAYADDFKG | 161 | --RAYYGHYLAY | 157 |
| | | Contact | ----TNYGMN | 162 | WMGYINTYTGEPA------- | 163 | ATRAYYGHYLA- | 164 |
| | | IMGT | GYDFTNYG-- | 165 | ----INTYTGEP-------- | 166 | ATRAYYGHYLAY | 167 |
| 3B6 | 16 | Chothia | GFTFSDF--- | 197 | -----RNKVNDYT--------- | 198 | --NVPWDGWYFDV | 199 |
| | | AbM | GFTFSDFYVE | 200 | ---ASRNKVNDYTTE------- | 201 | --NVPWDGWYFDV | 199 |
| | | Kabat | -----DFYVE | 202 | ---ASRNKVNDYTTEYSESVKG | 203 | --NVPWDGWYFDV | 199 |
| | | Contact | ----SDFYVE | 204 | WIAASRNKVNDYTTE------- | 205 | ARNVPWDGWYFD- | 206 |
| | | IMGT | GFTFSDFY-- | 207 | ----SRNKVNDYTT-------- | 208 | ARNVPWDGWYFDV | 209 |

TABLE E1b
Variable light chain (VL) and CDR sequences

| | VL | | CDR-L1 | | CDR-L2 | | CDR-L3 | |
|---|---|---|---|---|---|---|---|---|
| | SEQ | Numbering | | SEQ | | SEQ | | SEQ |
| Clone | ID NO | Scheme | AA sequence | ID NO | AA sequence | ID NO | AA sequence | ID NO |
| 1P1 | 17 | Chothia | RASQGIRNYLA-- | 210 | ----AASTLQS | 211 | QKYNSALWT | 212 |
| | | AbM | RASQGIRNYLA-- | 210 | ----AASTLQS | 211 | QKYNSALWT | 212 |
| | | Kabat | RASQGIRNYLA-- | 210 | ----AASTLQS | 211 | QKYNSALWT | 212 |
| | | Contact | ------RNYLAWY | 213 | LLIYAASTLQ- | 214 | QKYNSALW- | 215 |
| | | IMGT | ---QGIRNY---- | 216 | ----AA----- | 217 | QKYNSALWT | 212 |
| 1P1.2 | 18 | Chothia | RASQGISNYLA-- | 218 | ----AASTLQS | 211 | QKYNSALWT | 212 |
| | | AbM | RASQGISNYLA-- | 218 | ----AASTLQS | 211 | QKYNSALWT | 212 |
| | | Kabat | RASQGISNYLA-- | 218 | ----AASTLQS | 211 | QKYNSALWT | 212 |
| | | Contact | ------SNYLAWY | 219 | LLIYAASTLQ- | 214 | QKYNSALW- | 215 |
| | | IMGT | ---QGISNY---- | 220 | ----AA----- | 217 | QKYNSALWT | 212 |
| 1P2 | 19 | Chothia | RASQSVSSTYLA-- | 221 | ----GASSRAT | 222 | QQYGSSPT | 223 |
| | | AbM | RASQSVSSTYLA-- | 221 | ----GASSRAT | 222 | QQYGSSPT | 223 |
| | | Kabat | RASQSVSSTYLA-- | 221 | ----GASSRAT | 222 | QQYGSSPT | 223 |
| | | Contact | ------SSTYLAWY | 224 | LLIFGASSRA- | 225 | QQYGSSP- | 226 |
| | | IMGT | ---QSVSSTY---- | 227 | ----GA----- | 228 | QQYGSSPT | 223 |
| 1P2_ | 19 | Chothia | RASQSVSSTYLA-- | 221 | ----GASSRAT | 222 | QQYGSSPT | 223 |
| N73D | | AbM | RASQSVSSTYLA-- | 221 | ----GASSRAT | 222 | QQYGSSPT | 223 |
| | | Kabat | RASQSVSSTYLA-- | 221 | ----GASSRAT | 222 | QQYGSSPT | 223 |
| | | Contact | ------SSTYLAWY | 224 | LLIFGASSRA- | 225 | QQYGSSP- | 226 |
| | | IMGT | ---QSVSSTY---- | 227 | ----GA----- | 228 | QQYGSSPT | 223 |
| 1P2.2 | 20 | Chothia | RASQSVSSSYLA-- | 229 | ----GASSRAT | 222 | QQYGSSPT | 223 |
| | | AbM | RASQSVSSSYLA-- | 229 | ----GASSRAT | 222 | QQYGSSPT | 223 |
| | | Kabat | RASQSVSSSYLA-- | 229 | ----GASSRAT | 222 | QQYGSSPT | 223 |
| | | Contact | ------SSSYLAWY | 230 | LLIYGASSRA- | 231 | QQYGSSP- | 226 |
| | | IMGT | ---QSVSSSY---- | 232 | ----GA----- | 228 | QQYGSSPT | 223 |
| 1P4 | 21 | Chothia | KSSQSVLYRSSNKNYLA-- | 233 | ----WASTRES | 234 | HQYYTTPLT | 235 |
| | | AbM | KSSQSVLYRSSNKNYLA-- | 233 | ----WASTRES | 234 | HQYYTTPLT | 235 |
| | | Kabat | KSSQSVLYRSSNKNYLA-- | 233 | ----WASTRES | 234 | HQYYTTPLT | 235 |
| | | Contact | ------LYRSSNKNYLAWY | 236 | LLIYWASTRE- | 237 | HQYYTTPL- | 238 |
| | | IMGT | ---QSVLYRSSNKNY---- | 239 | ----WA----- | 240 | HQYYTTPLT | 235 |
| 1P4.2 | 22 | Chothia | KSSQSVLYSSNNKNYLA-- | 241 | ----WASTRES | 234 | QQYYSTPLT | 242 |
| | | AbM | KSSQSVLYSSNNKNYLA-- | 241 | ----WASTRES | 234 | QQYYSTPLT | 242 |
| | | Kabat | KSSQSVLYSSNNKNYLA-- | 241 | ----WASTRES | 234 | QQYYSTPLT | 242 |
| | | Contact | ------LYSSNNKNYLAWY | 243 | LLIYWASTRE- | 237 | QQYYSTPL- | 244 |
| | | IMGT | ---QSVLYSSNNKNY---- | 245 | ----WA----- | 240 | QQYYSTPLT | 242 |
| 1P9 | 23 | Chothia | RASQGISSWLA-- | 246 | ----SVSSLQS | 247 | QQANSLPPT | 248 |
| | | AbM | RASQGISSWLA-- | 246 | ----SVSSLQS | 247 | QQANSLPPT | 248 |
| | | Kabat | RASQGISSWLA-- | 246 | ----SVSSLQS | 247 | QQANSLPPT | 248 |
| | | Contact | ------SSWLAWY | 249 | LLIYSVSSLQ- | 250 | QQANSLPP- | 251 |
| | | IMGT | ---QGISSW---- | 252 | ----SV----- | 253 | QQANSLPPT | 248 |
| 1P9.2 | 24 | Chothia | RASQGISSWLA-- | 246 | ----AASSLQS | 254 | QQANSFPPT | 255 |
| | | AbM | RASQGISSWLA-- | 246 | ----AASSLQS | 254 | QQANSFPPT | 255 |
| | | Kabat | RASQGISSWLA-- | 246 | ----AASSLQS | 254 | QQANSFPPT | 255 |
| | | Contact | ------SSWLAWY | 249 | LLIYAASSLQ- | 256 | QQANSFPP- | 257 |
| | | IMGT | ---QGISSW---- | 252 | ----AA----- | 217 | QQANSFPPT | 255 |
| 2M1 | 25 | Chothia | RASQGIGSWLA-- | 258 | ----AASSLQG | 259 | QQANSFPLT | 260 |
| | | AbM | RASQGIGSWLA-- | 258 | ----AASSLQG | 259 | QQANSFPLT | 260 |
| | | Kabat | RASQGIGSWLA-- | 258 | ----AASSLQG | 259 | QQANSFPLT | 260 |
| | | Contact | ------GSWLAWY | 261 | FLIYAASSLQ- | 262 | QQANSFPL- | 263 |
| | | IMGT | ---QGIGSW---- | 264 | ----AA----- | 217 | QQANSFPLT | 260 |
| 2M2 | 26 | Chothia | RASQSVSSYLA-- | 265 | ----DASNRAT | 266 | QQRSNWPPLT | 267 |
| | | AbM | RASQSVSSYLA-- | 265 | ----DASNRAT | 266 | QQRSNWPPLT | 267 |
| | | Kabat | RASQSVSSYLA-- | 265 | ----DASNRAT | 266 | QQRSNWPPLT | 267 |
| | | Contact | ------SSYLAWY | 268 | LLIYDASNRA- | 269 | QQRSNWPPL- | 270 |
| | | IMGT | ---QSVSSY---- | 271 | ----DA----- | 272 | QQRSNWPPLT | 267 |
| 2M3 | 27 | Chothia | RASQGISSWLA-- | 246 | ----TASSLQS | 273 | QQANSFPIT | 274 |
| | | AbM | RASQGISSWLA-- | 246 | ----TASSLQS | 273 | QQANSFPIT | 274 |
| | | Kabat | RASQGISSWLA-- | 246 | ----TASSLQS | 273 | QQANSFPIT | 274 |
| | | Contact | ------SSWLAWY | 249 | LLIYTASSLQ- | 275 | QQANSFPI- | 276 |
| | | IMGT | ---QGISSW---- | 252 | ----TA----- | 277 | QQANSFPIT | 274 |
| 2M5 | 28 | Chothia | RSSTGAVTTSNYAN-- | 278 | ----GTNNRAP | 279 | ALWYRNHWV | 280 |
| | | AbM | RSSTGAVTTSNYAN-- | 278 | ----GTNNRAP | 279 | ALWYRNHWV | 280 |
| | | Kabat | RSSTGAVTTSNYAN-- | 278 | ----GTNNRAP | 279 | ALWYRNHWV | 280 |
| | | Contact | ------VTTSNYANWV | 281 | GLIGGTNNRA- | 282 | ALWYRNHW- | 283 |
| | | IMGT | ---TGAVTTSNY---- | 284 | ----GT----- | 285 | ALWYRNHWV | 280 |
| 3B1 | 29 | Chothia | RSSQRIVHSNGNTYLE-- | 286 | ----KVSNRFS | 287 | FQGSHVPYT | 288 |
| | | AbM | RSSQRIVHSNGNTYLE-- | 286 | ----KVSNRFS | 287 | FQGSHVPYT | 288 |
| | | Kabat | RSSQRIVHSNGNTYLE-- | 286 | ----KVSNRFS | 287 | FQGSHVPYT | 288 |
| | | Contact | ------VHSNGNTYLEWY | 289 | LLIYKVSNRF- | 290 | FQGSHVPY- | 291 |
| | | IMGT | ---QRIVHSNGNTY---- | 292 | ----KV----- | 293 | FQGSHVPYT | 288 |
| 3B2 | 30 | Chothia | KSSQSLLYSTNQKNSLA-- | 294 | ----WASTRES | 234 | QQYYSYPFT | 295 |
| | | AbM | KSSQSLLYSTNQKNSLA-- | 294 | ----WASTRES | 234 | QQYYSYPFT | 295 |
| | | Kabat | KSSQSLLYSTNQKNSLA-- | 294 | ----WASTRES | 234 | QQYYSYPFT | 295 |
| | | Contact | ------LYSTNQKNSLAWY | 296 | LLIYWASTRE- | 237 | QQYYSYPF- | 297 |
| | | IMGT | ---QSLLYSTNQKNS---- | 298 | ----WA----- | 240 | QQYYSYPFT | 295 |
| 3B3 | 31 | Chothia | SASQGISNYLN-- | 299 | ----YTSSLHS | 300 | QQYSKLPWT | 301 |
| | | AbM | SASQGISNYLN-- | 299 | ----YTSSLHS | 300 | QQYSKLPWT | 301 |
| | | Kabat | SASQGISNYLN-- | 299 | ----YTSSLHS | 300 | QQYSKLPWT | 301 |
| | | Contact | ------SNYLNWY | 302 | LLIYYTSSLH- | 303 | QQYSKLPW- | 304 |
| | | IMGT | ---QGISNY---- | 220 | ----YT----- | 305 | QQYSKLPWT | 301 |
| 3B4 | 32 | Chothia | KSSQSLLYSSNQKNSLA-- | 306 | ----WASTRES | 234 | QQYYGYPFT | 307 |
| | | AbM | KSSQSLLYSSNQKNSLA-- | 306 | ----WASTRES | 234 | QQYYGYPFT | 307 |
| | | Kabat | KSSQSLLYSSNQKNSLA-- | 306 | ----WASTRES | 234 | QQYYGYPFT | 307 |
| | | Contact | ------LYSSNQKNSLAWY | 308 | LLIYWASTRE- | 237 | QQYYGYPF- | 309 |
| | | IMGT | ---QSLLYSSNQKNS---- | 310 | ----WA----- | 240 | QQYYGYPFT | 307 |
| 3B5 | 33 | Chothia | SASSSVSYMH-- | 311 | ----STSNLAS | 312 | QQRSSYPFT | 313 |
| | | AbM | SASSSVSYMH-- | 311 | ----STSNLAS | 312 | QQRSSYPFT | 313 |
| | | Kabat | SASSSVSYMH-- | 311 | ----STSNLAS | 312 | QQRSSYPFT | 313 |
| | | Contact | ------SYMHWF | 314 | LWIYSTSNLA- | 315 | QQRSSYPF- | 316 |
| | | IMGT | ---SSVSY---- | 317 | ----ST----- | 318 | QQRSSYPFT | 313 |
| 3B6 | 34 | Chothia | KASQNVRTAVA-- | 319 | ----LASTRHT | 320 | LQHWNYPRT | 321 |
| | | AbM | KASQNVRTAVA-- | 319 | ----LASTRHT | 320 | LQHWNYPRT | 321 |
| | | Kabat | KASQNVRTAVA-- | 319 | ----LASTRHT | 320 | LQHWNYPRT | 321 |
| | | Contact | ------RTAVAWE | 322 | ALIYLASTRH- | 323 | LQHWNYPR- | 324 |
| | | IMGT | ---QNVRTA---- | 325 | ----LA----- | 326 | LQHWNYPRT | 321 |

### B. Anti-PD-L1 Antibody Production

Nucleotide sequences encoding the anti-PD-L1 antibodies were designed to remove restriction sites and cryptic splice sites and optimized for codon usage. In some cases, restriction sites were introduced for cloning purposes. The generated nucleotide sequences were cloned into a dual gene expression vector pD2535nt-HDP_v2 (ATUM) with and without effector knockout mutations L234F, L235E, P331S. Antibodies were expressed by transfecting CHO K1 GS KO cells (Horizon Discovery; Cat. No. HD-BIOP3) with the resulting expression vectors. The antibodies were then purified by Drip Column or fast performance liquid chromatography (FPLC) as described below.

### 1. Drip Column Purification

For PrismA Drip Column Purification, the harvest cell culture fluid (HCCF) for each construct was then loaded onto three pre-packed 2.5 mL CV PrismA Resin drip columns. The resin was regenerated with 20 mL 0.5 N NaOH, and then equilibrated with 40 mL 1×PBS. The HCCF was loaded onto the column, followed by a column wash with 20 mL 1×PBS. The antibody was eluted with 10 mL 40 mM acetic acid, pH 3.1. Each elution pool was neutralized with 0.32 mL 1 M Tris Base to pH 7 and then concentrated using a 30 kDa Amicon filter (Millipore) to less than 15 mL. The eluates were then dialyzed into 1×PBS overnight. The pools were then combined and sterile filtered with a 0.2 μm membrane.

### 2. FPLC Purification

For FPLC purification, the HCCF for each construct was loaded onto a pre-packed 6.8 mL CV PrismA Resin column pre-equilibrated with 5 column volumes 1×PBS. The column was then washed with 3 column volumes 1×PBS. The antibody was eluted with 3.5 column volumes of 40 mM acetic acid, pH 3.1 into 2 mL fractions. Fractions were then pooled and dialyzed into 1×PBS using 30 mL 20 kDa dialysis cassettes overnight. The dialyzed pool was and then sterile filtered with a 0.2 μm membrane.

### 3. Characterization

The filtered antibodies were then analyzed by size exclusion chromatography-high performance liquid chromatography (SEC-HPLC) at 280 nm to determine the abundance of high molecular weight species (HMW) and percent monomer; non-reduced denaturing capillary electrophoresis (CE-SDS) to determine the purity; reduced CE-SDS to determine the percentage of heavy chain and light chain; and mass spectrometric (Q-TOF) determination of intact mass to verify the identities of the heavy chain and light chain. The determined profiles of exemplary clones are provided in Table E2.

TABLE E2

| | | | Non- | | | |
|---|---|---|---|---|---|---|
| | SEC-HPLC | | Reduced | | | |
| | @ 280 nm | | CE-SDS | Reduced CE-SDS | | |
| Molecule | HMW | Monomer | Purity | LC | HC | LC + HC |
| 1P9 | 1.2% | 98.8% | 88.4% | 32.2% | 65.2% | 97.4% |
| 1P4 | ND | 100.0% | 89.9% | 34.7% | 61.9% | 96.6% |
| 1P9 (L234F/L235E/P331S) | 0.9% | 99.1% | 89.2% | 32.4% | 65.2% | 97.6% |
| 1P4 (L234F/L235E/P331S) | ND | 100.0% | 89.1% | 34.5% | 62.4% | 96.6% |

### Example 2:PD-L1 Binding Activity of Anti-PD-L1 Antibodies

### A. PD-L1 Binding

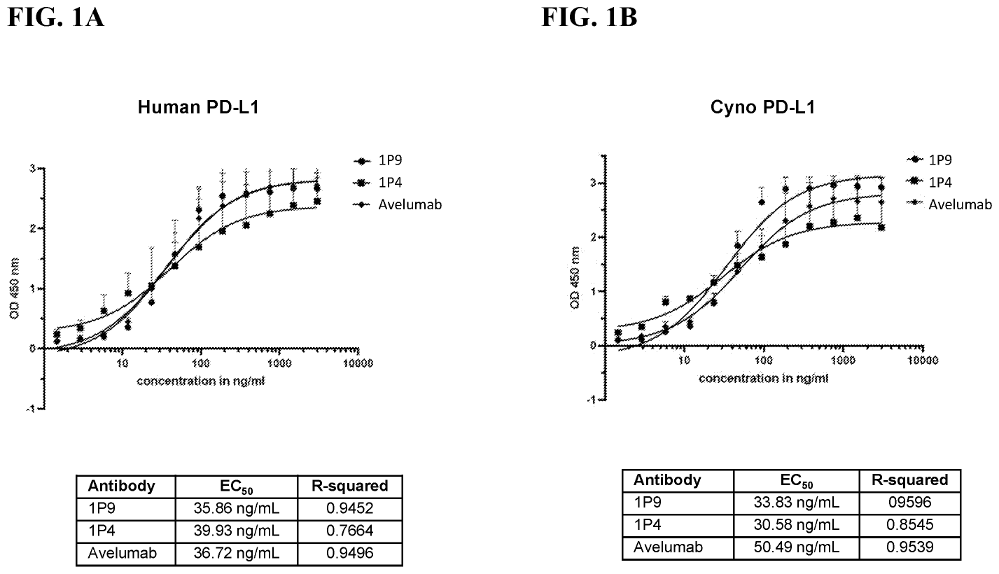

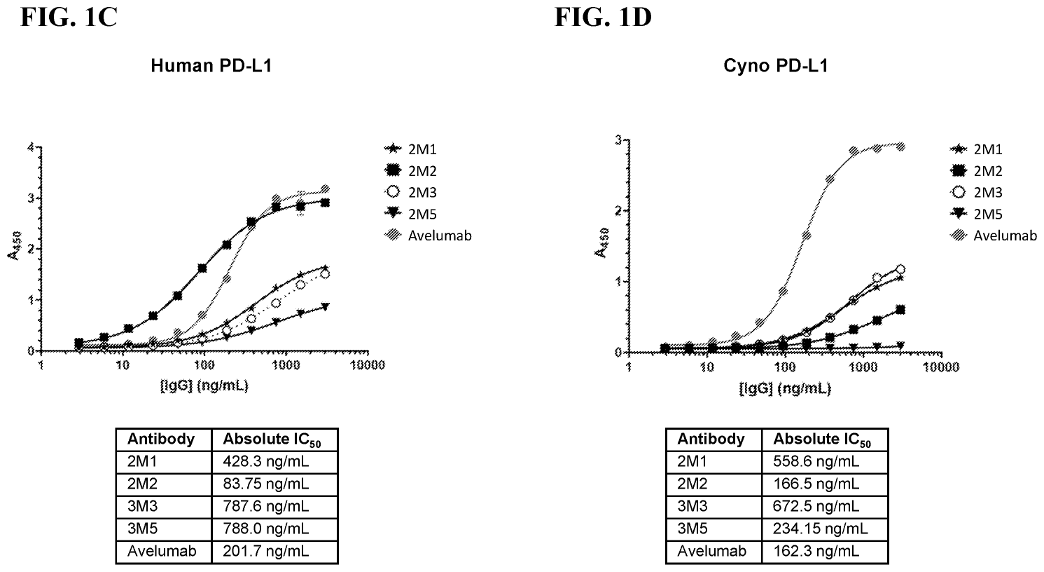

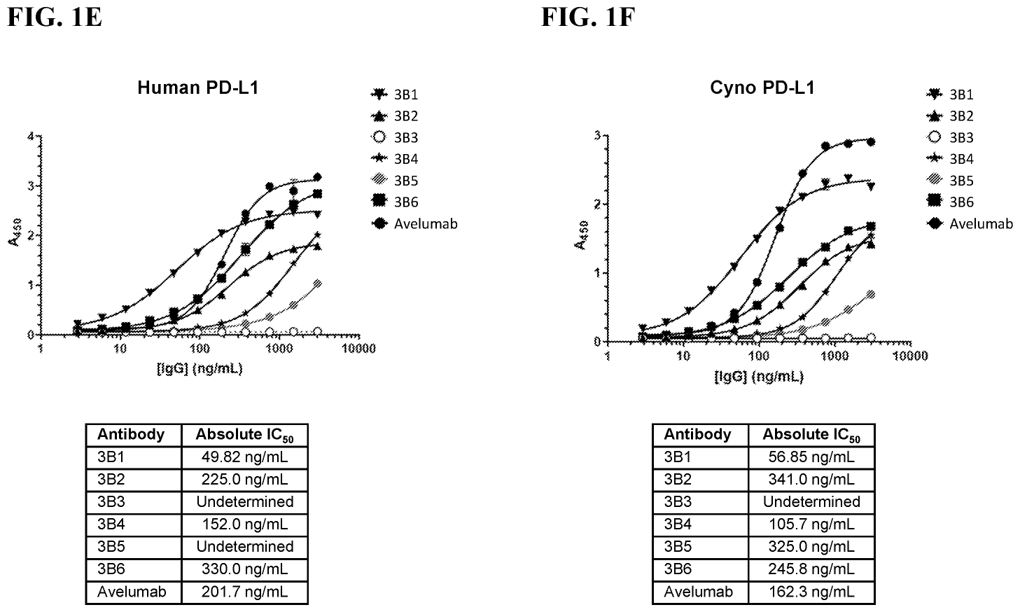

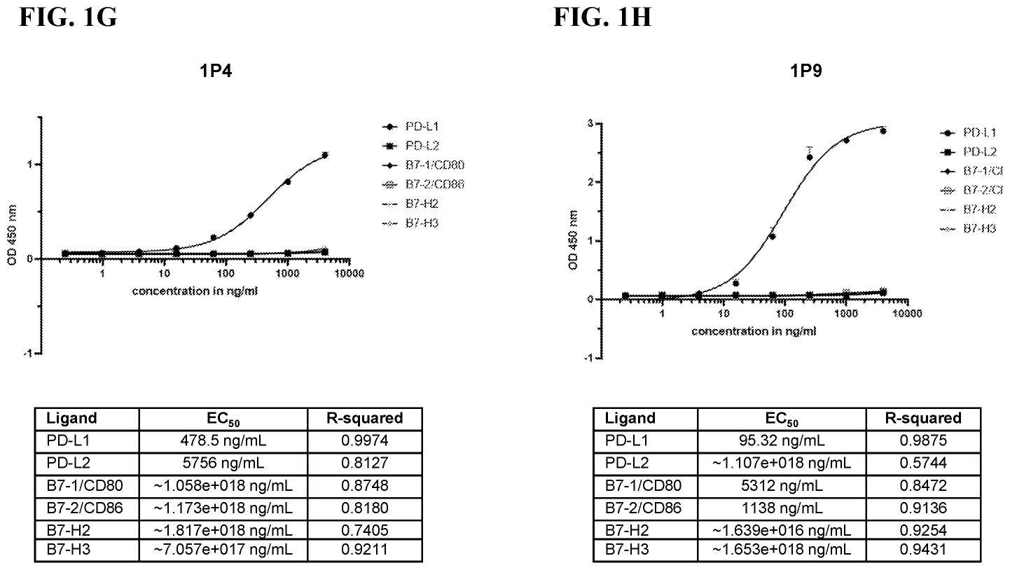

Anti-PD-L1 antibodies, described in Example 1A and manufactured substantially as described in Example 1B, were tested for to huPD-L1 and cyPD-L1 by ELISA substantially as described in Example 1A, using 12 dilutions of antibody ranging from 1 ng/ml to 3 mg/mL. Avelumab binding was also tested as a reference anti-PD-L1 antibody (ref.). The dose response curve and corresponding EC₅₀ and R² (R-squared) values for exemplary anti-PD-L1 antibodies, 1P4 and 1P9, and avelumab are presented in FIG. 1A (huPD-L1) and FIG. 1B (cyPD-L1); the dose response curve and corresponding Absolute IC₅₀ values form exemplary anti-PD-L1 antibodies, 2M1, 2M2, 2M3, 2M5, and avelumab (ref.) are presented in FIG. 1C (huPD-L1) and FIG. 1D (cyPD-L1); and the dose response curve and corresponding Absolute IC₅₀ values form exemplary anti-PD-L1 antibodies, 3B1, 3B2, 3B3, 3B4, 3B5, 3B6, and avelumab (ref.) are presented in FIG. 1E (huPD-L1) and FIG. 1F (cyPD-L1). These results confirmed binding of the purified antibodies to huPD-L1 and cyPD-L1.

### B. PD-L1 Specificity

Exemplary antibodies, 1P4 and 1P9, from the generated anti-PD-L1 antibodies were tested for binding specificity to PD-L1 compared to other B7 ligands using ELISA, substantially as described in Example 1A. Briefly, wells of a 96-well plate (Corning 3690) were incubated with 30 μL human PD-L1 (RD Systems 9049-B7), human PD-L2 (RD Systems 9075-PL), human B7-1/CD80 (RD Systems 9050-B1), human B7-2/CD86 (RD Systems 9090-B2), human B7-H2 (RD Systems 8206-B7), or human B7-H3 (RD Systems 1949-B3), at a concentration of 1 μg/mL in PBS, overnight at 4C, to coat the wells with ligand. After washing and blocking steps, 30 μL of antibody at 8 concentrations ranging from 1 ng/mL to 3 mg/mL were added to the plate and incubated at room temperature for 1 hr. Bound human antibody was detected as described above. The dose response curves and corresponding EC₅₀ and R² (R-squared) values are shown in FIG. 1G (1P4) and FIG. 1H (1P9). Both antibodies bound human PD-L1 but had little to no binding to any of the other B7 ligands, indicating these antibodies are specific for PD-L1.

### C. Binding PD-L1 on the Surface of Engineered Cells

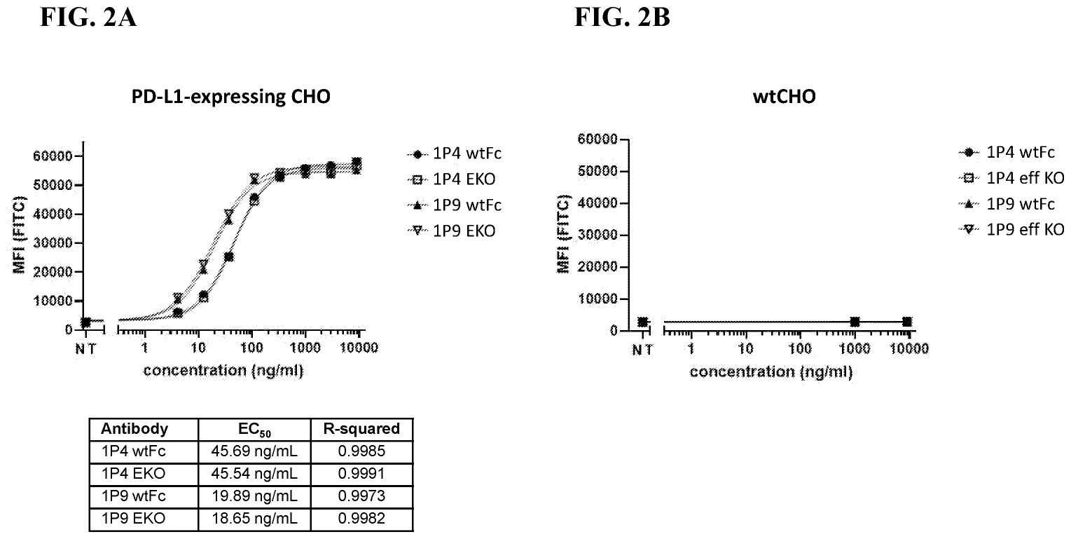

Exemplary anti-PD-L1 antibodies, 1P4 and 1P9 with wild-type Fc and effector knock-out Fc regions, were tested for their abilities to bind huPD-L1 expressed on the surface of engineered CHO cells using flow cytometry, substantially as described in Example 1A2, using 9 concentrations of antibody, ranging from 9 to 0 μg/mL. Binding to wild-type (wt) Chinese hamster ovary (CHO) cells, not expressing PD-L1, was also determined as a negative control, using antibody concentrations of 9, 1, and 0 μg/mL. The binding of the antibodies is plotted as a function of antibody concentration in FIGS. 2A-2B. The exemplary antibodies tested bound the PD-L1-expressing cells (FIG. 2A) but did not bind the wt CHO cells, not expressing PD-L1 (FIG. 2B). The effector knock-out (EKO) mutations did not substantially affect the binding of the antibodies, as indicated by comparable EC₅₀ values for binding between wtFc and EKO-Fc versions of the same antibody.

### D. Binding A431 Cancer Cells

Exemplary anti-PD-L1 antibodies, 1P4 and 1P9, were tested for their abilities to bind unstimulated A431 squamous cell carcinoma cells, which express huPD-L1 (Horita et al., Neoplasia (2017); 19:346-353). Binding of commercially available anti-PD-L1 antibody, avelumab, was also measured for comparison.

Approximately 2.5×10⁵ A431 squamous cell carcinoma cells were plated in a final volume of 50 μL FACS buffer (phosphate buffered saline, 1% fetal bovine serum, 5 mM EDTA) in 96-well plates. 50 μL 2× Zombie Violet (BioLegend) were added to each well, and the plates were incubated 20-30 minutes at room temperature, protected from light. After incubation, 150 μL FACS buffer were added to each well. The plates were centrifuged at 500×g for 5 minutes. The supernatant was removed, and the cell pellet were resuspended in 200 μL FACS buffer. The wash procedure was repeated for a total of 2-3 washes. The washed cells were resuspended in 100 μL of 9 μg/mL, 3 μg/mL, 1 μg/mL, 333 ng/ml, 111 ng/ml, 37 ng/ml, 12 ng/mL, 4.1 ng/mL, or 1 ng/mL, anti PD-L1 antibody: 1P4, 1P9, or avelumab, or buffer only. After the cells were incubated with the primary anti-PD-L1 antibody 1 hour on ice, protected from light, 150 μL FACS buffer were added to each well. The plates were then centrifuged, the supernatant was removed, and the cells were resuspended in 250 μL FACS buffer three times. After the third wash, the cells were resuspended in 100 μL goat anti-human IgG-Alexa Fluor 488 secondary antibody, and incubated for 30 minutes on ice, protected from light. After 3 washes as previously described, the cells were resuspended in 200 μL FACS buffer. The cells were then analyzed using a CytoFLEX flow cytometer.

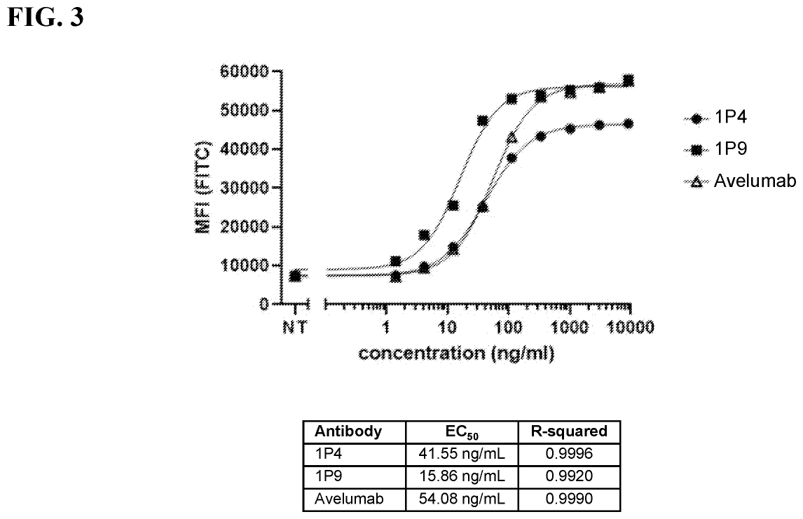

The binding of the antibodies (MFI), plotted as a function of antibody concentration, is shown in FIG. 3. All antibodies bound the PD-L1-expressing A431 cancer cells, with 1P9 and commercially available avelumab having similar maximum binding, but 1P9 having a lower EC₅₀ than avelumab (15.86 vs. 54.08 ng/mL). The 1P9 antibody was 3.5-fold more potent than avelumab. The 1P4 antibody also had a reduced EC₅₀ compared to avelumab (41.55 vs. 54.08 ng/mL) but had a lower maximum binding (MFI) compared to 1P9 and avelumab.

### Example 3: Anti-PD-L1 Binding Kinetics

The binding affinities of the generated antibodies to human PD-L1 were determined by surface plasmon resonance. Exemplary 1P4 and 1P9 antibodies, generated as described above, were diluted 1/300 and captured onto an anti-human (Life Technologies H10500 goat anti-human) coated CM4 sensor chip within a Biacore 4000. PD-L1-His (Acro Cat PDL-H5229) was tested for binding in a 3-fold titration series up to 100 nM. Data were collected in HBS-P (pH 7.4) at 37° C. and 25° C. Responses from triplicate studies were globally fit to a 1:1 interaction model to extract estimates of the binding constants shown in the figures and summarized in Table E3 below.

TABLE E3

| | | | |
|---|---|---|---|
| Parameter | 1P4 | 1P9 | Avelumab |

Kinetic and Affinity Summary at 25° C.

| | | | |
|---|---|---|---|
| Kₐ (M⁻¹s⁻¹) | 2.5 × 10⁵ | 1.9 × 10⁵ | 1.3 × 10⁵ |
| Kₐ (s⁻¹) | 4.5 × 10⁻³ | 5.1 × 10⁻⁵ | 2.3 × 10⁻⁵ |
| K_{D} (M) | 1.8 × 10⁻⁸ | 2.7 × 10⁻¹⁰ | 1.7 × 10⁻¹⁰ |

Kinetic and Affinity Summary at 37° C.

| | | | |
|---|---|---|---|
| Kₐ (M⁻¹s⁻¹) | 3.6 × 10⁵ | 3.3 × 10⁵ | 1.9 × 10⁵ |
| Kₐ (s⁻¹) | 9.7 × 10⁻³ | 3.7 × 10⁻⁴ | 4.5 × 10⁻⁵ |
| K_{D} (M) | 2.7 × 10⁻⁸ | 1.1 × 10⁻⁹ | 2.4 × 10⁻¹⁰ |

### Example 4: ADCC Activity

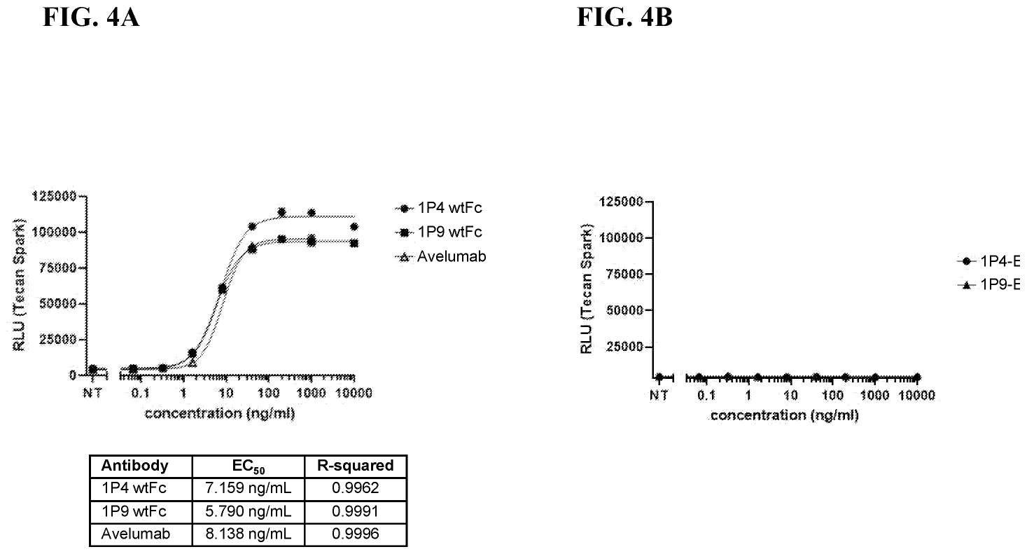

Antibodies were tested for ADCC activity using an mFcγRIII ADCC Reporter Bioassay (Promega). CHO-hPD-L1 cells were plated, 7,500 cells/well in 100 μL complete medium in 96-well plates. After incubating 18-24 hours, the media were removed and 25 μL assay buffer (RMPI-1640 (Promega+4% low IgG Serum) were added. 1P4, 1P4 with effector knockout (EKO) mutations L234F/L235E/P331S (1P4-EKO), 1P9, 1P9 with effector knockout mutations L234F/L235E/P331S (1P9-EKO), and control anti-PD-L1 antibody avelumab were added to wells with cells to achieve final antibody concentrations of 10,000, 1,000, 200, 40, 8, 1.6, 0.32, or 0.064 ng/mL in assay buffer, or assay buffer only, in a final volume of 50 μL. The cells were incubated at 37° C. for 10-15 min. Meanwhile, previously frozen mFcγRIII (effector) Jurkat cells were thawed and added dropwise into assay buffer until a final concentration of 3×10⁶ cells/mL was reached. 25 μL of the effector cell suspension was added to wells of the 96-well plates to achieve a 10:1 effector cell to target cell ratio. The plates were incubated at 37° C. for 6 hours. After equilibrating to room temperature, 75 μL Bio-Glo were added to all wells with cells and 2 cell-free media only control wells were used to subtract out background. After incubating at room temperature for 15-20 minutes, luminescence was read on a Tecan Spark plate reader. Luminescence, corresponding to ADCC activity, was plotted against anti-PD-L1 antibody (FIGS. 4A and 4B). The results demonstrate the anti-PD-L1 antibodies with a functional Fc exhibited ADCC activity (FIG. 4A), while the anti-PD-L1 antibodies with effector knockout mutations did not (FIG. 4B).

### Example 5: Generation of anti-PD-L1 Antibody-IRDye 700 Conjugates

Exemplary anti-PD-L1 antibodies conjugated to IRDye 700DX (IR700) to produce anti-PD-L1-IR700 conjugates. 1P4 and 1P9 antibodies, with a wild-type human Fc or effector knockout (EKO) Fc domain, were buffer exchanged into 1×PBS pH 7.1 then concentrated to 8.2 mg/mL. The antibodies (16 mg) were diluted to 3 mg/mL with 100 mM sodium phosphate pH 8.6 to achieve a target pH 8.0-8.5. IR700 NHS ester (1 mg, IR700; LI-COR Bioscience, Lincoln, NE) was solubilized into DMSO at a concentration of 10 g/L. The solubilized dye was then added to the antibodies for a target dye to antibody ratio of 1 mg IR700 NHS ester to 16 mg of antibody. The reaction occurred for 2 hours at room temperature. The reaction was quenched by the addition of 1 M glycine to a target batch concentration of 20 mM glycine for 1 hour at room temperature. Buffer exchange was performed using Millipore 30 kDa molecular weight cut-off Amicon centrifuge filters by concentration and dilution of up to 3 cycles at ˜3500 RPM.

The mixture was purified using a Sephadex G50 column (PD-10; GE Healthcare, Piscataway, NJ). Protein concentration was determined with Coomassie Plus protein assay kit (Pierce Biotechnology, Rockford, IL) by measuring the absorption at 595 nm with a UV-Vis system (8453 Value System; Agilent Technologies, Palo Alto, CA). The concentration of IR700 was measured by absorption with the UV-Vis system to confirm the number of fluorophore molecules conjugated to each anti-PD-L1 antibody molecule. The number of IR700 molecules per antibody (or dye: antibody ratio; DAR) was determined to be about 2.5-3. The concentration and DAR were comparable for all antibodies regardless of the effector KO-Fc region.

Purity of the anti-PD-L1-IR700 conjugates was confirmed by analytical size-exclusion chromatography HPLC (SEC-HPLC). SEC-HPLC was performed using an Agilent 1100 HPLC system (Santa Clara, CA) equipped with a PDA detector controlled by Chemstation software. SEC was performed on a Shodex KW-803 column (New Yok, NY) eluted for 20 minutes using phosphate buffered saline (PBS) at 1.0 mL/min. The SEC results are set forth in Table E4. Purity was observed to be at least 97% for all conjugates at both 280 nm and 690 nm.

TABLE E4
Dye-Antibody Ratio (DAR), Concentration, and Purity
of Exemplary anti-PD-L1-IR700 Conjugates

| | | Concentration | SEC-HPLC @690 nm | | | SEC-HPLC @280 nm | | |
|---|---|---|---|---|---|---|---|---|
| Antibody | DAR | (mg/mL) | HMW | Monomer | LMW | HMW | Monomer | LMW |
| 1P9 | 2.5 | 3.3 | 2.3% | 97.7% | ND | 1.9% | 98.0% | 0.1% |
| 1P4 | 2.9 | 3.4 | 0.5% | 98.8% | 0.7% | 0.2% | 99.1% | 0.7% |
| 1P9-EKO | 2.5 | 3.3 | 1.1% | 98.0% | ND | 1.7% | 98.1% | 0.1% |
| 1P4-EKO | 2.7 | 3.4 | 0.7% | 98.6% | 0.7% | 0.6% | 98.5% | 0.8% |

### Example 6:PD-L1 Binding Activity of anti-PD-L1 Conjugates

### A. ELISA Assay to Determine PD-L1 Binding

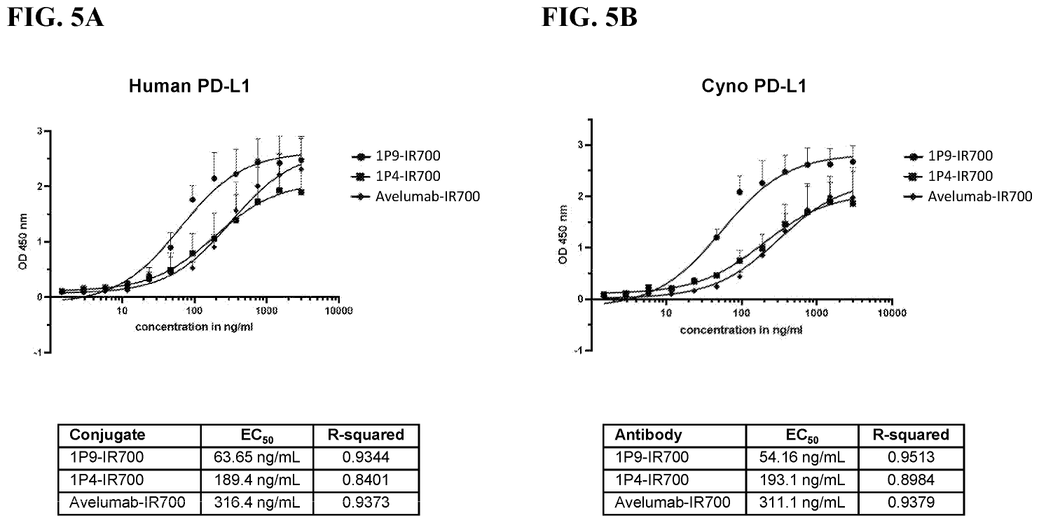

Exemplary anti-PD-L1 conjugates, 1P4-IR700 and 1P9-IR700, were tested for binding to huPD-L1 and cyPD-L1 by ELISA substantially as described in Example 1A, using 12 dilutions of conjugate ranging from 1 ng/mL to 3 mg/mL. Avelumab, conjugated to IR700 (Avelumab-IR700) was used as a reference. The dose response curve and corresponding EC₅₀ and R² values for exemplary anti-PD-L1 conjugates are presented in FIG. 5A (huPD-L1) and FIG. 5B (cyPD-L1). These results confirmed binding of the anti-PD-L1 conjugates to huPD-L1 and cyPD-L1.

### B. Cell Binding PD-L1 on the Surface of Engineered Cells by Flow Cytometry

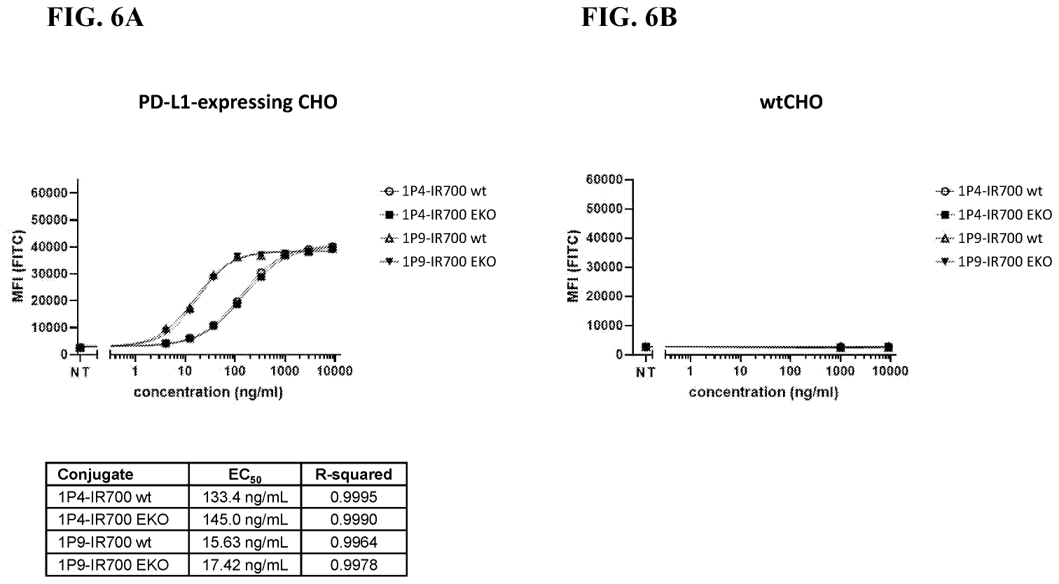

Exemplary anti-PD-L1 conjugates, containing a wild-type Fc domain or a mutant Fc domain lacking effector activity (EKO), were tested for their abilities to bind huPD-L1 expressed on the surface of engineered CHO cells using flow cytometry, substantially as described in Example 1A2, using 9 concentrations of conjugate, ranging from 9 to 0 μg/mL. Binding to wild-type (wt) Chinese hamster ovary (CHO) cells, not expressing PD-L1, was also determined as a negative control, using conjugate concentrations of 9, 1, and 0 μg/mL. The binding of the conjugates is plotted as a function of conjugate concentration in FIGS. 6A-6B. The exemplary conjugates tested bound the PD-L1-expressing cells (FIG. 6A) but did not bind the wt CHO cells, not expressing PD-L1 (FIG. 6B). The effector knock-out (EKO) mutations did not substantially affect the binding of the conjugates.

### C. Binding A431 Cancer Cells by Flow Cytometry

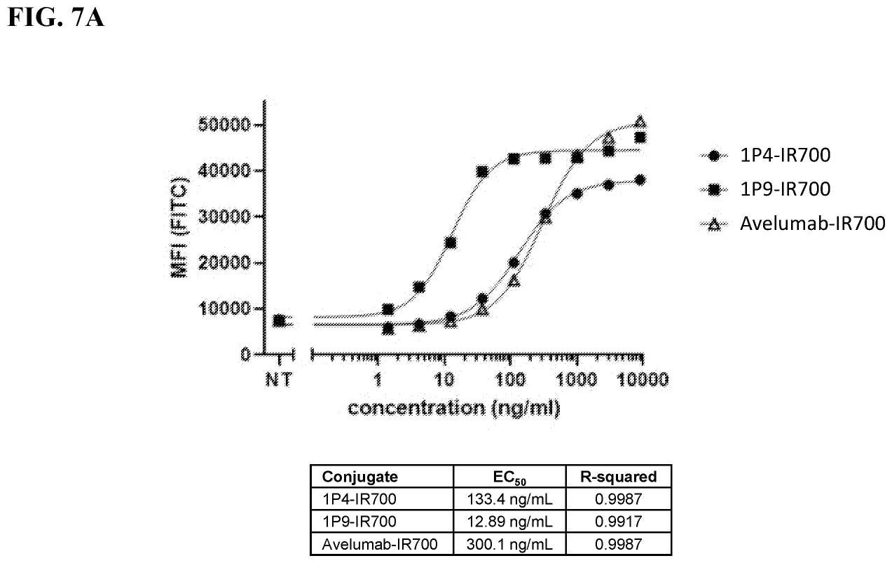

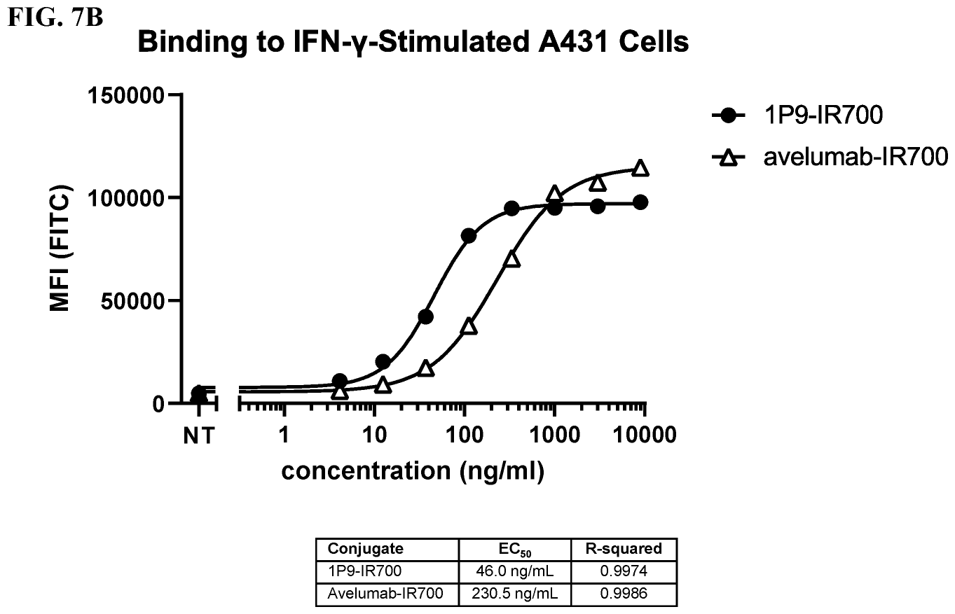

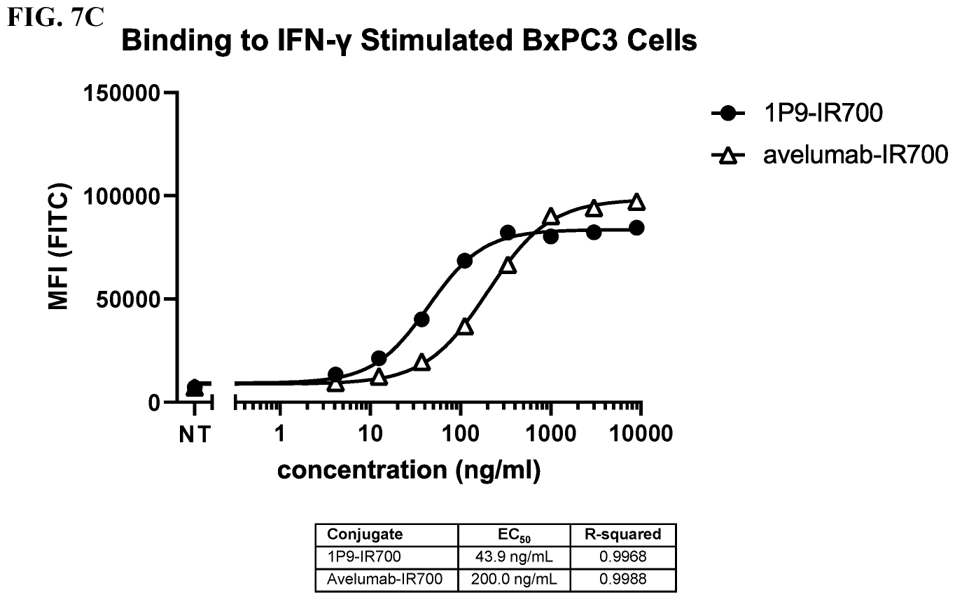

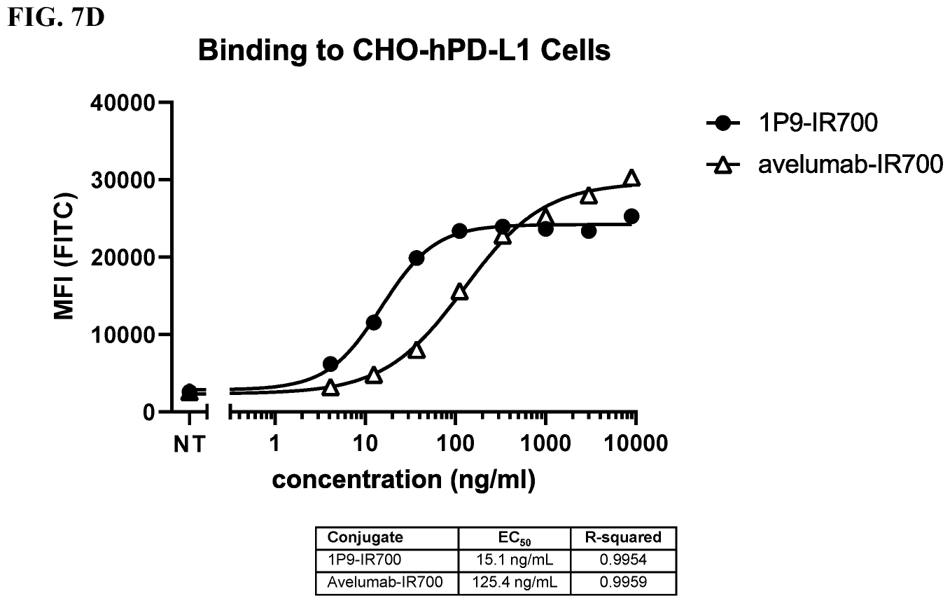

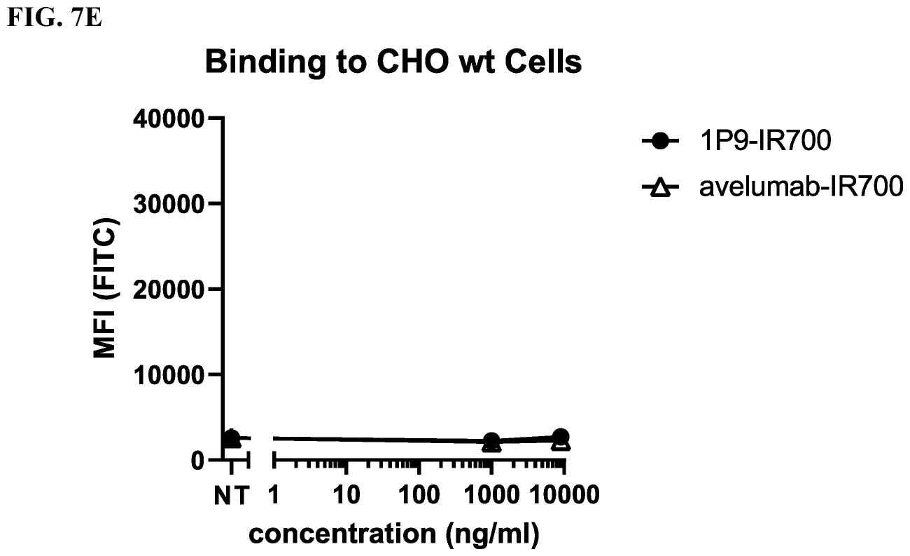

Exemplary anti-PD-L1 antibody-IR700 conjugates, 1P4-IR700 and 1P9-IR700, were tested for their abilities to bind unstimulated A431 squamous cell carcinoma cells, which express huPD-L1 (Horita et al., Neoplasia (2017); 19:346-353). Binding of a conjugate containing commercially available anti-PD-L1 antibody, avelumab, conjugated to IR700 was also measured for comparison. Binding was assessed as described in Example 2D, using 10 concentrations of each conjugate. The binding of the conjugates, plotted as a function of conjugate concentration, is shown in FIG. 7A. All conjugates bound the PD-L1-expressing A431 cancer cells in a dose-dependent manner. The 1P9-IR700 conjugate exhibited much lower EC₅₀ for binding A431 cells than the avelumab-IR700 conjugate (12.89 ng/mL vs. 300.1 ng/mL), while exhibiting similar binding maxima. The 1P9-IR700 conjugate was about 23-fold more potent than the avelumab-IR700 conjugate. The 1P4-IR700 conjugate also had a reduced EC₅₀ compared to avelumab (133.4 ng/mL vs. 300.1 ng/mL) but exhibited lower comparative maximum binding.

### D. Binding of IFN-γ-Stimulated A431, IFN-γ-Stimulated BxPC3, CHO-HPD-L1, and Wild-Type (Wt) CHO Cells

1P9-IR700 and avelumab-IR700 conjugates were tested for their abilities to bind IFN-γ-stimulated A431 cells, IFN-γ-stimulated BxPC3 cells, Chinese hamster ovary (CHO) cells engineered to express human PD-L1 (CHO-HPD-L1), and wild-type (wt) CHO cells by flow cytometry.

A431 and BxPC3 cells were treated with 10 ng/ml human IFN-γ for 16 to 24 hours prior to the assay to increase PD-L1 expression on the cell surface. The assay was performed generally as described in Example 2D.

The binding of the conjugates (MFI), plotted as a function of conjugate concentration, is shown in FIGS. 7B-7E. The 1P9-IR700 conjugate bound the PD-L1-expressing cells stronger than the avelumab-IR700 conjugate (FIGS. 7B-7D). Neither conjugate bound wt CHO cells, which do not express PD-L1, indicating the binding of the conjugates is specific for PD-L1 (FIG. 7E).

### Example 7: Anti-PD-L1 Conjugate Binding Kinetics

The binding affinities of the generated anti-PD-L1 conjugates were determined by surface plasmon resonance, as described in Example 3. The kinetics and affinities of exemplary conjugates, 1P4-IR700 and 1P9-IR700 and avelumab-IR700 are provided in Table E5 below. Conjugation of 1P4 and 1P9 antibodies did not substantially alter the binding affinity (K_{D}) of the antibodies compared to the unconjugated (unconj.) antibody at 25° C. (unconjugated antibody data reproduced in Table E5 from Table E3 in Example 3). In contrast, conjugation to IR700 reduced the binding affinity (K_{D}) of the avelumab antibody by almost 50-fold at 25° C.

TABLE E5
Kinetic and Affinity Summary at 25° C.

| | Unconj. | 1P4- | Unconj. | 1P9- | Unconj. | Avelumab- |
|---|---|---|---|---|---|---|
| Parameter | 1P4 | IR700 | 1P9 | IR700 | Avelumab | IR700 |
| Kₐ (M⁻¹s⁻¹) | 2.5 × 10⁵ | 3.5 × 10⁵ | 1.9 × 10⁵ | 2.1 × 10⁵ | 1.3 × 10⁵ | 3.1 × 10⁴ |
| K_{d} (s⁻¹) | 4.5 × 10⁻³ | 5.6 × 10⁻³ | 5.1 × 10⁻⁵ | 5.4 × 10⁻⁵ | 2.3 × 10⁻⁵ | 2.5 × 10⁻⁵ |
| K_{D} (M) | 1.8 × 10⁻⁸ | 1.6. × 10⁻⁸ | 2.7 × 10⁻¹⁰ | 2.6 × 10⁻¹⁰ | 1.7 × 10⁻¹⁰ | 8.0 × 10⁻⁹ |

Kinetic and Affinity Summary at 37° C.

| | 1P4- | 1P9- |
|---|---|---|
| Parameter | IR700 | IR700 |
| Kₐ (M⁻¹s⁻¹) | 4.7 × 10⁵ | 3.9 × 10⁵ |
| K_{d} (s⁻¹) | 1.1 × 10⁻² | 3.8 × 10⁻⁴ |
| K_{D} (M) | 2.3 × 10⁻⁸ | 0.97 × 10⁻⁹ |

### Example 8: Photoimmunotherapy (PIT) with Anti-PD-L1 Conjugates

### A. Photoimmunotherapy (PIT) Killing of A431 Cancer Cells

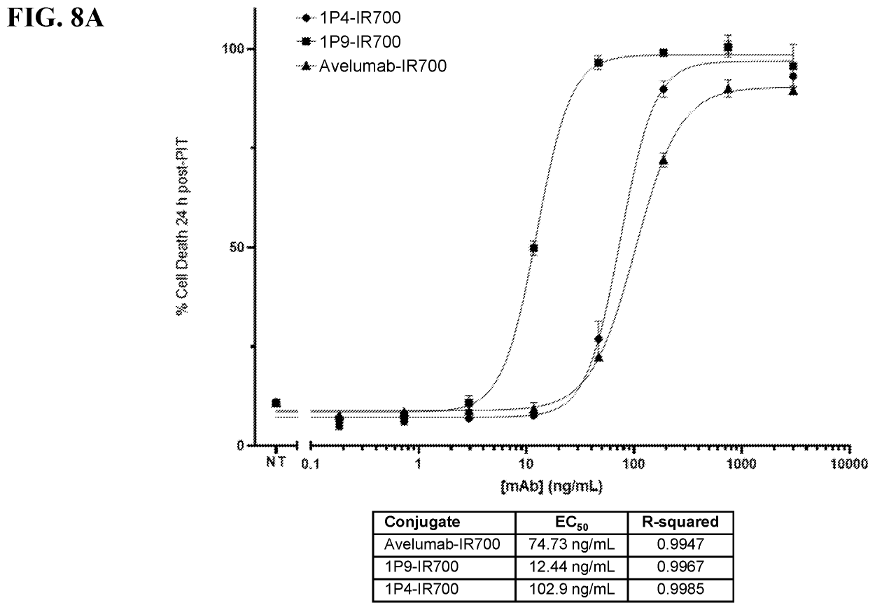

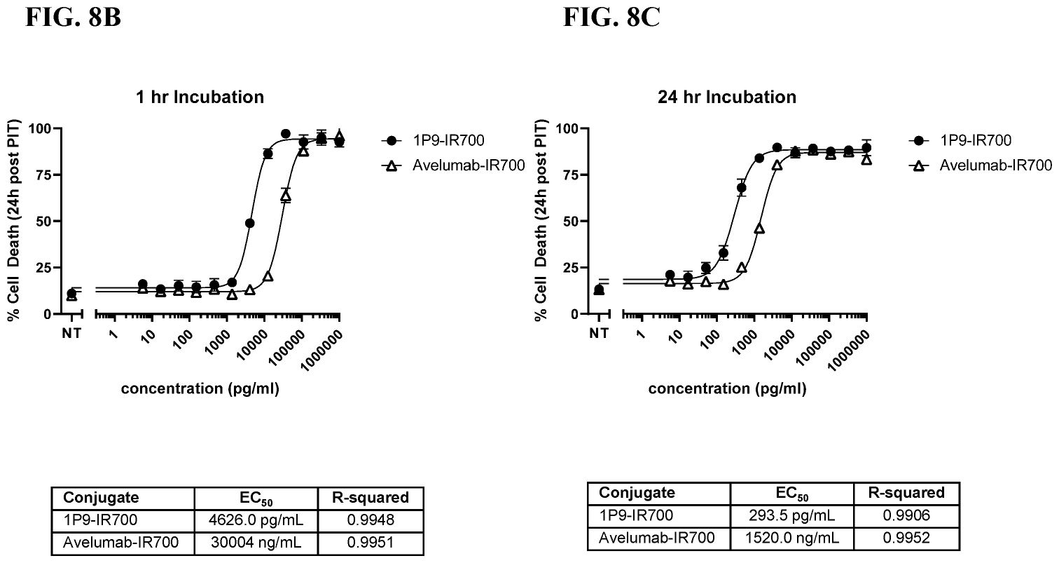

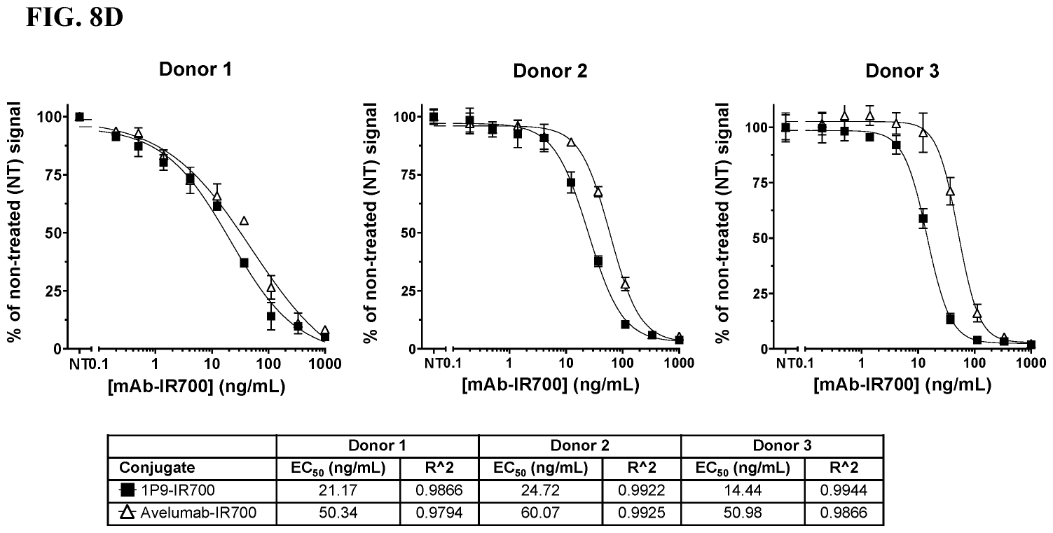

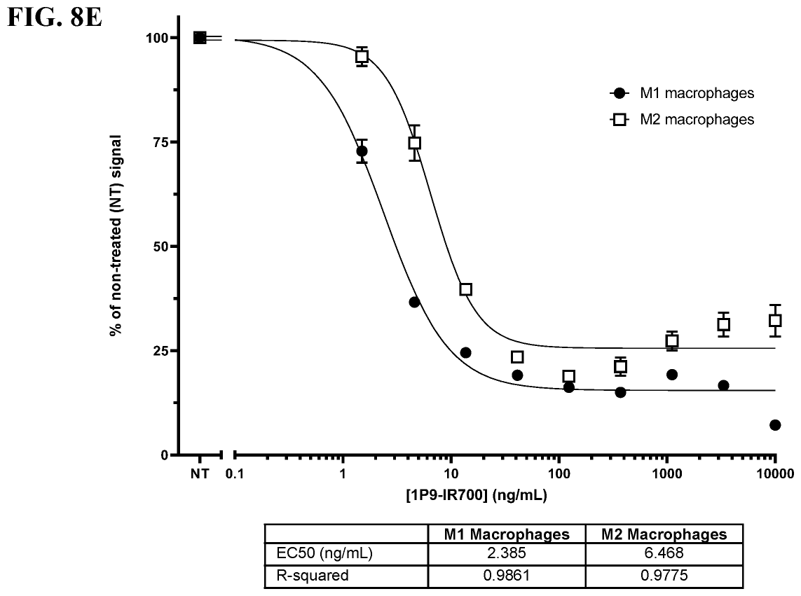

Exemplary anti-PD-L1 conjugates were tested for their abilities to induce killing of tumor cells following illumination. A431 squamous cell carcinoma cells were plated 5,000 cells/well of a 96-well plate in 100 μL complete medium and incubated overnight at 37° C. 1P4-IR700, 1P9-IR700, and avelumab-IR700 conjugates were serially diluted (3-fold dilutions) and added to the cells with final concentrations of 3,000, 750, 187.5, 46.9, 11.7, 2.9, 0.73, 0.18, and 0 ng/mL. The cells were incubated in the presence of the conjugate for 1 hour at 37° C. Following incubation, the cells were illuminated at 128 J/cm² in an ILLUBOX laser system using Omicron software. After illumination, the media was removed from the wells and 100 μL 1×Cell Tox Green (GTG) Cytotoxicity Assay (Promega) reagent in standard culture media were added to each well. After incubating the plates 24 hours at 37° C., fluorescence of each well was measured using a SpectraMax M5 plate reader (Molecular Devices) using an excitation and emission wavelengths of 485 nm and 535 nm, respectively. The cells were then lysed by adding 5 μL 40% Lysis solution (Promega Cat. No. G182A), 60% complete culture medium, and incubating 30 minutes at 37° C. The fluorescence was then measured again to obtain 100% cell death values. The percent cell death was then calculated for each condition for each conjugate and plotted over conjugate concentration (FIG. 8A).

All three anti-PD-L1 conjugates exhibited dose-dependent killing of A431 tumor cells, with 80-100% cell killing at the highest doses examined. Avelumab-IR700 and 1P4-IR700 conjugates exhibited similar dose-response curves with EC₅₀ values of 74.7 ng/ml and 102.9 ng/mL, respectively. 1P9-IR700 exhibited more potent killing of A431 cells than Avelumab-IR700 and 1P4-IR700, with an EC₅₀ value of 12.4 ng/mL.

### B. Photoimmunotherapy (PIT) Killing of IFN-γ-Stimulated A431 Cancer Cells

Exemplary anti-PD-L1 conjugate, 1P9-IR700 and avelumab-IR700 were tested for their ability to induce killing of A431 cells, stimulated by IFN-γ to increase PD-L1 expression. A431 cells, stimulated with 10 ng/mL IFN-γ for 16-24 hours, were plated 10,000 cells/well of a 96-well plate in 100 μL complete medium and incubated overnight at 37° C. The cells were incubated in the presence of the 7 concentrations of each conjugate (3-fold dilutions, with a maximum final concentration of 1,000 ng/mL) for 1 hour or 24 hours at 37° C. Following incubation, the cells were illuminated at 64 J/cm² in an ILLUBOX laser system using Omicron software. After illumination, the media was removed from the wells and cell toxicity was measured using the Cell Tox Green (CTG) Cytotoxicity Assay (Promega) as described previously. The percent cell death was then calculated for each conjugate and plotted over conjugate concentration (FIGS. 8B-8C).

1P9-IR700 conjugates exhibited more potent killing of the target cells than avelumab-IR700 conjugate following incubation for 1 hour (FIG. 8B) and 24 hours (FIG. 8C). The longer incubation with the conjugate resulted in increased potency of target cell killing (approximately 20-fold) for both conjugates.

### C. Photoimmunotherapy (PIT) Killing of Human PBMCs

Exemplary anti-PD-L1 conjugates were tested for their abilities to induce killing of peripheral blood mononuclear cells (PBMCs) following illumination. Cryopreserved human PBMCS from three donors were thawed, washed, and plated 100,000 cells/well of a 96-well plate in 100 μL complete medium, supplemented with 10 μg/mL phytohemagglutinin (PHA). The cells were cultured in the presence of PHA at 37° C. for 48 hours prior to the assay. 1P9-IR700 and avelumab-IR700 conjugates were serially diluted (3-fold dilutions) and added to the cells with final concentrations of 1,000, 333, 111, 37.0, 12.3, 4.1, 1.4, 0.5, 0.2, and 0 ng/mL. The cells were incubated in the presence of the conjugate for 1 hour at 37° C. Following incubation, the cells were illuminated at 64 J/cm² in an ILLUBOX laser system using Omicron software. After illumination, the cells were incubated at 37° C. for 24 hours. The cells were brought to room temperature for 30 minutes and then assayed for viability using CellTiter-Glo 2.0 (Promega). The percent of non-treated (NT) signal was calculated for each conjugate and plotted as a function of conjugate concentration, and EC₅₀ values were determined.

As shown in FIG. 8D, 1P9-IR700 (closed squares) exhibited more potent killing of PBMCs than Avelumab-IR700 (open triangles) with EC₅₀ values that were less than half of those for the Avelumab conjugate.

### D. Photoimmunotherapy (PIT) Killing of Primary Human Macrophages

Exemplary anti-PD-L1 conjugate, 1P9-IR700 was assessed for photoimmunotherapy-induced killing of M1 and M2 primary human macrophages. Primary human monocytes were purified from a fresh human buffy coat layer fractionated from whole blood. The cells were counted and analyzed for monocyte content, resuspended at 100×10⁶ PBMCs/mL in Monocyte Attachment Medium (MAM; PromoCell Catalog #C-28051), and plated at a density of 1×10⁶ cells/cm² for mononuclear cells with a monocyte content of >25% and 1.5 million/cm² for a monocyte content of <25% in a T-75 flask. The medium was aspirated after the cells were allowed to adhere to the tissue culture plate for 1-1.5 hours at 37° C. and 5% CO₂, and the cells were washed three times with MAM.

To initiate monocyte differentiation into macrophages, complete M1- or M2-Macrophage Generation Medium DXF (PromoCell Catalog #C-28055 M1 or Catalog #C-28056 M2) was prepared and added to the cells, e.g., 20 mL per T-75 flask and incubated for 6 days at 37° C. and 5% CO₂. On Day 6 of differentiation, another 50% to 75% by volume of fresh complete M1- or M2-Macrophage Generation Medium DXF was added to the cells. The immature macrophages were then incubated for another 3 days at 37° C. and 5% CO₂.

On Day 7, the differentiated macrophages were activated by adding 50 ng/ml human IFN-γ and 10 ng/mL LPS to M1 macrophages and 20 ng/ml IL-4 to M2 macrophages. After incubating for 2 days, the medium was refreshed, collecting and readding any cells in suspension back to the culture. The cells were then incubated overnight at 37° C. and 5% CO₂.

The day of the assay, the adherent macrophages were harvested by removing the medium, washing the cells twice with phosphate buffered saline lacking Ca²⁺ and Mg²⁺, and incubating 40 minutes in cold 250-300 mL/cm² Macrophage Detachment Solution DXF (PromoCell) at 2-8° C. Any remaining adherent cells were dislodged using a cell scraper and collected in a centrifugation tube. An equal volume of PBS containing 2 mM EDTA and 0.1% HSA was added to the tube. The cells were centrifuged for 15 minutes at 350×g at room temperature and washed with PBS, 2 mM EDTA, 0.1% HSA twice. The M1 and M2 cells were counted and plated 50,000 cells/well in 96-well plates.

1P9-IR700 conjugate was serially diluted (3-fold dilutions) and added to the cells with final concentrations of 1,000, 333, 111, 37.0, 12.3, 4.1, 1.4, 0.5, 0.2, and 0 ng/mL. The cells were incubated in the presence of the conjugate for 1 hour at 37° C. Following incubation, the cells were illuminated at 128 J/cm² in an ILLUBOX laser system using Omicron software. After illumination, the cells were incubated at 37° C. and 5% CO₂ for 24 hours. The cells were brought to room temperature for 30 minutes and then assayed for viability using CellTiter-Glo 2.0 (Promega). The percent of non-treated (NT) signal was calculated for each conjugate and plotted as a function of conjugate concentration, and EC₅₀ values were determined.

As shown in FIG. 8E, 1P9-IR700 binds and kills PD-L1-expressing M1 and M2 macrophages, regardless of subtype.

### Example 9: Photoimmunotherapy (PIT) Light Dose Titration

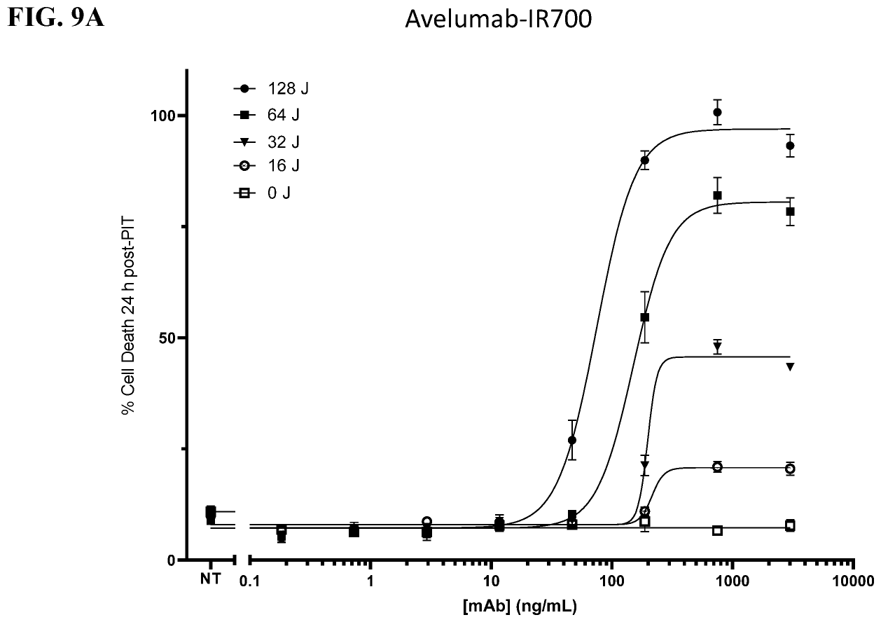

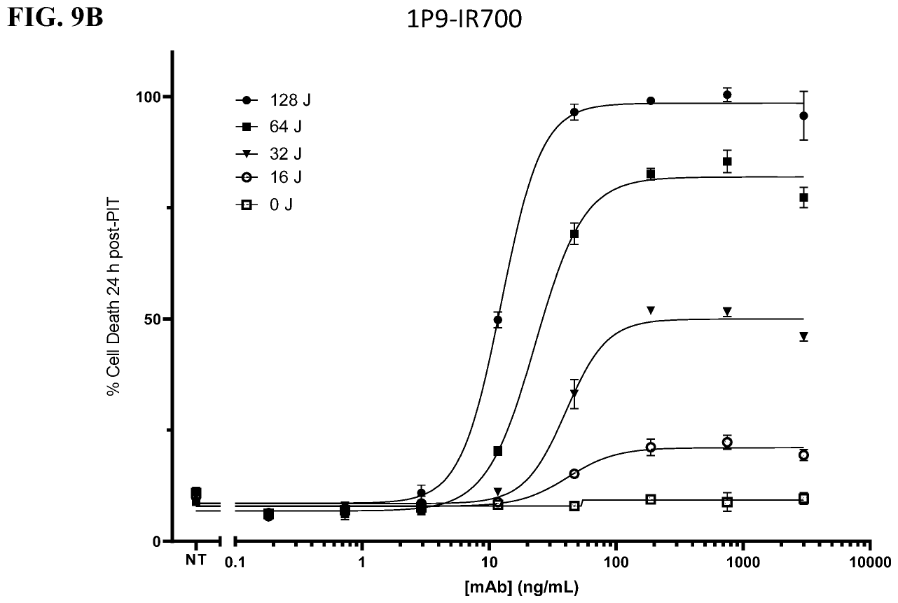

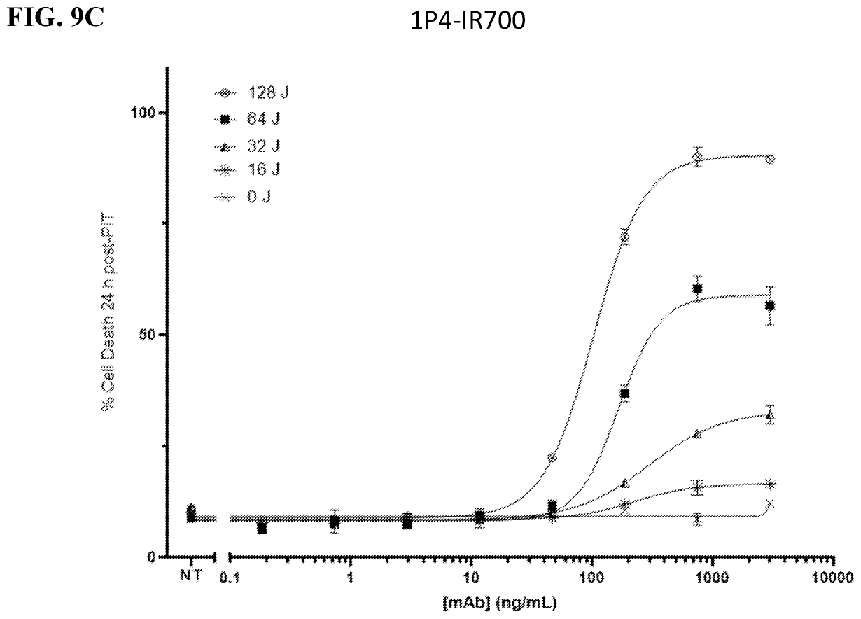

Exemplary anti-PD-L1 conjugates were tested for their abilities to induce killing of tumor cells following illumination at different light fluencies. A431 squamous cell carcinoma cells were plated 5,000 cells/well of a 96-well plate in 100 μL complete medium and incubated overnight at 37° C. 1P4-IR700, 1P9-IR700, and avelumab-IR700 were serially diluted (3-fold dilutions) and added to the cells with final concentrations of 3,000, 750, 187.5, 46.9, 11.7, 2.9, 0.73, 0.18, and 0 ng/mL. The cells were incubated in the presence of antibody for 1 hour at 37° C. Following incubation, the cells were illuminated at 128, 64, 32, 16, or 8 J/cm² in an ILLUBOX laser system using Omicron software. After illumination, the media was removed from the wells and 100 μL 1×Cell Tox Green (GTG) Cytotoxicity Assay (Promega) reagent in standard culture media were added to each well. After incubating the plates 24 hours at 37° C., fluorescence of each well was measured using a SpectraMax M5 plate reader (Molecular Devices) using an excitation and emission wavelengths of 485 nm and 535 nm, respectively. The cells were then lysed by adding 5 μL 40% Lysis solution (Promega Cat. No. G182A), 60% complete culture medium, and incubating 30 minutes at 37° C. The fluorescence was then measured again to obtain 100% cell death values. The percent cell death was then calculated for each condition for each antibody and plotted over conjugate concentration for each light dose (FIGS. 9A-9C). All three anti-PD-L1 conjugates exhibited conjugate and light dose-dependent killing of A431 tumor cells, with 1P9-IR700 exhibiting the highest potency at all light doses tested (FIG. 9B).

### Example 10:PD-1:PD-L1 Blocking Activity of Anti-PD-L1 Antibodies and Conjugates

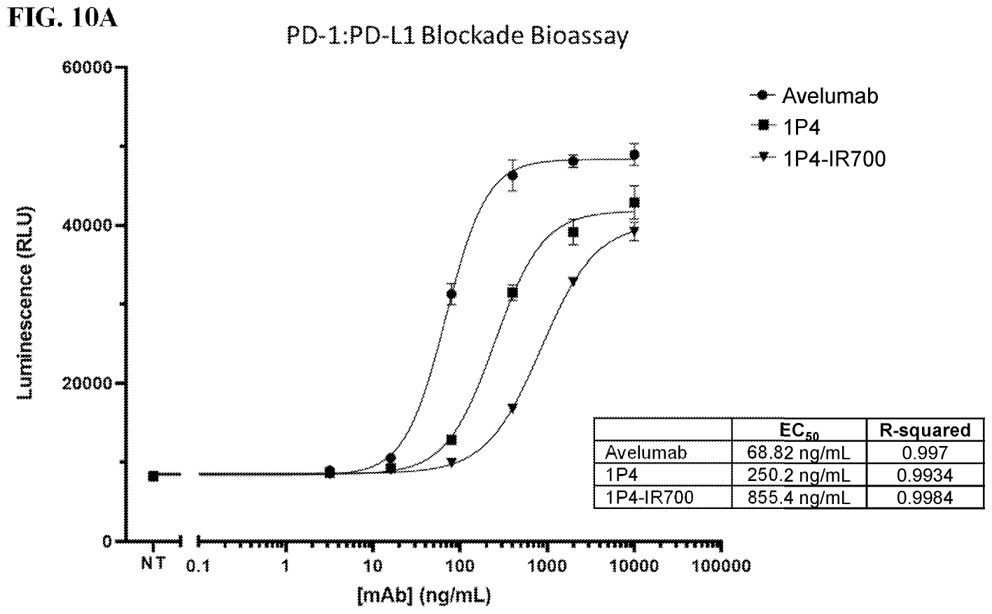

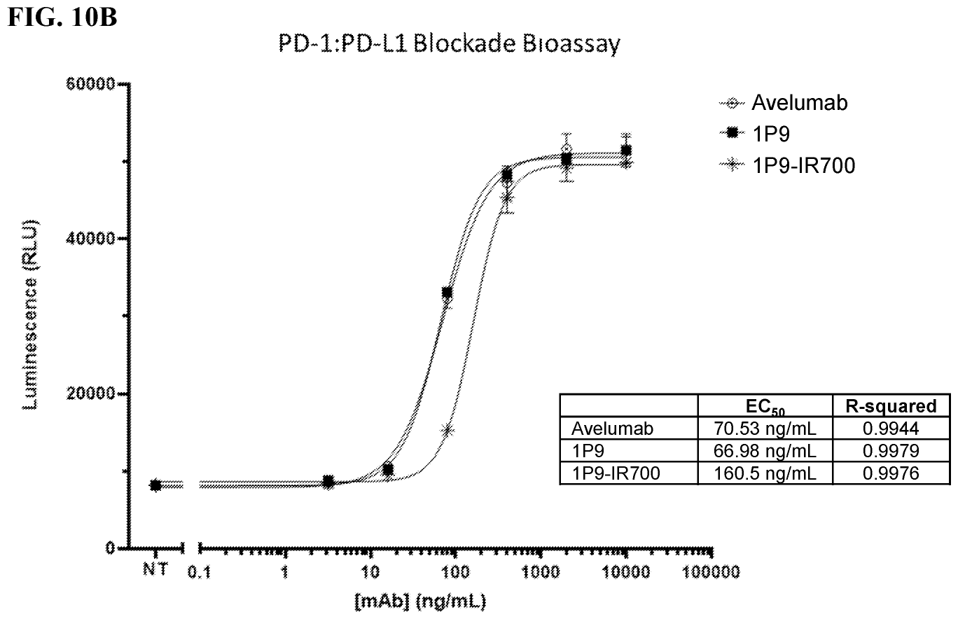

Exemplary anti-PD-L1 antibodies, 1P4 and 1P9, and anti-PD-L1 conjugates, 1P4-IR700 and 1P9-IR700, were tested for their abilities to block the interaction of PD-1 with its ligand, PD-L1, using a PD-1:PD-L1 Blockade Bioassay kit (Promega, Cat. No. J1250). Briefly, PD-L1 aAPC/CHO-K1 cells, CHO-K1 cells expressing human PD-L1 and an engineered cell surface protein designed to activate cognate TCRs in an antigen-independent manner, were thawed and plated in flat-bottom, white 96-well plates in 200 μL 90% Haim's F-12 medium, 10% FBS. After incubating overnight at 37° C., 5% CO₂, the culture medium was removed, and 40 μL anti-PD-L1 antibody (1P4, 1P9, or Avelumab (control)) or anti-PD-L1 conjugate (1P4-IR700 or 1P9-IR700) diluted in Assay Buffer (99% RPMI-1640, 1% FBS) were added. The plates were covered with a lid and kept at room temperature while the PD-1 Effector cells, Jurkat T cells expressing human PD-1 and a luciferase reporter driven by an NFAT response element (NFAT-RE), were thawed and resuspended in Assay Buffer. 40 μL of PD-1 Effector cells were added to the assay wells, and the plates were incubated at 37° C., 5% CO₂ for 6 hours. The plates were removed from the incubator and equilibrated to room temperature for 5-10 minutes before adding 80 μL Bio-Glo™ Reagent. The plates incubated at room temperature for 5-30 minutes and then the luminescence was measured using a Tecan Spark plate reader. Luminescence (RLU) were plotted vs. Log10 antibody concentration, after subtracting background signal. Fit curves and EC₅₀ values were determined using GraphPad Prism. The results, provided in FIGS. 10A and 10B, demonstrated that the anti-PD-L1 antibodies exhibit blocking activity of the PD-1:PD-L1 interaction.

### Example 11: Internalization of Anti-PD-L1 Antibody Conjugates

Exemplary anti-PD-L1 antibody conjugates were tested for internalization into PD-L1 expressing cells upon binding. The exemplary anti-PD-L1 conjugates, 1P9-IR700 and 1P4-IR700, avelumab-IR700, a negative control IgG1 isotype control antibody, and a positive control anti-EphA2-IR700 conjugate were conjugated to a pH sensor dye (pHAb Reactive Dye, Promega Cat. No. G9845) to monitor antibody internalization.

Human pancreatic cancer BxPC3 cells were plated in a 48-well, flat bottom plate at 50,000 cells/well and incubated in the presence of 1 μg/mL or 10 μg/mL conjugated antibody at 37° C. and 5% CO₂ for 24 hours prior to IFN-gamma stimulation. After incubation, cell suspensions were transferred to 96-well U-bottom plates and centrifuged at 500×g for 5 minutes at 4° C. After centrifugation, the supernatant was removed, and cell pellets were resuspended in PBS (50 μL/cell pellet sample). Cell viability Zombie Violet stain was then added to all wells at 50 μL/well except for the unstained control cells wells; the plates were then incubated at room temperature for 20-30 minutes, protected from light. Meanwhile, dead cell control samples were prepared by subjecting the samples to two rounds of freeze-thaw cycles in a −80° C. freezer and 37° C. water bath, respectively. After the freeze-thaw cycles, 50 μL Zombie Violet stain were added, followed by incubation for 20-30 minutes at room temperature. After incubation, the dead cell control samples were left on ice until all samples were ready to analyze by flow cytometry.

After the Zombie Violet incubation of the cells in the plates, 150 μL/well FACS buffer was added to all wells of the plates, and the plates were centrifuged at 500×g for 5 minutes. After centrifugation, the supernatant was removed, and cell pellets were resuspended with FACS buffer. The plates were then incubated on ice, protected from light, for 30 minutes. The cells were then washed and resuspended in FACS buffer at 200 μL/well, and 100 μL added FACS buffer were added to the dead cells control sample.

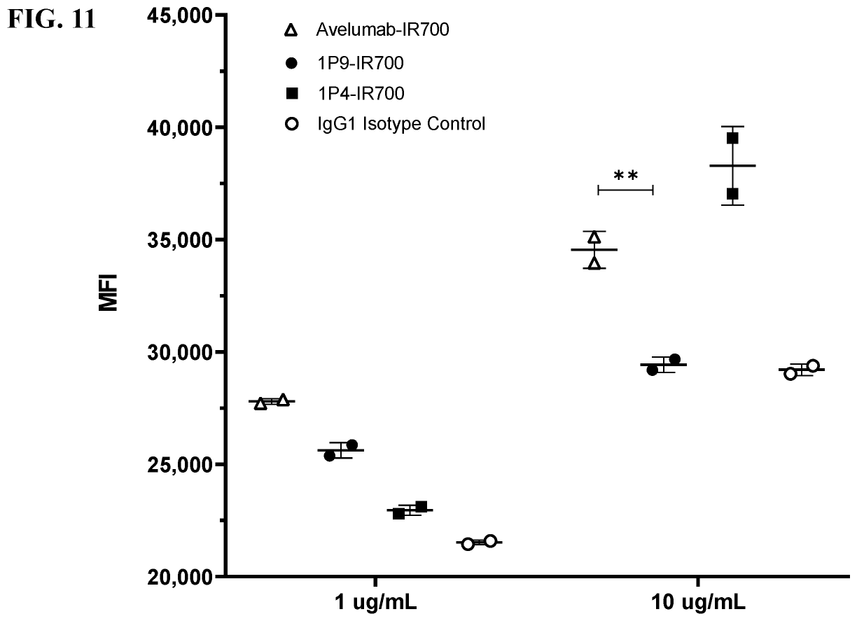

Data acquisition was then performed on the CytoFLEX flow cytometer, using the unstained control cell samples to set forward scatter (FSC) and side scatter (SSC) gating parameters and the live and dead cell samples to set the viable cell gating parameters. Fluorescence was measured in the phycoerythrin (PE) channel for the pH sensor dye and Zombie Violet channel for cell viability. The antibody internalization, normalized to viable cells is provided in FIG. 11. As shown in FIG. 11, anti-PD-L1 conjugates 1P4-IR700 and 1P9-IR700 were internalized less than avelumab-IR700 at 1 μg/mL. At 10 μg/mL 1P4-IR700 was internalized significantly less than avelumab-IR700 while 1P9-IR700 was internalized more than either avelumab or 1P4-IR700. The non-specific isotype control antibody exhibited the least internalization at both concentrations. The EphA2-IR700 (positive control) conjugate exhibited the most internalization at both concentrations tested (data not shown).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID | | |
|---|---|---|
| NO. | Sequence | Description |
| 1 | QVQLVESGGGVVQPGRSLRLSCAASGFTFSNYGMHWVR | 1P1/1P4_VH |
| | QAPGKGLEWVSLILYDGSNKYYADSVKGRFTISRDNSK | |
| | NTLYLQMNSLRAEDTAVYYCARDRVWLDVWGKGTTVTV | |
| | SS | |
| 2 | QVQLVESGGGVVQPGRSLRLSCAASGFTFSSYGMHWVR | 1P1.2/1P4.2_VH |
| | QAPGKGLEWVAVIWYDGSNKYYADSVKGRFTISRDNSK | |
| | NTLYLQMNSLRAEDTAVYYCARDRVWLDVWGKGTTVTV | |
| | SS | |
| 3 | QVQLVQSGAEVKKPGASVKISCKASGFTFSRYDFNWVR | 1P2_VH |
| | QATGQGLEWMGWMNPNNGNTGYSQKFQGRVTMTRNTSI | |
| | STAYMELSSLRSEDTAVYYCARGWTFFDYWGQGTLVTV | |
| | SS | |
| 4 | QVQLVQSGAEVKKPGASVKISCKASGFTFSRYDFNWVR | 1P2_N73D_VH |
| | QATGQGLEWMGWMNPNNGNTGYSQKFQGRVTMTRDTSI | |
| | STAYMELSSLRSEDTAVYYCARGWTFFDYWGQGTLVTV | |
| | SS | |
| 5 | QVQLVQSGAEVKKPGASVKVSCKASGYTFTSYDINWVR | 1P2.2_VH |
| | QATGQGLEWMGWMNPNSGNTGYAQKFQGRVTMTRNTSI | |
| | STAYMELSSLRSEDTAVYYCARGWTFFDYWGQGTLVTV | |
| | SS | |
| 6 | EVQLVESGGGLVQPGGSLRLSCAASGFTFSTYWMSWVR | 1P9_VH |
| | QAPGKGLEWVANIKQDGGERYYVDSVKGRFTISRDNAK | |
| | SSLYLQMNSLRAEDTALYYCARLRLYSDYEDYWGQGTL | |
| | VTVSS | |
| 7 | EVQLVESGGGLVQPGGSLRLSCAASGFTFSSYWMSWVR | 1P9.2_VH |
| | QAPGKGLEWVANIKQDGSEKYYVDSVKGRFTISRDNAK | |
| | NSLYLQMNSLRAEDTAVYYCARLRLYSDYEDYWGQGTL | |
| | VTVSS | |
| 8 | EVQLVESGGGLVKPGGSLRLSCAASGFTFSSYTMNWVR | 2M1_VH |
| | QAPGKGLEWVSSITSSTSYIYYADSVKGRFTISRDNAK | |
| | NSLYLQMNSLRAEDTSVYYCVRDWSIAARGAWFDPWGQ | |
| | GTLVTVSS | |
| 9 | QVQLVQSGAEVKKPGASVKVSCKASGYTFTNYGISWVR | 2M2_VH |
| | QAPGQGLEWMGWISAYNGHTNYAQKVQGRVTMTTDTST | |
| | STAFMELRSLRSDDSAVYYCARGTSGDYWGQGTLVTVS | |
| | S | |
| 10 | EVQLVESGGGFVQTGGSLRLSCAASGENFRSYDMHWVR | 2M3_VH |
| | QVIGKGLEWVSGIGPGGDTYYPGSVKGRFTISRENAKN | |
| | SLYLQMNSLRAGDTAVYYCAREQFEGHYFDYWGQGTLV | |
| | TVSS | |
| 11 | EVQLVESGGGLVKPGGSLRVSCAASGFTLSSYSMNWVR | 2M5_VH |
| | QAPGKGLEWVSSISSSSSYIYYADSVKGRFTISRDNAK | |
| | NLLYLQMNSLRAEDTAVYYCAREGGAARPSWFDPWGQG | |
| | TLVTVSS | |
| 12 | QIQLVQSGPELKKPGETVKISCKASGYDFTNYGMNWVK | 3B1_3B5_VH |
| | QAPGKGLKWMGYINTYTGEPAYADDFKGRFAFSLETSA | |
| | RTAYLRINNLKNEDTATYFCATRAYYGHYLAYWGQGTL | |
| | VTVSA | |
| 13 | EVQLQESGPSLVKPSQTLSLTCSVTGDSITSGYWNWIR | 3B2_VH |
| | KFPGNKLEYMGYISYTGSTYYNPSLKSRISITRDTSKN | |
| | QYYLQLDSVTTEDTATYYCARKGGWLLPFAYWGQGTLV | |
| | TVSA | |
| 14 | QVQLVETGGGLVRPGNSLKLSCVTSGFTFSNYRMHWLR | 3B3_VH |
| | QPPGKRLEWIAVITVKSDNYGANYAESVKGRFTISRDD | |
| | SKSSVYLQMNRLREEDTATYYCSLGRYFDYWGQGTTLT | |
| | VSS | |
| 15 | EVQLQESGPSLVKPSQTLSLTCSVTGDSITSGYWNWIR | 3B4_VH |
| | KFPGNKLESMGYISYTGSTYYNPSLKSRLSITRDTSKN | |
| | QFYLQLNSVTTEDTATYYCASGLLWLAWFAYWGQGTLV | |
| | TVSA | |
| 16 | EVKLVESGGGLVQPGGSLRLSCGTSGFTFSDFYVEWVR | 3B6_VH |
| | QPPGKRLEWIAASRNKVNDYTTEYSESVKGRFIVSRDT | |
| | SQNILYLQMNALRAEDSAIYYCARNVPWDGWYFDVWGA | |
| | GTAVTVSS | |
| 17 | DIQMTQSPSSLSASVGDRVTITCRASQGIRNYLAWYQQ | 1P1_VL |
| | KPGKVPKLLIYAASTLQSGVPSRFSGSGSGTDFTLTIS | |
| | GLQPEDVATYYCQKYNSALWTFGQGTKVEIK | |
| 18 | DIQMTQSPSSLSASVGDRVTITCRASQGISNYLAWYQQ | 1P1.2_VL |
| | KPGKVPKLLIYAASTLQSGVPSRFSGSGSGTDFTLTIS | |
| | SLQPEDVATYYCQKYNSALWTFGQGTKVEIK | |
| 19 | EIVLTQSPGTLSLSPGERATLSCRASQSVSSTYLAWYQ | 1P2_VL |
| | QKPGQAPRLLIFGASSRATGIPDRFSGSGSGTDFTLTI | |
| | SRLEPEDFAVYYCQQYGSSPTFGQGTKVEIK | |
| 20 | EIVLTQSPGTLSLSPGERATLSCRASQSVSSSYLAWYQ | 1P2.2_VL |
| | QKPGQAPRLLIYGASSRATGIPDRFSGSGSGTDFTLTI | |
| | SRLEPEDFAVYYCQQYGSSPTFGQGTKVEIK | |
| 21 | DIVMTQSPDSLAVSLGERATINCKSSQSVLYRSSNKNY | 1P4_VL |
| | LAWYQQKPGQPPKLLIYWASTRESGVPDRFSGSGSGTD | |
| | FTLTISSLQAEDVAVYYCHQYYTTPLTFGGGTKVEIR | |
| 22 | DIVMTQSPDSLAVSLGERATINCKSSQSVLYSSNNKNY | 1P4.2_VL |
| | LAWYQQKPGQPPKLLIYWASTRESGVPDRFSGSGSGTD | |
| | FTLTISSLQAEDVAVYYCQQYYSTPLTFGGGTKVEIR | |
| 23 | DIQMTQSPSSVSASVGDRVTITCRASQGISSWLAWYQQ | 1P9_VL |
| | KPGKAPKLLIYSVSSLQSGVPSRFSGSGSGTDFTLTIS | |
| | SLQPEDSATYYCQQANSLPPTFGGGTKVEIK | |
| 24 | DIQMTQSPSSVSASVGDRVTITCRASQGISSWLAWYQQ | 1P9.2_VL |
| | KPGKAPKLLIYAASSLQSGVPSRFSGSGSGTDFTLTIS | |
| | SLQPEDFATYYCQQANSFPPTFGGGTKVEIK | |
| 25 | DIQMTQSPSSVSASVGDRVTITCRASQGIGSWLAWYQQ | 2M1_VL |
| | KPGKAPKFLIYAASSLQGGVPSRFSGSGSGTDFTLTIS | |
| | SLQPEDFATYYCQQANSFPLTFGGGTKVEIK | |
| 26 | EIVLTQSPATLSLSPGERATLSCRASQSVSSYLAWYQQ | 2M2_VL |
| | KPGQAPRLLIYDASNRATGIPGRFSGSGSGTDFTLTIS | |
| | SLEPEDFAVYYCQQRSNWPPLTFGGGTKVEIK | |
| 27 | DIQMTQSPSSVSASVGDRVNITCRASQGISSWLAWYQQ | 2M3_VL |
| | KPWKAPKLLIYTASSLQSGVPSRFSGSGSGTDFTLTIS | |
| | SLQPEDFATYYCQQANSFPITFGQGTRLEIK | |
| 28 | QAVVTQESALTTSPGETVTLTCRSSTGAVTTSNYANWV | 2M5_VL |
| | QEKPDHLFTGLIGGINNRAPGVPARFSGSLIGDKAALT | |
| | ITGAQTEDEAIYFCALWYRNHWVFGGGTKLTVL | |
| 29 | DVLMTQTPLSLPVSLGDQASISCRSSQRIVHSNGNTYL | 3B1_VL |
| | EWYLQKPGQSPKLLIYKVSNRFSGVPDRFSGSGSGTDF | |
| | TLKISRVEAEDLGVYYCFQGSHVPYTFGGGTKLEIK | |
| 30 | DIVMSQSPSSLAVSFGEKVTMSCKSSQSLLYSTNQKNS | 3B2_VL |
| | LAWYQQKPGQSPKLLIYWASTRESGVPDRFTGSGSGTD | |
| | FTLTISSVKAEDLAVYYCQQYYSYPFTFGSGTKLEIK | |
| 31 | DIQMTQTTSSLSASLGDRVTISCSASQGISNYLNWYQQ | 3B3_VL |
| | KPDGTVKLLIYYTSSLHSGVPSRFSGSGSGTDYSLTIS | |
| | NLEPEDIATYYCQQYSKLPWTFGGGTKLEIK | |
| 32 | DIVMSQSPSSLAVSVGEKVTMSCKSSQSLLYSSNQKNS | 3B4_VL |
| | LAWYQQKPGQSPKLLIYWASTRESGVPDRFTGSGSGTD | |
| | FTLTISSVRAEDLAVYYCQQYYGYPFTFGGGTKLEIK | |
| 33 | QIVLTQSPAIMSASPGEKVTITCSASSSVSYMHWFQQK | 3B5_VL |
| | PGTSPKLWIYSTSNLASGVPARFSGSGSGTSYSLTISR | |
| | MEAEDAATYYCQQRSSYPFTFGSGTKLEIK | |
| 34 | DIVMTQSQKFMSTSVGDRVSITCKASQNVRTAVAWFQQ | 3B6_VL |
| | KPGQSPKALIYLASTRHTGVPDRFTGSGSGTDFTLTIS | |
| | NVQSEDLADYFCLQHWNYPRTFGGGTKLEIK | |
| 35 | GFTFSNY | 1P1/1P4_CDR-H1_Chothia |
| 36 | LYDGSN | 1P1/1P4_CDR-H2_Chothia |
| 37 | DRVWLDV | 1P1/1P4/1P1.2/1P4.2_CDR- |
| | | H3_Chothia_AbM_Kabat |
| 38 | GFTFSNYGMH | 1P1/1P4_CDR-H1_AbM |
| 39 | LILYDGSNKY | 1P1/1P4_CDR-H2_AbM |
| 40 | NYGMH | 1P1/1P4_CDR-H1_Kabat |
| 41 | LILYDGSNKYYADSVKG | 1P1/1P4_CDR-H2_Kabat |
| 42 | SNYGMH | 1P1/1P4_CDR-H1_Contact |
| 43 | WVSLILYDGSNKY | 1P1/1P4_CDR-H2_Contact |
| 44 | ARDRVWLD | 1P1/1P4/1P1.2/1P4.2_CDR- |
| | | H3_Contact |
| 45 | GFTFSNYG | 1P1/1P4_CDR-H1_IMGT |
| 46 | ILYDGSNK | 1P1/1P4_CDR-H2_IMGT |
| 47 | ARDRVWLDV | 1P1/1P4/1P1.2/1P4.2_CDR- |
| | | H3_IMGT |
| 48 | GFTFSSY | 1P1.2/1P4.2/1P9.2/2M1_CDR- |
| | | H1_Chothia |
| 49 | WYDGSN | 1P1.2/1P4.2 CDR-H2_Chothia |
| 50 | GFTFSSYGMH | 1P1.2/1P4.2_CDR-H1_AbM |
| 51 | VIWYDGSNKY | 1P1.2/1P4.2 CDR-H2_AbM |
| 52 | SYGMH | 1P1.2/1P4.2 CDR-H1_Kabat |
| 53 | VIWYDGSNKYYADSVKG | 1P1.2/1P4.2 CDR-H2_Kabat |
| 54 | SSYGMH | 1P1.2/1P4.2 CDR-H1_Contact |
| 55 | WVAVIWYDGSNKY | 1P1.2/1P4.2 CDR-H2_Contact |
| 56 | GFTFSSYG | 1P1.2/1P4.2_ _CDR-H1_IMGT |
| 57 | IWYDGSNK | 1P1.2/1P4.2_CDR-H2_IMGT |
| 58 | GFTFSRY | 1P2/1P2_N73D_CDR-H1_Chothia |
| 59 | NPNNGN | 1P2/1P2_N73D_CDR-H2_Chothia |
| 60 | GWTFFDY | 1P2/1P2_N73D_CDR- |
| | | H3_Chothia_AbM, Kabat; |
| | | 1P2.2_Kabat |
| 61 | GFTFSRYDFN | 1P2/1P2_N73D_CDR-H1_AbM |
| 62 | WMNPNNGNTG | 1P2/1P2_N73D_CDR-H2_AbM |
| 63 | RYDEN | 1P2/1P2_N73D_CDR-H1_Kabat |
| 64 | WMNPNNGNTGYSQKFQG | 1P2/1P2_N73D_CDR-H2_Kabat |
| 65 | SRYDEN | 1P2/1P2_N73D_CDR-H1_Contact |
| 66 | WMGWMNPNNGNTG | 1P2/1P2_N73D_CDR-H2_Contact |
| 67 | ARGWTFFD | 1P2/1P2_N73D/1P2.2_CDR- |
| | | H3_Contact |
| 68 | GFTFSRYD | 1P2/1P2_N73D_CDR-H1_IMGT |
| 69 | MNPNNGNT | 1P2/1P2_N73D_CDR-H2_IMGT |
| 70 | ARGWTFFDY | 1P2/1P2_N73D_CDR-H3_IMGT |
| 71 | GYTFTSY | 1P2.2_CDR-H1_Chothia |
| 72 | NPNSGN | 1P2.2_CDR-H2_Chothia |
| 73 | GYTFTSYDIN | 1P2.2_CDR-H1_AbM |
| 74 | WMNPNSGNTG | 1P2.2_CDR-H2_AbM |
| 75 | SYDIN | 1P2.2_CDR-H1_Kabat |
| 76 | WMNPNSGNTGYAQKFQG | 1P2.2_CDR-H2_Kabat |
| 77 | TSYDIN | 1P2.2_CDR-H1_Contact |
| 78 | WMGWMNPNSGNTG | 1P2.2_CDR-H2_Contact |
| 79 | GYTFTSYD | 1P2.2_CDR-H1_IMGT |
| 80 | MNPNSGNT | 1P2.2_CDR-H2_IMGT |
| 81 | ARGWTFFDY | 1P2.2_CDR-H3_IMGT |
| 82 | GFTFSTY | 1P9_CDR-H1_Chothia |
| 83 | KQDGGE | 1P9_CDR-H2_Chothia |
| 84 | LRLYSDYEDY | 1P9/1P9.2_CDR- |
| | | H3_Chothia_AbM_Kabat |
| 85 | GFTFSTYWMS | 1P9_CDR-H1_AbM |
| 86 | NIKQDGGERY | 1P9_CDR-H2_AbM |
| 87 | TYWMS | 1P9_CDR-H1_Kabat |
| 88 | NIKQDGGERYYVDSVKG | 1P9_CDR-H2_Kabat |
| 89 | STYWMS | 1P9_CDR-H1_Contact |
| 90 | WVANIKQDGGERY | 1P9_CDR-H2_Contact |
| 91 | ARLRLYSDYED | 1P9/1P9.2_CDR-H3_Contact |
| 92 | GFTFSTYW | 1P9_CDR-H1_IMGT |
| 93 | IKQDGGER | 1P9_CDR-H2_IMGT |
| 94 | ARLRLYSDYEDY | 1P9_CDR-H3_IMGT |
| 95 | KQDGSE | 1P9.2_CDR-H2_Chothia |
| 96 | GFTFSSYWMS | 1P9.2_CDR-H1_AbM |
| 97 | NIKQDGSEKY | 1P9.2_CDR-H2_AbM |
| 98 | SYWMS | 1P9.2_CDR-H1_Kabat |
| 99 | NIKQDGSEKYYVDSVKG | 1P9.2_CDR-H2_Kabat |
| 100 | SSYWMS | 1P9.2_CDR-H1_Contact |
| 101 | WVANIKQDGSEKY | 1P9.2_CDR-H2_Contact |
| 102 | GFTFSSYW | 1P9.2_CDR-H1_IMGT |
| 103 | IKQDGSEK | 1P9.2_CDR-H2_IMGT |
| 104 | TSSTSY | 2M1_CDR-H2_Chothia |
| 105 | DWSIAARGAWFDP | 2M1_CDR- |
| | | H3_Chothia_AbM_Kabat |
| 106 | GFTFSSYTMN | 2M1_CDR-H1_AbM |
| 107 | SITSSTSYIY | 2M1_CDR-H2_AbM |
| 108 | SYTMN | 2M1_CDR-H1_Kabat |
| 109 | SITSSTSYIYYADSVKG | 2M1_CDR-H2_Kabat |
| 110 | SSYTMN | 2M1_CDR-H1_Contact |
| 111 | WVSSITSSTSYIY | 2M1_CDR-H2_Contact |
| 112 | VRDWSIAARGAWED | 2M1_CDR-H3_Contact |
| 113 | GFTFSSYT | 2M1_CDR-H1_IMGT |
| 114 | ITSSTSYI | 2M1_CDR-H2_IMGT |
| 115 | VRDWSIAARGAWFDP | 2M1_CDR-H3_IMGT |
| 116 | GYTFTNY | 2M2_CDR-H1_Chothia |
| 117 | SAYNGH | 2M2_CDR-H2_Chothia |
| 118 | GTSGDY | 2M2_CDR- |
| | | H3_Chothia_AbM_Kabat |
| 119 | GYTFTNYGIS | 2M2_CDR-H1_AbM |
| 120 | WISAYNGHTN | 2M2_CDR-H2_AbM |
| 121 | NYGIS | 2M2_CDR-H1_Kabat |
| 122 | WISAYNGHTNYAQKVQG | 2M2_CDR-H2_Kabat |
| 123 | TNYGIS | 2M2_CDR-H1_Contact |
| 124 | WMGWISAYNGHIN | 2M2_CDR-H2_Contact |
| 125 | ARGTSGD | 2M2_CDR-H3_Contact |
| 126 | GYTFTNYG | 2M2_CDR-H1_IMGT |
| 127 | ISAYNGHT | 2M2_CDR-H2_IMGT |
| 128 | ARGTSGDY | 2M2_CDR-H3_IMGT |
| 129 | GFNFRSY | 2M3_CDR-H1_Chothia |
| 130 | GPGGD | 2M3_CDR-H2_Chothia |
| 131 | EQFEGHYFDY | 2M3_CDR- |
| | | H3_Chothia_AbM_Kabat |
| 132 | GFNFRSYDMH | 2M3_CDR-H1_AbM |
| 133 | GIGPGGDTY | 2M3_CDR-H2_AbM |
| 134 | SYDMH | 2M3_CDR-H1_Kabat |
| 135 | GIGPGGDTYYPGSVKG | 2M3_CDR-H2_Kabat |
| 136 | RSYDMH | 2M3_CDR-H1_Contact |
| 137 | WVSGIGPGGDTY | 2M3_CDR-H2_Contact |
| 138 | AREQFEGHYFD | 2M3_CDR-H3_Contact |
| 139 | GFNFRSYD | 2M3_CDR-H1_IMGT |
| 140 | IGPGGDT | 2M3_CDR-H2_IMGT |
| 141 | AREQFEGHYFDY | 2M3_CDR-H3_IMGT |
| 142 | GFTLSSY | 2M5_CDR-H1_Chothia |
| 143 | SSSSSY | 2M5_CDR-H2_Chothia |
| 144 | EGGAARPSWEDP | 2M5_CDR- |
| | | H3_Chothia_AbM_Kabat |
| 145 | GFTLSSYSMN | 2M5_CDR-H1_AbM |
| 146 | SISSSSSYIY | 2M5_CDR-H2_AbM |
| 147 | SYSMN | 2M5_CDR-H1_Kabat |
| 148 | SISSSSSYIYYADSVKG | 2M5_CDR-H2_Kabat |
| 149 | SSYSMN | 2M5_CDR-H1_Contact |
| 150 | WVSSISSSSSYIY | 2M5_CDR-H2_Contact |
| 151 | AREGGAARPSWFD | 2M5_CDR-H3_Contact |
| 152 | GFTLSSYS | 2M5_CDR-H1_IMGT |
| 153 | ISSSSSYI | 2M5_CDR-H2_IMGT |
| 154 | AREGGAARPSWFDP | 2M5_CDR-H3_IMGT |
| 155 | GYDFTNY | 3B1/3B5_CDR-H1_Chothia |
| 156 | NTYTGE | 3B1/3B5_CDR-H2_Chothia |
| 157 | RAYYGHYLAY | 3B1/3B5_CDR- |
| | | H3_Chothia_AbM_Kabat |
| 158 | GYDFTNYGMN | 3B1/3B5_CDR-H1_AbM |
| 159 | YINTYTGEPA | 3B1/3B5_CDR-H2_AbM |
| 160 | NYGMN | 3B1/3B5_CDR-H1_Kabat |
| 161 | YINTYTGEPAYADDFKG | 3B1/3B5_CDR-H2_Kabat |
| 162 | TNYGMN | 3B1/3B5_CDR-H1_Contact |
| 163 | WMGYINTYTGEPA | 3B1/3B5_CDR-H2_Contact |
| 164 | ATRAYYGHYLA | 3B1/3B5_CDR-H3_Contact |
| 165 | GYDFTNYG | 3B1/3B5_CDR-H1_IMGT |
| 166 | INTYTGEP | 3B1/3B5_CDR-H2_IMGT |
| 167 | ATRAYYGHYLAY | 3B1/3B5_CDR-H3_IMGT |
| 168 | GDSITSG | 3B2/3B4_CDR-H1_Chothia |
| 169 | SYTGS | 3B2/3B4_CDR-H2_Chothia |
| 170 | KGGWLLPFAY | 3B2_CDR- |
| | | H3_Chothia_AbM_Kabat |
| 171 | GDSITSGYWN | 3B2/3B4_CDR-H1_AbM |
| 172 | YISYTGSTY | 3B2/3B4_CDR-H2_AbM |
| 173 | SGYWN | 3B2/3B4_CDR-H1_Kabat |
| 174 | YISYTGSTYYNPSLKS | 3B2/3B4_CDR-H2_Kabat |
| 175 | TSGYWN | 3B2/3B4_CDR-H1_Contact |
| 176 | YMGYISYTGSTY | 3B2_CDR-H2_Contact |
| 177 | ARKGGWLLPFA | 3B2_CDR-H3_Contact |
| 178 | GDSITSGY | 3B2/3B4_CDR-H1_IMGT |
| 179 | ISYTGST | 3B2/3B4_CDR-H2_IMGT |
| 180 | ARKGGWLLPFAY | 3B2_CDR-H3_IMGT |
| 181 | TVKSDNYG | 3B3_CDR-H2_Chothia |
| 182 | GRYFDY | 3B3_CDR- |
| | | H3_Chothia_AbM_Kabat |
| 183 | GFTFSNYRMH | 3B3_CDR-H1_AbM |
| 184 | VITVKSDNYGAN | 3B3_CDR-H2_AbM |
| 185 | NYRMH | 3B3_CDR-H1_Kabat |
| 186 | VITVKSDNYGANYAESVKG | 3B3_CDR-H2_Kabat |
| 187 | SNYRMH | 3B3_CDR-H1_Contact |
| 188 | WIAVITVKSDNYGAN | 3B3_CDR-H2_Contact |
| 189 | SLGRYFD | 3B3_CDR-H3_Contact |
| 190 | GFTFSNYR | 3B3_CDR-H1_IMGT |
| 191 | ITVKSDNYGA | 3B3_CDR-H2_IMGT |
| 192 | SLGRYFDY | 3B3_CDR-H3_IMGT |
| 193 | GLLWLAWFAY | 3B4_CDR- |
| | | H3_Chothia_AbM_Kabat |
| 194 | SMGYISYTGSTY | 3B4_CDR-H2_Contact |
| 195 | ASGLLWLAWFA | 3B4_CDR-H3_Contact |
| 196 | ASGLLWLAWFAY | 3B4_CDR-H3_IMGT |
| 197 | GFTFSDF | 3B6_CDR-H1_Chothia |
| 198 | RNKVNDYT | 3B6_CDR-H2_Chothia |
| 199 | NVPWDGWYFDV | 3B6_CDR- |
| | | H3_Chothia_AbM_Kabat |
| 200 | GFTFSDFYVE | 3B6_CDR-H1_AbM |
| 201 | ASRNKVNDYTTE | 3B6_CDR-H2_AbM |
| 202 | DFYVE | 3B6_CDR-H1_Kabat |
| 203 | ASRNKVNDYTTEYSESVKG | 3B6_CDR-H2_Kabat |
| 204 | SDFYVE | 3B6_CDR-H1_Contact |
| 205 | WIAASRNKVNDYTTE | 3B6_CDR-H2_Contact |
| 206 | ARNVPWDGWYFD | 3B6_CDR-H3_Contact |
| 207 | GFTFSDFY | 3B6_CDR-H1_IMGT |
| 208 | SRNKVNDYTT | 3B6_CDR-H2_IMGT |
| 209 | ARNVPWDGWYFDV | 3B6_CDR-H3_IMGT |
| 210 | RASQGIRNYLA | 1P1_CDR- |
| | | L1_Chothia_AbM_Kabat |
| 211 | AASTLQS | 1P1/P1.2_CDR- |
| | | L2_Chothia AbM Kabat |
| 212 | QKYNSALWT | 1P1/1P1.2_CDR- |
| | | L3_Chothia_AbM_Kabat_IMGT |
| 213 | RNYLAWY | 1P1_CDR-L1_Contact |
| 214 | LLIYAASTLQ | 1P1_CDR-L2_Contact |
| 215 | QKYNSALW | 1P1_CDR-L3_Contact |
| 216 | QGIRNY | 1P1_CDR-L1_IMGT |
| 217 | AA | 1P1/1P9.2/2M1_CDR-L2_IMGT |
| 218 | RASQGISNYLA | 1P1.2_CDR-L1_Chothia |
| 219 | SNYLAWY | 1P1.2_CDR-L1_Contact |
| 220 | QGISNY | 1P1.2/3B3_CDR-L1_IMGT |
| 221 | RASQSVSSTYLA | 1P2/1P2_N73D_CDR- |
| | | L1_Chothia_AbM_Kabat |
| 222 | GASSRAT | 1P2/1P2_N73D/1P2.2_CDR- |
| | | L2_Chothia_AbM_Kabat |
| 223 | QQYGSSPT | 1P2/1P2_N73D1P2.2_CDR- |
| | | L3_Chothia_AbM_Kabat_IMGT |
| 224 | SSTYLAWY | 1P2/1P2_N73D_CDR-L1_Contact |
| 225 | LLIFGASSRA | 1P2/1P2_N73D_CDR-L2_Contact |
| 226 | QQYGSSP | 1P2/1P2/1P2.2_N73D_CDR- |
| | | L3_Contact |
| 227 | QSVSSTY | 1P2/1P2_N73D_CDR-L1_IMGT |
| 228 | GA | 1P2/1P2_N73D_CDR-L2_IMGT |
| 229 | RASQSVSSSYLA | 1P2.2_CDR- |
| | | L1_Chothia_AbM_Kabat |
| 230 | SSSYLAWY | 1P2.2_CDR-L1_Contact |
| 231 | LLIYGASSRA | 1P2.2_CDR-L2_Contact |
| 232 | QSVSSSY | 1P2.2_CDR-L1_IMGT |
| 233 | KSSQSVLYRSSNKNYLA | 1P4_CDR- |
| | | L1_Chothia_AbM_Kabat |
| 234 | WASTRES | 1P4/1P4.2/3B2/3B4_CDR- |
| | | L2_Chothia_AbM_Kabat |
| 235 | HQYYTTPLT | 1P4_CDR- |
| | | L3_Chothia_AbM_Kabat_IMGT |
| 236 | LYRSSNKNYLAWY | 1P4_CDR-L1_Contact |
| 237 | LLIYWASTRE | 1P4/1P4.2/3B2/3B4_CDR- |
| | | L2_Contact |
| 238 | HQYYTTPL | 1P4_CDR-L3_Contact |
| 239 | QSVLYRSSNKNY | 1P4_CDR-LH1_IMGT |
| 240 | WA | 1P4/3B2/3B4_CDR-L2_IMGT |
| 241 | KSSQSVLYSSNNKNYLA | 1P4.2_CDR- |
| | | L1_Chothia_AbM_Kabat |
| 242 | QQYYSTPLT | 1P4.2_CDR- |
| | | L3_Chothia_AbM_Kabat_IMGT |
| 243 | LYSSNNKNYLAWY | 1P9_CDR-L1_Contact |
| 244 | QQYYSTPL | 1P9_CDR-L3_Contact |
| 245 | QSVLYSSNNKNY | 1P9_CDR-L1_IMGT |
| 246 | RASQGISSWLA | 1P9/1P9.2/2M3_CDR- |
| | | L1_Chothia_AbM_Kabat |
| 247 | SVSSLQS | 1P9_CDR- |
| | | L2_Chothia_AbM_Kabat |
| 248 | QQANSLPPT | 1P9_CDR- |
| | | L3_Chothia_AbM_Kabat_IMGT |
| 249 | SSWLAWY | 1P9/1P9.2/2M3_CDR-L1_Contact |
| 250 | LLIYSVSSLQ | 1P9_CDR-L2_Contact |
| 251 | QQANSLPP | 1P9_CDR-L3_Contact |
| 252 | QGISSW | 1P9/2M3_CDR-L1_IMGT |
| 253 | SV | 1P9_CDR-L2_IMGT |
| 254 | AASSLQS | 1P9.2_CDR- |
| | | L2_Chothia_AbM_Kabat |
| 255 | QQANSFPPT | 1P9.2_CDR- |
| | | L3_Chothia_AbM_Kabat_IMGT |
| 256 | LLIYAASSLQ | 1P9.2_CDR-L2_Contact |
| 257 | QQANSFPP | 1P9.2_CDR-L3_Contact |
| 258 | RASQGIGSWLA | 2M1_CDR- |
| | | L1_Chothia_AbM_Kabat |
| 259 | AASSLQG | 2M1_CDR- |
| | | L2_Chothia_AbM_Kabat |
| 260 | QQANSFPLT | 2M1_CDR- |
| | | L3_Chothia_AbM_Kabat_IMGT |
| 261 | GSWLAWY | 2M1_CDR-L1_Contact |
| 262 | FLIYAASSLQ | 2M1_CDR-L2_Contact |
| 263 | QQANSFPL | 2M1_CDR-L3_Contact |
| 264 | QGIGSW | 2M1_CDR-L1_IMGT |
| 265 | RASQSVSSYLA | 2M2_CDR- |
| | | L1_Chothia_AbM_Kabat |
| 266 | DASNRAT | 2M2_CDR- |
| | | L2_Chothia_AbM_Kabat |
| 267 | QQRSNWPPLT | 2M2_CDR- |
| | | L3_Chothia_AbM_Kabat_IMGT |
| 268 | SSYLAWY | 2M2_CDR-L1_Contact |
| 269 | LLIYDASNRA | 2M2_CDR-L2_Contact |
| 270 | QQRSNWPPL | 2M2_CDR-L3_Contact |
| 271 | QSVSSY | 2M2_CDR-L1_IMGT |
| 272 | DA | 2M2_CDR-L2_IMGT |
| 273 | TASSLQS | 2M3_CDR- |
| | | L2_Chothia_AbM_Kabat |
| 274 | QQANSFPIT | 2M3_CDR- |
| | | L3_Chothia_AbM_Kabat_IMGT |
| 275 | LLIYTASSLQ | 2M3_CDR-L2_Contact |
| 276 | QQANSFPI | 2M3_CDR-L3_Contact |
| 277 | TA | 2M3_CDR-L2_IMGT |
| 278 | RSSTGAVTTSNYAN | 2M5_CDR- |
| | | L1_Chothia_AbM_Kabat |
| 279 | GTNNRAP | 2M5_CDR- |
| | | L2_Chothia_AbM_Kabat |
| 280 | ALWYRNHWV | 2M5_CDR- |
| | | L3_Chothia_AbM_Kabat_IMGT |
| 281 | VTTSNYANWV | 2M5_CDR-L1_Contact |
| 282 | GLIGGTNNRA | 2M5_CDR-L2_Contact |
| 283 | ALWYRNHW | 2M5_CDR-L3_Contact |
| 284 | TGAVTTSNY | 2M5_CDR-L1_IMGT |
| 285 | GT | 2M5_CDR-L2_IMGT |
| 286 | RSSQRIVHSNGNTYLE | 3B1_CDR- |
| | | L1_Chothia_AbM_Kabat |
| 287 | KVSNRFS | 3B1_CDR- |
| | | L2_Chothia_AbM_Kabat |
| 288 | FQGSHVPYT | 3B1_CDR- |
| | | L3_Chothia_AbM_Kabat_IMGT |
| 289 | VHSNGNTYLEWY | 3B1_CDR-L1_Contact |
| 290 | LLIYKVSNRF | 3B1_CDR-L2_Contact |
| 291 | FQGSHVPY | 3B1_CDR-L3_Contact |
| 292 | QRIVHSNGNTY | 3B1_CDR-L1_IMGT |
| 293 | KV | 3B1_CDR-L2_IMGT |
| 294 | KSSQSLLYSTNQKNSLA | 3B2_CDR- |
| | | L1_Chothia_AbM_Kabat |
| 295 | QQYYSYPFT | 3B2_CDR- |
| | | L3_Chothia_AbM_Kabat_IMGT |
| 296 | LYSTNQKNSLAWY | 3B2_CDR-L1_Contact |
| 297 | QQYYSYPF | 3B2_CDR-L3_Contact |
| 298 | QSLLYSTNQKNS | 3B2_CDR-L1_IMGT |
| 299 | SASQGISNYLN | 3B3_CDR- |
| | | L1_Chothia_AbM_Kabat |
| 300 | YTSSLHS | 3B3_CDR- |
| | | L2_Chothia_AbM_Kabat |
| 301 | QQYSKLPWT | 3B3_CDR- |
| | | L3_Chothia_AbM_Kabat_IMGT |
| 302 | SNYLNWY | 1P9_CDR-L1_Contact |
| 303 | LLIYYTSSLH | 1P9_CDR-L2_Contact |
| 304 | QQYSKLPW | 1P9_CDR-L3_Contact |
| 305 | YT | 1P9_CDR-L2_IMGT |
| 306 | KSSQSLLYSSNQKNSLA | 1P9_CDR- |
| | | L1_Chothia_AbM_Kabat |
| 307 | QQYYGYPFT | 1P9_CDR- |
| | | L3_Chothia_AbM_Kabat_IMGT |
| 308 | LYSSNQKNSLAWY | 1P9_CDR-L1_Contact |
| 309 | QQYYGYPF | 1P9_CDR-L3_Contact |
| 310 | QSLLYSSNQKNS | 1P9_CDR-L1_IMGT |
| 311 | SASSSVSYMH | 1P9_CDR- |
| | | L1_Chothia_AbM_Kabat |
| 312 | STSNLAS | 1P9_CDR- |
| | | L2_Chothia_AbM_Kabat |
| 313 | QQRSSYPFT | 1P9_CDR- |
| | | L3_Chothia_AbM_Kabat_IMGT |
| 314 | SYMHWF | 1P9_CDR-L1_Contact |
| 315 | LWIYSTSNLA | 1P9_CDR-L2_Contact |
| 316 | QQRSSYPF | 1P9_CDR-L3_Contact |
| 317 | SSVSY | 1P9_CDR-L1_IMGT |
| 318 | ST | 1P9_CDR-L2_IMGT |
| 319 | KASQNVRTAVA | 1P9_CDR- |
| | | L1_Chothia_AbM_Kabat |
| 320 | LASTRHT | 1P9_CDR- |
| | | L2_Chothia_AbM_Kabat |
| 321 | LQHWNYPRT | 1P9_CDR- |
| | | L3_Chothia_AbM_Kabat_IMGT |
| 322 | RTAVAWF | 1P9_CDR-L1_Contact |
| 323 | ALIYLASTRH | 1P9_CDR-L2_Contact |
| 324 | LQHWNYPR | 1P9_CDR-L3_Contact |
| 325 | QNVRTA | 1P9_CDR-L1_IMGT |
| 326 | LA | 1P9_CDR-L2_IMGT |
| 327 | FTVTVPKDLYVVEYGSNMTIECKFPVEKQLDLAALIVY | huPD-L1 ECD (UniProt Accession |
| | WEMEDKNIIQFVHGEEDLKVQHSSYRQRARLLKDQLSL | # Q9NZQ7-Phe19-Thr239) |
| | GNAALQITDVKLQDAGVYRCMISYGGADYKRITVKVNA | |
| | PYNKINQRILVVDPVTSEHELTCQAEGYPKAEVIWTSS | |
| | DHQVLSGKTTTTNSKREEKLFNVTSTLRINTTTNEIFY | |
| | CTFRRLDPEENHTAELVIPELPLAHPPNERT | |
| 328 | FTVTVPKDLYVVEYGSNMTIECKFPVEKQLDLTSLIVY | cyPD-L1 ECD (UniProt Accession |
| | WEMEDKNIIQFVHGEEDLKVQHSNYRQRAQLLKDQLSL | # G7PSE7-Phe19-Thr239) |
| | GNAALRITDVKLQDAGVYRCMISYGGADYKRITVKVNA | |
| | PYNKINQRILVVDPVTSEHELTCQAEGYPKAEVIWTSS | |
| | DHQVLSGKTTTTNSKREEKLLNVTSTLRINTTANEIFY | |
| | CIFRRLDPEENHTAELVIPELPLALPPNERT | |
| 329 | FTITAPKDLYVVEYGSNVTMECRFPVERELDLLALVVY | muPD-L1 ECD (UniProt |
| | WEKEDEQVIQFVAGEEDLKPQHSNFRGRASLPKDQLLK | Accession # Q9EP73-Phe19- |
| | GNAALQITDVKLQDAGVYCCIISYGGADYKRITLKVNA | Thr238) |
| | PYRKINQRISVDPATSEHELICQAEGYPEAEVIWINSD | |
| | HQPVSGKRSVTTSRTEGMLLNVTSSLRVNATANDVFYC | |
| | TFWRSQPGQNHTAELIIPELPATHPPQNRT | |
| 330 | QVQLVQSGSELKKPGASVKVSCKASGYDFTNYGMNWVR | 3B1_3B5_VH_Humanized |
| | QAPGQGLEWMGYINTYTGEPAYAQGFTGRFVFSLDTSV | |
| | STAYLQISSLKAEDTAVYYCATRAYYGHYLAYWGQGTL | |
| | VTVSS | |
| 331 | QVQLQQSGPGLVKPSQTLSLTCAVSGDSITSGYWNWIR | 3B2_VH_Humanized |
| | QSPSRGLEYMGYISYTGSTYYAVSLKSRITINRDTSKN | |
| | QFSLQLNSVTPEDTAVYYCARKGGWLLPFAYWGQGTLV | |
| | TVSS | |
| 332 | EVQLVESGGGLVKPGGSLRLSCAASGFTFSNYRMHWLR | 3B3_VH_Humanized |
| | QAPGKGLEWVGVITVKSDNYGANYAAPVKGRFTISRDD | |
| | SKNTVYLQMNSLKTEDTAVYYCSLGRYFDYWGQGTTVT | |
| | VSS | |
| 333 | QVQLQESGPGLVKPSQTLSLTCTVSGDSITSGYWNWIR | 3B4_VH_Humanized |
| | QHPGKGLESMGYISYTGSTYYNPSLKSLLTISRDTSKN | |
| | QFSLKLSSVTAADTAVYYCASGLLWLAWFAYWGQGTLV | |
| | TVSS | |
| 334 | EVQLVESGGGLVQPGGSLRLSCAASGFTFSDFYVEWVR | 3B6_VH_Humanized |
| | QAPGKGLEWVAASRNKVNDYTTEYVDSVKGRFTVSRDN | |
| | AKNSLYLQMNSLRAEDTAVYYCARNVPWDGWYFDVWGQ | |
| | GTLVTVSS | |
| 335 | DIVMTQTPPSLPVNPGEPASISCRSSQRIVHSNGNTYL | 3B1_VL_Humanized |
| | EWYLQKPGQSPQLLIYKVSNRFSGVPDRFSGSGSGSDF | |
| | TLKISWVEAEDLGVYYCFQGSHVPYTFGQGTKLEIK | |
| 336 | DIVMTQTPLSLPVTPGEPASMSCRSSQSLLYSTNQKNS | 3B2_VL_Humanized |
| | LAWYLQKPGQSPQLLIYWASTRESGVPDRFSGSGSGTD | |
| | FTLKISRVEAEDLGVYYCQQYYSYPFTFGQGTKLEIK | |
| 337 | DIQMTQSPSSLSASVGDRVTITCQASQGISNYLNWYQQ | 3B3_VL_Humanized |
| | KPGKTVKLLIYYTSSLHSGVPSRFSGSGSGTDFTLTIS | |
| | SLQPEDIATYYCQQYSKLPWTFGQGTKLEIK | |
| 338 | DIVMTQTPLSLPVTPGEPVSMSCRSSQSLLYSSNQKNS | 3B4_VL_Humanized |
| | LAWYLQKPGQSPQLLIYWASTRESGVPDRFSGSGSGTD | |
| | FTLKISRVEAEDLGVYYCQQYYGYPFTFGQGTKLEIK | |
| 339 | AIQLTQSPSSLSASVGDRVTITCRASSSVSYMHWFQQK | 3B5_VL_Humanized |
| | PGKAPKLWIYSTSNLASGVPSRFSGSGSGTDFTLTISS | |
| | MQPEDAATYYCQQRSSYPFTFGQGTKLEIK | |
| 340 | DVVMTQSPLSLPVTLGQPASISCKASQNVRTAVAWFQQ | 3B6_VL_Humanized |
| | RPGQSPRALIYLASTRHTGVPDRFSGSGSGTDFTLKIS | |
| | RVEAEDLGVYFCLQHWNYPRTFGQGTKLEIK | |

The invention claimed is:

1. An antibody or antigen-binding fragment that specifically binds a Programmed Death-Ligand (PD-L1) protein, wherein the antibody or antigen-binding fragment comprises:
(a) a heavy chain variable (VH) region comprising a heavy chain complementarity determining region 1 (CDR-H1), a CDR-H2, and a CDR-H3, respectively, comprising a CDR-H1, a CDR-H2, and a CDR-H3, respectively, contained within the VH region amino acid sequence set forth in SEQ ID NO:1; and a light chain variable (VL) region comprising a light chain complementarity determining region 1 (CDR-L1), a CDR-L2, and a CDR-L3, respectively, comprising a CDR-L1, a CDR-L2, and a CDR-L3, respectively, contained within the VL region amino acid sequence set forth in SEQ ID NO:17;
(b) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:18;
(c) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:3; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:19;
(d) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:4; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:19;
(e) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:5; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:20;
(f) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:1; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:21;
(g) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:2; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:22;
(h) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:6; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:23;
(i) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:7; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:24;
(j) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:8; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:25;
(k) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:9; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:26;
(l) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:10; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:27;
(m) a VH region comprising a CDR-H1 a CDR-H2, and a CDR-H3, respectively, comprising a CDR-H1, a CDR-H2, and a CDR-H3, respectively, contained within the VH region amino acid sequence set forth in SEQ ID NO:11; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:28;
(n) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:12; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:29;
(o) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:13; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:30;
(p) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:14; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:31;
(q) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:15; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:32;
(r) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:12; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:33; or
(s) a VH region comprising a CDR-H1, a CDR-H2, and a CDR-H3, respectively, comprising a CDR-H1, a CDR-H2, and a CDR-H3, respectively, contained within the VH region amino acid sequence set forth in SEQ ID NO:16; and a VL region comprising a CDR-L1, a CDR-L2, and a CDR-L3, respectively, comprising a CDR-L1, a CDR-L2, and a CDR-L3, respectively, contained within the VL region amino acid sequence set forth in SEQ ID NO:34.

2. The antibody or antigen-binding fragment of claim 1, wherein:
(a) the VH region comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:37; and the VL region comprises a CDR-L1 comprising the amino acid sequence set forth in SEQ ID NO:210, a CDR-L2 comprises the amino acid sequence set forth in SEQ ID NO:211, and a CDR-L3 comprising the amino acid sequence set forth in SEQ ID NO:212;
(b) the VH region comprises a CDR-H1 comprising the amino acid sequence set forth in SEQ ID NO:52, a CDR-H2 comprising the amino acid sequence set forth in SEQ ID NO:53, and a CDR-H3 comprising the amino acid sequence set forth in SEQ ID NO:37; and the VL region comprises a CDR-L1 comprising the amino acid sequence set forth in SEQ ID NO:218, a CDR-L2 comprises the amino acid sequence set forth in SEQ ID NO:211, and a CDR-L3 comprising the amino acid sequence set forth in SEQ ID NO:212;
(c) the VH region comprises a CDR-H1 comprising the amino acid sequence set forth in SEQ ID NO:63, a CDR-H2 comprising the amino acid sequence set forth in SEQ ID NO:64, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(d) the VH region comprises a CDR-H1 comprising the amino acid sequence set forth in SEQ ID NO:63, a CDR-H2 comprising the amino acid sequence set forth in SEQ ID NO:64, and a CDR-H3 comprising the amino acid sequence set forth in SEQ ID NO:60; and the VL region comprises a CDR-L1 comprising the amino acid sequence set forth in SEQ ID NO:221, a CDR-L2 comprises the amino acid sequence set forth in SEQ ID NO:222, and a CDR-L3 comprising the amino acid sequence set forth in SEQ ID NO:223;
(e) the VH region comprises a CDR-H1 comprising the amino acid sequence set forth in SEQ ID NO:75, a CDR-H2 comprising the amino acid sequence set forth in SEQ ID NO:76, and a CDR-H3 comprising the amino acid sequence set forth in SEQ ID NO:60; and the VL region comprises a CDR-L1 comprising the amino acid sequence set forth in SEQ ID NO:229, a CDR-L2 comprises the amino acid sequence set forth in SEQ ID NO:222, and a CDR-L3 comprising the amino acid sequence set forth in SEQ ID NO:223;
(f) the VH region comprises a CDR-H1 comprising the amino acid sequence set forth in SEQ ID NO:40, a CDR-H2 comprising the amino acid sequence set forth in SEQ ID NO:41, and a CDR-H3 comprising the amino acid sequence set forth in SEQ ID NO:37; and the VL region comprises a CDR-L1 comprising the amino acid sequence set forth in SEQ ID NO:233, a CDR-L2 comprises the amino acid sequence set forth in SEQ ID NO:234, and a CDR-L3 comprising the amino acid sequence set forth in SEQ ID NO:235;
(g) the VH region comprises a CDR-H1 comprising the amino acid sequence set forth in SEQ ID NO:52, a CDR-H2 comprising the amino acid sequence set forth in SEQ ID NO:53, and a CDR-H3 comprising the amino acid sequence set forth in SEQ ID NO:37; and the VL region comprises a CDR-L1 comprising the amino acid sequence set forth in SEQ ID NO:241, a CDR-L2 comprises the amino acid sequence set forth in SEQ ID NO:234, and a CDR-L3 comprising the amino acid sequence set forth in SEQ ID NO:242;
(h) the VH region comprises a CDR-H1 comprising the amino acid sequence set forth in SEQ ID NO:87, a CDR-H2 comprising the amino acid sequence set forth in SEQ ID NO:88, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:247, and a CDR-L3 comprising the amino acid sequence set forth in SEQ ID NO:248;
(i) the VH region comprises a CDR-H1 comprising the amino acid sequence set forth in SEQ ID NO:98, a CDR-H2 comprising the amino acid sequence set forth in SEQ ID NO:99, and a CDR-H3 comprising the amino acid sequence set forth in SEQ ID NO:84; and the VL region comprises a CDR-L1 comprising the amino acid sequence set forth in SEQ ID NO:246, a CDR-L2 comprises the amino acid sequence set forth in SEQ ID NO:254, and a CDR-L3 comprising the amino acid sequence set forth in SEQ ID NO:255;
(j) the VH region comprises a CDR-H1 comprising the amino acid sequence set forth in SEQ ID NO:108, a CDR-H2 comprising the amino acid sequence set forth in SEQ ID NO:109, and a CDR-H3 comprising the amino acid sequence set forth in SEQ ID NO:105; and the VL region comprises a CDR-L1 comprising the amino acid sequence set forth in SEQ ID NO:258, a CDR-L2 comprises the amino acid sequence set forth in SEQ ID NO:259, and a CDR-L3 comprising the amino acid sequence set forth in SEQ ID NO:260;
(k) the VH region comprises a CDR-H1 comprising the amino acid sequence set forth in SEQ ID NO:121, a CDR-H2 comprising the amino acid sequence set forth in SEQ ID NO:122, and a CDR-H3 comprising the amino acid sequence set forth in SEQ ID NO:118; and the VL region comprises a CDR-L1 comprising the amino acid sequence set forth in SEQ ID NO:265, a CDR-L2 comprises the amino acid sequence set forth in SEQ ID NO:266, and a CDR-L3 comprising the amino acid sequence set forth in SEQ ID NO:267;
(l) the VH region comprises a CDR-H1 comprising the amino acid sequence set forth in SEQ ID NO:134, a CDR-H2 comprising the amino acid sequence set forth in SEQ ID NO:135, and a CDR-H3 comprising the amino acid sequence set forth in SEQ ID NO:131; and the VL region comprises a CDR-L1 comprising the amino acid sequence set forth in SEQ ID NO:246, a CDR-L2 comprises the amino acid sequence set forth in SEQ ID NO:273, and a CDR-L3 comprising the amino acid sequence set forth in SEQ ID NO:274;
(m) the VH region comprises a CDR-H1 comprising the amino acid sequence set forth in SEQ ID NO:147, a CDR-H2 comprising the amino acid sequence set forth in SEQ ID NO:148, and a CDR-H3 comprising the amino acid sequence set forth in SEQ ID NO:144; and the VL region comprises a CDR-L1 comprising the amino acid sequence set forth in SEQ ID NO:278, a CDR-L2 comprises the amino acid sequence set forth in SEQ ID NO:279, and a CDR-L3 comprising the amino acid sequence set forth in SEQ ID NO:280;
(n) the VH region comprises a CDR-H1 comprising the amino acid sequence set forth in SEQ ID NO:160, a CDR-H2 comprising the amino acid sequence set forth in SEQ ID NO:161, and a CDR-H3 comprising the amino acid sequence set forth in SEQ ID NO:157; and the VL region comprises a CDR-L1 comprising the amino acid sequence set forth in SEQ ID NO:286, a CDR-L2 comprises the amino acid sequence set forth in SEQ ID NO:287, and a CDR-L3 comprising the amino acid sequence set forth in SEQ ID NO:288;
(o) the VH region comprises a CDR-H1 comprising the amino acid sequence set forth in SEQ ID NO:173, a CDR-H2 comprising the amino acid sequence set forth in SEQ ID NO:174, and a CDR-H3 comprising the amino acid sequence set forth in SEQ ID NO:170; and the VL region comprises a CDR-L1 comprising the amino acid sequence set forth in SEQ ID NO:294, a CDR-L2 comprises the amino acid sequence set forth in SEQ ID NO:234, and a CDR-L3 comprising the amino acid sequence set forth in SEQ ID NO:295;
(p) the VH region comprises a CDR-H1 comprising the amino acid sequence set forth in SEQ ID NO:185, a CDR-H2 comprising the amino acid sequence set forth in SEQ ID NO:186, and a CDR-H3 comprising the amino acid sequence set forth in SEQ ID NO:182; and the VL region comprises a CDR-L1 comprising the amino acid sequence set forth in SEQ ID NO:299, a CDR-L2 comprises the amino acid sequence set forth in SEQ ID NO:300, and a CDR-L3 comprising the amino acid sequence set forth in SEQ ID NO:301;
(q) the VH region comprises a CDR-H1 comprising the amino acid sequence set forth in SEQ ID NO:173, a CDR-H2 comprising the amino acid sequence set forth in SEQ ID NO:174, and a CDR-H3 comprising the amino acid sequence set forth in SEQ ID NO: 193; and the VL region comprises a CDR-L1 comprising the amino acid sequence set forth in SEQ ID NO:306, a CDR-L2 comprises the amino acid sequence set forth in SEQ ID NO:234, and a CDR-L3 comprising the amino acid sequence set forth in SEQ ID NO:307;
(r) the VH region comprises a CDR-H1 comprising the amino acid sequence set forth in SEQ ID NO:160, a CDR-H2 comprising the amino acid sequence set forth in SEQ ID NO:161, and a CDR-H3 comprising the amino acid sequence set forth in SEQ ID NO:157; and the VL region comprises a CDR-L1 comprising the amino acid sequence set forth in SEQ ID NO:311, a CDR-L2 comprises the amino acid sequence set forth in SEQ ID NO:312, and a CDR-L3 comprising the amino acid sequence set forth in SEQ ID NO:313; or
(s) the VH region comprises a CDR-H1 comprising the amino acid sequence set forth in SEQ ID NO:202, a CDR-H2 comprising the amino acid sequence set forth in SEQ ID NO:203, and a CDR-H3 comprising the amino acid sequence set forth in SEQ ID NO:199; and the VL region comprises a CDR-L1 comprising the amino acid sequence set forth in SEQ ID NO:319, a CDR-L2 comprises the amino acid sequence set forth in SEQ ID NO:320, and a CDR-L3 comprising the amino acid sequence set forth in SEQ ID NO:321.

3. The antibody or antigen-binding fragment of claim 1 or 2, wherein:
(a) the VH region comprises an amino acid sequence that has at least 95% sequence identity to the amino acid sequence set forth in SEQ ID NO:1; and the VL region comprises an amino acid sequence that has at least 95% sequence identity to the amino acid sequence set forth in SEQ ID NO:17;
(b) the VH region comprises an amino acid sequence that has at least 95% sequence identity to the amino acid sequence set forth in SEQ ID NO:2; and the VL region comprises an amino acid sequence that has at least 95% sequence identity to the amino acid sequence set forth in SEQ ID NO:18;
(c) the VH region comprises an amino acid sequence that has at least 95% sequence identity to the amino acid sequence set forth in SEQ ID NO:3; and the VL region comprises an amino acid sequence that has at least 95% sequence identity to the amino acid sequence set forth in SEQ ID NO:19;
(d) the VH region comprises an amino acid sequence that has at least 95% sequence identity to the amino acid sequence set forth in SEQ ID NO:4; and the VL region comprises an amino acid sequence that has at least 95% sequence identity to the amino acid sequence set forth in SEQ ID NO:19;
(e) the VH region comprises an amino acid sequence that has at least 95% sequence identity to the amino acid sequence set forth in SEQ ID NO:5; and the VL region comprises an amino acid sequence that has at least 95% sequence identity to the amino acid sequence set forth in SEQ ID NO:20;
(f) the VH region comprises an amino acid sequence that has at least 95% sequence identity to the amino acid sequence set forth in SEQ ID NO:1; and the VL region comprises an amino acid sequence that has at least 95% sequence identity to the amino acid sequence set forth in SEQ ID NO:21;
(g) the VH region comprises an amino acid sequence that has at least 95% sequence identity to the amino acid sequence set forth in SEQ ID NO:2; and the VL region comprises an amino acid sequence that has at least 95% sequence identity to the amino acid sequence set forth in SEQ ID NO:22;
(h) the VH region comprises an amino acid sequence that has at least 95% sequence identity to the amino acid sequence set forth in SEQ ID NO:6; and the VL region comprises an amino acid sequence that has at least 95% sequence identity to the amino acid sequence set forth in SEQ ID NO:23;
(i) the VH region comprises an amino acid sequence that has at least 95% sequence identity to the amino acid sequence set forth in SEQ ID NO:7; and the VL region comprises an amino acid sequence that has at least 95% sequence identity to the amino acid sequence set forth in SEQ ID NO:24;
(j) the VH region comprises an amino acid sequence that has at least 95% sequence identity to the amino acid sequence set forth in SEQ ID NO:8; and the VL region comprises an amino acid sequence that has at least 95% sequence identity to the amino acid sequence set forth in SEQ ID NO:25;
(k) the VH region comprises an amino acid sequence that has at least 95% sequence identity to the amino acid sequence set forth in SEQ ID NO:9; and the VL region comprises an amino acid sequence that has at least 95% sequence identity to the amino acid sequence set forth in SEQ ID NO:26;
(l) the VH region comprises an amino acid sequence that has at least 95% sequence identity to the amino acid sequence set forth in SEQ ID NO:10; and the VL region comprises an amino acid sequence that has at least 95% sequence identity to the amino acid sequence set forth in SEQ ID NO:27;
(m) the VH region comprises an amino acid sequence that has at least 95% sequence identity to the amino acid sequence set forth in SEQ ID NO:11; and the VL region comprises an amino acid sequence that has at least 95% sequence identity to the amino acid sequence set forth in SEQ ID NO:28;
(n) the VH region comprises an amino acid sequence that has at least 95% sequence identity to the amino acid sequence set forth in SEQ ID NO:12; and the VL region comprises an amino acid sequence that has at least 95% sequence identity to the amino acid sequence set forth in SEQ ID NO:29;
(o) the VH region comprises an amino acid sequence that has at least 95% sequence identity to the amino acid sequence set forth in SEQ ID NO:13; and the VL region comprises an amino acid sequence that has at least 95% sequence identity to the amino acid sequence set forth in SEQ ID NO:30;
(p) the VH region comprises an amino acid sequence that has at least 95% sequence identity to the amino acid sequence set forth in SEQ ID NO:14; and the VL region comprises an amino acid sequence that has at least 95% sequence identity to the amino acid sequence set forth in SEQ ID NO:31;
(q) the VH region comprises an amino acid sequence that has at least 95% sequence identity to the amino acid sequence set forth in SEQ ID NO:15; and the VL region comprises an amino acid sequence that has at least 95% sequence identity to the amino acid sequence set forth in SEQ ID NO:32;
(r) the VH region comprises an amino acid sequence that has at least 95% sequence identity to the amino acid sequence set forth in SEQ ID NO:12; and the VL region comprises an amino acid sequence that has at least 95% sequence identity to the amino acid sequence set forth in SEQ ID NO:33; or
(s) the VH region comprises an amino acid sequence that has at least 95% sequence identity to the amino acid sequence set forth in SEQ ID NO:16; and the VL region comprises an amino acid sequence that has at least 95% sequence identity to the amino acid sequence set forth in SEQ ID NO:34.

4. The antibody or antigen-binding fragment of any of claims 1-3, wherein:
(a) the VH region comprises the amino acid sequence set forth in SEQ ID NO:1; and the VL region comprises the amino acid sequence set forth in SEQ ID NO:17;
(b) the VH region comprises the amino acid sequence set forth in SEQ ID NO:2; and the VL region comprises the amino acid sequence set forth in SEQ ID NO:18;
(c) the VH region comprises the amino acid sequence set forth in SEQ ID NO:3; and the VL region comprises the amino acid sequence set forth in SEQ ID NO:19;
(d) the VH region comprises the amino acid sequence set forth in SEQ ID NO:4; and the VL region comprises the amino acid sequence set forth in SEQ ID NO:19;
(e) the VH region comprises the amino acid sequence set forth in SEQ ID NO:5; and the VL region comprises the amino acid sequence set forth in SEQ ID NO:20;
(f) the VH region comprises the amino acid sequence set forth in SEQ ID NO:1; and the VL region comprises the amino acid sequence set forth in SEQ ID NO:21;
(g) the VH region comprises the amino acid sequence set forth in SEQ ID NO:2; and the VL region comprises the amino acid sequence set forth in SEQ ID NO:22;
(h) the VH region comprises the amino acid sequence set forth in SEQ ID NO:6; and the VL region comprises the amino acid sequence set forth in SEQ ID NO:23;
(i) the VH region comprises the amino acid sequence set forth in SEQ ID NO:7; and the VL region comprises the amino acid sequence set forth in SEQ ID NO:24;
(j) the VH region comprises the amino acid sequence set forth in SEQ ID NO:8; and the VL region comprises the amino acid sequence set forth in SEQ ID NO:25;
(k) the VH region comprises the amino acid sequence set forth in SEQ ID NO:9; and the VL region comprises the amino acid sequence set forth in SEQ ID NO:26;
(l) the VH region comprises the amino acid sequence set forth in SEQ ID NO:10; and the VL region comprises the amino acid sequence set forth in SEQ ID NO:27;
(m) the VH region comprises the amino acid sequence set forth in SEQ ID NO:11; and the VL region comprises the amino acid sequence set forth in SEQ ID NO:28;
(n) the VH region comprises the amino acid sequence set forth in SEQ ID NO:12; and the VL region comprises the amino acid sequence set forth in SEQ ID NO:29;
(o) the VH region comprises the amino acid sequence set forth in SEQ ID NO: 13; and the VL region comprises the amino acid sequence set forth in SEQ ID NO:30;
(p) the VH region comprises the amino acid sequence set forth in SEQ ID NO:14; and the VL region comprises the amino acid sequence set forth in SEQ ID NO:31;
(q) the VH region comprises the amino acid sequence set forth in SEQ ID NO:15; and the VL region comprises the amino acid sequence set forth in SEQ ID NO:32;
(r) the VH region comprises the amino acid sequence set forth in SEQ ID NO:12; and the VL region comprises the amino acid sequence set forth in SEQ ID NO:33; or
(s) the VH region comprises the amino acid sequence set forth in SEQ ID NO:16; and the VL region comprises the amino acid sequence set forth in SEQ ID NO:34.

5. The antibody or antigen-binding fragment of any of claims 1-4, wherein the PD-L1 protein is a human PD-L1 protein.

6. The antibody or antigen-binding fragment of any of claims 1-5, wherein the antibody or antigen-binding fragment is recombinant.

7. The antibody or antigen-binding fragment of any of claims 1-6, wherein the antibody or antigen-binding fragment is monoclonal.

8. The antibody or antigen-binding fragment of any of claims 1-7, wherein the antibody or antigen-binding fragment is a human, chimeric, or humanized antibody or antigen-binding fragment.

9. The antibody or antigen-binding fragment of any of claims 1-8, wherein the antibody or antigen binding fragment comprises an Fc region of a human immunoglobulin and/or human antibody framework regions.

10. The antibody or antigen-binding fragment of any of claims 1-9, that is a single chain antibody fragment.

11. The antibody or antigen-binding fragment of claim 10, wherein the antibody fragment comprises a single chain Fv (scFv).

12. The antibody or antigen-binding fragment of any of claims 1-11, that is a whole or intact antibody.

13. The antibody or antigen-binding fragment of any of claims 1-12, that is a bispecific antibody that further specifically binds to a second antigen.

14. The antibody or antigen-binding fragment of claim 13, wherein the second antigen is an antigen expressed on a tumor cell or an immune cell.

15. The antibody or antigen-binding fragment of claim 13 or 14, wherein the second antigen is CD25.

16. The antibody or antigen-binding fragment of any of claims 1-15, wherein the antibody or antigen-binding fragment thereof comprises an Fc region that exhibits one or more Fc-mediated effector function(s).

17. The antibody or antigen-binding fragment of any of claims 1-15, wherein the antibody or antigen-binding fragment thereof comprises an Fc region that lacks Fc-mediated effector function(s), exhibits substantially reduced Fc-mediated effector function(s) or does not exhibit substantial Fc-mediated effector function(s).

18. The antibody or antigen-binding fragment of any of claims 1-15, wherein the antibody or antigen-binding fragment thereof comprises an Fc region that exhibits enhanced Fc-mediated effector function(s).

19. The antibody or antigen-binding fragment of any of claims 16-18, wherein the Fc-mediated effector function is selected from one or more of an antibody-dependent cellular cytotoxicity (ADCC), antibody-dependent cellular phagocytosis (ADCP) or complement-dependent cytotoxicity (CDC).

20. The antibody or antigen-binding fragment of any of claims 1-19, wherein the antibody or antigen binding fragment comprises an IgG1 Fc region or an IgG1 isotype, an IgG2 Fc region or an IgG2 isotype, IgG3 Fc region or an IgG3 isotype, or an IgG4 Fc region or an IgG4 isotype.

21. A conjugate, comprising the antibody or antigen-binding fragment of any of claims 1-20 and a heterologous molecule or moiety.

22. The conjugate of claim 21, wherein the heterologous molecule or moiety is a protein, peptide, nucleic acid, dye, or small molecule.

23. The conjugate of claim 21 or 22, wherein the heterologous molecule or moiety is a cytotoxic agent, a toxin, a radioisotope, a chemotherapeutic agent, a lytic peptide, a cytokine, or a photoactivatable dye.

24. The conjugate of claim 23, wherein the photoactivatable dye is a phthalocyanine dye.

25. The conjugate of claim 24, wherein the phthalocyanine dye is a Si-phthalocyanine dye.

26. The conjugate of claim 24 or 25, wherein the phthalocyanine dye is IR700.

27. The conjugate of claim 24 or 25, wherein the phthalocyanine dye has the structure of Formula (I): 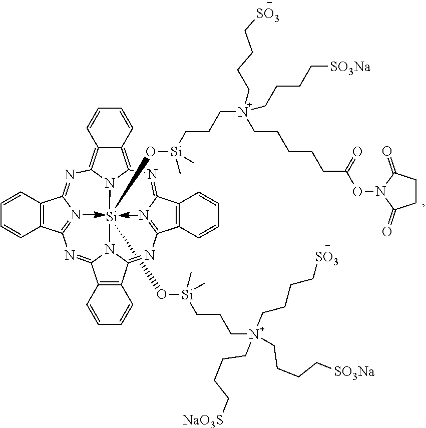
or is a salt, stereoisomer, or tautomer thereof.

28. The conjugate of any of claims 21-27, wherein the conjugate is activated by illumination at a wavelength between at or at about 600 nm and at or about 850 nm to effect cell killing.

29. The conjugate of claim 28, wherein the activated conjugate affects tumor growth inhibition or killing at a higher level, activity, or potency than the unconjugated antibody.

30. The conjugate of any of claims 21-29, wherein the antibody or antigen-binding fragment and the moiety are linked directly or indirectly via a linker.

31. The conjugate of any of claims 21-29, wherein the antibody or antigen-binding fragment is covalently attached to the heterologous molecule or moiety.

32. The conjugate of any of claims 21-31, wherein when contacted with a cell expressing a PD-L1 protein, the conjugate exhibits increased internalization compared to an unconjugated antibody or antigen-binding fragment, or a conjugate comprising a reference antibody.

33. The conjugate of any of claims 21-31, wherein when contacted with a cell expressing a PD-L1 protein, the conjugate exhibits reduced internalization compared to an unconjugated antibody or antigen-binding fragment, or a conjugate comprising a reference antibody.

34. The conjugate of claim 32 or 33, wherein the reference antibody is avelumab.

35. The conjugate of any of claims 21-34, wherein the conjugate does not exhibit substantially reduced binding affinity to a PD-L1 protein compared to the unconjugated antibody, or exhibits at least 50%, at least 60%, at least 70%, at least 80%, at least 90%, at least 95%, at least 96%, at least 97%, at least 98%, or at least 99% of the binding affinity of the unconjugated antibody to the PD-L1 protein.

36. A polynucleotide encoding the antibody or antigen-binding fragment of any of claims 1-20.

37. A vector, comprising the polynucleotide of claim 36.

38. The vector of claim 37, wherein the vector is an expression vector.

39. An engineered cell comprising the vector of claim 37 or 38.

40. An engineered cell expressing the antibody or antigen binding fragment of any of claims 1-20.

41. A composition comprising the antibody or antigen-binding fragment thereof of any of claims 1-20, or the conjugate of any of claims 21-35.

42. The composition of claim 41, further comprising a pharmaceutically acceptable excipient.

43. A method of treatment, comprising administering the antibody or antigen-binding fragment thereof of any of claims 1-20, the conjugate of any of claims 21-35, or the composition of claim 41 or 42 to a subject having a disease or disorder.

44. The method of claim 43, wherein the disease or disorder is a tumor or a cancer.

45. A method of treating a tumor or a lesion in a subject, comprising:
a) administering to the subject the conjugate of any of claims 21-35 or the composition of claim 41 or 42; and
b) illuminating a target area within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.

46. The method of any of claims 43-45, wherein the method results in the killing of a PD-L1-expressing cell in the target area.

47. The method of any of claims 43-46, wherein the subject has a tumor or lesion that has had a low response to, was unresponsive to, was resistant to, was refractory to, had failed to respond to or has relapsed after, a prior immunotherapy.

48. The method of claim 47, wherein the prior immunotherapy is a treatment with an immune checkpoint inhibitor.

49. The method of claim 47 or 48, wherein the subject has primary resistance or acquired resistance to a prior immunotherapy that comprises a PD-1:PD-L1 blockade therapy.

50. The method of any of claims 43-49, wherein the subject is treatment-naïve for an immune checkpoint inhibitor or that has not previously received a treatment with an immune checkpoint inhibitor.

51. The method of any of claims 43-50, wherein the subject is administered the conjugate to treat, inhibit the growth of and/or reduce the size of a first tumor or lesion; and the method inhibits, delays or prevents the appearance, growth or establishment of one or more second tumors or lesions, located distally to the first tumor or lesion.

52. A method of immunizing a subject having a first tumor or lesion, the method comprising:
(a) administering to the subject the conjugate of any of claims 21-35 or the composition of claim 41 or 42 to a subject having a tumor or lesion; and
(b) illuminating a target area within the first tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein the first tumor or lesion is inhibited in growth and/or reduced in size; and the appearance, growth or establishment of one or more second tumors or lesions, located distally to the treated first tumor or lesion, is inhibited, delayed or prevented.

53. The method of claim 51 or 52, wherein the second tumor or lesion is a metastasis of the first tumor or lesion.

54. The method of any of claims 46-53, wherein the PD-L1 expressing cell is an immune cell.

55. The method of claim 54, wherein the immune cell is a monocyte, a macrophage, a dendritic cell (DC), or a myeloid-derived suppressor cell (MDSC).

56. The method of any of claims 45-55, wherein the illuminating is carried out between 30 minutes and 96 hours after administering the conjugate or at or about 24 hours±4 hours after administering the conjugate.

57. The method of any of claims 45-56, wherein the target area is illuminated at a wavelength of 690±40 nm.

58. The method of any of claims 45-57, wherein the target area is illuminated at a wavelength of 670±50 nm.

59. The method of any of claims 45-58, wherein the target area is illuminated at a dose of at or about of 50 J/cm² or at or about 100 J/cm of fiber length.

60. The method of any of claims 44-59, wherein the tumor, lesion or cancer is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

61. The method of any of claims 43-60, wherein one or more of steps of the method are repeated.

62. The method of any of claims 43-61, wherein the administration of the antibody or antigen-binding fragment, the conjugate or the composition is repeated one or more times, optionally wherein after each repeated administration of the conjugate or the composition, the illuminating step is repeated.

63. The method of any of claims 43-62, further comprising administering an additional therapeutic agent or anti-cancer treatment.


SEQUENCE LISTING (340 sequences)

SEQ ID NO: 1 - protein, 116 aa, synthetic construct
  REGION 1..116: note = 1P1/1P4_VH
QVQLVESGGGVVQPGRSLRLSCAASGFTFSNYGMHWVRQAPGKGLEWVSLILYDGSNKYYADSVKGRFTISRDNSKNTLYLQMNSLRAEDTAVYYCARDRVWLDVWGKGTTVTVSS

SEQ ID NO: 2 - protein, 116 aa, synthetic construct
  REGION 1..116: note = 1P1.2/1P4.2_VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDRVWLDVWGKGTTVTVSS

SEQ ID NO: 3 - protein, 116 aa, synthetic construct
  REGION 1..116: note = 1P2_VH
QVQLVQSGAEVKKPGASVKISCKASGFTFSRYDFNWVRQATGQGLEWMGWMNPNNGNTGYSQKFQGRVTMTRNTSISTAYMELSSLRSEDTAVYYCARGWTFFDYWGQGTLVTVSS

SEQ ID NO: 4 - protein, 116 aa, synthetic construct
  REGION 1..116: note = 1P2_N73D_VH
QVQLVQSGAEVKKPGASVKISCKASGFTFSRYDFNWVRQATGQGLEWMGWMNPNNGNTGYSQKFQGRVTMTRDTSISTAYMELSSLRSEDTAVYYCARGWTFFDYWGQGTLVTVSS

SEQ ID NO: 5 - protein, 116 aa, synthetic construct
  REGION 1..116: note = 1P2.2_VH
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGWTFFDYWGQGTLVTVSS

SEQ ID NO: 6 - protein, 119 aa, synthetic construct
  REGION 1..119: note = 1P9_VH
EVQLVESGGGLVQPGGSLRLSCAASGFTFSTYWMSWVRQAPGKGLEWVANIKQDGGERYYVDSVKGRFTISRDNAKSSLYLQMNSLRAEDTALYYCARLRLYSDYEDYWGQGTLVTVSS

SEQ ID NO: 7 - protein, 119 aa, synthetic construct
  REGION 1..119: note = 1P9.2_VH
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARLRLYSDYEDYWGQGTLVTVSS

SEQ ID NO: 8 - protein, 122 aa, synthetic construct
  REGION 1..122: note = 2M1_VH
EVQLVESGGGLVKPGGSLRLSCAASGFTFSSYTMNWVRQAPGKGLEWVSSITSSTSYIYYADSVKGRFTISRDNAKNSLYLQMNSLRAEDTSVYYCVRDWSIAARGAWFDPWGQGTLVTVSS

SEQ ID NO: 9 - protein, 115 aa, synthetic construct
  REGION 1..115: note = 2M2_VH
QVQLVQSGAEVKKPGASVKVSCKASGYTFTNYGISWVRQAPGQGLEWMGWISAYNGHTNYAQKVQGRVTMTTDTSTSTAFMELRSLRSDDSAVYYCARGTSGDYWGQGTLVTVSS

SEQ ID NO: 10 - protein, 118 aa, synthetic construct
  REGION 1..118: note = 2M3_VH
EVQLVESGGGFVQTGGSLRLSCAASGFNFRSYDMHWVRQVIGKGLEWVSGIGPGGDTYYPGSVKGRFTISRENAKNSLYLQMNSLRAGDTAVYYCAREQFEGHYFDYWGQGTLVTVSS

SEQ ID NO: 11 - protein, 121 aa, synthetic construct
  REGION 1..121: note = 2M5_VH
EVQLVESGGGLVKPGGSLRVSCAASGFTLSSYSMNWVRQAPGKGLEWVSSISSSSSYIYYADSVKGRFTISRDNAKNLLYLQMNSLRAEDTAVYYCAREGGAARPSWFDPWGQGTLVTVSS

SEQ ID NO: 12 - protein, 119 aa, synthetic construct
  REGION 1..119: note = 3B1_3B5_VH
QIQLVQSGPELKKPGETVKISCKASGYDFTNYGMNWVKQAPGKGLKWMGYINTYTGEPAYADDFKGRFAFSLETSARTAYLRINNLKNEDTATYFCATRAYYGHYLAYWGQGTLVTVSA

SEQ ID NO: 13 - protein, 118 aa, synthetic construct
  REGION 1..118: note = 3B2_VH
EVQLQESGPSLVKPSQTLSLTCSVTGDSITSGYWNWIRKFPGNKLEYMGYISYTGSTYYNPSLKSRISITRDTSKNQYYLQLDSVTTEDTATYYCARKGGWLLPFAYWGQGTLVTVSA

SEQ ID NO: 14 - protein, 117 aa, synthetic construct
  REGION 1..117: note = 3B3_VH
QVQLVETGGGLVRPGNSLKLSCVTSGFTFSNYRMHWLRQPPGKRLEWIAVITVKSDNYGANYAESVKGRFTISRDDSKSSVYLQMNRLREEDTATYYCSLGRYFDYWGQGTTLTVSS

SEQ ID NO: 15 - protein, 118 aa, synthetic construct
  REGION 1..118: note = 3B4_VH
EVQLQESGPSLVKPSQTLSLTCSVTGDSITSGYWNWIRKFPGNKLESMGYISYTGSTYYNPSLKSRLSITRDTSKNQFYLQLNSVTTEDTATYYCASGLLWLAWFAYWGQGTLVTVSA

SEQ ID NO: 16 - protein, 122 aa, synthetic construct
  REGION 1..122: note = 3B4_VH
EVKLVESGGGLVQPGGSLRLSCGTSGFTFSDFYVEWVRQPPGKRLEWIAASRNKVNDYTTEYSESVKGRFIVSRDTSQNILYLQMNALRAEDSAIYYCARNVPWDGWYFDVWGAGTAVTVSS

SEQ ID NO: 17 - protein, 107 aa, synthetic construct
  REGION 1..107: note = 1P1_VL
DIQMTQSPSSLSASVGDRVTITCRASQGIRNYLAWYQQKPGKVPKLLIYAASTLQSGVPSRFSGSGSGTDFTLTISGLQPEDVATYYCQKYNSALWTFGQGTKVEIK

SEQ ID NO: 18 - protein, 107 aa, synthetic construct
  REGION 1..107: note = 1P1.2_VL
DIQMTQSPSSLSASVGDRVTITCRASQGISNYLAWYQQKPGKVPKLLIYAASTLQSGVPSRFSGSGSGTDFTLTISSLQPEDVATYYCQKYNSALWTFGQGTKVEIK

SEQ ID NO: 19 - protein, 107 aa, synthetic construct
  REGION 1..107: note = 1P2_VL
EIVLTQSPGTLSLSPGERATLSCRASQSVSSTYLAWYQQKPGQAPRLLIFGASSRATGIPDRFSGSGSGTDFTLTISRLEPEDFAVYYCQQYGSSPTFGQGTKVEIK

SEQ ID NO: 20 - protein, 107 aa, synthetic construct
  REGION 1..107: note = 1P2.2_VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKVEIK

SEQ ID NO: 21 - protein, 113 aa, synthetic construct
  REGION 1..113: note = 1P4_VL
DIVMTQSPDSLAVSLGERATINCKSSQSVLYRSSNKNYLAWYQQKPGQPPKLLIYWASTRESGVPDRFSGSGSGTDFTLTISSLQAEDVAVYYCHQYYTTPLTFGGGTKVEIR

SEQ ID NO: 22 - protein, 113 aa, synthetic construct
  REGION 1..113: note = 1P4.2_VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIR

SEQ ID NO: 23 - protein, 107 aa, synthetic construct
  REGION 1..107: note = 1P9_VL
DIQMTQSPSSVSASVGDRVTITCRASQGISSWLAWYQQKPGKAPKLLIYSVSSLQSGVPSRFSGSGSGTDFTLTISSLQPEDSATYYCQQANSLPPTFGGGTKVEIK

SEQ ID NO: 24 - protein, 107 aa, synthetic construct
  REGION 1..107: note = 1P9.2_VL
DIQMTQSPSSVSASVGDRVTITCRASQGISSWLAWYQQKPGKAPKLLIYAASSLQSGVPSRFSGSGSGTDFTLTISSLQPEDFATYYCQQANSFPPTFGGGTKVEIK

SEQ ID NO: 25 - protein, 107 aa, synthetic construct
  REGION 1..107: note = 2M1_VL
DIQMTQSPSSVSASVGDRVTITCRASQGIGSWLAWYQQKPGKAPKFLIYAASSLQGGVPSRFSGSGSGTDFTLTISSLQPEDFATYYCQQANSFPLTFGGGTKVEIK

SEQ ID NO: 26 - protein, 108 aa, synthetic construct
  REGION 1..108: note = 2M2_VL
EIVLTQSPATLSLSPGERATLSCRASQSVSSYLAWYQQKPGQAPRLLIYDASNRATGIPGRFSGSGSGTDFTLTISSLEPEDFAVYYCQQRSNWPPLTFGGGTKVEIK

SEQ ID NO: 27 - protein, 107 aa, synthetic construct
  REGION 1..107: note = 2M3_VL
DIQMTQSPSSVSASVGDRVNITCRASQGISSWLAWYQQKPWKAPKLLIYTASSLQSGVPSRFSGSGSGTDFTLTISSLQPEDFATYYCQQANSFPITFGQGTRLEIK

SEQ ID NO: 28 - protein, 109 aa, synthetic construct
  REGION 1..109: note = 2M5_VL
QAVVTQESALTTSPGETVTLTCRSSTGAVTTSNYANWVQEKPDHLFTGLIGGTNNRAPGVPARFSGSLIGDKAALTITGAQTEDEAIYFCALWYRNHWVFGGGTKLTVL

SEQ ID NO: 29 - protein, 112 aa, synthetic construct
  REGION 1..112: note = 3B1_VL
DVLMTQTPLSLPVSLGDQASISCRSSQRIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 30 - protein, 113 aa, synthetic construct
  REGION 1..113: note = 3B2_VL
DIVMSQSPSSLAVSFGEKVTMSCKSSQSLLYSTNQKNSLAWYQQKPGQSPKLLIYWASTRESGVPDRFTGSGSGTDFTLTISSVKAEDLAVYYCQQYYSYPFTFGSGTKLEIK

SEQ ID NO: 31 - protein, 107 aa, synthetic construct
  REGION 1..107: note = 3B3_VL
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 32 - protein, 113 aa, synthetic construct
  REGION 1..113: note = 3B4_VL
DIVMSQSPSSLAVSVGEKVTMSCKSSQSLLYSSNQKNSLAWYQQKPGQSPKLLIYWASTRESGVPDRFTGSGSGTDFTLTISSVRAEDLAVYYCQQYYGYPFTFGGGTKLEIK

SEQ ID NO: 33 - protein, 106 aa, synthetic construct
  REGION 1..106: note = 3B5_VL
QIVLTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 34 - protein, 107 aa, synthetic construct
  REGION 1..107: note = 3B6_VL
DIVMTQSQKFMSTSVGDRVSITCKASQNVRTAVAWFQQKPGQSPKALIYLASTRHTGVPDRFTGSGSGTDFTLTISNVQSEDLADYFCLQHWNYPRTFGGGTKLEIK

SEQ ID NO: 35 - protein, 7 aa, synthetic construct
  REGION 1..7: note = 1P1/1P4_CDR-H1_Chothia
GFTFSNY

SEQ ID NO: 36 - protein, 6 aa, synthetic construct
  REGION 1..6: note = 1P1/1P4_CDR-H2_Chothia
LYDGSN

SEQ ID NO: 37 - protein, 7 aa, synthetic construct
  REGION 1..7: note = 1P1/1P4/1P1.2/1P4.2_CDR-H3_Chothia_AbM_Kabat
DRVWLDV

SEQ ID NO: 38 - protein, 10 aa, synthetic construct
  REGION 1..10: note = 1P1/1P4_CDR-H1_AbM
GFTFSNYGMH

SEQ ID NO: 39 - protein, 10 aa, synthetic construct
  REGION 1..10: note = 1P1/1P4_CDR-H2_AbM
LILYDGSNKY

SEQ ID NO: 40 - protein, 5 aa, synthetic construct
  REGION 1..5: note = 1P1/1P4_CDR-H1_Kabat
NYGMH

SEQ ID NO: 41 - protein, 17 aa, synthetic construct
  REGION 1..17: note = 1P1/1P4_CDR-H2_Kabat
LILYDGSNKYYADSVKG

SEQ ID NO: 42 - protein, 6 aa, synthetic construct
  REGION 1..6: note = 1P1/1P4_CDR-H1_Contact
SNYGMH

SEQ ID NO: 43 - protein, 13 aa, synthetic construct
  REGION 1..13: note = 1P1/1P4_CDR-H2_Contact
WVSLILYDGSNKY

SEQ ID NO: 44 - protein, 8 aa, synthetic construct
  REGION 1..8: note = 1P1/1P4/1P1.2/1P4.2_CDR-H3_Contact
ARDRVWLD

SEQ ID NO: 45 - protein, 8 aa, synthetic construct
  REGION 1..8: note = 1P1/1P4_CDR-H1_IMGT
GFTFSNYG

SEQ ID NO: 46 - protein, 8 aa, synthetic construct
  REGION 1..8: note = 1P1/1P4_CDR-H2_IMGT
ILYDGSNK

SEQ ID NO: 47 - protein, 9 aa, synthetic construct
  REGION 1..9: note = 1P1/1P4/1P1.2/1P4.2_CDR-H3_IMGT
ARDRVWLDV

SEQ ID NO: 48 - protein, 7 aa, synthetic construct
  REGION 1..7: note = 1P1.2/1P4.2/1P9.2/2M1_CDR-H1_Chothia
GFTFSSY

SEQ ID NO: 49 - protein, 6 aa, synthetic construct
  REGION 1..6: note = 1P1.2/1P4.2__CDR-H2_Chothia
WYDGSN

SEQ ID NO: 50 - protein, 10 aa, synthetic construct
  REGION 1..10: note = 1P1.2/1P4.2__CDR-H1_AbM
GFTFSSYGMH

SEQ ID NO: 51 - protein, 10 aa, synthetic construct
  REGION 1..10: note = 1P1.2/1P4.2__CDR-H2_AbM
VIWYDGSNKY

SEQ ID NO: 52 - protein, 5 aa, synthetic construct
  REGION 1..5: note = 1P1.2/1P4.2__CDR-H1_Kabat
SYGMH

SEQ ID NO: 53 - protein, 17 aa, synthetic construct
  REGION 1..17: note = 1P1.2/1P4.2__CDR-H2_Kabat
VIWYDGSNKYYADSVKG

SEQ ID NO: 54 - protein, 6 aa, synthetic construct
  REGION 1..6: note = 1P1.2/1P4.2__CDR-H1_Contact
SSYGMH

SEQ ID NO: 55 - protein, 13 aa, synthetic construct
  REGION 1..13: note = 1P1.2/1P4.2__CDR-H2_Contact
WVAVIWYDGSNKY

SEQ ID NO: 56 - protein, 8 aa, synthetic construct
  REGION 1..8: note = 1P1.2/1P4.2__CDR-H1_IMGT
GFTFSSYG

SEQ ID NO: 57 - protein, 8 aa, synthetic construct
  REGION 1..8: note = 1P1.2/1P4.2__CDR-H2_IMGT
IWYDGSNK

SEQ ID NO: 58 - protein, 7 aa, synthetic construct
  REGION 1..7: note = 1P2/1P2_N73D_CDR-H1_Chothia
GFTFSRY

SEQ ID NO: 59 - protein, 6 aa, synthetic construct
  REGION 1..6: note = 1P2/1P2_N73D_CDR-H2_Chothia
NPNNGN

SEQ ID NO: 60 - protein, 7 aa, synthetic construct
  REGION 1..7: note = 1P2/1P2_N73D_CDR-H3_Chothia_AbM, Kabat; 1P2.2_Kabat
GWTFFDY

SEQ ID NO: 61 - protein, 10 aa, synthetic construct
  REGION 1..10: note = 1P2/1P2_N73D_CDR-H1_AbM
GFTFSRYDFN

SEQ ID NO: 62 - protein, 10 aa, synthetic construct
  REGION 1..10: note = 1P2/1P2_N73D_CDR-H2_AbM
WMNPNNGNTG

SEQ ID NO: 63 - protein, 5 aa, synthetic construct
  REGION 1..5: note = 1P2/1P2_N73D_CDR-H1_Kabat
RYDFN

SEQ ID NO: 64 - protein, 17 aa, synthetic construct
  REGION 1..17: note = 1P2/1P2_N73D_CDR-H2_Kabat
WMNPNNGNTGYSQKFQG

SEQ ID NO: 65 - protein, 6 aa, synthetic construct
  REGION 1..6: note = 1P2/1P2_N73D_CDR-H1_Contact
SRYDFN

SEQ ID NO: 66 - protein, 13 aa, synthetic construct
  REGION 1..13: note = 1P2/1P2_N73D_CDR-H2_Contact
WMGWMNPNNGNTG

SEQ ID NO: 67 - protein, 8 aa, synthetic construct
  REGION 1..8: note = 1P2/1P2_N73D/1P2.2_CDR-H3_Contact
ARGWTFFD

SEQ ID NO: 68 - protein, 8 aa, synthetic construct
  REGION 1..8: note = 1P2/1P2_N73D_CDR-H1_IMGT
GFTFSRYD

SEQ ID NO: 69 - protein, 8 aa, synthetic construct
  REGION 1..8: note = 1P2/1P2_N73D_CDR-H2_IMGT
MNPNNGNT

SEQ ID NO: 70 - protein, 9 aa, synthetic construct
  REGION 1..9: note = 1P2/1P2_N73D_CDR-H3_IMGT
ARGWTFFDY

SEQ ID NO: 71 - protein, 7 aa, synthetic construct
  REGION 1..7: note = 1P2.2_CDR-H1_Chothia
GYTFTSY

SEQ ID NO: 72 - protein, 6 aa, synthetic construct
  REGION 1..6: note = 1P2.2_CDR-H2_Chothia
NPNSGN

SEQ ID NO: 73 - protein, 10 aa, synthetic construct
  REGION 1..10: note = 1P2.2_CDR-H1_AbM
GYTFTSYDIN

SEQ ID NO: 74 - protein, 10 aa, synthetic construct
  REGION 1..10: note = 1P2.2_CDR-H2_AbM
WMNPNSGNTG

SEQ ID NO: 75 - protein, 5 aa, synthetic construct
  REGION 1..5: note = 1P2.2_CDR-H1_Kabat
SYDIN

SEQ ID NO: 76 - protein, 17 aa, synthetic construct
  REGION 1..17: note = 1P2.2_CDR-H2_Kabat
WMNPNSGNTGYAQKFQG

SEQ ID NO: 77 - protein, 6 aa, synthetic construct
  REGION 1..6: note = 1P2.2_CDR-H1_Contact
TSYDIN

SEQ ID NO: 78 - protein, 13 aa, synthetic construct
  REGION 1..13: note = 1P2.2_CDR-H2_Contact
WMGWMNPNSGNTG

SEQ ID NO: 79 - protein, 8 aa, synthetic construct
  REGION 1..8: note = 1P2.2_CDR-H1_IMGT
GYTFTSYD

SEQ ID NO: 80 - protein, 8 aa, synthetic construct
  REGION 1..8: note = 1P2.2_CDR-H2_IMGT
MNPNSGNT

SEQ ID NO: 81 - protein, 9 aa, synthetic construct
  REGION 1..9: note = 1P2.2_CDR-H3_IMGT
ARGWTFFDY

SEQ ID NO: 82 - protein, 7 aa, synthetic construct
  REGION 1..7: note = 1P9_CDR-H1_Chothia
GFTFSTY

SEQ ID NO: 83 - protein, 6 aa, synthetic construct
  REGION 1..6: note = 1P9_CDR-H2_Chothia
KQDGGE

SEQ ID NO: 84 - protein, 10 aa, synthetic construct
  REGION 1..10: note = 1P9/1P9.2_CDR-H3_Chothia_AbM_Kabat
LRLYSDYEDY

SEQ ID NO: 85 - protein, 10 aa, synthetic construct
  REGION 1..10: note = 1P9_CDR-H1_AbM
GFTFSTYWMS

SEQ ID NO: 86 - protein, 10 aa, synthetic construct
  REGION 1..10: note = 1P9_CDR-H2_AbM
NIKQDGGERY

SEQ ID NO: 87 - protein, 5 aa, synthetic construct
  REGION 1..5: note = 1P9_CDR-H1_Kabat
TYWMS

SEQ ID NO: 88 - protein, 17 aa, synthetic construct
  REGION 1..17: note = 1P9_CDR-H2_Kabat
NIKQDGGERYYVDSVKG

SEQ ID NO: 89 - protein, 6 aa, synthetic construct
  REGION 1..6: note = 1P9_CDR-H1_Contact
STYWMS

SEQ ID NO: 90 - protein, 13 aa, synthetic construct
  REGION 1..13: note = 1P9_CDR-H2_Contact
WVANIKQDGGERY

SEQ ID NO: 91 - protein, 11 aa, synthetic construct
  REGION 1..11: note = 1P9/1P9.2_CDR-H3_Contact
ARLRLYSDYED

SEQ ID NO: 92 - protein, 8 aa, synthetic construct
  REGION 1..8: note = 1P9_CDR-H1_IMGT
GFTFSTYW

SEQ ID NO: 93 - protein, 8 aa, synthetic construct
  REGION 1..8: note = 1P9_CDR-H2_IMGT
IKQDGGER

SEQ ID NO: 94 - protein, 12 aa, synthetic construct
  REGION 1..12: note = 1P9_CDR-H3_IMGT
ARLRLYSDYEDY

SEQ ID NO: 95 - protein, 6 aa, synthetic construct
  REGION 1..6: note = 1P9.2_CDR-H2_Chothia
KQDGSE

SEQ ID NO: 96 - protein, 10 aa, synthetic construct
  REGION 1..10: note = 1P9.2_CDR-H1_AbM
GFTFSSYWMS

SEQ ID NO: 97 - protein, 10 aa, synthetic construct
  REGION 1..10: note = 1P9.2_CDR-H2_AbM
NIKQDGSEKY

SEQ ID NO: 98 - protein, 5 aa, synthetic construct
  REGION 1..5: note = 1P9.2_CDR-H1_Kabat
SYWMS

SEQ ID NO: 99 - protein, 17 aa, synthetic construct
  REGION 1..17: note = 1P9.2_CDR-H2_Kabat
NIKQDGSEKYYVDSVKG

SEQ ID NO: 100 - protein, 6 aa, synthetic construct
  REGION 1..6: note = 1P9.2_CDR-H1_Contact
SSYWMS

SEQ ID NO: 101 - protein, 13 aa, synthetic construct
  REGION 1..13: note = 1P9.2_CDR-H2_Contact
WVANIKQDGSEKY

SEQ ID NO: 102 - protein, 8 aa, synthetic construct
  REGION 1..8: note = 1P9.2_CDR-H1_IMGT
GFTFSSYW

SEQ ID NO: 103 - protein, 8 aa, synthetic construct
  REGION 1..8: note = 1P9.2_CDR-H2_IMGT
IKQDGSEK

SEQ ID NO: 104 - protein, 6 aa, synthetic construct
  REGION 1..6: note = 2M1_CDR-H2_Chothia
TSSTSY

SEQ ID NO: 105 - protein, 13 aa, synthetic construct
  REGION 1..13: note = 2M1_CDR-H3_Chothia_AbM_Kabat
DWSIAARGAWFDP

SEQ ID NO: 106 - protein, 10 aa, synthetic construct
  REGION 1..10: note = 2M1_CDR-H1_AbM
GFTFSSYTMN

SEQ ID NO: 107 - protein, 10 aa, synthetic construct
  REGION 1..10: note = 2M1_CDR-H2_AbM
SITSSTSYIY

SEQ ID NO: 108 - protein, 5 aa, synthetic construct
  REGION 1..5: note = 2M1_CDR-H1_Kabat
SYTMN

SEQ ID NO: 109 - protein, 17 aa, synthetic construct
  REGION 1..17: note = 2M1_CDR-H2_Kabat
SITSSTSYIYYADSVKG

SEQ ID NO: 110 - protein, 6 aa, synthetic construct
  REGION 1..6: note = 2M1_CDR-H1_Contact
SSYTMN

SEQ ID NO: 111 - protein, 13 aa, synthetic construct
  REGION 1..13: note = 2M1_CDR-H2_Contact
WVSSITSSTSYIY

SEQ ID NO: 112 - protein, 14 aa, synthetic construct
  REGION 1..14: note = 2M1_CDR-H3_Contact
VRDWSIAARGAWFD

SEQ ID NO: 113 - protein, 8 aa, synthetic construct
  REGION 1..8: note = 2M1_CDR-H1_IMGT
GFTFSSYT

SEQ ID NO: 114 - protein, 8 aa, synthetic construct
  REGION 1..8: note = 2M1_CDR-H2_IMGT
ITSSTSYI

SEQ ID NO: 115 - protein, 15 aa, synthetic construct
  REGION 1..15: note = 2M1_CDR-H3_IMGT
VRDWSIAARGAWFDP

SEQ ID NO: 116 - protein, 7 aa, synthetic construct
  REGION 1..7: note = 2M2_CDR-H1_Chothia
GYTFTNY

SEQ ID NO: 117 - protein, 6 aa, synthetic construct
  REGION 1..6: note = 2M2_CDR-H2_Chothia
SAYNGH

SEQ ID NO: 118 - protein, 6 aa, synthetic construct
  REGION 1..6: note = 2M2_CDR-H3_Chothia_AbM_Kabat
GTSGDY

SEQ ID NO: 119 - protein, 10 aa, synthetic construct
  REGION 1..10: note = 2M2_CDR-H1_AbM
GYTFTNYGIS

SEQ ID NO: 120 - protein, 10 aa, synthetic construct
  REGION 1..10: note = 2M2_CDR-H2_AbM
WISAYNGHTN

SEQ ID NO: 121 - protein, 5 aa, synthetic construct
  REGION 1..5: note = 2M2_CDR-H1_Kabat
NYGIS

SEQ ID NO: 122 - protein, 17 aa, synthetic construct
  REGION 1..17: note = 2M2_CDR-H2_Kabat
WISAYNGHTNYAQKVQG

SEQ ID NO: 123 - protein, 6 aa, synthetic construct
  REGION 1..6: note = 2M2_CDR-H1_Contact
TNYGIS

SEQ ID NO: 124 - protein, 13 aa, synthetic construct
  REGION 1..13: note = 2M2_CDR-H2_Contact
WMGWISAYNGHTN

SEQ ID NO: 125 - protein, 7 aa, synthetic construct
  REGION 1..7: note = 2M2_CDR-H3_Contact
ARGTSGD

SEQ ID NO: 126 - protein, 8 aa, synthetic construct
  REGION 1..8: note = 2M2_CDR-H1_IMGT
GYTFTNYG

SEQ ID NO: 127 - protein, 8 aa, synthetic construct
  REGION 1..8: note = 2M2_CDR-H2_IMGT
ISAYNGHT

SEQ ID NO: 128 - protein, 8 aa, synthetic construct
  REGION 1..8: note = 2M2_CDR-H3_IMGT
ARGTSGDY

SEQ ID NO: 129 - protein, 7 aa, synthetic construct
  REGION 1..7: note = 2M3_CDR-H1_Chothia
GFNFRSY

SEQ ID NO: 130 - protein, 5 aa, synthetic construct
  REGION 1..5: note = 2M3_CDR-H2_Chothia
GPGGD

SEQ ID NO: 131 - protein, 10 aa, synthetic construct
  REGION 1..10: note = 2M3_CDR-H3_Chothia_AbM_Kabat
EQFEGHYFDY

SEQ ID NO: 132 - protein, 10 aa, synthetic construct
  REGION 1..10: note = 2M3_CDR-H1_AbM
GFNFRSYDMH

SEQ ID NO: 133 - protein, 9 aa, synthetic construct
  REGION 1..9: note = 2M3_CDR-H2_AbM
GIGPGGDTY

SEQ ID NO: 134 - protein, 5 aa, synthetic construct
  REGION 1..5: note = 2M3_CDR-H1_Kabat
SYDMH

SEQ ID NO: 135 - protein, 16 aa, synthetic construct
  REGION 1..16: note = 2M3_CDR-H2_Kabat
GIGPGGDTYYPGSVKG

SEQ ID NO: 136 - protein, 6 aa, synthetic construct
  REGION 1..6: note = 2M3_CDR-H1_Contact
RSYDMH

SEQ ID NO: 137 - protein, 12 aa, synthetic construct
  REGION 1..12: note = 2M3_CDR-H2_Contact
WVSGIGPGGDTY

SEQ ID NO: 138 - protein, 11 aa, synthetic construct
  REGION 1..11: note = 2M3_CDR-H3_Contact
AREQFEGHYFD

SEQ ID NO: 139 - protein, 8 aa, synthetic construct
  REGION 1..8: note = 2M3_CDR-H1_IMGT
GFNFRSYD

SEQ ID NO: 140 - protein, 7 aa, synthetic construct
  REGION 1..7: note = 2M3_CDR-H2_IMGT
IGPGGDT

SEQ ID NO: 141 - protein, 12 aa, synthetic construct
  REGION 1..12: note = 2M3_CDR-H3_IMGT
AREQFEGHYFDY

SEQ ID NO: 142 - protein, 7 aa, synthetic construct
  REGION 1..7: note = 2M5_CDR-H1_Chothia
GFTLSSY

SEQ ID NO: 143 - protein, 6 aa, synthetic construct
  REGION 1..6: note = 2M5_CDR-H2_Chothia
SSSSSY

SEQ ID NO: 144 - protein, 12 aa, synthetic construct
  REGION 1..12: note = 2M5_CDR-H3_Chothia_AbM_Kabat
EGGAARPSWFDP

SEQ ID NO: 145 - protein, 10 aa, synthetic construct
  REGION 1..10: note = 2M5_CDR-H1_AbM
GFTLSSYSMN

SEQ ID NO: 146 - protein, 10 aa, synthetic construct
  REGION 1..10: note = 2M5_CDR-H2_AbM
SISSSSSYIY

SEQ ID NO: 147 - protein, 5 aa, synthetic construct
  REGION 1..5: note = 2M5_CDR-H1_Kabat
SYSMN

SEQ ID NO: 148 - protein, 17 aa, synthetic construct
  REGION 1..17: note = 2M5_CDR-H2_Kabat
SISSSSSYIYYADSVKG

SEQ ID NO: 149 - protein, 6 aa, synthetic construct
  REGION 1..6: note = 2M5_CDR-H1_Contact
SSYSMN

SEQ ID NO: 150 - protein, 13 aa, synthetic construct
  REGION 1..13: note = 2M5_CDR-H2_Contact
WVSSISSSSSYIY

SEQ ID NO: 151 - protein, 13 aa, synthetic construct
  REGION 1..13: note = 2M5_CDR-H3_Contact
AREGGAARPSWFD

SEQ ID NO: 152 - protein, 8 aa, synthetic construct
  REGION 1..8: note = 2M5_CDR-H1_IMGT
GFTLSSYS

SEQ ID NO: 153 - protein, 8 aa, synthetic construct
  REGION 1..8: note = 2M5_CDR-H2_IMGT
ISSSSSYI

SEQ ID NO: 154 - protein, 14 aa, synthetic construct
  REGION 1..14: note = 2M5_CDR-H3_IMGT
AREGGAARPSWFDP

SEQ ID NO: 155 - protein, 7 aa, synthetic construct
  REGION 1..7: note = 3B1/3B5_CDR-H1_Chothia
GYDFTNY

SEQ ID NO: 156 - protein, 6 aa, synthetic construct
  REGION 1..6: note = 3B1/3B5_CDR-H2_Chothia
NTYTGE

SEQ ID NO: 157 - protein, 10 aa, synthetic construct
  REGION 1..10: note = 3B1/3B5_CDR-H3_Chothia_AbM_Kabat
RAYYGHYLAY

SEQ ID NO: 158 - protein, 10 aa, synthetic construct
  REGION 1..10: note = 3B1/3B5_CDR-H1_AbM
GYDFTNYGMN

SEQ ID NO: 159 - protein, 10 aa, synthetic construct
  REGION 1..10: note = 3B1/3B5_CDR-H2_AbM
YINTYTGEPA

SEQ ID NO: 160 - protein, 5 aa, synthetic construct
  REGION 1..5: note = 3B1/3B5_CDR-H1_Kabat
NYGMN

SEQ ID NO: 161 - protein, 17 aa, synthetic construct
  REGION 1..17: note = 3B1/3B5_CDR-H2_Kabat
YINTYTGEPAYADDFKG

SEQ ID NO: 162 - protein, 6 aa, synthetic construct
  REGION 1..6: note = 3B1/3B5_CDR-H1_Contact
TNYGMN

SEQ ID NO: 163 - protein, 13 aa, synthetic construct
  REGION 1..13: note = 3B1/3B5_CDR-H2_Contact
WMGYINTYTGEPA

SEQ ID NO: 164 - protein, 11 aa, synthetic construct
  REGION 1..11: note = 3B1/3B5_CDR-H3_Contact
ATRAYYGHYLA

SEQ ID NO: 165 - protein, 8 aa, synthetic construct
  REGION 1..8: note = 3B1/3B5_CDR-H1_IMGT
GYDFTNYG

SEQ ID NO: 166 - protein, 8 aa, synthetic construct
  REGION 1..8: note = 3B1/3B5_CDR-H2_IMGT
INTYTGEP

SEQ ID NO: 167 - protein, 12 aa, synthetic construct
  REGION 1..12: note = 3B1/3B5_CDR-H3_IMGT
ATRAYYGHYLAY

SEQ ID NO: 168 - protein, 7 aa, synthetic construct
  REGION 1..7: note = 3B2/3B4_CDR-H1_Chothia
GDSITSG

SEQ ID NO: 169 - protein, 5 aa, synthetic construct
  REGION 1..5: note = 3B2/3B4_CDR-H2_Chothia
SYTGS

SEQ ID NO: 170 - protein, 10 aa, synthetic construct
  REGION 1..10: note = 3B2_CDR-H3_Chothia_AbM_Kabat
KGGWLLPFAY

SEQ ID NO: 171 - protein, 10 aa, synthetic construct
  REGION 1..10: note = 3B2/3B4_CDR-H1_AbM
GDSITSGYWN

SEQ ID NO: 172 - protein, 9 aa, synthetic construct
  REGION 1..9: note = 3B2/3B4_CDR-H2_AbM
YISYTGSTY

SEQ ID NO: 173 - protein, 5 aa, synthetic construct
  REGION 1..5: note = 3B2/3B4_CDR-H1_Kabat
SGYWN

SEQ ID NO: 174 - protein, 16 aa, synthetic construct
  REGION 1..16: note = 3B2/3B4_CDR-H2_Kabat
YISYTGSTYYNPSLKS

SEQ ID NO: 175 - protein, 6 aa, synthetic construct
  REGION 1..6: note = 3B2/3B4_CDR-H1_Contact
TSGYWN

SEQ ID NO: 176 - protein, 12 aa, synthetic construct
  REGION 1..12: note = 3B2_CDR-H2_Contact
YMGYISYTGSTY

SEQ ID NO: 177 - protein, 11 aa, synthetic construct
  REGION 1..11: note = 3B2_CDR-H3_Contact
ARKGGWLLPFA

SEQ ID NO: 178 - protein, 8 aa, synthetic construct
  REGION 1..8: note = 3B2/3B4_CDR-H1_IMGT
GDSITSGY

SEQ ID NO: 179 - protein, 7 aa, synthetic construct
  REGION 1..7: note = 3B2/3B4_CDR-H2_IMGT
ISYTGST

SEQ ID NO: 180 - protein, 12 aa, synthetic construct
  REGION 1..12: note = 3B2/3B4_CDR-H2_IMGT
ARKGGWLLPFAY

SEQ ID NO: 181 - protein, 8 aa, synthetic construct
  REGION 1..8: note = 3B2/3B4_CDR-H2_IMGT
TVKSDNYG

SEQ ID NO: 182 - protein, 6 aa, synthetic construct
  REGION 1..6: note = 3B3_CDR-H3_Chothia_AbM_Kabat
GRYFDY

SEQ ID NO: 183 - protein, 10 aa, synthetic construct
  REGION 1..10: note = 3B3_CDR-H1_AbM
GFTFSNYRMH

SEQ ID NO: 184 - protein, 12 aa, synthetic construct
  REGION 1..12: note = 3B3_CDR-H2_AbM
VITVKSDNYGAN

SEQ ID NO: 185 - protein, 5 aa, synthetic construct
  REGION 1..5: note = 3B3_CDR-H1_Kabat
NYRMH

SEQ ID NO: 186 - protein, 19 aa, synthetic construct
  REGION 1..19: note = 3B3_CDR-H2_Kabat
VITVKSDNYGANYAESVKG

SEQ ID NO: 187 - protein, 6 aa, synthetic construct
  REGION 1..6: note = 3B3_CDR-H1_Contact
SNYRMH

SEQ ID NO: 188 - protein, 15 aa, synthetic construct
  REGION 1..15: note = 3B3_CDR-H2_Contact
WIAVITVKSDNYGAN

SEQ ID NO: 189 - protein, 7 aa, synthetic construct
  REGION 1..7: note = 3B3_CDR-H3_Contact
SLGRYFD

SEQ ID NO: 190 - protein, 8 aa, synthetic construct
  REGION 1..8: note = 3B3_CDR-H1_IMGT
GFTFSNYR

SEQ ID NO: 191 - protein, 10 aa, synthetic construct
  REGION 1..10: note = 3B3_CDR-H2_IMGT
ITVKSDNYGA

SEQ ID NO: 192 - protein, 8 aa, synthetic construct
  REGION 1..8: note = 3B3_CDR-H3_IMGT
SLGRYFDY

SEQ ID NO: 193 - protein, 10 aa, synthetic construct
  REGION 1..10: note = 3B4_CDR-H3_Chothia_AbM_Kabat
GLLWLAWFAY

SEQ ID NO: 194 - protein, 12 aa, synthetic construct
  REGION 1..12: note = 3B4_CDR-H2_Contact
SMGYISYTGSTY

SEQ ID NO: 195 - protein, 11 aa, synthetic construct
  REGION 1..11: note = 3B4_CDR-H3_Contact
ASGLLWLAWFA

SEQ ID NO: 196 - protein, 12 aa, synthetic construct
  REGION 1..12: note = 3B4_CDR-H3_IMGT
ASGLLWLAWFAY

SEQ ID NO: 197 - protein, 7 aa, synthetic construct
  REGION 1..7: note = 3B6_CDR-H1_Chothia
GFTFSDF

SEQ ID NO: 198 - protein, 8 aa, synthetic construct
  REGION 1..8: note = 3B6_CDR-H2_Chothia
RNKVNDYT

SEQ ID NO: 199 - protein, 11 aa, synthetic construct
  REGION 1..11: note = 3B6_CDR-H3_Chothia_AbM_Kabat
NVPWDGWYFDV

SEQ ID NO: 200 - protein, 10 aa, synthetic construct
  REGION 1..10: note = 3B6_CDR-H1_AbM
GFTFSDFYVE

SEQ ID NO: 201 - protein, 12 aa, synthetic construct
  REGION 1..12: note = 3B6_CDR-H2_AbM
ASRNKVNDYTTE

SEQ ID NO: 202 - protein, 5 aa, synthetic construct
  REGION 1..5: note = 3B6_CDR-H1_Kabat
DFYVE

SEQ ID NO: 203 - protein, 19 aa, synthetic construct
  REGION 1..19: note = 3B6_CDR-H2_Kabat
ASRNKVNDYTTEYSESVKG

SEQ ID NO: 204 - protein, 6 aa, synthetic construct
  REGION 1..6: note = 3B6_CDR-H1_Contact
SDFYVE

SEQ ID NO: 205 - protein, 15 aa, synthetic construct
  REGION 1..15: note = 3B6_CDR-H2_Contact
WIAASRNKVNDYTTE

SEQ ID NO: 206 - protein, 12 aa, synthetic construct
  REGION 1..12: note = 3B6_CDR-H3_Contact
ARNVPWDGWYFD

SEQ ID NO: 207 - protein, 8 aa, synthetic construct
  REGION 1..8: note = 3B6_CDR-H1_IMGT
GFTFSDFY

SEQ ID NO: 208 - protein, 10 aa, synthetic construct
  REGION 1..10: note = 3B6_CDR-H2_IMGT
SRNKVNDYTT

SEQ ID NO: 209 - protein, 13 aa, synthetic construct
  REGION 1..13: note = 3B6_CDR-H3_IMGT
ARNVPWDGWYFDV

SEQ ID NO: 210 - protein, 11 aa, synthetic construct
  REGION 1..11: note = 1P1_CDR-L1_Chothia_AbM_Kabat
RASQGIRNYLA

SEQ ID NO: 211 - protein, 7 aa, synthetic construct
  REGION 1..7: note = 1P1/P1.2_CDR-L2_Chothia_AbM_Kabat
AASTLQS

SEQ ID NO: 212 - protein, 9 aa, synthetic construct
  REGION 1..9: note = 1P1/1P1.2_CDR-L3_Chothia_AbM_Kabat_IMGT
QKYNSALWT

SEQ ID NO: 213 - protein, 7 aa, synthetic construct
  REGION 1..7: note = 1P1_CDR-L1_Contact
RNYLAWY

SEQ ID NO: 214 - protein, 10 aa, synthetic construct
  REGION 1..10: note = 1P1_CDR-L2_Contact
LLIYAASTLQ

SEQ ID NO: 215 - protein, 8 aa, synthetic construct
  REGION 1..8: note = 1P1_CDR-L3_Contact
QKYNSALW

SEQ ID NO: 216 - protein, 6 aa, synthetic construct
  REGION 1..6: note = 1P1_CDR-L1_IMGT
QGIRNY

SEQ ID NO: 217 - skipped

SEQ ID NO: 218 - protein, 11 aa, synthetic construct
  REGION 1..11: note = 1P1.2_CDR-L1_Chothia
RASQGISNYLA

SEQ ID NO: 219 - protein, 7 aa, synthetic construct
  REGION 1..7: note = 1P1.2_CDR-L1_Contact
SNYLAWY

SEQ ID NO: 220 - protein, 6 aa, synthetic construct
  REGION 1..6: note = 1P1.2/3B3_CDR-L1_IMGT
QGISNY

SEQ ID NO: 221 - protein, 12 aa, synthetic construct
  REGION 1..12: note = 1P2/1P2_N73D_CDR-L1_Chothia_AbM_Kabat
RASQSVSSTYLA

SEQ ID NO: 222 - protein, 7 aa, synthetic construct
  REGION 1..7: note = 1P2/1P2_N73D/1P2.2_CDR-L2_Chothia_AbM_Kabat
GASSRAT

SEQ ID NO: 223 - protein, 8 aa, synthetic construct
  REGION 1..8: note = 1P2/1P2_N73D1P2.2_CDR-L3_Chothia_AbM_Kabat_IMGT
QQYGSSPT

SEQ ID NO: 224 - protein, 8 aa, synthetic construct
  REGION 1..8: note = 1P2/1P2_N73D_CDR-L1_Contact
SSTYLAWY

SEQ ID NO: 225 - protein, 10 aa, synthetic construct
  REGION 1..10: note = 1P2/1P2_N73D_CDR-L2_Contact
LLIFGASSRA

SEQ ID NO: 226 - protein, 7 aa, synthetic construct
  REGION 1..7: note = 1P2/1P2/1P2.2_N73D_CDR-L3_Contact
QQYGSSP

SEQ ID NO: 227 - protein, 7 aa, synthetic construct
  REGION 1..7: note = 1P2/1P2_N73D_CDR-L1_IMGT
QSVSSTY

SEQ ID NO: 228 - skipped

SEQ ID NO: 229 - protein, 12 aa, synthetic construct
  REGION 1..12: note = 1P2.2_CDR-L1_Chothia_AbM_Kabat
RASQSVSSSYLA

SEQ ID NO: 230 - protein, 8 aa, synthetic construct
  REGION 1..8: note = 1P2.2_CDR-L1_Contact
SSSYLAWY

SEQ ID NO: 231 - protein, 10 aa, synthetic construct
  REGION 1..10: note = 1P2.2_CDR-L2_Contact
LLIYGASSRA

SEQ ID NO: 232 - protein, 7 aa, synthetic construct
  REGION 1..7: note = 1P2.2_CDR-L1_IMGT
QSVSSSY

SEQ ID NO: 233 - protein, 17 aa, synthetic construct
  REGION 1..17: note = 1P4_CDR-L1_Chothia_AbM_Kabat
KSSQSVLYRSSNKNYLA

SEQ ID NO: 234 - protein, 7 aa, synthetic construct
  REGION 1..7: note = 1P4/1P4.2/3B2/3B4_CDR-L2_Chothia_AbM_Kabat
WASTRES

SEQ ID NO: 235 - protein, 9 aa, synthetic construct
  REGION 1..9: note = 1P4_CDR-L3_Chothia_AbM_Kabat_IMGT
HQYYTTPLT

SEQ ID NO: 236 - protein, 13 aa, synthetic construct
  REGION 1..13: note = 1P4_CDR-L1_Contact
LYRSSNKNYLAWY

SEQ ID NO: 237 - protein, 10 aa, synthetic construct
  REGION 1..10: note = 1P4/1P4.2/3B2/3B4_CDR-L2_Contact
LLIYWASTRE

SEQ ID NO: 238 - protein, 8 aa, synthetic construct
  REGION 1..8: note = 1P4_CDR-L3_Contact
HQYYTTPL

SEQ ID NO: 239 - protein, 12 aa, synthetic construct
  REGION 1..12: note = 1P4_CDR-LH1_IMGT
QSVLYRSSNKNY

SEQ ID NO: 240 - skipped

SEQ ID NO: 241 - protein, 17 aa, synthetic construct
  REGION 1..17: note = 1P4.2_CDR-L1_Chothia_AbM_Kabat
KSSQSVLYSSNNKNYLA

SEQ ID NO: 242 - protein, 9 aa, synthetic construct
  REGION 1..9: note = 1P4.2_CDR-L3_Chothia_AbM_Kabat_IMGT
QQYYSTPLT

SEQ ID NO: 243 - protein, 13 aa, synthetic construct
  REGION 1..13: note = 1P9_CDR-L1_Contact
LYSSNNKNYLAWY

SEQ ID NO: 244 - protein, 8 aa, synthetic construct
  REGION 1..8: note = 1P9_CDR-L3_Contact
QQYYSTPL

SEQ ID NO: 245 - protein, 12 aa, synthetic construct
  REGION 1..12: note = 1P9_CDR-L1_IMGT
QSVLYSSNNKNY

SEQ ID NO: 246 - protein, 11 aa, synthetic construct
  REGION 1..11: note = 1P9/1P9.2/2M3_CDR-L1_Chothia_AbM_Kabat
RASQGISSWLA

SEQ ID NO: 247 - protein, 7 aa, synthetic construct
  REGION 1..7: note = 1P9_CDR-L2_Chothia_AbM_Kabat
SVSSLQS

SEQ ID NO: 248 - protein, 9 aa, synthetic construct
  REGION 1..9: note = 1P9_CDR-L3_Chothia_AbM_Kabat_IMGT
QQANSLPPT

SEQ ID NO: 249 - protein, 7 aa, synthetic construct
  REGION 1..7: note = 1P9/1P9.2/2M3_CDR-L1_Contact
SSWLAWY

SEQ ID NO: 250 - protein, 10 aa, synthetic construct
  REGION 1..10: note = 1P9_CDR-L2_Contact
LLIYSVSSLQ

SEQ ID NO: 251 - protein, 8 aa, synthetic construct
  REGION 1..8: note = 1P9_CDR-L3_Contact
QQANSLPP

SEQ ID NO: 252 - protein, 6 aa, synthetic construct
  REGION 1..6: note = 1P9/2M3_CDR-L1_IMGT
QGISSW

SEQ ID NO: 253 - skipped

SEQ ID NO: 254 - protein, 7 aa, synthetic construct
  REGION 1..7: note = 1P9.2_CDR-L2_Chothia_AbM_Kabat
AASSLQS

SEQ ID NO: 255 - protein, 9 aa, synthetic construct
  REGION 1..9: note = 1P9.2_CDR-L3_Chothia_AbM_Kabat_IMGT
QQANSFPPT

SEQ ID NO: 256 - protein, 10 aa, synthetic construct
  REGION 1..10: note = 1P9.2_CDR-L2_Contact
LLIYAASSLQ

SEQ ID NO: 257 - protein, 8 aa, synthetic construct
  REGION 1..8: note = 1P9.2_CDR-L3_Contact
QQANSFPP

SEQ ID NO: 258 - protein, 11 aa, synthetic construct
  REGION 1..11: note = 2M1_CDR-L1_Chothia_AbM_Kabat
RASQGIGSWLA

SEQ ID NO: 259 - protein, 7 aa, synthetic construct
  REGION 1..7: note = 2M1_CDR-L2_Chothia_AbM_Kabat
AASSLQG

SEQ ID NO: 260 - protein, 9 aa, synthetic construct
  REGION 1..9: note = 2M1_CDR-L3_Chothia_AbM_Kabat_IMGT
QQANSFPLT

SEQ ID NO: 261 - protein, 7 aa, synthetic construct
  REGION 1..7: note = 2M1_CDR-L1_Contact
GSWLAWY

SEQ ID NO: 262 - protein, 10 aa, synthetic construct
  REGION 1..10: note = 2M1_CDR-L2_Contact
FLIYAASSLQ

SEQ ID NO: 263 - protein, 8 aa, synthetic construct
  REGION 1..8: note = 2M1_CDR-L3_Contact
QQANSFPL

SEQ ID NO: 264 - protein, 6 aa, synthetic construct
  REGION 1..6: note = 2M1_CDR-L1_IMGT
QGIGSW

SEQ ID NO: 265 - protein, 11 aa, synthetic construct
  REGION 1..11: note = 2M2_CDR-L1_Chothia_AbM_Kabat
RASQSVSSYLA

SEQ ID NO: 266 - protein, 7 aa, synthetic construct
  REGION 1..7: note = 2M2_CDR-L2_Chothia_AbM_Kabat
DASNRAT

SEQ ID NO: 267 - protein, 10 aa, synthetic construct
  REGION 1..10: note = 2M2_CDR-L3_Chothia_AbM_Kabat_IMGT
QQRSNWPPLT

SEQ ID NO: 268 - protein, 7 aa, synthetic construct
  REGION 1..7: note = 2M2_CDR-L1_Contact
SSYLAWY

SEQ ID NO: 269 - protein, 10 aa, synthetic construct
  REGION 1..10: note = 2M2_CDR-L2_Contact
LLIYDASNRA

SEQ ID NO: 270 - protein, 9 aa, synthetic construct
  REGION 1..9: note = 2M2_CDR-L3_Contact
QQRSNWPPL

SEQ ID NO: 271 - protein, 6 aa, synthetic construct
  REGION 1..6: note = 2M2_CDR-L1_IMGT
QSVSSY

SEQ ID NO: 272 - skipped

SEQ ID NO: 273 - protein, 7 aa, synthetic construct
  REGION 1..7: note = 2M3_CDR-L2_Chothia_AbM_Kabat
TASSLQS

SEQ ID NO: 274 - protein, 9 aa, synthetic construct
  REGION 1..9: note = 2M3_CDR-L3_Chothia_AbM_Kabat_IMGT
QQANSFPIT

SEQ ID NO: 275 - protein, 10 aa, synthetic construct
  REGION 1..10: note = 2M3_CDR-L2_Contact
LLIYTASSLQ

SEQ ID NO: 276 - protein, 8 aa, synthetic construct
  REGION 1..8: note = 2M3_CDR-L3_Contact
QQANSFPI

SEQ ID NO: 277 - skipped

SEQ ID NO: 278 - protein, 14 aa, synthetic construct
  REGION 1..14: note = 2M5_CDR-L1_Chothia_AbM_Kabat
RSSTGAVTTSNYAN

SEQ ID NO: 279 - protein, 7 aa, synthetic construct
  REGION 1..7: note = 2M5_CDR-L2_Chothia_AbM_Kabat
GTNNRAP

SEQ ID NO: 280 - protein, 9 aa, synthetic construct
  REGION 1..9: note = 2M5_CDR-L3_Chothia_AbM_Kabat_IMGT
ALWYRNHWV

SEQ ID NO: 281 - protein, 10 aa, synthetic construct
  REGION 1..10: note = 2M5_CDR-L1_Contact
VTTSNYANWV

SEQ ID NO: 282 - protein, 10 aa, synthetic construct
  REGION 1..10: note = 2M5_CDR-L2_Contact
GLIGGTNNRA

SEQ ID NO: 283 - protein, 8 aa, synthetic construct
  REGION 1..8: note = 2M5_CDR-L3_Contact
ALWYRNHW

SEQ ID NO: 284 - protein, 9 aa, synthetic construct
  REGION 1..9: note = 2M5_CDR-L1_IMGT
TGAVTTSNY

SEQ ID NO: 285 - skipped

SEQ ID NO: 286 - protein, 16 aa, synthetic construct
  REGION 1..16: note = 3B1_CDR-L1_Chothia_AbM_Kabat
RSSQRIVHSNGNTYLE

SEQ ID NO: 287 - protein, 7 aa, synthetic construct
  REGION 1..7: note = 3B1_CDR-L2_Chothia_AbM_Kabat
KVSNRFS

SEQ ID NO: 288 - protein, 9 aa, synthetic construct
  REGION 1..9: note = 3B1_CDR-L3_Chothia_AbM_Kabat_IMGT
FQGSHVPYT

SEQ ID NO: 289 - protein, 12 aa, synthetic construct
  REGION 1..12: note = 3B1_CDR-L1_Contact
VHSNGNTYLEWY

SEQ ID NO: 290 - protein, 10 aa, synthetic construct
  REGION 1..10: note = 3B1_CDR-L2_Contact
LLIYKVSNRF

SEQ ID NO: 291 - protein, 8 aa, synthetic construct
  REGION 1..8: note = 3B1_CDR-L3_Contact
FQGSHVPY

SEQ ID NO: 292 - protein, 11 aa, synthetic construct
  REGION 1..11: note = 3B1_CDR-L1_IMGT
QRIVHSNGNTY

SEQ ID NO: 293 - skipped

SEQ ID NO: 294 - protein, 17 aa, synthetic construct
  REGION 1..17: note = 3B2_CDR-L1_Chothia_AbM_Kabat
KSSQSLLYSTNQKNSLA

SEQ ID NO: 295 - protein, 9 aa, synthetic construct
  REGION 1..9: note = 3B2_CDR-L3_Chothia_AbM_Kabat_IMGT
QQYYSYPFT

SEQ ID NO: 296 - protein, 13 aa, synthetic construct
  REGION 1..13: note = 3B2_CDR-L1_Contact
LYSTNQKNSLAWY

SEQ ID NO: 297 - protein, 8 aa, synthetic construct
  REGION 1..8: note = 3B2_CDR-L3_Contact
QQYYSYPF

SEQ ID NO: 298 - protein, 12 aa, synthetic construct
  REGION 1..12: note = 3B2_CDR-L1_IMGT
QSLLYSTNQKNS

SEQ ID NO: 299 - protein, 11 aa, synthetic construct
  REGION 1..11: note = 3B3_CDR-L1_Chothia_AbM_Kabat
SASQGISNYLN

SEQ ID NO: 300 - protein, 7 aa, synthetic construct
  REGION 1..7: note = 3B3_CDR-L2_Chothia_AbM_Kabat
YTSSLHS

SEQ ID NO: 301 - protein, 9 aa, synthetic construct
  REGION 1..9: note = 3B3_CDR-L3_Chothia_AbM_Kabat_IMGT
QQYSKLPWT

SEQ ID NO: 302 - protein, 7 aa, synthetic construct
  REGION 1..7: note = 1P9_CDR-L1_Contact
SNYLNWY

SEQ ID NO: 303 - protein, 10 aa, synthetic construct
  REGION 1..10: note = 1P9_CDR-L2_Contact
LLIYYTSSLH

SEQ ID NO: 304 - protein, 8 aa, synthetic construct
  REGION 1..8: note = 1P9_CDR-L3_Contact
QQYSKLPW

SEQ ID NO: 305 - skipped

SEQ ID NO: 306 - protein, 17 aa, synthetic construct
  REGION 1..17: note = 1P9_CDR-L1_Chothia_AbM_Kabat
KSSQSLLYSSNQKNSLA

SEQ ID NO: 307 - protein, 9 aa, synthetic construct
  REGION 1..9: note = 1P9_CDR-L3_Chothia_AbM_Kabat_IMGT
QQYYGYPFT

SEQ ID NO: 308 - protein, 13 aa, synthetic construct
  REGION 1..13: note = 1P9_CDR-L1_Contact
LYSSNQKNSLAWY

SEQ ID NO: 309 - protein, 8 aa, synthetic construct
  REGION 1..8: note = 1P9_CDR-L3_Contact
QQYYGYPF

SEQ ID NO: 310 - protein, 12 aa, synthetic construct
  REGION 1..12: note = 1P9_CDR-L1_IMGT
QSLLYSSNQKNS

SEQ ID NO: 311 - protein, 10 aa, synthetic construct
  REGION 1..10: note = 1P9_CDR-L1_Chothia_AbM_Kabat
SASSSVSYMH

SEQ ID NO: 312 - protein, 7 aa, synthetic construct
  REGION 1..7: note = 1P9_CDR-L2_Chothia_AbM_Kabat
STSNLAS

SEQ ID NO: 313 - protein, 9 aa, synthetic construct
  REGION 1..9: note = 1P9_CDR-L3_Chothia_AbM_Kabat_IMGT
QQRSSYPFT

SEQ ID NO: 314 - protein, 6 aa, synthetic construct
  REGION 1..6: note = 1P9_CDR-L1_Contact
SYMHWF

SEQ ID NO: 315 - protein, 10 aa, synthetic construct
  REGION 1..10: note = 1P9_CDR-L2_Contact
LWIYSTSNLA

SEQ ID NO: 316 - protein, 8 aa, synthetic construct
  REGION 1..8: note = 1P9_CDR-L3_Contact
QQRSSYPF

SEQ ID NO: 317 - protein, 5 aa, synthetic construct
  REGION 1..5: note = 1P9_CDR-L1_IMGT
SSVSY

SEQ ID NO: 318 - skipped

SEQ ID NO: 319 - protein, 11 aa, synthetic construct
  REGION 1..11: note = 1P9_CDR-L1_Chothia_AbM_Kabat
KASQNVRTAVA

SEQ ID NO: 320 - protein, 7 aa, synthetic construct
  REGION 1..7: note = 1P9_CDR-L2_Chothia_AbM_Kabat
LASTRHT

SEQ ID NO: 321 - protein, 9 aa, synthetic construct
  REGION 1..9: note = 1P9_CDR-L3_Chothia_AbM_Kabat_IMGT
LQHWNYPRT

SEQ ID NO: 322 - protein, 7 aa, synthetic construct
  REGION 1..7: note = 1P9_CDR-L1_Contact
RTAVAWF

SEQ ID NO: 323 - protein, 10 aa, synthetic construct
  REGION 1..10: note = 1P9_CDR-L2_Contact
ALIYLASTRH

SEQ ID NO: 324 - protein, 8 aa, synthetic construct
  REGION 1..8: note = 1P9_CDR-L3_Contact
LQHWNYPR

SEQ ID NO: 325 - protein, 6 aa, synthetic construct
  REGION 1..6: note = 1P9_CDR-L1_IMGT
QNVRTA

SEQ ID NO: 326 - skipped

SEQ ID NO: 327 - protein, 221 aa, synthetic construct [sequence omitted]

SEQ ID NO: 328 - protein, 221 aa, synthetic construct
  REGION 1..221: note = cyPD-L1 ECD (UniProt Accession # G7PSE7 - Phe19-Thr239)
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERT

SEQ ID NO: 329 - protein, 220 aa, synthetic construct
  REGION 1..220: note = muPD-L1 ECD (UniProt Accession # Q9EP73 - Phe19-Thr238)
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRT

SEQ ID NO: 330 - protein, 119 aa, synthetic construct
  REGION 1..119: note = 3B1_3B5_VH_Humanized
QVQLVQSGSELKKPGASVKVSCKASGYDFTNYGMNWVRQAPGQGLEWMGYINTYTGEPAYAQGFTGRFVFSLDTSVSTAYLQISSLKAEDTAVYYCATRAYYGHYLAYWGQGTLVTVSS

SEQ ID NO: 331 - protein, 118 aa, synthetic construct
  REGION 1..118: note = 3B2_VH_Humanized
QVQLQQSGPGLVKPSQTLSLTCAVSGDSITSGYWNWIRQSPSRGLEYMGYISYTGSTYYAVSLKSRITINRDTSKNQFSLQLNSVTPEDTAVYYCARKGGWLLPFAYWGQGTLVTVSS

SEQ ID NO: 332 - protein, 117 aa, synthetic construct
  REGION 1..117: note = 3B3_VH_Humanized
EVQLVESGGGLVKPGGSLRLSCAASGFTFSNYRMHWLRQAPGKGLEWVGVITVKSDNYGANYAAPVKGRFTISRDDSKNTVYLQMNSLKTEDTAVYYCSLGRYFDYWGQGTTVTVSS

SEQ ID NO: 333 - protein, 118 aa, synthetic construct
  REGION 1..118: note = 3B4_VH_Humanized
QVQLQESGPGLVKPSQTLSLTCTVSGDSITSGYWNWIRQHPGKGLESMGYISYTGSTYYNPSLKSLLTISRDTSKNQFSLKLSSVTAADTAVYYCASGLLWLAWFAYWGQGTLVTVSS

SEQ ID NO: 334 - protein, 122 aa, synthetic construct
  REGION 1..122: note = 3B6_VH_Humanized
EVQLVESGGGLVQPGGSLRLSCAASGFTFSDFYVEWVRQAPGKGLEWVAASRNKVNDYTTEYVDSVKGRFTVSRDNAKNSLYLQMNSLRAEDTAVYYCARNVPWDGWYFDVWGQGTLVTVSS

SEQ ID NO: 335 - protein, 112 aa, synthetic construct
  REGION 1..112: note = 3B1_VL_Humanized
DIVMTQTPPSLPVNPGEPASISCRSSQRIVHSNGNTYLEWYLQKPGQSPQLLIYKVSNRFSGVPDRFSGSGSGSDFTLKISWVEAEDLGVYYCFQGSHVPYTFGQGTKLEIK

SEQ ID NO: 336 - protein, 113 aa, synthetic construct
  REGION 1..113: note = 3B2_VL_Humanized
DIVMTQTPLSLPVTPGEPASMSCRSSQSLLYSTNQKNSLAWYLQKPGQSPQLLIYWASTRESGVPDRFSGSGSGTDFTLKISRVEAEDLGVYYCQQYYSYPFTFGQGTKLEIK

SEQ ID NO: 337 - protein, 107 aa, synthetic construct
  REGION 1..107: note = 3B3_VL_Humanized
DIQMTQSPSSLSASVGDRVTITCQASQGISNYLNWYQQKPGKTVKLLIYYTSSLHSGVPSRFSGSGSGTDFTLTISSLQPEDIATYYCQQYSKLPWTFGQGTKLEIK

SEQ ID NO: 338 - protein, 113 aa, synthetic construct
  REGION 1..113: note = 3B4_VL_Humanized
DIVMTQTPLSLPVTPGEPVSMSCRSSQSLLYSSNQKNSLAWYLQKPGQSPQLLIYWASTRESGVPDRFSGSGSGTDFTLKISRVEAEDLGVYYCQQYYGYPFTFGQGTKLEIK

SEQ ID NO: 339 - protein, 106 aa, synthetic construct
  REGION 1..106: note = 3B5_VL_Humanized
AIQLTQSPSSLSASVGDRVTITCRASSSVSYMHWFQQKPGKAPKLWIYSTSNLASGVPSRFSGSGSGTDFTLTISSMQPEDAATYYCQQRSSYPFTFGQGTKLEIK

SEQ ID NO: 340 - protein, 107 aa, synthetic construct
  REGION 1..107: note = 3B6_VL_Humanized
DVVMTQSPLSLPVTLGQPASISCKASQNVRTAVAWFQQRPGQSPRALIYLASTRHTGVPDRFSGSGSGTDFTLKISRVEAEDLGVYFCLQHWNYPRTFGQGTKLEIK